(19) United States
(12) Patent Application Publication
(10) Pub. No.: US 2009/0291923 A1
(43) Pub. Date: Nov. 26, 2009

(54) TRIOXANE DIMERS HAVING HIGH ANTICANCER AND LONG-LASTING ANTIMALARIAL ACTIVITIES

(75) Inventors: Gary H. Posner, Baltimore, MD (US); Ikhyeon Paik, New Haven, CT (US); Kristina Borstnik, State College, PA (US); Wonsuk Chang, Baltimore, MD (US); Sandra Sinishtaj, Baltimore, MD (US); William Malo, Katonah, NY (US); John Gaetano D'Angelo, Lindenhurst, NY (US); Lauren Elaine Woodard, Abingdon, MD (US); Alvin Solomon Kalinda, Baltimore, MD (US); Aimee R. Usera, Odenton, MD (US); Lindsey Catherine Hess, Baltimore, MD (US); Andrew Scott Rosenthal, Baltimore, MD (US); Seongho Oh, Cockeysville, MD (US); Astrid C. Baege, McLean, VA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Johns Hopkins Universoty, Baltimore, MD (US); The Government of the United States of America as Represented by the Secretary of the DHHS NIH, Offi, Rockville, MD (US)

(21) Appl. No.: 12/096,015

(22) PCT Filed: Nov. 17, 2006
(86) PCT No.: PCT/US06/44845

§ 371 Date: Jan. 6, 2009

Related U.S. Application Data
(60) Provisional application No. 60/748,548, filed on Dec. 8, 2005.
(60) Provisional application No. 60/765,125, filed on Feb. 3, 2006.
(60) Provisional application No. 60/794,811, filed on Apr. 25, 2006.

(51) Int. Cl. A61K 31/665; C07D 493/18; C07F 9/6561; A61K 31/357; C07D 495/10; C07D 317/12; C07D 317/22; C07D 493/10; A61P 35/00; C07D 263/12

(52) U.S. Cl. 514/100; 549/348; 549/220; 514/450; 549/23; 549/430; 549/453; 549/334; 548/239

(57) ABSTRACT

The invention provides novel trioxane dimers having formulae III, IV or V: methods for their preparation, pharmaceutical compositions containing these compounds, and methods for treating cancer and/or malaria using these compounds and compositions.

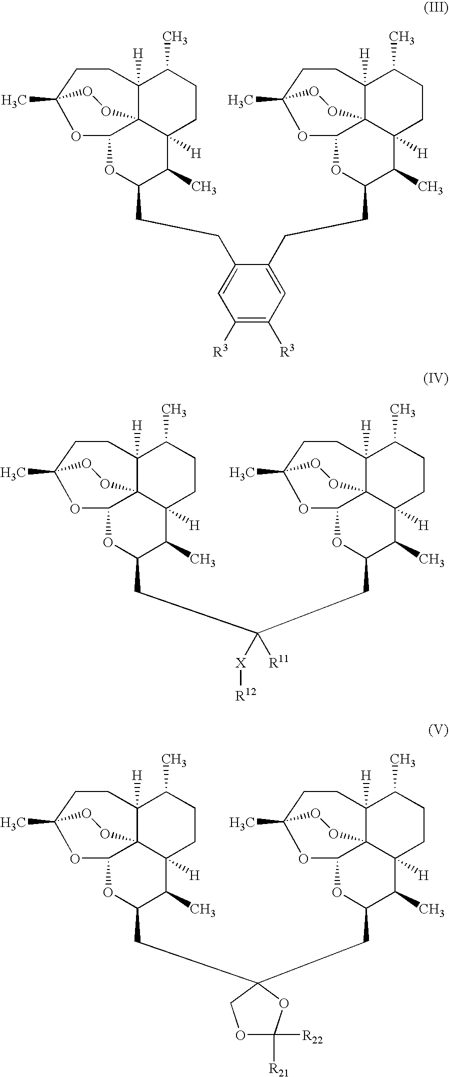

### CROSS-REFERENCE TO RELATED APPLICATIONS

This application claims the benefit of U.S. Provisional Patent Application Ser. Nos. 60/748,548, filed Dec. 8, 2005; 60/765,125 filed Feb. 3, 2006; 60/794,811 filed Apr. 25, 2006, all titled: “Trioxane Dimers Having Surprisingly High Anticancer Activities and Surprisingly Long-Lasting Antimalarial Activities.” Priority of these filing dates are hereby claimed, and the disclosure of each of these applications are hereby incorporated by reference as if fully set forth.

### STATEMENT AS TO RIGHTS TO INVENTIONS MADE UNDER FEDERALLY SPONSORED RESEARCH AND DEVELOPMENT

This research was in part supported by the Intramural Research Program of the National Institute of Health (NIH, Grant AI34885 and RR00052), National Cancer Institute, and Center for Cancer Research.

### FIELD OF THE INVENTION

The invention provides novel trioxane dimers, methods for their preparation, pharmaceutical compositions containing these compounds, and methods for treating cancer and/or malaria using these compounds and compositions.

### BACKGROUND OF THE INVENTION

Cervical cancer is the second most common malignancy related cause of death in women worldwide. Although population wide screening in most Western countries has led to a remarkable reduction in incidence and mortality, with approximately 470,000 new cases diagnosed each year, cervical cancer remains a global public health problem and a significant economic burden to health care systems (Parkin, D. M. et al., Int. J. Cancer 94:153-156 (2001)). Nearly all cervical cancers are etiologically attributable to persistent high risk human papillomavirus (HPV) infection (Zur Hausen, H. Acta Biochem. Biophys. 1288:F55-F78 (1996)). Potent antiviral agents to treat these infections have not been developed. Prophylactic HPV vaccines are in clinical trials, but will, when approved, be costly and prevent infection with only a very limited number of HPV types in women who have not been infected previously (Schiller, J. T. et al., Nature Rev. 2:343-347 (2004)). Surgical intervention is currently the standard of care for pre-invasive cervical lesions, and over-treatment out of concern for progression or underlying high grade lesions is found frequently. The successful therapy of cervical cancer, utilizing available approaches, such as radiation therapy, surgery and chemotherapy, still represents a challenge (Waggoner, S. E. Lancet 361:2217-2225 (2003)).

Cancer chemotherapy is limited by the development of drug resistance in tumors and adverse side effects in patients. It has been reported that the natural trioxane artemisinin, the active principle of the Chinese medicinal herb Artemisia annua, and its monomeric derivatives such as artesunate (ART) and dihydroartemisinin (DHA) distinguish themselves as a new generation of very effective blood schizontocidal antimalarials with fewer toxic side effects than any other antimalarial (Hien, T. T. et al., Lancet 341:603-608 (1993)). Recently, these artemisinin derivatives were also shown to be active against human cancer cell lines, including drug-resistant cancer cells (Efferth, T. et al., Int. J. Oncol. 18, 767-773 (2001); Singh, N. P. et al., Life Sci. 70:49-56 (2001); Singh, N. P. et al., Anticancer Res. 24:2277-2280 (2004); Disbrow, G. L. et al., Cancer Res. 65:10854-10861 (2005)).

Moreover, 1,2,4-trioxanes in the artemisinin family of endoperoxides are fast-acting antimalarials which unfortunately, do not have long-lasting antimalarial activity. This characteristic is recognized worldwide as indicated by the international use of artimisinin-combination-therapy (ACT). Such ACT effectively combines a fast-acting antimalarial trioxane with a long-lasting alkaloidal antimalarial to avoid malaria parasite recrudescence which usually occurs when a trioxane alone is used for malaria chemotherapy.

Therefore, there is a continuing need for new therapies for treating cancer and malaria which are safer, more effective, longer lasting and less costly than the currently used cancer and ACT regiments of chemotherapy.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE INVENTION

The invention provides novel trioxane dimers, methods for their preparation, pharmaceutical compositions comprising these compounds, and methods for treating cancer, as well as other diseases and conditions caused by abnormal hyperproliferation of cells, and/or malaria, as well as other infectious diseases and/or parasitic diseases, using these compounds and compositions.

In one embodiment, the invention relates to novel trioxane dimers having formula I:

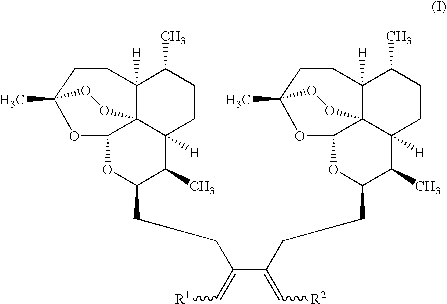

or a pharmaceutically acceptable, salt or solvate thereof, wherein:
R¹ and R² are each independently H, or substituted or unsubstituted alkyl, or R¹ and R² together form a substituted or unsubstituted ar34, or a substituted or unsubstituted cycloalkyl group.

In another aspect, the invention provides trioxane dimers having formula I, wherein R¹ and R² are hydrogen.

In another aspect, the invention provides trioxane dimers having formula I, wherein R¹ and R² form a substituted or unsubstituted phenyl group.

In another aspect, the invention provides trioxane dimers having formula I,
wherein R¹ and R² form a substituted phenyl group, wherein the phenyl group is substituted with 1 or 2 R³ groups;
each R³ group is independently selected from —C(═O)OR⁴, —CH₂OR⁴, —C(═O)NR⁵R⁶, and —OP(═O)(OR⁴)₂, or
together each R³ group forms a cyclic ring with —OP(═O)O(R⁴)O—;
R⁴ hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted arylalkyl, or substituted or unsubstituted heteroaryl; and
R⁵ and R⁶ are each independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted arylalkyl, or substituted or unsubstituted heteroaryl.

In another aspect, the invention provides trioxane dimers having formula I, wherein the R¹ and R² form a substituted or unsubstituted phenyl group which is disubstituted with the same R³ group; and each R³ group is —C(═O)OH, —C(═O)OCH₃, —CH₂OH, —OP(═O)O(C₂H₅)₂, or together each R³ group forms a cyclic ring with —OP(═O)O(Ph)O—.

In another aspect, the invention provides trioxane dimers of formula I, having formula II:

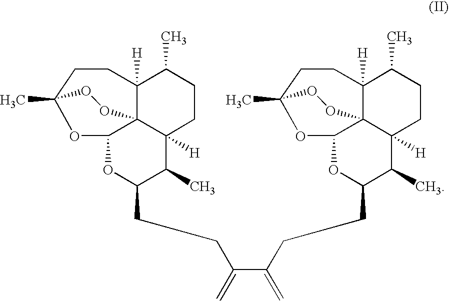

In another aspect, the invention provides trioxane dimers of formula I, having formula III:

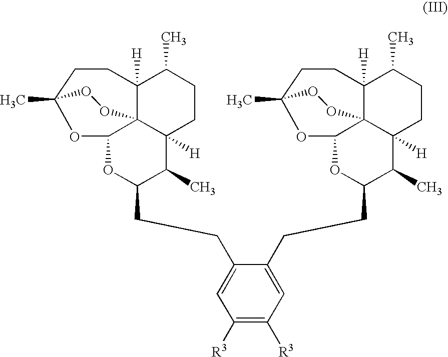

wherein each R³ group is —C(═O)OH, —C(═O)OCH₃, —CH₂OH, —OP(═O)O(C₂H₅)₂, or together each R³ group forms a cyclic ring with —OP(═O)O(Ph)O—.

In another aspect, the invention provides trioxane dimers of formula I, having formula:

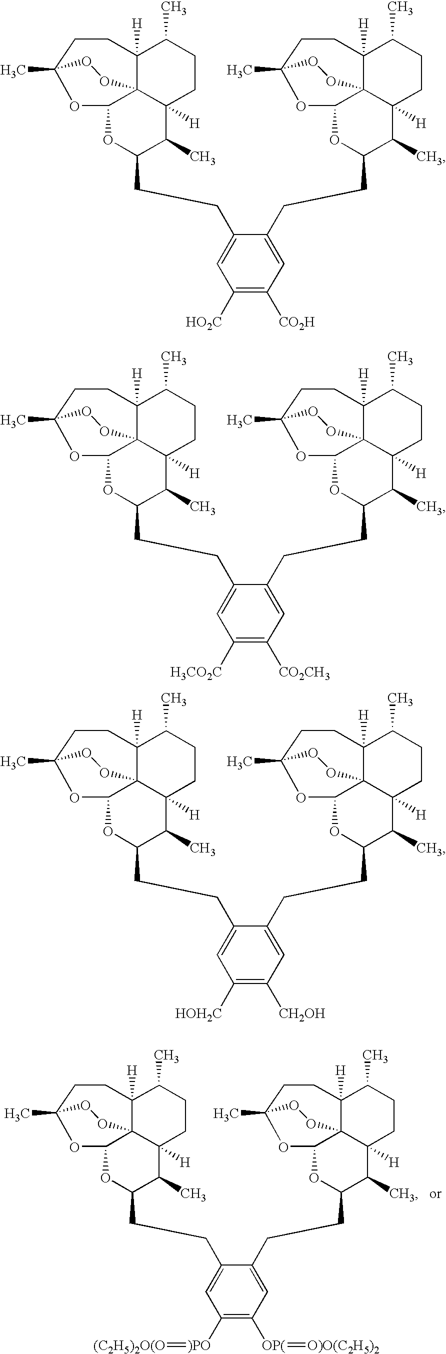

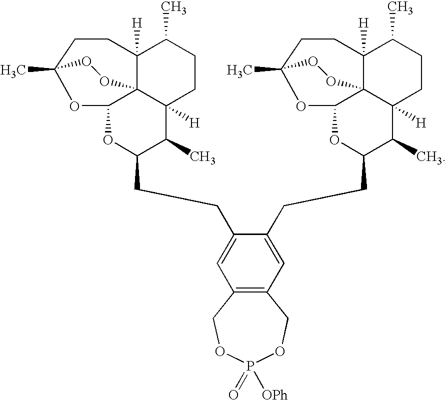

In another embodiment, the invention relates to novel trioxane dimers having formula IV:

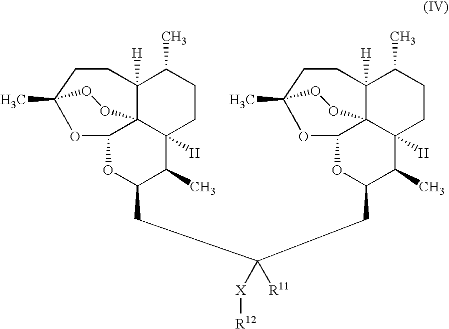

or a pharmaceutically acceptable, salt or solvate thereof, wherein:
X is (CH₂)ₙ—Y or is a direct bond;
Y is O, (CH₂)ₘO, C(═O), C(═O)(CH₂)ₘO, C(═O)O, OC(═O)O, OC(═O)NR¹³, NR¹³C(═O)NR¹³, C(═S), C(═O)S, C(═S)O, OC(═S)O, C(═O)(NR¹³)ₙ, C(═O)O(NR¹³)ₙ, C(═O)O(NR¹³)ₙC(═O), C(═O)(NR¹³)ₙC(═O), C(═O)(NR¹³)ₙ(CH₂)ₘC(═O), C(═O)(NR¹³)ₙ(CH₂)ₘC(═O)(NR¹³), (NR¹³)ₙ, (NR¹³)ₙO, C(═O)(NR¹³)ₙO, C(═O)(NR¹³)ₙS(O)ₚ, C(═O)O(NR¹³)ₙS(O)ₚ, OC(═O)(NR¹³)ₙS(O)ₚ; OP(═O)(OR¹³)₂, OP(═S)(OR¹³)₂, OP(═O)(NR¹³)₂, OP(═S)(NR¹³)₂, OS(O)ₚ, S(O)ₙNR¹³, (NR¹³) CH₂C(═O)(NR¹³)ₙ, or Y is a direct bond;
m is an integer from 0, 1, 2 or 3;
n is an integer from 1 or 2;
p is an integer from 0, 1 or 2;
R¹¹ is H, OH, or R¹¹ together with R¹² forms a substituted or unsubstituted cyclic ring;
R¹² is optionally H, substituted or unsubstituted alkyl, substituted or unsubstituted heteroalkyl, substituted or unsubstituted aryl, substituted or unsubstituted heteroaryl, substituted or unsubstituted arylalkyl, substituted or unsubstituted heteroarylalkyl, or R¹² together with R¹¹ forms a substituted or unsubstituted cyclic ring; or
R¹¹ and R¹² form a substituted or unsubstituted double bond or a substituted or unsubstituted oxime group; and
R¹³ is H, substituted or unsubstituted alkyl, substituted or unsubstituted heteroalkyl, substituted or unsubstituted aryl, substituted or unsubstituted heteroaryl, substituted or unsubstituted arylalkyl, substituted or unsubstituted heteroarylalkyl, substituted or unsubstituted phosphonate, substituted or unsubstituted sulfonate.

In another aspect, the invention provides trioxane dimer compounds having formula IV, wherein X is CH₂—Y; and R¹¹ is H.

In another aspect, the invention provides trioxane dimer compounds having formula IV, wherein Y is O; and R¹² is H, CH₂CH═CH₂, CH₂(C₆H₄)CH₃, CH₂(C₅H₄N), CH₂(C₆H₄)CH(CH₃)₂, CH₂(C₆H₄)CF₃,

In another aspect, the invention provides trioxane dimer compounds having formula IV, wherein Y is O; and R¹² is P(═S)(OCH₂CH₃)₂, P(═O)(OC₆H₅)₂, P(═O)(NCH₂CH₃)₂ or P(═S)(OCH₃)₂.

In another aspect, the invention provides trioxane dimer compounds having formula IV, wherein Y is OC(═O)O or OC(═S)O; and R¹² is C₆H₅.

In another aspect, the invention provides trioxane dimer compounds having formula IV, wherein Y is O(C═O); and R¹² is (CH₂)₂C(═O)OH, C₆H₄C(═O)CH₃, N(CH₂CH₃)₂, N(C₅H₁₀),

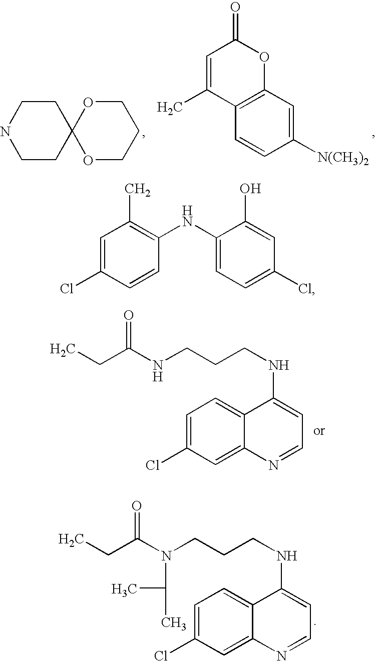

In another aspect, the invention provides trioxane dimer compounds having formula IV, wherein Y is NR¹³; and R¹³ is —C₅H₁₀—.

In another aspect, the invention provides trioxane dimer compounds having formula IV, wherein Y is OSO₂; and R¹² is

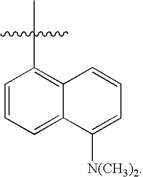

In another aspect, the invention provides trioxane dimer compounds having formula IV, wherein X is Y; and R¹¹ is H.

In another aspect, the invention provides trioxane dimer compounds having formula IV, wherein Y is C(═O)O; and R¹² is H, (C₆H₅), CH₂(C₆H₅), CH₃,

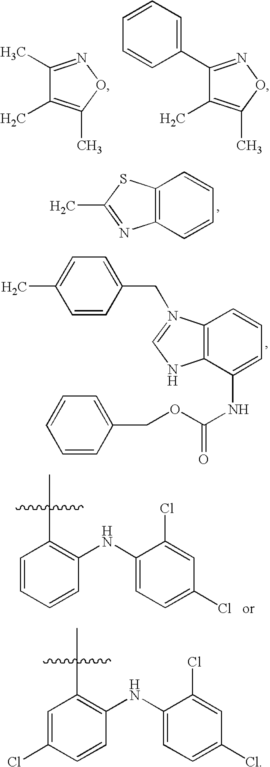

In some embodiments, the invention provides trioxane dimer compounds having formula IV, wherein Y is (C═O)O(NR¹³)ₙS(O)ₚ, wherein R¹² is (C₆H₅) or as described above for formula IV. In other embodiments, the invention provides trioxane dimer compounds having formula IV, wherein Y is C(═O)O(NR¹³)ₙC(═O), wherein R¹² is (C₆H₅) or as described above for formula IV.

In another aspect, the invention provides trioxane dimer compounds having formula IV, wherein Y is C(═O)(NR¹³)ₙ; R¹³ is H or substituted or unsubstituted alkyl; and R¹³ is (C₆H₅), CH₂(C₆H₅), CH(CO₂H)CH₂(C₆H₅), (C₆H₄N), CH₂(C₆H₄N), CH(CO₂CH₃)(C₆H₅), CH₂(C₆H₄)CO₂CH₃, CH₂(C₆H₄)C(═O)OH, CH₂(C₆H₄)NO₂, CH₂(C₆H₄)CF₃, CH₂(C₆H₄)F, (CH₂)₂SO₃H, C(CH₃)₃, C(CH₃)₂(C₆H₅), C(CH₃)₂CH₂C(CH₃)₃, CH₂C(CH₃)₂NHC(═O)(C₆H₅), CH₂CH₃, CH₂(C₆H₄)(CH₂)₇CH₃, CH₃, CH(CH₃)₂, CH₂C(CH₃)₂NH₂, (CH₂)₉CH₃, CH₂C(CH₃)₃, (CH₂)₃NHCH(CH₃)₂, CH₂C(═O)OH, C(CH₃)₂C(CH₃)₃, (C₆H₄)SO₂(C₆H₄)NH₂, CH₂CH(CH₃)₂,

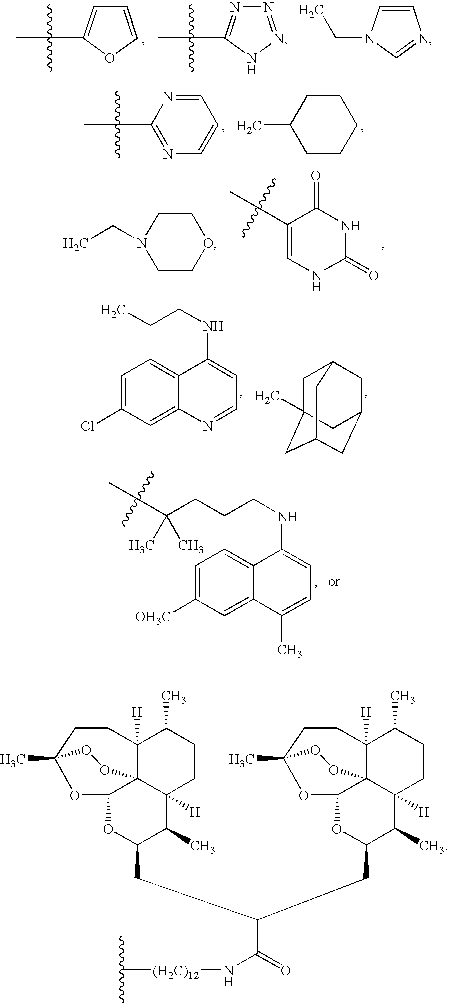

In some embodiments, R¹² is
not

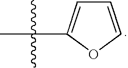

In other embodiments, the invention provides trioxane dimer compounds having formula IV wherein Y is C(═O)(NR¹³)ₙC(═O) and wherein n is 1, R¹³ is H, and R¹² is

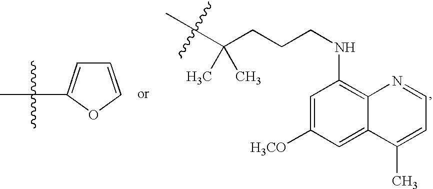

or wherein n is 2, R¹³ is H, and R¹² is

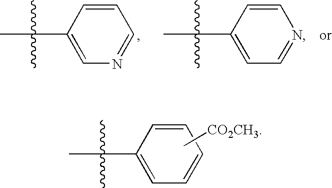

In another aspect, the invention provides trioxane dimer compounds having formula IV, wherein Y is C(—O)(NR¹³)ₙO; R¹³ is H or substituted or unsubstituted alkyl; and R¹² is (C₆H₅).

In another aspect, the invention provides trioxane dimer compounds having formula IV, wherein Y is C(═O)(NR¹³)ₙS(O)ₚ; R¹³ is H; and R¹² is (C₆H₅) or (C₆H₄)NH₂.

In another aspect, the invention provides trioxane dimer compounds having formula IV, wherein Y is C(—O)(NR¹³)ₙ; and R¹² and R¹³ together form a substituted or unsubstituted cyclic ring.

In another aspect, the invention provides trioxane dimer compounds having formula IV, wherein Y is C(═O)(NR¹³)ₙ; and R¹² and R¹³ together form a substituted or unsubstituted cyclic ring wherein the cyclic ring is

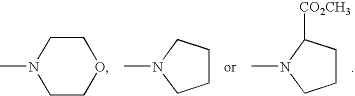

In another aspect, the invention provides trioxane dimer compounds having formula IV, wherein Y is (NR¹³)ₙC(═O)(NR¹³)ₙ or (NR¹³)ₙCH₂C(═O)(NR¹³)ₙ; each R¹³ is H or substituted or unsubstituted alkyl; and R¹² is

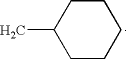

In another aspect, the invention provides trioxane dimer compounds having formula IV, wherein X is CH₂—Y; and R¹¹ is OH.

In another aspect, the invention provides trioxane dimer compounds having formula IV, wherein Y is O; and R¹² is H, (CH₂)(C₆H₄)CH₃, CH₂CH═CH₂, CH₂CH═C(CH₃)₂, CH₂(C₆H₄N), CH₂C(═O)NH(C₆H₄)OH or

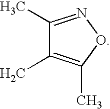

In another aspect, the invention provides trioxane dimer compounds having formula IV, wherein Y is C(═O); and R¹² is (C₆H₄)C(═O)OCH₃.

In another aspect, the invention provides trioxane dimer compounds having formula IV, wherein Y is C(═O)(NR³)ₙ; and R¹² is (CH₃).

In another aspect, the invention provides trioxane dimer compounds having formula IV, wherein Y is C(═O)O or OC(═O); and R¹² is (C₆H₅), (C₆H₄)C(═O)N(CH₂CH₃)₂, (C₆H₄)F or (C₆H₄N).

In another aspect, the invention provides trioxane dimer compounds having formula IV, wherein Y is OC(═O)(NR¹³)ₙS(O)ₚ; R¹³ is H or substituted or unsubstituted alkyl; and R¹² is (C₆H₅).

In another aspect, the invention provides trioxane dimer compounds having formula IV, wherein X is a direct bond; and R¹¹ and R¹² together form a substituted or unsubstituted cyclic ring.

In another aspect, the invention provides trioxane dimer compounds of formula IV, having formula V:

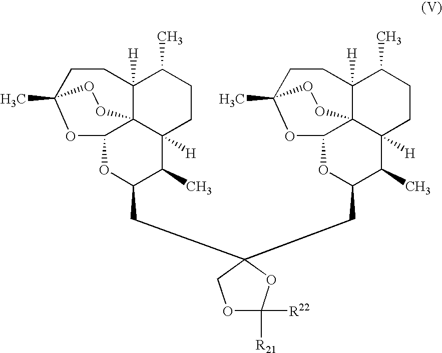

wherein:
R²¹ and R²² are each independently H, OH, OR¹³, substituted or unsubstituted alkyl, substituted or unsubstituted heteroalkyl, substituted or unsubstituted aryl, substituted or unsubstituted heteroaryl, substituted or unsubstituted arylalkyl, substituted or unsubstituted heteroarylalkyl, or R²¹ and R²² together form ═O, or R²¹ and R²² together form a substituted or unsubstituted cycloalkyl or substituted or unsubstituted heterocycloalkyl ring.

In another aspect, the invention provides trioxane dimer compounds having formula V, wherein R²¹ and R²² together form a substituted or unsubstituted cyclobutyl ring, substituted or unsubstituted cyclohexyl ring, substituted or unsubstituted piperidinyl ring, substituted or unsubstituted tetrahydropyranyl ring; substituted or unsubstituted sulfonylcyclohexyl ring, substituted or unsubstituted 1,3-dioxanyl ring, or a substituted or unsubstituted 1,3-dioxepanyl ring. A representation of a sulfonylcyclohexyl ring is

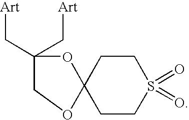

In another aspect, the invention provides trioxane dimer compounds having formula V, wherein R²¹ and R²² together form a substituted or unsubstituted cyclohexyl ring.

In another aspect, the invention provides trioxane dimer compounds having formula V, wherein the cyclohexyl ring is substituted with 1 to 2 groups each independently selected from F, OH, ═O, C(═O)OCH₃, C(═O)OCH₂CH₃, C(═O)CH₃, C(═O)OCH₂(C₆HS), C(═O)NHCH₂CH₃, C(CH₃)₃, CH₂(C₆H₁₁), SO₂N(CH₃)₂, SO₂(C₆H₄)CH₃, P(═O)(CH₃)₂, P(═O)(OCH₃)₂, P(═O)(OCH₂CH₃)₂, and P(═O)(OC₆H₅)₂.

In another aspect, the invention provides trioxane dimer compounds of formula V, wherein R²¹ and R²² form a substituted or unsubstituted piperidinyl ring.

In another aspect, the invention provides trioxane dimer compounds of formula V, wherein the piperidinyl ring is substituted with 1 to 2 groups each independently selected from F, OH, ═O, C(═O)OCH₃, C(═O)OCH₂CH₃, C(═O)OCH₂(C₆H₅), C(═O)CH₃, C(═O)CH₃(C₆H₅), C(═O)NHCH₂CH₃, C(CH₃)₃, CH₂(C₆H₁), SO₂N(CH₃)₂, SO₂(C₆H₄)CH₃, P(═O)(CH₃)₂, P(—O)(OCH₃)₂, P(═O)(OCH₂CH₃)₂ and P(═O)(OC₆H₅)₂. In some embodiments, the substituent is not C(═O)CH₃(C₆Hs).

In another aspect, the invention provides trioxane dimer compounds of formula IV, wherein X is a direct bond; and R¹¹ and R¹² together form a substituted or unsubstituted double bond.

In another aspect, the invention provides trioxane dimer compounds of formula IV, wherein the double bond is substituted with a substituted or unsubstituted phenyl group.

In another aspect, the invention provides trioxane dimer compounds of formula IV, wherein the double bond is a substituted or unsubstituted oxime group.

In another aspect, the invention provides trioxane dimer compounds of formula IV, wherein the oxime group is substituted with CH₃ or NHC(═O)(C₆H₅).

In another aspect, the invention relates to pharmaceutical compositions, comprising a pharmaceutically acceptable excipient and a compound of the invention.

In another aspect, the invention relates to methods for treating cancer, or other disease or unwanted condition caused by abnormal hyperproliferation of cells, in a subject in need of such treatment, comprising administering to the subject a therapeutically effective amount of a compound of the invention. Briefly, a disease or unwanted condition caused by abnormal hyperproliferation of cells refers to cancer and other conditions where cells have lost the ability to be controlled by normal cell signals that regulate proliferation. Non-limiting examples include carcinomas, sarcomas, leukemias/lymphomas, and psoriasis. Thus the cells undergoing abnormal hyperproliferation include those of epithelial tissue, such as those of a gland or the lining of an organ; connective tissue, such as that of bone or muscle; or immune or hematopoietic cells.

In another aspect, the invention relates to methods for treating cancer in a subject in need of such treatment, comprising administering to the subject a therapeutically effective amount of a compound of the invention, wherein the cancer is cervical cancer, breast cancer, prostate cancer, leukemia, or lymphoma. In an alternative embodiment, the cancer is one characterized by a solid tumor or disseminated cancer dispersed throughout the vascular system.

In another aspect, the invention relates to methods for treating malaria, or other infectious disease, in a subject in need of such treatment, comprising administering to the subject a therapeutically effective amount of a compound of the invention. The invention thus includes treatment of a disease or condition caused by infection by a parasite or pathogen. Representative pathogens include bacteria, fungi, viruses, and protozoa. Non-limiting examples include treatment of malaria and other protozoic diseases.

### DETAILED DESCRIPTION OF THE INVENTION

### Definitions

Abbreviations used herein have their conventional meaning within the chemical and biological arts.

Where substituent groups, e.g., linking groups, are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

The term “alkyl,” by itself or as part of another substituent, means, unless otherwise stated, a straight (i.e. unbranched) or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. Also included in the definition of alkyl and cycloalkyl are bicyclic ring structures such as norbornyl and adamantyl and the like, and fused ring systems such as dihydro- and tetrahydronaphthalene, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl”.

The term “alkylene” by itself or as part of another substituent means a divalent radical derived from an alkyl, as exemplified, but not limited, by —CH₂CH₂CH₂CH₂—, —CH₂CH═CHCH₂—, —CH₂C≡CCH₂—, —CH₂CH₂CH(CH₂CH₂CH₃)CH₂—. Typically, an alkyl (or alkylene) group will have from 1 to 24 carbon atoms, with those groups having 10 or fewer carbon atoms being some embodiments of the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (such as from 1 to 3 rings) which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent radicals of aryl and heteroaryl, respectively.

For brevity, the term “aryl” when used in combination with other terms (e.g., aryloxo, arylthioxo, arylalkyl) includes both aryl and heteroaryl rings as defined above. Thus, the terms “arylalkyl” and “heteroarylalkyl” are meant to include those radicals in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

The term “oxo” as used herein means an oxygen that is double bonded to a carbon atom.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent radical derivatives) are meant to include both substituted and unsubstituted forms of the indicated radical. Optional substituents for each type of radical are provided below.

Substituents for alkyl, heteroalkyl, cycloalkyl, heterocycloalkyl monovalent and divalent derivative radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, —N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—CNR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen radical. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl radicals above, exemplary substituents for aryl and heteroaryl groups (as well as their divalent derivatives) are varied and are selected from, for example: halogen, —OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C′″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

An “aminoalkyl” as used herein refers to an amino group covalently bound to an alkylene linker. The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

A “substituent group,” as used herein, means a group selected from the following moieties:

(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and

(B) alkyl, heteroalkyl, cycloalkyl, heterocycloalkyl, aryl, and heteroaryl, substituted with at least one substituent selected from:

(i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and

(ii) alkyl, heteroalkyl, cycloalkyl, heterocycloalkyl, aryl, and heteroaryl, substituted with at least one substituent selected from:

(a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and

(b) alkyl, heteroalkyl, cycloalkyl, heterocycloalkyl, aryl, or heteroaryl, substituted with at least one substituent selected from oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein means a group selected from all of the substituents described above for a “substituent group,” wherein each substituted or unsubstituted alkyl is a substituted or unsubstituted C₁-C₉ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

The compounds of the present invention may exist as salts. The present invention includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C or ¹⁴C-enriched carbon are within the scope of this invention.

The compounds of the present invention may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

The term “pharmaceutically acceptable salts” is meant to include salts of active compounds which are prepared with relatively nontoxic acids or bases, depending on the particular substituent moieties found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The terms “a,” “an,” or “a(n)”, when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

Description of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “treating” or “treatment” in reference to a particular disease includes prevention of the disease.

The symbol [symbol] denotes the point of attachment of a moiety to the remainder of the molecule.

### Trioxane Dimers

We provide herein the design, synthesis and biological evaluation of a new series of hydrolytically stable, C-10 non-acetal, 3 and 4-carbon atom linked trioxane dimers. Monomeric 1; 2,4-trioxanes such as natural artemisinin 1, have shown both antimalarial activity as well as anticancer activity. For example, monomeric 1,2,4-trioxanes such as dihydroartemisinin (DHA) has shown anticancer activity against human HeLa cervical cancer cells in vitro (IC₅₀=5-10 μM.

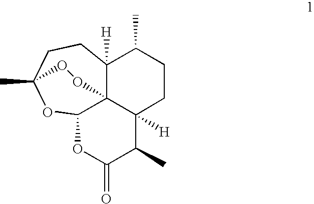

Also provided herein, are 1,2,4-trioxane dimers which have high in vitro antimalarial, antiproliferative and antitumor activity as well in vivo anticancer activity. These new stable artemisinin derived trioxane dimers have long lasting antimalarial and considerably higher selective anticancer activity in vitro that monomeric artemisinin and its derivatives. Our inventive compounds also express a rapid, dose dependent and more than 500-fold higher cytotoxic activity towards human cervical cancer cells than ART and DHA, whereas normal cervical cells are virtually unaffected. These findings suggest that the our novel inventive trioxane dimers are clinically useful as potent chemotherapeutic agents for the treatment of cervical cancer, its precursors and potentially other mucosal and epidermal tumors. The stability and hydrophobicity of our inventive dimers make them excellent candidates for not only systemic but also topical (e.g. intravaginal) application, a route of administration which would permit also high dosaging without the risk of systemic side effects.

The synthesis of these novel 4-carbon atom linked trioxane dimer analogs of the present invention is outlined below in Scheme I.

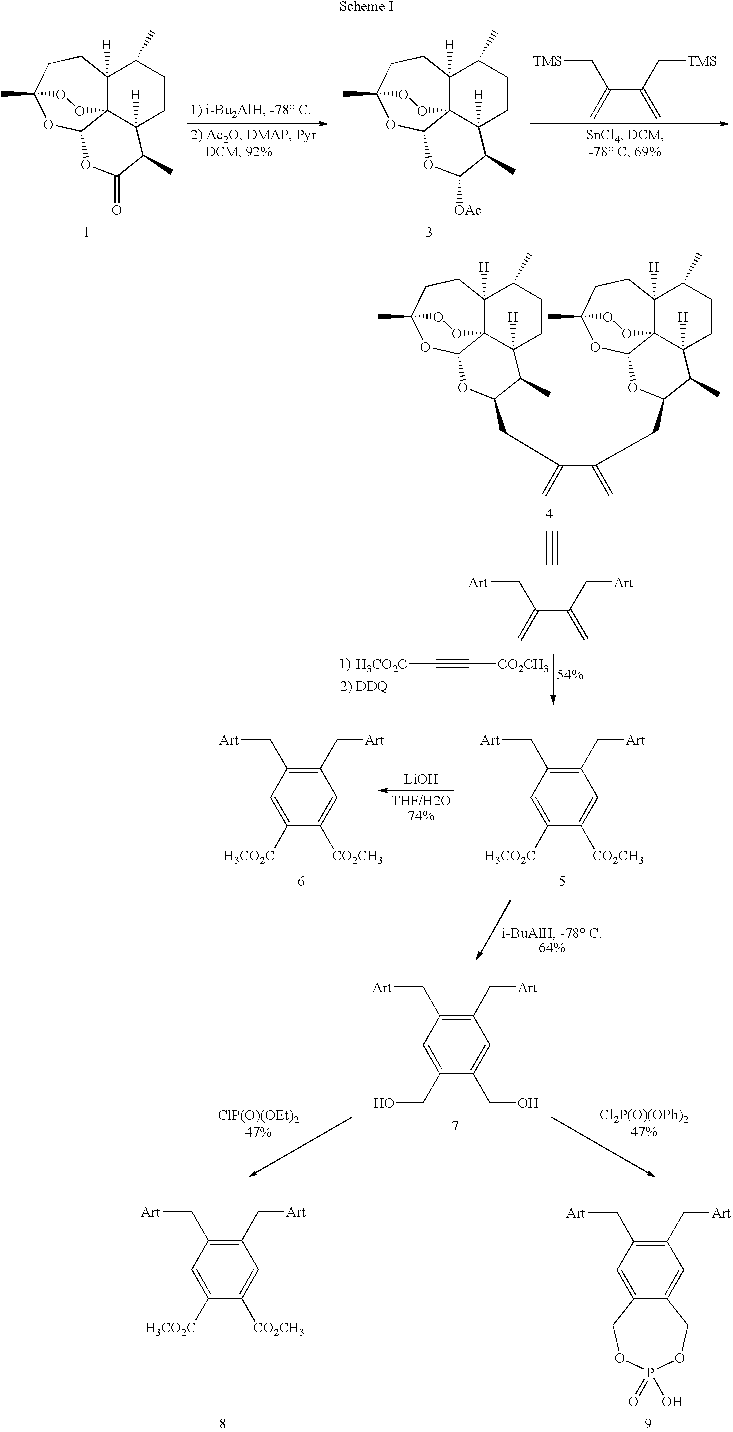

In Scheme 1, the conjugated trioxane diene dimer 4 proceeds in overall 63% yield from artemisinin 1 via formation of two new carbon-carbon bonds, using the linker 2,3-bis(trimethylsilylmethyl)-1,3-butadiene. Conjugated diene dimer 4 undergoes a Diels-Alder cycloaddition with dimethyl acetylenedicarboxylate followed by dichlorodicyanoquinone (DDQ) oxidation to provide phthalate dimer 5. Bis-ester 5 is hydrolyzed into phthalic acid 6, which may be separately reduced to bis-benzyl alcohol 7. Bis-benzyl alcohol 7 may be phosphorylated to bis-phosphate 8 or into cyclic phosphate 9. None of the reactions destroys the crucial peroxide pharmacophore in these trioxane dimers. All of the aromatic 4-carbon linked dimers are thermally stable even upon accelerated aging in the absence of solvent at 60° C. for 24 hours wherein less than 5% decomposition was observed by ¹H NMR spectroscopy. Of these new trioxane dimers, phthalic acid 6 is the most soluble in aqueous pH 7.4 buffer solution (≅14 mg/mL) at 25° C. As C-10 non-acetal analogs of artemisinin 1, we have found that all of these trioxane dimers are hydrolytically stable for at least 4 days in pH 7.4 buffer at 25° C.

In Scheme II, the X-ray crystallography of crystalline phthalate diester 5 shows that the two peroxide units in this trioxane dimer are oriented in opposite directions. Whether this structural feature affects the mechanism of action of this dimer remains to be determined.

(Supporting Information Available: X-ray crystallographic data for compound 5 and a crystallographic file in CIF format. ¹H and ¹³C NMR spectra of compounds 5 and 7. This material is available free of charge via the Internet at http://pubs.acs.org).

Using our standard assay (Posner, G. H. et al., Tetrahedron 53:37-50 (1997)), we determined the antimalarial potencies of these dimers in vitro against chloroquine-sensitive Plasmodium falciparum (NF 54) parasites (Table 1). Except for water-soluble phthalic acid dimer 6, all of the other dimers in Table 1 are considerably more potent antimalarials than natural artemisinin (1, IC₅₀=6.6±0.76 nM). Bis-benzyl alcohol dimer 7 stands out as the most potent, being approximately 10-times more antimalarially active than artemisinin (1).

TABLE 1
Antimalarial Activities in vitro⁸

| trioxane dimer | IC₅₀ (nM) |
|---|---|
| 4 | 2.9 |
| 5 | 1.6 |
| 6 | 360 |
| 7 | 0.77 |
| 8 | 3.0 |
| 9 | 3.7 |
| Artemisinin | 6.6 |

⁸The standard deviation for each set of quadruplicates was an average of 7.8% (≦18%) of the mean.
R² values for the fitted curves were ≧0.967.
Artemisinin activity is the mean ± standard deviation of the concurrent control (n = 6).

As measured in mice according to a published protocol involving single administration at dose of 3, 10, or 30 mg/kg, either subcutaneously (SC) or orally (PO) (Fidock, D. A. et al., Nat. Rev. Drug Discov. 3:509-520 (2004)), bis-ester dimer 5 has SC ED₅₀=0.71 mg/kg and diol dimer 7 has SC ED₅₀=0.06 mg/kg and PO ED₅₀=2.6 mg/kg. Under these test conditions, the clinically used monomeric trioxane sodium artesunate has SC ED₅₀=2.2 mg/kg and PO ED₅₀=4.0 mg/kg. Thus, these two dimers 5 and 7 are approximately 3-37 times, more efficacious than the antimalarial drug sodium artesunate administered SC, and diol dimer 7 is approximately 1.5 times more efficacious than sodium artesunate administered PO. Neither over toxicity nor behavioral modification was observed in the mice due to drug administration.

Preliminary growth inhibitory activities at nanomolar to micromolar concentrations; measured in vitro as described previously using a diverse panel of 60 human cancer cell lines in the National Cancer Institute's (NCI's) Development and Therapeutic Program (Boyd, M. R. et al., Drug Dev. Rev. 34:91-109 (1995)) showed phthalate dimer 5 to be extremely selective and highly potent at inhibiting the growth of only non-small cell lung carcinoma HOP-92 cells, melanoma SK-MEL-5 cells, and breast cancer BT-549 cells. Employing a tetrazolium salt (XTT) based calorimetric proliferation assay (Roche Diagnostics, Mannheim, Germany) and using a modified version of a recently reported protocol for in vitro evaluation of the growth inhibitory activity of DHA toward the human cervical cancer cell line HeLa (IC₅₀=5-10 micromolar) (Disbrow, G. L. et al., Cancer Res. 65:10854-10861 (2005)), we have found unexpectedly but importantly that trioxane phthalate dimer 5 (IC₅₀=500 nM) is approximately 10-20 times more potent than trioxane monomer DHA and that trioxane diol dimer 7 (IC₅₀=46.5 nM) is approximately 110-220 times more potent than DHA, without being toxic to primary normal cervical cells. Cell growth was inhibited in a dose-dependent manner.

The synthesis of the 3-carbon atom linked trioxane dimer analogs of the present invention is outlined in Scheme III.

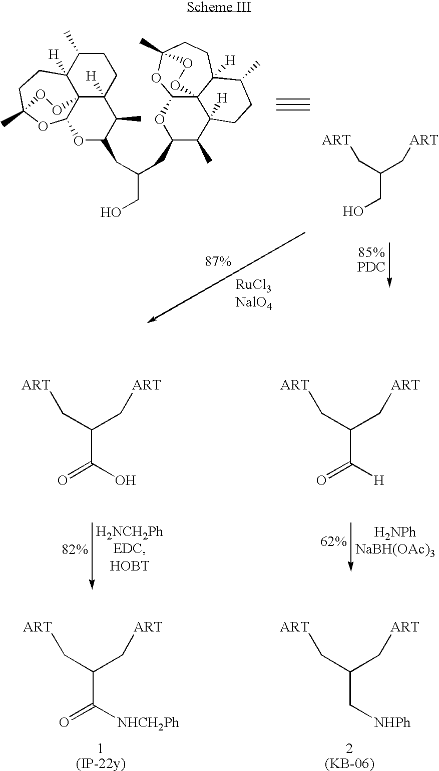

Starting from the natural trioxane artemisinin, the hydrolytically stable C-10-carba trioxane dimer primary alcohol on the top of Scheme 1 is prepared in very good overall yield. Complete oxidation of this primary alcohol provided the corresponding carboxylic acid and partial oxidation using pyridinium dichromate (PDC) forms the corresponding aldehyde. Conversion of the carboxylic acid into the target trioxane dimer amide 1 was achieved in high yield using benzylamine in the presence of 1-[3-(dimethylamino)propyl]-3-ethylcarbodiimide (EDC) and 1-hydroxybenzotriazole (HOBT). Conversion of the aldehyde into the target trioxane dimer amine 2 was achieved in good yield via reductive amination using aniline in the presence of sodium triacetoxyborohydride. The important peroxide functionality in these trioxane dimers survived both amide and amine formation.

The inventive artemisinin-derived trioxane dimers described and evaluated herein show great promise as novel candidates for the treatment of cervical pre-malignant and malignant lesions and potentially other mucosal and epidermal tumors. The topical and/or systemic administration of these exceptionally potent artemisinin dimers may be a very effective and economical addition or even alternative to traditional treatment options for these neoplasias.

Using a standard protocol in Plasmodium berghei infected mice, trioxane dimers IP-IV-22y and KB-06 were administered subcutaneously only once at a dose of 3, 10, or mg/kg body weight. Both dimers at the single dose of 30 mg/kg dose rapidly killed more than 98% of the malaria parasites. The currently used antimalarial drug sodium artesunate at 30 mg/kg was similarly efficacious. Sodium artesunate at 30 mg/kg prolonged the life of the mice from 7 days (no drug) to only 14 days. Unexpectedly but of great medical importance, both dimers at 30 mg/kg prolonged the life of the mice to at least 30 days at which time the mice were considered cured (i.e. no parasites detected in blood smears)! Neither overt toxicity nor behavioral modification was observed in the mice due to drug administration.

Primary human ectocervical keratinocytes were derived from fresh cervical tissue obtained from the Cooperative Human Tissue Network (CHTN) within 24 hours after removal from patients undergoing hysterectomies for benign non-cervical uterine diseases. Standard overnight dispase treatment and subsequent trypsinization procedures were used to isolate ectocervical epithelial cells, which were cultured in serum-free keratinocyte medium (KSFM) supplemented with bovine pituitary extract and epidermal growth factor according to the manufacturer's protocol (Invitrogen, Carlsbad, Calif.). The cervical cancer cell lines HeLa and C33A were obtained from the American Type Culture Collection (ATCC) and maintained in Dulbecco's Modified Eagle Medium (DMEM) (Invitrogen).

Cell viability was determined using 2.5×10³ cells were plated in triplicates in 96 well tissue culture microplates in the appropriate culture medium and incubated for 24 hours in a humidified atmosphere at 37° C., 5% CO₂. The medium was subsequently replaced by 100_{—}1 medium containing either the solvent control ethanol or various concentrations of dimers dissolved in ethanol. After a 96 hour treatment period, 50_{—}1 of the XTT labeling mixture, prepared according to the manufacturer's protocol (Roche Diagnostics GmbH, Penzberg, Germany), was added to each well, followed by an additional 16 hour incubation period. Cell viability (absorbance) was measured using an ELISA reader at 450 nm with a reference wavelength at 650 nm. Results were calculated as the percentage of cultures exposed to solvent control only. The assay was repeated twice with similar results.

To evaluate the cytotoxic effects of our newly synthesized trioxane dimers, the cervical cancer cell lines HeLa and C33A were exposed to various concentrations of these compounds, and cell viability was quantified after a three day treatment period using a colorimetric XTT based assay as described in Materials and Methods. Dimer 1 and 2 were nearly equally potent, inducing rapid dose-dependent cell killing in both cervical cancer cell lines. As shown in Scheme IV, at a drug concentration of 100 nM an approximate 90% loss of viability was determined after treatment with either dimer.

Based on the data in FIG. 1, IC₅₀ values for dimer 1 and 2 of approximately 7.5 nM and 8.6 nM for C33A cells and approximately 8.4 nM and 9 nM for HeLa cells were determined. In contrast, normal ectocervical cells HCX were, even at a dimer concentration of 100 nM, virtually unaffected. Cell death in treated cancer cells was also easily observed with a phase contrast microscope whereas normal cells showed no significant morphological changes (data not shown).

The compounds of the invention gave unexpectedly high and long-lasting oral in vivo antimalarial activity in mouse model studies, higher and longer than those of prior art: For example, complete cure (survival with no detectable parasitemia at 30 days post-infection) of malaria-infected mice with Just 30 mg/1 kg dose over three days was achieved with each of the following new inventive dimers. Other compounds of the invention are provided in the Examples section.

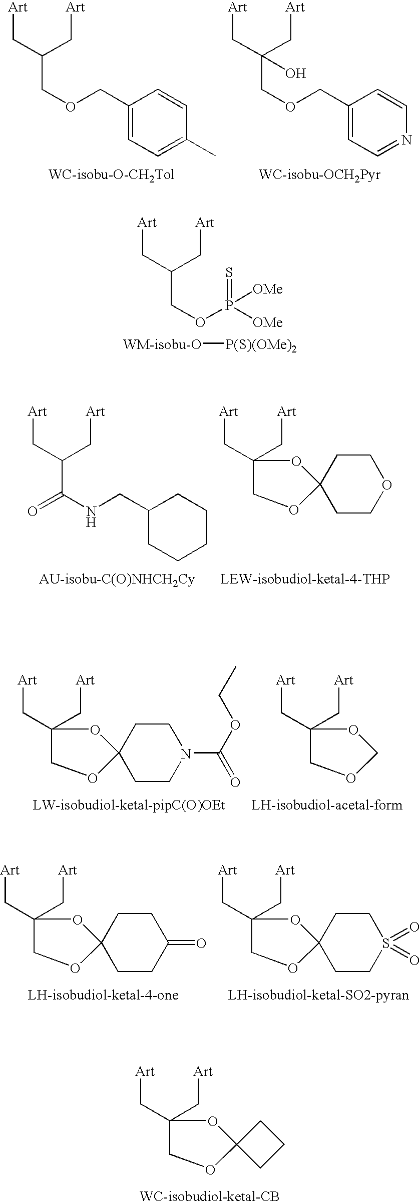

The term “protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butytdimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(0)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking/protecting groups include, but are not limited to the following moieties:

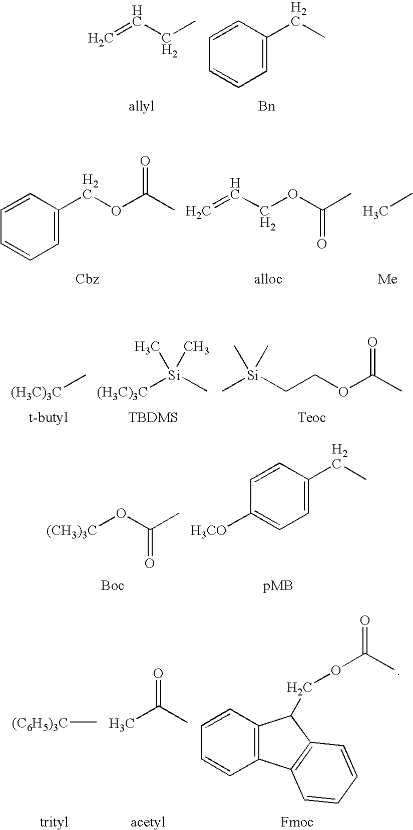

### Pharmaceutical Compositions and Administration

In another aspect, the present invention provides a pharmaceutical composition including a pyrimidinyl-thiophene kinase modulator in admixture with a pharmaceutically acceptable excipient. One of skill in the art will recognize that the pharmaceutical compositions include the pharmaceutically acceptable salts of the pyrimidinyl-thiophene kinase modulators described above.

In therapeutic and/or diagnostic applications, the compounds of the invention can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

The compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, and the preference and experience of the attending physician.

Pharmaceutically acceptable salts are generally well known to those of ordinary skill in the art, and may include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, parnoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). Pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

Depending on the specific conditions being treated, such agents may be formulated into liquid or solid dosage forms and administered systemically or locally. The agents may be delivered, for example, in a timed- or sustained-low release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intrahasal, or intraocular injections or other modes of delivery.

For injection, the agents of the invention may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a subject (e.g. patient) to be treated.

For nasal or inhalation delivery, the agents of the invention may also be formulated by methods known to those of skill in the art, and may include, for example, but not limited to, examples of solubilizing, diluting, or dispersing substances such as, saline, preservatives, such as benzyl alcohol, absorption promoters, and fluorocarbons.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Depending upon the particular condition, or disease state, to be treated or prevented, additional therapeutic agents, which are normally administered to treat or prevent that condition, may be administered together with the inhibitors of this invention. For example, chemotherapeutic agents or other anti-proliferative agents may be combined with the inhibitors of this invention to treat proliferative diseases and cancer. Examples of known chemotherapeutic agents include, but are not limited to, adriamycin, dexamethasone, vincristine, cyclophosphamide, fluorouracil, topotecan, taxol, interferons, and platinum derivatives.

Other examples of agents the inhibitors of this invention may also be combined with include, without limitation, anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents for treating diabetes such as insulin, insulin analogues, alpha glucosidase inhibitors, biguanides, and insulin sensitizers; and agents for treating immunodeficiency disorders such as gamma globulin.

These additional agents may be administered separately, as part of a multiple dosage regimen, from the inhibitor-containing composition. Alternatively, these agents may be part of a single dosage form, mixed together with the inhibitor in a single composition.

The present invention is not to be limited in scope by the exemplified embodiments, which are intended as illustrations of single aspects of the invention. Indeed, various modifications of the invention in addition to those described herein will become apparent to those having skill in the art from the foregoing description. Such modifications are intended to fall within the scope of the invention. Moreover, any one or more features of any embodiment of the invention may be combined with any one or more other features of any other embodiment of the invention, without departing from the scope of the invention. For example, the pyrimidinyl-thiophene kinase modulators described in the Pyrimidinyl-thiophene Kinase Modulators section are equally applicable to the methods of treatment and methods of inhibiting kinases described herein. References cited throughout this application are examples of the level of skill in the art and are hereby incorporated by reference herein in their entirety for all purposes, whether previously specifically incorporated or not.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes

### EXAMPLES

The following examples are offered to illustrate, but not to limit the claimed invention.

All air- and moisture-sensitive reactions were performed under argon in oven-dried or flame-dried glassware. Tetrahydrofuran (THF) and diethyl ether (ether) were distilled from sodium-benzophenone ketyl and dichloromethane was distilled from calcium hydride under nitrogen. Dimethyl sulfoxide and hexamethylphosphoric triamide were distilled from calcium hydride over 4 Å molecular sieves under reduced pressure. Solvents and solutions for air- and moisture-sensitive reactions were transferred via syringe or cannula. All experiments were monitored by thin layer chromatography (tic) performed on EM Science precoated silica gel 60 F-254 glass supported plates with 0.25 mm thickness. Flash chromatography was performed with EMD silica gel (40-63 μm). Yields are not optimized. Purity of final products was confirmed by two diverse high performance liquid chromatography (HPLC) trace analyses. HPLC was performed with a Rainin HPLX gradient system equipped with two 25 mL/min preparative pump heads using Phenomenex 10 mm×250 mm (semi-preparative) column packed with 60 Å silica gel. Melting points were measured using a MeI-Temp metal-block apparatus and are uncorrected. Infrared (IR) spectra were recorded on a Bruker Vector 33 FT-IR spectrophotometer or a Perkin Elmer 1600 FT-IR spectrometer. Nuclear Magnetic Resonance (NMR) spectra were recorded on a Bruker Avance 400 MHz FT-NMR spectrometers (400 MHz for ¹H, 100 MHz for ¹³C) or Bruker Avance 300 MHz FT-NMR spectrometer (300 MHz for ¹H, 282 MHz for ¹⁹F, 75 MHz for ¹³C). Residual signals [¹H, 7.26 ppm, ¹³C: 77.0 ppm for CDCl₃; ¹H, 2.50 ppm, ¹³C: 39.52 ppm for (CD₃)₂SO; ¹H: 3.31 ppm, ¹³C: 49.0 ppm for CD₃OD; ¹H, 2.05 ppm, ¹³C: 29.84 ppm for (CD₃)₂CO] were used as internal standards. The following abbreviations are used in the experimental section for the description of ¹H NMR spectra: singlet (s), doublet (d), triplet (t), quartet (q), multiplet (m), broad singlet (bs), doublet of doublets (dd), doublet of triplets (dt), and doublet of quartets (dq). Low and high resolution mass spectra (LRMS and HRMS) were obtained on a VG70S magnetic sector mass spectrometer at Johns Hopkins University with fast atom bombardment (FAB) ionization or a 3-Tesla Finnigan FTMS-2000 Fourier Transform mass spectrometer at Ohio State University with electrospray ionization (ESI). Combustion analyses were conducted by Atlantic Microlab (Norcross, Ga.). Reagents were purchased from Aldrich Chemical Company unless otherwise noted. Various methods of purifying the products of the present invention known and understood by those skilled in the art and the purification methods presented are solely listed by way of example and are not intended to limit the invention.

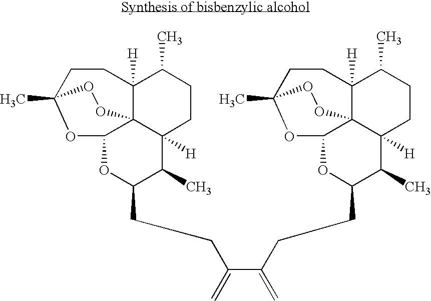

A solution of dihydroartemisinin acetate (DHA acetate, 3) (835 mg, 2.56 mmol) and the bissilane butadiene linker (346 mg, 1.53 mmol, 0.6 equiv) in dichloromethane (45 mL) was cooled to −78° C. Tin(IV) tetrachloride (1M solution in CH₂Cl₂, 1.53 mmol, 0.6 equiv diluted in 4 mL dichloromethane and pre-cooled to −78° C.) was added quickly to the reaction mixture. The reaction was stirred nt. −78° C. for a further 45 minutes at which time TLC analysis confirmed complete consumption of starting material. Distilled water (3 mL) was then added and the reaction was allowed to warm to room temperature. Distilled water (10 mL) and dichloromethane (30 mL) were added and organics were extracted with dichloromethane (3×20 mL), dried (MgSO₄) and concentrated in vacuo to give a yellow solid. Gradient column chromatography on silica eluting with 5-10% ethyl acetate/hexanes isolated trioxane butadiene dimer 4 as a white solid (541 mg, 0.88 mmol, 69%). Mp=68-72° C.; ¹H NMR (400 MHz, CDCl₃) δ 5.35 (s, 2H), 5.19 (s, 2H), 5.16 (s, 2H), 4.48 (ddd, J=9.6, 6.0, 3.2 Hz, 2H), 2.73-2.63 (m, 2H), 2.5.5-2.26 (m, 6H), 2.06-1.96 (m, 2H), 1.95-1:86 (m, 2H), 1.86-1.77 (m, 2H), 1.69-1.58 (m, 4H), 1.52-1.20 (m, 14H including singlet at 1.39), 0.96 (d, J=6.0 Hz, 6H), 0.91 (d, J=7.2 Hz, 61-1), 0.98-0.86 (m, 2H); ¹³C NMR (100 MHz, CDCl₃) δ 145.07, 113.58, 103.00, 89.16, 81.05, 72.74, 52.23, 44.36, 37.45, 36.64, 34.44, 33.79, 30.46, 25.98, 24.85, 24.73, 20.15, 13.00; IR (film, cm⁻¹) 2991, 2950, 2909, 2854, 1366, 1085, 1044, 872, 729; HRMS (ES) m/z calc'd for C₃₆H₅₄O₈Na (M+Na) 637.3711, found 637.3683; [α]_{D}^{23.6} 65.9 (CHCl₃, c=0.28).

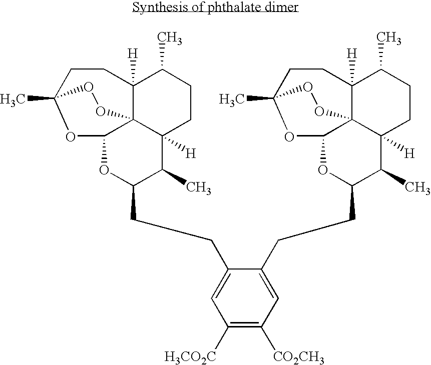

To a solution of trioxane butadiene dimer 4 (235 mg, 0.382 mmol) in anhydrous benzene (12.0 mL) was added dimethylacetylene dicarboxylate (0.094 mL, 0.764 mmol, 2.0 equiv). Then, the reaction mixture was heated to 80-85° C. for 18 hours, at which time TLC analysis showed full consumption of starting material. The reaction mixture was cooled to room temperature and treated with dichloro dicyanoquinone (DDQ) (43.4 mg, 0.191 mmol, 0.5 equiv) and heated to 80-85° C. for 20 mins. Brine (15 mL) and ethyl ether (30 mL) were added and organics were extracted with ethyl ether (3×30 mL), dried (MgSO₄) and concentrated in vacuo to give a yellow sticky solid. Gradient column chromatography on silica eluting with 20-30% ethyl acetate/hexanes isolated bis-trioxane 5 as a white solid (157 mg, 0.208 mmol, 54%). Mp=108-111° C.; ¹H NMR (CDCl₃, 400 MHz) δ 7.62 (s, 2H), 5.40 (s, 2H), 4.56-4.49 (m, 2H), 3.85 (s, 6H), 3.10 (dd, J=14.8, 9.6 Hz, 2H), 2.82-2.70 (m, 4H), 2.35-2.25 (m, 2H), 2.05-1.82 (m, 6H), 1.74-1.62 (m, 5H), 1.45-1.21 (m, 13H, including singlet at 1.29), 1.00-0.81 (m, 14H, including two doublets at 1.00 (J=7.6 Hz) and 0.97 (J=6.4 Hz); ¹³C NMR (CDCl₃, 100 MHz) δ 168.39, 142.99, 129.95, 129.48, 103.10, 89.17, 81.01, 75.47, 52.41, 52.16, 44.30, 37.45, 36.57, 34.46, 32.83, 30.81, 25.78, 24.75, 20.16, 13.16; HRMS (EI, m/z) for C₄₂H₅₈O₁₂Na calc'd 777.3820, found 777.3824; IR (film, cm⁻¹) 2932, 2866, 1726, 1443, 1389, 1284, 1238, 1120, 1054, 988; [α]_{D}^{24.5}=112.8 (CHCl₃, c=0.51).

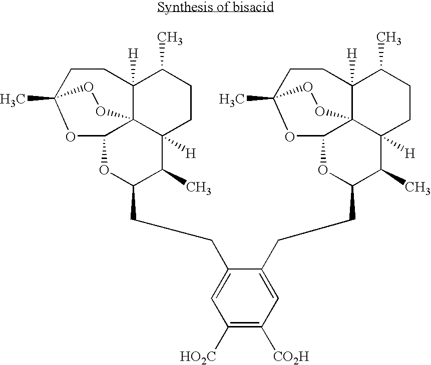

Bis-trioxane phthalate bis-ester 5 (53 mg, 7.0 μmol) was dissolved in tetrahydrofuran (0.7 mL) and distilled water (0.3 mL) and treated with lithium hydroxide monohydrate (5.9 mg, 0.14 mmol, 20 equiv). The reaction mixture was stirred for 18 hours, at which time TLC analysis showed full consumption of starting material. 0.3% Hydrochloric acid (10 mL) and ethyl ether (10 mL) were added. Then, aqueous layer was acidified with 10% hydrochloric acid (upon addition white precipitates were shown) and extracted with ethyl acetate (3×20 mL), dried (MgSO₄) and concentrated in vacuo. Flash column chromatography on silica eluting with 40% ethyl acetate/hexanes (2% acetic acid) to isolated bis-trioxane phthalic acid 6 as a white solid (3.8 mg, 5.2 μmol, 74%). mp=139-140° C.; ¹H NMR (CDCl₃, 400 MHz) δ 7.81 (s, 2H), 5.45 (s, 2H), 4.51-4.45 (m, 2H), 3.20-3.02 (m, 2H), 2.82-2.70 (m, 4H), 2.35-2.25 (m, 2H), 2.05-1.82 (m, 6H), 1.74-1.62 (m, 5H), 1.45-1.21 (m, 14H, including singlet at 1.43), 1.00-0.81 (m, 14H, including two doublets at 1.00 (J=6.8 Hz) and 0.95 (J=6.0 Hz); ¹³C NMR (CDCl₃, 100 MHz) δ 171.66, 143.13, 131.85, 129.48, 103.42, 89.08, 80.91, 75.81, 52.22, 44.40, 37.32, 36.51, 34.47, 30.71, 29.68, 25.68, 24.70, 20.17, 13.30; HRNMS (EI, m/z) for C₄₀H₅₄O₁₂Na calc'd 7749.3507, found 749.3527; IR (film, cm⁻¹ 3200 (br), 2952, 291% 2879, 1712, 1462, 1383, 1277, 1146, 1047, 994; [α]_{D}^{24.4}=79.2 (CHCl₃, c=0.11).

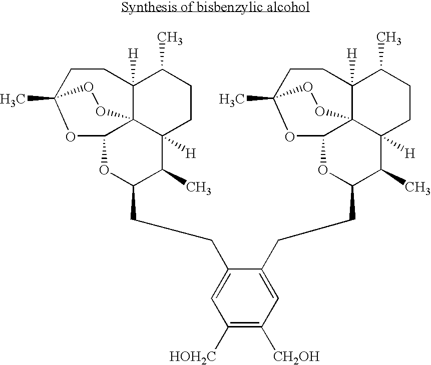

A solution of bis-trioxane phthalate bis-ester 5 (22.3 mg, 0.030 mmol) in dichloromethane (2.0 mL) was cooled to −78° C. Diisobutyl aluminum hydride (1.5 M solution in CH₂Cl₂, 0.2 mL, 0.30 mmol, 10 equiv) was added slowly dropwise to the reaction mixture. The reaction was stirred at −78° C. for further 30 minutes at which time TLC analysis confirmed complete consumption of starting material. Distilled water (0.5 mL) was then added and the reaction was allowed to warm to room temperature. Distilled water (5 mL) and dichloromethane (15 mL) were added and organics were extracted with dichloromethane (2×20 mL), dried (MgSO₄) and concentrated in vacuo to give yellow oil. Gradient column chromatography on silica eluting with 70-80% ethyl acetate/hexanes isolated bis-trioxane bis-benzyl alcohol 7 as a white solid (13.2 mg, 0.019 mmol, 64%). Mp=128-130° C.; ¹H NMR (CDCl₃, 400 MHz) δ 7.24 (s, 2H), 5.43 (s, 2H), 4.64 (s, br, 4H), 4.49-4.41 (m, 2H), 3.22 (s, br, 2H), 2.95 (dd, J=15.2, 10.0 Hz, 2H), 2.80-2.68 (m, 4H), 2.35-2.25 (m, 2H), 2.05-1.61 (m, 11H), 1.45-1.21 (m, 13H, including singlet at 1.32), 1.00-0.81 (m, 14H, including apparent triplet at 0.98 (J=8.4 Hz); ¹³C NMR (100 MHz, CDCl₃) δ 138.68, 137.17, 131.12, 103.24, 89.05, 81.01, 76.12, 63.99, 52.28, 44.47, 37.43, 36.58, 34.48, 31.96, 30.74, 25.89, 24.80, 24.71, 20.20, 13.32; HRMS (EI, m/z) for C₄₀H₅₈O₁₀Na calc'd 721.3922, found 721.3917; IR (film, cm⁻¹) 3401, 2949, 2875, 1454, 1377, 1205, 1188, 1124, 1090, 1042, 1013, 941, 877, 825, 735; [α]_{D}^{23.5}=63.7 (CHCl₃, C=0.10).

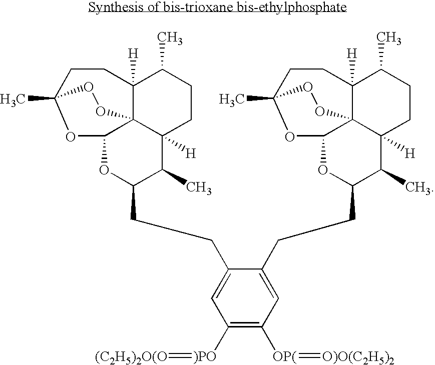

To a solution of bis-trioxane bis-benzyl alcohol 7 (20.0 mg, 0.029 mmol) in anhydrous dichloromethane (2.0 mL) was added pyridine (0.012 mL, 0.143 mmol, 5.0 equiv) and diethyl chlorophosphate (0.020 mL, 0.143 mmol, 5.0 equiv) at 0° C. The reaction mixture was stirred for 30 minutes at 0° C. and slowly warmed to room temperature for 1 hours, at which time TLC analysis showed full consumption of starting material. Brine (5 mL) and dichloromethane (10 mL) were added and organics were extracted with dichloromethane (2×20 mL), dried (MgSO₄) and concentrated in vacuo to give a sticky solid. Flash column chromatography on silica eluting with 2% methanol/dichloromethane isolated bis-trioxane bis-phosphate 8 as a white foam (14.4 mg, 0.015 mmol, 52%). ¹H NMR (CDCl₃, 400 MHz) δ 7.28 (s, 2H), 5.43 (s, 2H), 5.12 (d, J=7.6 Hz, 4H), 4.41-4.36 (m, 2H), 4.12-4.00 (m, 8H), 3.06 (dd, J=15.2, 9.6 Hz, 2H), 2.81-2.70 (m, 4H), 2.38-2.25 (m, 2H); 2.01-1.85 (m, 6H), 1.73-1.60 (m, 4H), 1.53-1.19 (m, 26H, including singlet at 1.30), 1.00-0.81 (m, 14H, including doublet at 0.99 (J=7.6 Hz) and doublet at 0.97 (J=6.4 Hz); ¹³C NMR (CDCl₃, 100 MHz) δ 140.16, 132.09, 132.02, 131.16, 103.18, 88.84, 80.99, 66.52, 66.47, 63.80, 63.75, 52.32, 44.56, 37.38, 36.58, 34.51, 32.35, 30.72, 25.96, 24.71, 20.20, 16.14, 16.07, 13.45; HRMS (EI. m/z) for C₄₈H₇₆O₁₆P₂Na requires 993.4501, found 993.45031; IR (film, cm⁻¹) 2965, 2932, 2879, 2860, 1390, 1271, 1027, 974; [α]_{D}^{24.6}=96.1 (CHCl₃, c=0.04).

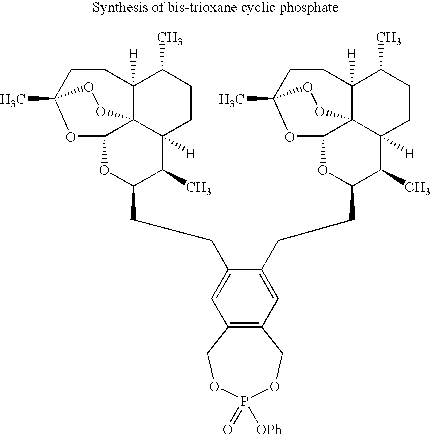

To a solution of bis-trioxane bis-benzyl alcohol 7 (20.0 mg, 0.029 mmol) in anhydrous dichloromethane (2.0 mL) was added pyridine (0.010 mL, 0.129 mmol, 4.5 equiv) and phenyl dichlorophosphate (0.013 mL, 0.086 mmol, 3.0 equiv) at room temperature. The reaction mixture was stirred for 18 hours, at which time TLC analysis showed full consumption of starting material. Brine (5 mL) and dichloromethane (10 mL) were added and organics were extracted with dichloromethane (2×20 mL), dried (MgSO₄) and concentrated in vacuo to give an sticky solid. Flash column chromatography on silica eluting with 40% ethyl acetate/hexanes isolated bis-trioxane cyclicphosphate 9 as a white solid (1.2 mg, 0.013 mmol, 47%). Mp=130-133° C.; ¹H NMR (CDCl₃, 400 MHz) δ 7.38-7.32 (m, 3H), 7.30-7.25 (m, 2H), 7.22-7.15 (m, 1H), 5.42 (s, 1H), 5.41 (s, 1H), 5.38-5.27 (m, 2H), 5.22-5.11 (m, 2H), 4.60-4.55 (m, 1H), 4.51-4.47 (m, 1H), 2.98-2.91 (m, 2H), 2.79-2.69 (m, 4H), 2.36-2.26 (m, 2H), 2.05-1.81 (m, 6H), 1.71-1.60 (m, 6H), 1.45-1.20 (m, 13H, including singlets at 1.31 and 1.30), 1.00-0.81 (m, 14H); ¹³C NMR (CDCl₃, 100 MHz) δ 140.09, 140.05, 132.61, 132.47, 130.54, 130.12, 129.82, 125.09, 119.80, 119.75, 103.08, 102.99, 89.45; 89.22, 81.01, 75.26, 74.81, 69.16 (d, J=4.5 Hz), 69.09 (d, J=4.5 Hz), 52.19, 52.10, 44.29, 44.19, 37.45, 36.57, 34.41, 32.23, 32.06, 30.84, 25.91, 24.82, 24.76, 24.73, 20.15, 20.13, 13.10, 12.97; HRMS (EI, m/z) for C₄₆H₆₁O₁₀PNa calc'd 859.3793, found 859.3793; IR (film, cm⁻¹) 2929, 2881, 1498, 1444, 1389, 1295, 1193, 1125, 1085, 1017, 1010, 935, 738; [α]_{D}^{24.1}=28.6 (CHCl₃, c=0.45).

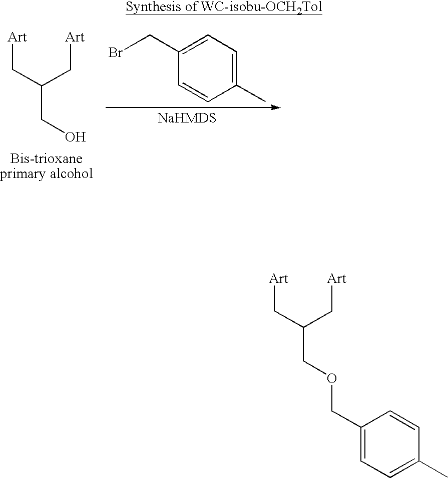

To a solution of bis-trioxane primary alcohol (97 mg, 0.16 mmol) in THF (1 mL) at 0° C. was added sodium bis(trimethylsilyl)amide (NaHMDS) in THF (1.0 M, 0.48 mL, 0.48 mmol) and 4-methylbenzyl bromide (59 mg, 0.32 mmol) in THF (0.5 mL). The reaction was warmed to rt and stirred for 16 h. It was quenched with saturated aq NH₄Cl (1 mL) and layers were separated. The aqueous layer was extracted with EtOAc (3×2 mL). The combined organic solution was dried (MgSO₄), and concentrated. The purification of the crude product by column chromatography (elution with EtOAc:hexanes=1:5) gave WC-isobu-OCH₂Tol (87 mg, 77%) as a white solid: [α]_{D}²⁴=+77 (c 0.30, CHCl₃); mp 51-52° C.; IR (thin film) 2938, 1451, 1376, 1101, 1008 cm⁻¹; ¹H NMR (400 MHz, CDCl₃) δ 7.23 (d, J=8.0 Hz, 2H), 7.12 (d, J=8.0 Hz, 2H), 5.33 (s, 1H), 5.31 (s, 1H), 4.49 (d, J=11.6 Hz, 1H), 4.42 (d, J=11.6 Hz, 1H), 4.30 (m, 1H), 4.20 (m, 1H), 3.66 (dd, J=9.2, 5.2 Hz, 1H), 3.60 (dd, J=9.2, 4.8 Hz, 1H), 2.72 (dq, J=15.2, 7.6 Hz, 1H), 2.65 (dq, J=14.4, 7.2 Hz, 1H), 2.38-2.26 (m, 5H including s at 2.33), 2.10 (m, 1H), 2.03 (m, 1H,), 1.99 (m, 1H), 1.90-1.20 (m, 26H including s at 1.41 and 1.38), 0.98-0.82 (m, 14H including d at 0.85 with J=7.2 Hz and 0.84 with J=7.6 Hz); ¹³C NMR (100 MHz, CDCl₃) δ 136.8, 136.0, 128.8, 127.8, 103.2, 103.0, 88.9, 88.5, 81.2, 81.2, 74.9, 72.8, 72.7, 71.8, 52.5, 52.4, 44.7, 44.5, 37.3, 37.3, 36.6, 36.6, 35.6, 34.5, 34.5, 30.6, 30.5, 30.0, 29.6, 26.2, 26.1, 24.8, 24.7, 24.6, 21.1, 20.2, 20.2, 13.4, 13.1; HRMS (FAB) calculated for C₄₂H₆₃O₉ [(M+H)⁺] 711.4472, found 711.4445.

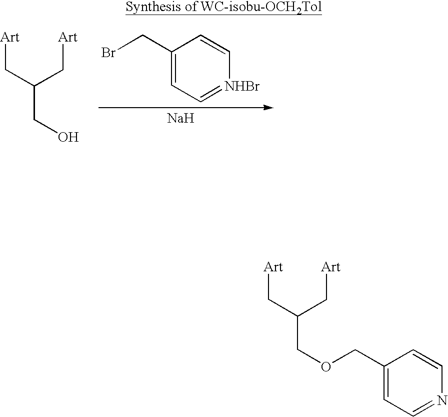

To a mixture of 4-(bromomethyl)pyridine hydrobromide (100 mg, 0.39 mmol) in THF (1 mL) at 0° C. was added sodium hydride (NaH, 60% dispersion in mineral oil, 39 mg, 0.98 mmol) and it was stirred for 30 min. To a solution of bis-trioxane primary alcohol (120 mg, 0.20 mmol) in THF (1 mL) at 0° C. was added sodium bis(trimethylsilyl)amide in THF (1.0 M, 0.20 mL, 0020 mmol) and the resulting solution was cannulated dropwise to the 4-(bromomethyl)pyridine mixture at 0° C. The reaction was stirred for 48 h at 0° C. and quenched by addition of water (0.5 mL) and saturated aq. NaHCO₃ (1 mL). Layers were separated and the aqueous layer was extracted with EtOAc (3×2 mL). The combined organic solution was dried (MgSO₄) and concentrated. The crude oil was subjected to flash column chromatography (elution with EtOAc:hexanes 1:1) on silica gel, that had been treated with Et₃N (1 mL per 100 mL gel) in hexanes before use. WC-isobu-OCH₂Pyr (79 mg, 57%) was obtained as a colorless oil: [α]_{D}²⁴=+64 (c 0.88, CHCl₃); IR (neat) 2937, 1455, 1375, 1103, 1007 cm⁻¹; ¹H NMR (400 MHz, CDCl₃) δ 8.55 (d, J=6.0 Hz, 2H), 7.33 (d, J=6.0 Hz, 2H), 5.31 (s, 1H), 5.28 (s, 1H), 4.59 (d, J=14.0 Hz, 1H), 4.52 (d, J=14.0 Hz, 1H), 4.36 (ddd, J=8.8, 6.0, 4.0 Hz, 1H), 4.23 (dd, J=8.8, 6.0 Hz, 1H), 3.75 (dd, J=9.2, 5.2 Hz, 1H), 3.68 (dd, J=9.2, 4.8 Hz, 1H), 2.69 (dq, J=14.4, 7.2 Hz, 1H), 2.61 (dq, J=13.6, 6.8 Hz, 1H), 2.37-2.25 (m, 2H), 2.12 (m, 1H), 2.04-1.18 (m, 28H including s at 1.38 and 1.35), 0.98-0.83 (m, 14H including d at 0.86 with J=7.6 Hz and 0.85 with J=7.6 Hz); ¹³C NMR (100 MHz, CDCl₃) δ 149.3, 148.5, 124.1, 103.2, 102.8, 89.7, 89.1, 81.3, 81.2, 74.5, 72.6, 70.7, 70.32, 52.2, 52.0, 44.4, 44.0, 37.6, 37.6, 36.6, 36.5, 35.8, 34.4, 34.4, 30.8, 30.7, 30.7, 30.5, 26.1, 24.9, 24.8, 24.8, 24.7, 20.2, 20.1, 13.1, 12.7; HRMS (FAB) calculated for C₄₀H₆₀NO₉ [(M+H)⁺] 698.4268, found 698.4299.

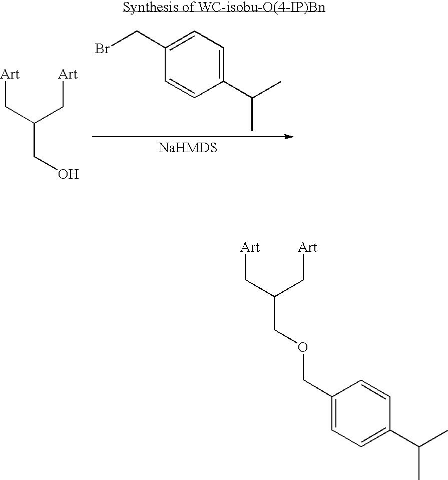

To a solution of bis-trioxane primary alcohol (118 mg, 0.19 mmol) in THF (2 mL) at 0° C. was added sodium bis(trimethylsilyl)amide in THF (1.0 M, 0.39 mL, 0.39 mmol) and isopropylbenzyl bromide (67 μL, 0.39 mmol) in THF (0.5 mL). The reaction was warmed to rt and stirred for 12 h. It was quenched with saturated aq NH₄Cl (1 mL) and layers were separated. The aqueous layer was extracted with EtOAc (3×2 mL). The combined organic solution was dried (MgSO₄), and concentrated. The purification of the crude product by column chromatography (elution with EtOAc:hexanes=1:5) gave WC-isobu-O (4-IP)Bn (106 mg, 74%) as a white solid: [α]_{D}²⁴=+61 (c 0.77, CHCl₃), mp 56-57° C.; IR (thin film) 2956, 28.73, 1513, 1377, 1093, 1054, 1009, 755 cm⁻¹; ¹H NMR (400 MHz, CDCl₃) δ 7.27 (d, J=12.0 Hz, 2H), 7.17 (d, J=12.0 Hz, 2H), 5.33 (s, 1H), 5.33 (s, 1H), 4.50 (d, J=11.6 Hz, 1H), 4.42 (d, J=11.6 Hz, 1H), 4.31 (m, 1H), 4.20 (m, 1H), 3.67 (dd, J=9.6, 5.2 Hz, 1H), 3.62 (dd, J=9.6, 5.2 Hz, 1H), 2.89 (septet, J=7.0 Hz, 1H), 2.72 (dq, J=14.4, 7.2 Hz, 1H), 2.65 (dq, J=15.2, 7.6 Hz, 1H), 2.38-2.27 (m, 2H), 2.11 (m, 1H), 2.03 (m, 1H,), 1.99 (m, 1H), 1.92-1.19 (m, 32H including s at 1.41 and 1.38, and d at 1.24 with J=7.2 Hz), 0.98-0.82 (m, 14H including dd at 0.85 with J=6.8, 7.6 Hz); ¹³C NMR (100 MHz, CDCl₃) δ 147.9, 136.5, 127.9, 126.2, 103.3, 103.0, 100.9, 99.1, 89.0, 88.5, 81.2, 74.9, 72.8, 71.9, 52.6, 52.4, 44.8, 44.5, 37.3, 37.3, 36.7, 36.6, 35.6, 34.6, 34.5, 33.8, 30.6, 30.6, 30.0, 29.6, 26.2, 26.2, 24.8, 24.7, 24.7, 24.0, 24.0, 20.3, 20.2, 13.5, 13.1; HRMS (FAB) calculated for C₄₄H₆₇O₉ [(M+H)⁺] 739.4780, found 739.4805.

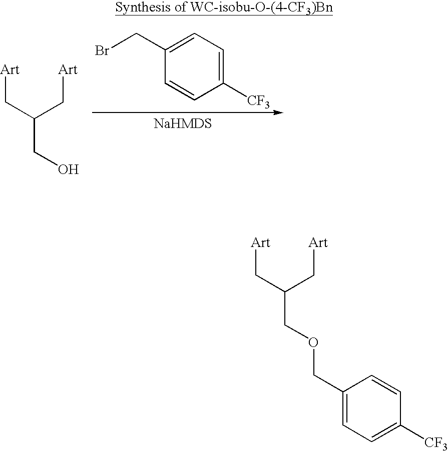

To a solution of bis-trioxane primary alcohol (62 mg, 0.10 mmol) in DMF (1 mL) at −20° C. was added sodium bis(trimethylsilyl)amide in THF (1.0 M, 0.21 mL, 0.21 mmol) dropwise. After 20 min, it was warmed to −10° C. and 4-(trifluoromethyl)benzyl bromide (49 mg, 0.21 mmol) in DMF (0.5 mL) was slowly added the reaction. The solution was warmed to rt over 2 h and stirred at rt for 1 h. The reaction mixture was diluted with ether (5 mL) and quenched with water (5 mL). Layers were separated and the aqueous layer was extracted with ether (3×3 mL). The combined organic solution was washed with water (1×2 mL), dried (MgSO₄), and concentrated. The purification of the crude product by column chromatography (elution with EtOAc:hexanes=1:5) gave WC-isobu-O-(4-CF₃)Bn (60 mg, 76%) as a white solid: [α]_{D}²⁴=+72 (c 0.42, CHCl₃); mp 61-63° C.; IR (thin film) 2922, 1325, 1124, 1163, 1066, 1011 cm⁻¹; ¹H NMR (400 MHz, CDCl₃) δ 7.57 (d, J=12.0 Hz, 2H), 7.46 (d, J=12.0 Hz, 2H), 5.31 (s, 1H), 5.28 (s, 1H), 4.60 (d, J=12.4 Hz, 1H), 4.53 (d, J=12.4 Hz, 1H), 4.35 (m, 1H), 4.22 (m, 1H), 3.73 (dd, J=9.2, 4.8 Hz, 1H), 3.66 (dd, J=9.2, 4.8 Hz, 1H), 2.71 (dq, J=15.2, 7.6 Hz, 1H), 2.62 (dq, J=14.4, 7.2 Hz, 1H), 2.37-2.26 (m, 2H), 2.12 (m, 1H), 2.02 (m, 1H,), 1.98 (m, 1H), 1.92-1.16 (m, 26H including s at 1.40 and 1.36), 0.98-0.83 (m, 14H including t at 0.93 with J=6.0 Hz, and d at 0.86 with J=7.2 Hz and 0.85 with J=7.6 Hz); ¹³C NMR (100 MHz, CDCl₃) δ 143.3, 129.2, 127.6, 125.1, 125.1, 103.2, 103.0, 100.1, 89.2, 88.6, 81.2, 74.8, 72.1, 72.1, 72.0, 52.5, 52.3, 44.7, 44.4, 37.4, 37.4, 36.6, 36.6, 35.6, 34.5, 34.5, 30.6, 30.6, 30.1, 30.0, 26.2, 26.1, 24.9, 24.7, 24.6, 20.2, 20.1, 13.4, 13.0; ¹⁹F NMR (282 MHz, CDCl₃) δ −62.4; HRMS (FAB) calculated for C₄₂H₆₀F₃O₉ [(M+H)⁺] 765.4184, found 765.4179.

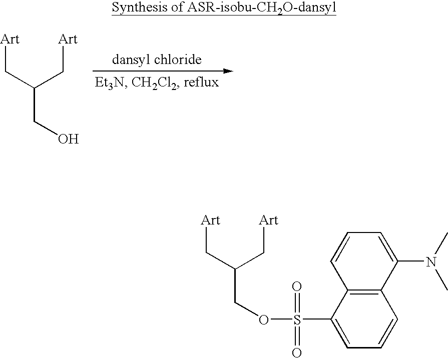

Dansyl chloride (134 mg, 0.50 mmol) was dissolved in dichloromethane (7 mL) with triethylamine (69 μL, 0.50 mmol) and stirred for 10 min. Bis-trioxane primary alcohol (100 mg, 0.17 mmol) was added to the solution and stirred at reflux for 18 h. The reaction was then allowed to cool and concentrated in vacuo. The crude product was purified by flash silica gel column chromatography (20% EtOAc in hexanes) to yield ASR-isobu-CH₂O-dansyl as a bright yellow solid (101 mg, 73%): [α]_{D}^{22.5} +41° (c=0.09, CHCl₃); mp=110-112° C.; IR (thin film) 2938, 2875, 1713, 1575, 1454, 1376, 1356, 1176, 1105, 1053, 1008, 943, 881, 842, 790, 735, 632, 575 cm⁻¹; ¹H NMR (400 MHz, CDCl₃) δ 8.61 (br s, 1H), 8.33-8.27 (m, 2H), 7.58-7.52 (m, 2H), 7.20 (br s, 1H), 5.10 (s, 1H), 5.09 (s, 1H), 4.27-4.06 (m, 4H), 2.91 (s, 6H), 2.58-2.50 (m, 1H), 2.31-2.10 (m, 41), 2.00-1.82 (m, 4H), 1.72-1.55 (m, 10H), 1.54-1.04 (m, 14H), 0.98-0.84 (m, 8H), 0.75-0.65 (m, 6H); ¹³CNMR (100 MHz, CDCl₃) δ 131.6, 131.1, 130.6, 130.0, 129.9, 128.3, 123.3, 119.8, 115.3, 103.0, 102.4, 89.4, 88.9, 81.1, 80.9, 73.6, 73.0, 69.7, 52.3, 52.0, 45.5, 44.2, 43.9, 37.4, 37.3, 36.6, 34.5, 34.4, 33.8, 30.9, 30.4, 30.3, 29.6, 26.0, 25.9, 24.7, 24.7, 20.2, 20.1, 12.8, 12.3; HRMS (FAB) m/z calc'd for C₄₆H₆₆NO₁₁S (M+H)⁺ 840.4357, found 840.4352; HPLC [Dynamax semi-preparative silica gel column (1×25 cm)], 20% EtOAc in hexanes, 2 mL/min, 264 nm, t_{R}=29.4 min).

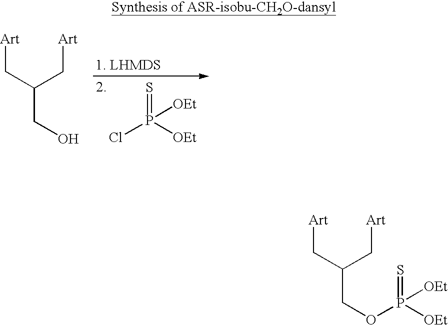

An oven dried 15 mL round bottom flask was charged with bis-trioxane primary alcohol (0.080 g, 0.13 mmol) and dissolved in 3 mL of anhydrous THF. To this solution at 0° C. was added lithium hexamethyldisilane (LHMDS, 1.0 M in THF, 0.20 mL, 0.20 mmol) dropwise over the course of about 1 min. After stirring for 10 min, diethyl chloro thiophosphate (52 μL, 0.33 mmol) was added neat. The reaction mixture was allowed to warm to room temperature and stir for 2 hr before being quenched by the slow addition of H₂O (5 mL). The contents of the flask were extracted with CH₂Cl₂ (2×25 mL), washed with a saturated aqueous solution of NaHCO₃ and H₂O, dried over MgSO₄, and concentrated in vacuo. The crude product was purified by silica gel chromatography (20% ethyl acetate in hexanes) to give WM-isobu-OP(S)(OEt)₂ as a white solid (0.056 g, 56%): [□]_{D}²³=59.2 (c 3.30, CHCl₃); mp=56-58° C.; IR (thin film) 2943, 2872, 1737, 1443, 1378, 1102, 1002, 967 cm⁻¹; ¹H NMR (400 MHz, CDCl₃) δ 5.31 (s, 1H), 5.28 (s, 1H), 4.40-4.35 (m, 1H), 4.23-3.21 (m, 3H), 4.20-4.06 (m, 4H), 2.70-2.53 (m, 2H), 2.35-2.19 (m, 3H), 2.02-1.17 (m, 34H), 0.97-0.82 (m, 14H); ¹³C NMR (100 MHz, CDCl₃) δ 103.1, 102.8, 89.3, 88.6, 81.14, 81.07, 73.9, 71.2, 70.34, 70.27, 64.13, 64.11, 64.05, 52.5, 52.2, 44.5, 44.2, 37.4, 37.3, 36.63, 36.57, 35.2, 35.1, 34.5, 34.4, 30.5, 30.4, 30.2, 29.6, 26.10, 26.06, 24.82, 24.75, 24.70, 24.6, 20.2, 20.1, 15.95, 15.94, 15.88, 15.86, 15.2, 13.2; HRMS (FAB, M+1) calc. 759.3907 for C₃₈H₆₄O₁₁PS, found 759.3896.

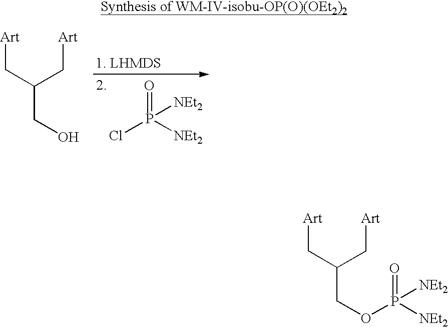

An oven dried 15 mL round bottom flask was charged with bis-trioxane primary alcohol (0.050 g, 0.08 mmol) and dissolved in 3 mL of anhydrous THF. To this solution at 0° C. was added lithium hexamethyldisilane in THF (LHMDS, 1.0 M, 0.12 mL, 0.12 mmol) dropwise over the course of about 1 min. After stirring for 10 min, Bis(diethylamino) chloro phosphate (44 μL, 0.21 mmol) was added neat. The reaction mixture was allowed to warm to room temperature and stir for 2 hr before being quenched by the slow addition of H₂O (5 mL). The contents of the flask were extracted with CH₂Cl₂ (2×25 mL), washed with a saturated aqueous solution of NaHCO₃ and H₂O, dried over MgSO₄, and concentrated in vacuo. The crude product was purified by silica gel chromatography (20% ethyl acetate in hexanes) to give WM-isobu-OP(O)(NEt₂)₂ as an amorphous solid (0.023 g, 35%): [□]_{D}²³=67.6 (c=1.10, CHCl₃); IR (thin film) 2937, 2361, 2341, 1457, 1377, 1210, 1105, 1011 cm⁻¹; ¹H NMR (400 MHz, CDCl₃) δ 5.30 (s, 1H), 5.29 (s, 1H), 4.34-4.33 (m, 1H), 4.20-4.19 (m, 1H), 4.06-4.04 (m, 1H), 3.95-3.93 (m, 1H), 3.19-3.14 (m, 1H), 3.08-3.00 (m, 8H), 2.72-2.70 (m, 1H), 2.60-2.59 (m, 1H), 2.32-2.25 (m, 3H), 2.14-1.14 (m, 25H), 1.10 (t, J=14.4 Hz, 16H), 0.96-0.83 (m, 16H); ¹³C NMR (100 MHz, CDCl₃) δ 102.9, 89.1, 88.5, 81.2, 81.1, 74.2, 72.2, 52.5, 52.3, 44.7, 44.3, 39.6, 39.5, 37.4, 36.6, 34.5, 30.5, 30.4, 26.1, 24.69, 24.65, 20.2, 20.1, 14.39, 14.37, 13.4; HRMS (FAB, M+1) calc. 797.5081 for C₄₂H₇₄N₂O₁₀P, found 797.5073.

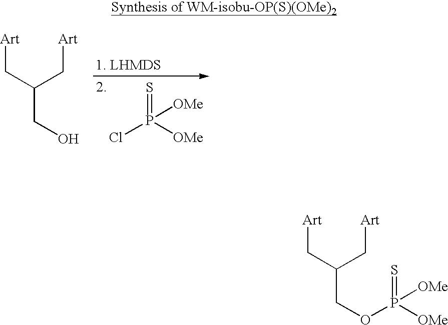

An oven dried 15 mL round bottom flask was charged with bis-trioxane primary alcohol (0.050 g, 0.08 mmol) and dissolved in 3 mL of anhydrous THF. To this solution at 0° C. was added lithium hexamethyldisilane in THF (LHMDS, 1.0 M, 0.12 mL, 0.12 mmol) dropwise over the course of about 1 min. After stirring for 10 min, dimethyl chloro thiophosphate (25 μL, 0.21 mmol) was added neat. The reaction mixture was allowed to warm to room temperature and stir for 2 hr before being quenched by the slow addition of H₂O (5 mL). The contents of the flask were extracted with CH₂Cl₂ (2×25 mL), washed with a saturated aqueous solution of NaHCO₃ and H₂O, dried over MgSO₄, and concentrated in vacuo. The crude product was purified by silica gel chromatography (20% ethyl acetate in hexanes) to give WM-isobu-OP(S)(OMe)₂ as an amorphous solid (0.039 g, 65%): [0] D²³=70.5 (c=1.95, CHCl₃); IR (thin film) 2943, 2872, 1449, 1373, 1185, 1102, 1032, 1008, 820, 756 cm⁻¹; ¹H NMR (400 MHz, CDCl₃) δ 5.32 (s, 1H), 5.29 (s, 1H), 4.40-4.39 (m, 1H), 4.26-4.18 (m, 3H), 3.75 (d, J=1.2 Hz, 3H), 3.72 (d, J=1.2 Hz, 3H), 2.70-2.68 (m, 1H), 2.57-2.55 (m, 1H), 2.32-2.52 (m, 2H), 2.25-1.20 (m, 29H), 0.96-0.82 (m, 16H); ¹³C NMR (100 MHz, CDCl₃) δ 103.1, 102.8, 89.4, 88.7, 81.2, 81.1, 73.8, 70.9, 70.6, 54.53, 54.47, 52.4, 52.1, 44.5, 44.1, 37.4, 37.3, 36.6, 36.6, 35.2, 35.1, 34.5, 34.4, 30.52, 30.45, 30.2, 29.6, 26.1, 26.0, 24.82, 24.76, 24.71, 24.65, 20.2, 20.1, 14.1, 13.2, 12.7.

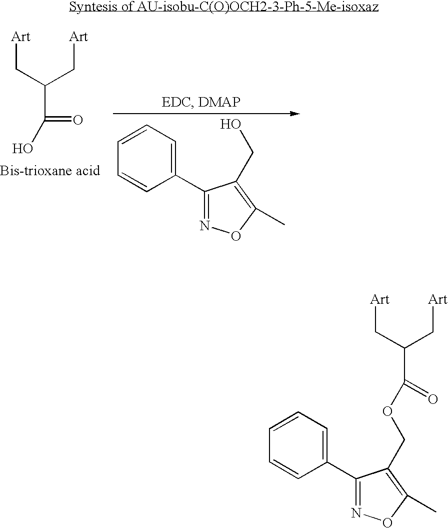

Bis-trioxane acid (100 mg, 0.66 mmol) was dissolved in CH₂Cl₂ (10 mL) in an oven dried 25 ml round bottom flask charged with magnetic stir bar and argon balloon. 1-(3-(dimethylamino)propyl)-3-ethylcarbodiimide hydrochloride (EDC, 130 mg, 0.660 mmol), dimethylaminopyridine (DMAP, 81 mg, 0.66 mmol), and the 5-methyl-3-phenyl-4-isoxazolylmethanol (125 mg, 0.66 mmol) were added. The sides of the flask were washed with CH₂Cl₂ (1 mL) and the reaction stirred 16 hours at room temperature. The reaction was quenched with H₂O (10 mL) and extracted with CH₂Cl₂ (3×20 mL). The organics were dried over magnesium sulfate, filtered and concentrated. Purification by column chromatography (20% Ethyl Acetate: 80% Hexanes) gave AU-isobu-C(O)OCH₂-3-Ph-5-Me-isoxaz (91 mg, 73%) as an amorphous solid: [α]_{D}²⁵=+59 (c=0.70, CHCl₃); IR (thin film) 2953 (s), 2878 (m), 1731(s), 1639(w), 1454(m), 1379(m), 1354(w), 1270(w), 1220(m), 1161(m), 1120(m), 1086(m), 1053(s), 1011(s), 960(w), 935(m), 868(m), 835(w), 810(w), 751(s), 693(m); ¹H NMR (400 MHz, CDCl₃) δ 7.80-7.69 (m, 2H), 7.49-7.43 (m, 3H), 5.19-5.14 (m, 2H), 4.97-4.93 (m, 1H), 4.20-4.10 (m, 2H), 2.78-2.71 (m, 2H), 2.59-2.52 (m including singlet at 2.53, 4H), 2.30-2.21 (m, 2H), 2.12-1.44 (m, 15H), 1.41-1.31 (m, 3H), 1.25-1.15 (m including singlets at 1.22 and 1.19, 11H), 1.31-1.13 (m, 6H), 0.94-0.87 (m, 7H), 0.84-0.78 (m, 7H); ¹³C NMR (100 MHz, CDCl₃) δ 176.4, 170.1, 129.6, 128.9, 128.9, 128.3, 109.1 103.3, 102.9, 89.1, 88.2, 81.0, 81.0, 75.1, 72.3, 55.7, 52.5, 52.1, 44.6, 44.0, 42.6, 37.4, 37.3, 36.5, 34.5, 34.4, 32.7, 31.6, 31.6, 30.5, 30.1, 25.8, 25.7, 25.3, 24.8, 24.8, 24.7, 24.6, 22.6, 20.2, 20.1, 14.1, 13.4, 12.5, 11.5; HRMS (FAB) calculated for C₄₅H₆₂NO₁₁ 792.4323, found 792.4376.

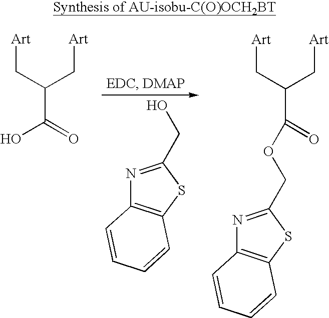

Bis-trioxane acid (80 mg, 0.13 mmol) was dissolved in CH₂Cl₂ (10 mL) in an oven dried 25 ml round bottom flask charged with magnetic stir bar and argon balloon. 1-(3-(dimethylamino)propyl)-3-ethylcarbodiimide hydrochloride (EDC, 102 mg, 0.53 mmol), dimethylaminopyridine (DMAP, 65 mg, 0.53 mmol), and the 2-hydroxymethylbenzothiazole (88 mg, 0.53 mmol) were added. The sides of the flask were washed with CH₂Cl₂ and the reaction stirred 16 hours at room temperature. The reaction was quenched with H₂O (10 mL) and extracted with CH₂Cl₂ (3×20 mL). The organics were dried over magnesium sulfate, filtered and concentrated. Purification by column chromatography (20% Ethyl Acetate: 80% Hexanes) gave AU-isobu-C(O)OCH₂BT (68 mg, 68%) as an amorphous solid: [α]_{D}²⁴=+75 (c=0.10, CHCl₃); IR (thin film) 2939(s), 2874(m), 1738(s), 1510(w), 1436(m), 1485(m), 1376(m), 1126(m), 1093(m), 1053(s), 1011 (s), 940(w), 878(m), 758(s); ¹H NMR (400 MHz, CDCl₃) δ 8.01 (d, 1H, J=8.0 Hz), 7.89 (d, 1H, J=7.6 Hz), 7.50-7.46 (m, 1H), 7.41-7.37 (m, 1H), 5.61-5.52 (m, 2H), 5.29 (s, 1H), 5.21 (s, 1H), 4.29-4.20 (m, 2H), 2.97-2.93 (m, 1H), 2.81-2.76 (m, 1H), 2.69-2.84 (m, 1H), 2.33-2.13 (m, 3H), 2.00-1.94 (m, 2H), 1.91-1.76 (m, 7H), 1.68-1.52 (m, 4H), 1.51-1.36 (m, 3H), 1.36-1.19 (m including singlets at 1.34 and 1.28, 11 H), 0.99-0.91 (m, 7H), 0.90-0.84 (m, 7H); ¹³C NMR (100 MHz, CDCl₃) δ 176.0, 126.1, 125.2, 123.0, 121.7, 103.4, 103.1, 89.0, 88.1, 81.1, 81.0, 75.2, 73.0, 64.0, 52.5, 52.2, 44.6, 44.2, 42.6, 37.4, 37.3, 36.5, 36.5, 34.7, 34.5, 34.4, 31.6, 31.6, 30.4, 30.1, 29.1, 26.0, 25.9, 24.9, 24.8, 24.7, 24.6, 20.7, 20.2, 20.1, 13.5, 12.8; HRMS (FAB) calculated for C₄₂H₅₈NO₁₀S 768.3781, found 768.3788.

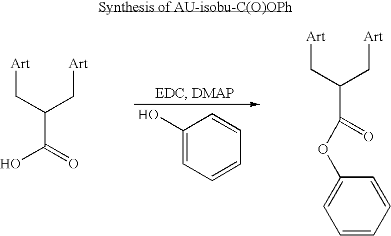

Bis-trioxane acid (100 mg, 0.16 mmol) was dissolved in CH₂Cl₂ (10 mL) in an oven dried 25 ml round bottom flask charged with magnetic stir bar and argon balloon. 1-(3-(dimethylamino)propyl)-3-ethylcarbodiimide hydrochloride (EDC, 130 mg, 0.66 mmol), dimethylaminopyridine (DMAP, 81 mg, 0.66 mmol), and phenol (62 mg, 0.66 mmol) were added. The sides of the flask were washed with CH₂Cl₂ and the reaction stirred 16 hours at room temperature. The reaction was quenched with H₂O (10 mL) and extracted with CH₂Cl₂ (3×20 mL). The organics were dried over magnesium sulfate, filtered and concentrated. Purification by column chromatography (20% Ethyl Acetate in Hexanes) gave AU-isobu-C(O)OPh (83 mg, 0.12 mmol, 76%) as an amorphous solid: [α]_{D}²³=+88 (c=3.9, CHCl₃); IR (thin film) 3072(w), 3016(m, sh), 2971(s), 2953(s), 2953(s, sh), 2874(m), 2848(m, sh), 1751(s), 1594(m), 1493(m), 1451(m), 1435(m, sh), 1377(m), 1279(w), 1252(m), 1225(s), 1191(m), 1143(m), 1127(s), 1093(2), 1053(s), 1010(s), 939(m), 927(m), 878(m), 848(w), 825(w), 762(w), 750(s), 692(m), 666(m); ¹H NMR (400 MHz, CDCl₃) δ 7.33-7.25 (m, 4H), 7.16-7.11 (m, 1H), 5.33 (s, 1H), 5.27 (s, 1H), 4.94-4.46 (m, 1H), 4.28-4.24 (m, 1H), 2.96-2.91 (m, 1H), 2.85-2.72 (m, 1H), 2.63-2.52 (m, 1H), 2.34-2.16 (m, 3H), 1.99-1.87 (m, 4H), 1.83-1.59 (m, 8H), 1.55-1.48 (m, 2H), 1.48-1.31 (m including singlets at 1.43 and 1.34, 8H), 1.31-1.13 (m, 5H), 0.96-0.88 (m, 7H), 0.88-0.80 (m, 7H); ¹³C NMR (100 MHz, CDCl₃) δ 175.5, 151.1, 128.8, 125.3, 122.4, 103.4, 102.9, 89.3, 88.2, 81.1, 81.0, 76.2, 72.2, 52.5, 52.0, 44.6, 43.9, 43.3, 37.4, 37.1, 36.5, 36.5, 34.6, 34.3, 34.2, 32.1, 31.5, 30.5, 30.2, 26.0, 25.8, 25.2, 24.7, 24.3, 22.5, 20.1, 20.0; HRMS (FAB) calculated for C₄₀H₅₇O₁₀ 697.3952, found 697.3970.

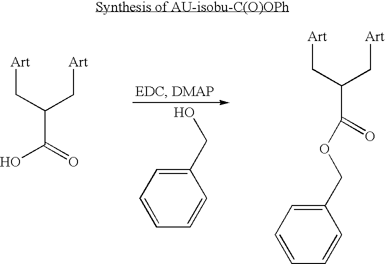

Bis-trioxane acid (100 mg, 0.16 mmol) was dissolved in CH₂Cl₂ (10 mL) in an oven dried 25 ml round bottom flask charged with magnetic stir bar and argon balloon. 1-(3-(dimethylamino)propyl)-3-ethylcarbodiimide hydrochloride (EDC, 130 mg, 0.66 mmol), dimethylaminopyridine (DMAP, 81 mg, 0.66 mmol), and benzyl alcohol (68 mg, 0.66 mmol) were added. The sides of the flask were washed with CH₂Cl₂ and the reaction stirred 16 hours at room temperature. The reaction was quenched with H₂O (10 mL) and extracted with CH₂Cl₂ (3×20 mL). The organics were dried over magnesium sulfate, filtered and concentrated. Purification by column chromatography (25% Ethyl Acetate in Hexanes) gave AU-isobu-C(O)OCH₂Ph (84 mg, 75%) as an amorphous solid: [α]_{D}²⁵=+70 (c=0.75, CHCl₃); IR (thin film) 2945(s), 2878(m), 1722(s), 1446(m), 1371(m), 1354(m), 1279(w), 1253(m), 1228(m), 1195(m), 1178(m), 1128(m), 1086(m), 1053(s), 1002(s), 952(w), 927(m), 877(m), 835(w), 815(w), 743(s). ¹H NMR (400 MHz, CDCl₃) δ 7.42-7.39 (m, 2H), 7.35-7.28 (m, 3H), 5.33 (s, 1H), 5.29-5.07 (m, 4H), 4.21-4.13 (m, 2H), 2.85-2.74 (m, 2H), 2.64-2.59 (m, 1H), 2.34-2.25 (m, 2H), 2.17-2.08 (m, 1H), 2.04-1.95 (m, 2H), 1.90-1.35 (m, 15H), 1.35-1.27 (m including singlets at 1.31 and 1.29, 8H), 1.25-1.19 (m, 3H), 0.94-0.93 (m, 7H), 0.86-0.80 (m, 6H); ¹³C NMR (100 MHz, CDCl₃) δ 176.6, 136.4, 128.4, 128.2, 127.7, 103.3, 103.1, 88.8, 88.0, 81.1, 80.9, 75.6, 73.3, 66.7, 52.5, 52.2, 44.7, 44.2, 43.0, 37.4, 37.8, 36.5, 34.6, 34.5, 34.3, 32.4, 31.7, 31.5, 30.3, 30.0, 26.0, 25.9, 25.2, 24.8, 24.7, 24.7, 24.5, 20.2, 20.1, 13.5, 12.7; HRMS (FAB) calculated for C₄₁H₅₉O₁₀ 711.4108, found: 711.4099.

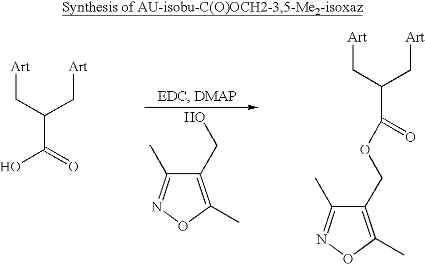

Bis-trioxane acid (100 mg, 0.66 mmol) was dissolved in CH₂C₂ (10 mL) in an oven dried 25 ml round bottom flask charged with magnetic stir bar and argon balloon. 1-(3-(dimethylamino)propyl)-3-ethylcarbodiimide hydrochloride (EDC, 130 mg, 0.66 mmol), dimethylaminopyridine (DMAP, 81 mg, 0.66 mmol), and the 3,5-dimethyl-4-isoxazolylmethanol (83 mg, 0.66 mmol) were added. The sides of the flask were washed with CH₂Cl₂ (1 mL) and the reaction stirred 16 hours at room temperature. The reaction was quenched with H₂O (10 mL) and extracted with CH₂Cl₂ (3×20 mL). The organics were dried over magnesium sulfate, filtered and concentrated. Purification by column chromatography (25% Ethyl Acetate in Hexanes) gave AU-isoC(O)OCH2-3,5-Me₂-isoxaz (75 mg, 0.43 mmol, 65%) as an amorphous solid: [α]_{D}²⁵=+76 (c=0.48, CHCl₃); IR (thin film) 2936(s), 2878(m), 1714(s), 1630(w), 1605(w), 1546(w), 1454(m), 1379(m), 1279(w), 1262(m), 1220(m), 1161(m), 1120(m), 1086(m), 1053(m), 1101(m), 969(w), 935(w), 877(m), 843(w), 828(w), 760(m); ¹H NMR (400 MHz, CDCl₃) δ 5.25 (s, 1H), 5.18 (s, 1H), 5.03-4.98 (m, 1H), 4.90-4.85 (m, 1H), 4.22-4.13 (m, 2H), 2.77-2.72 (m, 1H), 2.64-2.54 (m, 1H), 2.43 (s, 3H), 2.36-2.27 (m including singlet at 2.31, 4H), 2.14-1.98 (m, 3H), 1.89-1.53 (m, 13H), 1.48-1.24 (m including singlets at 1.35 and 1.29, 1511), 0.99-0.96 (m, 7H), 0.92-0.82 (m, 6H); ¹³CNMR (100 MHz, CDCl₃) δ 176.5, 168.1, 110.0, 103.3, 102.9, 89.2, 88.2, 81.1, 81.0, 75.3, 72.5, 55.7, 52.5, 52.1, 44.6, 44.0, 42.7, 37.5, 37.3, 36.5, 34.5, 34.4, 32.9, 31.9, 30.5, 30.1, 26.1, 25.7, 24.8, 24.8, 24.8, 24.6, 22.6, 20.2, 20.1, 14.1, 13.4, 12.5, 11.1, 10.9; HRMS (FAB) calculated for C₄₀H₆₀NO₁₁ 730.4167, found 730.4164.

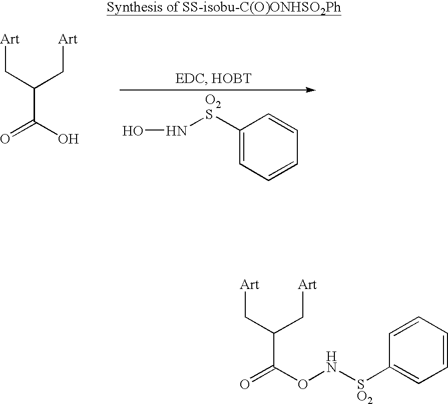

A flame-dried 20 mL recovery flask equipped with a magnetic stir bar, a septum along with an Ar balloon was charged with bis-trioxane acid (50 mg, 0.10 mmol) and it was dissolved in 2.0 mL freshly distilled CH₂Cl₂. Then the flask was cooled down to 0° C., and 1-(3-(dimethylamino)propyl)-3-ethylcarbodiimide hydrochloride (EDC, 23 mg, 0.12 mmol, 1.5 eq) and hydroxybenzotriazole (HOBT, 16 mg, 0.12 mmol, 1.5 eq) were added respectively. The mixture was allowed to stir for 2 hr. In a separate flame dried flask, charged with an argon balloon, phenylsulfonylhydroxamic acid (28 mg, 0.16 mmol, 2.0 eq) was dissolved in DMF (1 mL) and cooled down to 0° C. where NaH (0.021 mg, 0.84 mmol) was added, generating a yellow colored solution. To the yellow colored solution the intermediate mixture was added via cannula, and left stirring for an hour. The reaction was quenched by addition of 10 mL cold distilled water and then rinsed into a separatory funnel with ethyl ether (10 mL). The mixture was extracted with ethyl ether (3×30 mL). The combined extracts were washed with water (5 mL), and brine solution (5 mL), dried over Na₂SO₄ and filtered. The filtrate was concentrated in vacuo to give the crude product that was purified by flash column chromatography, which was eluted with 25% ethyl acetate in hexanes to afford SS-isobu-C(O)ONHSO₂Ph (52 mg, 69%) as an amorphous solid: [α]²⁵_{D} +29 (c 0.65, CHCl₃); IR (thin film) 3175, 2932, 2870, 1765, 1524, 1549, 1434, 1376, 1325, 1178, 1088, 1049, 1002, 933, 870 cm⁻¹; ¹H NMR (400 MHz, CDCl₃) δ 8.71 (s, 1H), 8.07-8.01 (m, 2H), 7.66-7.52 (m, 3H), 5.22 (s, 1H), 5.10 (s, 1H), 4.07-4.01 (m, 3H), 3.86-3.81 (m, 1H), 2.83-2.79 (m, 1H), 2.73-2.45 (m, 2H), 2.37-2.27 (m, 2H), 2.04-1.73 (m, 9H), 1.70-1.16 (m, 17H, including two singlets at 1.42, and 1.41), 1.00-0.91 (m, 8H), 0.81-0.73 (m, 6H); ¹³C NMR (100 MHz, CDCl₃) δ 174.8, 136.3, 133.8, 129.1, 129.0, 103.6, 103.28, 88.87, 88.38, 80.95, 80.85, 77.20, 74.02, 72.79, 52.24, 52.19, 44.29, 44.12, 37.44, 37.33, 36.50, 36.46, 34.38, 32.63, 31.85, 30.07, 30.00, 25.99, 25.84, 24.72, 24.62, 20.16, 20.13, 13.04, 12.59; LRMS (FAB) calc'd for C₄₀H₅₇NO₁₂SH⁺ [M+H]776.34, found 776.34.

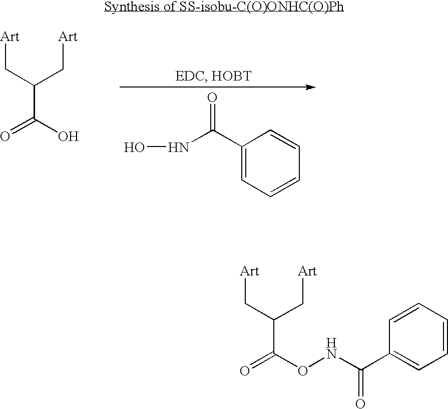

A flame-dried 20 mL recovery flask equipped with a magnetic stir bar, a septum along with an Ar balloon was charged with bis-trioxane acid (25 mg, 0.05 mmol) and dissolved in 2.0 mL freshly distilled CH₂Cl₂. Then the flask was cooled down to 0° C., and 1-(3-(dimethylamino)propyl)-3-ethylcarbodiimide hydrochloride (EDC, 12 mg, 0.06 mmol, 1.5 eq) and hydroxybenzotriazole (HOBT, 8 mg, 0.06 mmol, 1.5 eq) were added respectively. The mixture was allowed to stir for 2 hr. In a separate flame dried flask, charged with an argon balloon, phenylhydroxamic acid (16 mg, 0.12 mmol, 3.0 eq) was dissolved in DMF (1 mL) and cooled down to 0° C. where NaH (1 mg, 0.46 mmol) was added, generating a yellow colored solution. To this colored solution the mixture was added via cannula, and left stirring for an hour. The reaction was quenched by addition of 10 mL cold distilled water and then rinsed into a separatory funnel with ethyl ether. The mixture was extracted with ethyl ether (3×30 mL). The combined extracts were washed with water (5 mL), and brine solution (5 mL), dried over Na₂SO₄ and filtered. The filtrate was concentrated in vacuo to give the crude product that was purified by flash column chromatography, which was eluted with 40% ethyl acetate in hexanes to afford SS-isobu-C (O)ONHC(O)Ph (27 mg, 75%) as an amorphous solid: [α]²⁵_{D} +61 (c 0.10, CHCl₃); IR (thin film) 3451, 2921, 2856, 1708, 1634, 1592, 1460, 1377, 1237, 1096, 1047, 1007, 734 cm⁻¹; ¹H NMR (400 MHz, CDCl₃) δ 8.71 (s, 1H), 7.75 (d, J=7.2 Hz, 2H), 7.57-7.52 (m, 1H), 7.47-7.43 (M, 2H), 5.31 (s, 1H), 5.30 (s, 1H), 4.35-4.42 (m, 2H), 2.95-2.82 (m, 1H), 2.72-2.59 (m, 2H), 2.34-2.26 (m, 3H), 2.11-1.56 (m, 23H), 1.43-1.18 (m, 13H, including two singlet at 1.40, and 1.39), 0.97-0.86 (m, 14H).

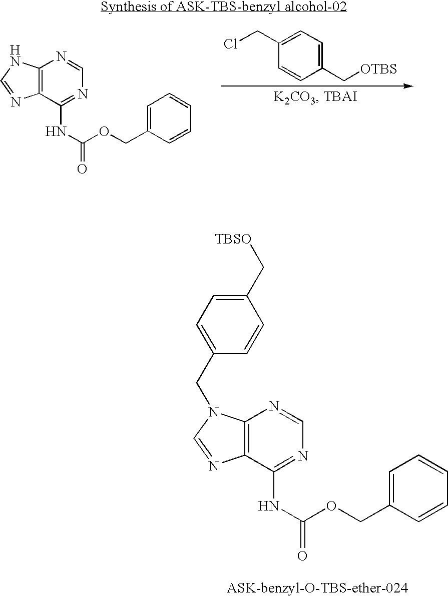

A dry 50 mL round bottom flask was charged with of C6-(NH)CBz-adenine (0.37 g, 1.10 mmol, 1.0 equiv), p-chloromethyl-t-butyldimethylsilylbenzyl alcohol (00.10 g, 0.37 mmol, 0.30 equiv), potassium carbonate (0.15 g, 1.10 mmol, 1 equiv) and tetrabutylamonium iodide (TBAI, 0.015 g, 0.04 mmol) in anhydrous DMF (20 mL). The reaction was stirred at room temperature for 13 hours. The reaction was observed to be complete via TLC and quenched with water (20 mL). Et₂O (100 mL) was added to the reaction mixture which was then poured to a separatory funnel. The mixture was washed with ice water (5×150 mL), dried over MgSO₄, and concentrated in vacuo. The crude yellow material was purified via silica column chromatography eluting with 80% EtOAc in hexanes to give pure ASK-benzyl-O-TBS-ether-024 as a white solid (0.18 mg, 98%): mp=1135-136° C.; IR (thin film) 2955, 2929, 2856, 1757, 1615, 1583, 1465, 1254, 1202, 1156, 1090, 838, 778, 697 cm⁻¹; ¹H NMR (400 MHz, CDCl₃) δ8.80 (s, 1H), 7.88 (s, 1H), 7.42-7.29 (m, 7H), 7.25-7.22 (r, 2H), 5.33 (s, 2H), 5.28 (s, 2H), 4.71 (s, 2H), 0.92 (s, 9H), 0.08 (s, 6H); ¹³C NMR (100 MHz, CDCl₃) δ 152.9, 151.5, 151.0, 149.3, 142.6, 142.1, 135.4, 133.4, 128.6, 128.5, 127.8, 126.6, 121.7, 77.3, 67.7, 64.4, 47.2, 25.8, 18.3, −5.30; HRMS (FAB) m/z calc'd for C₂₇H₃₄N₅O₃Si (M+H⁺) 504.2431, found 504.2433.

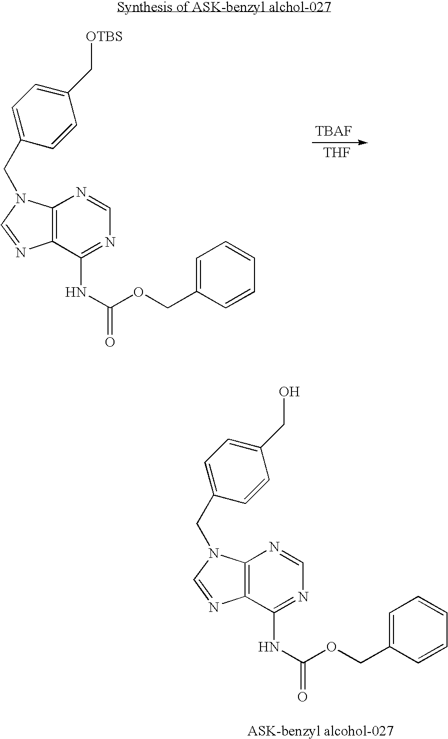

A dry 50 mL round bottom flask was charged with of ASK-benzyl-O-TBS-ether-024 (0.18 g, 0.36 mmol, 1.0 equiv) and tetrabutylamonium iodide (TBAF, 1.1 mL, 1.10 mmol, 3 equiv) in anhydrous THF (10 mL). The reaction was stirred at room temperature for 5 hours. The reaction was observed to be complete via TLC and quenched with water (10 mL). Et₂O (100 mL) was added to the reaction mixture which was then poured to a separatory funnel. The mixture was washed with brine (2×50 mL), dried over MgSO₄, and concentrated in vacuo. The crude yellow material was purified via silica column chromatography eluting with 100% EtOAc to give pure ASK-benzyl alcohol-027 as a white solid (0.13 mg, 90%): mp=148-149° C.; IR (film) 3270, 3052, 2929, 1752, 1617, 1585, 1466, 1405, 1322, 1215, 1159, 752 cm⁻¹; ¹H NMR (400 MHz, CDCl₃) δ 8.78 (s, 1H), 7.89 (s, 1H), 7.48-7.28 (m, 9H), 5.38 (s, 2H), 5.29 (s, 2H), 4.69 (s, 2H), 2.50-1.50 (bs, 1H); ¹³C NMR (100 MHz, CDCl₃) δ 152.6, 150.2, 146.5, 141.6, 141.6, 135.4, 128.6, 128.5, 128.2, 127.6, 126.5, 77.3, 67.8, 64.7, 47.3, 14.0, 12.7; HRMS (FAB) m/z calc'd for C₂₁H₂₀N₅O₃ (M+H⁺) 390.1566, found 390.1557.

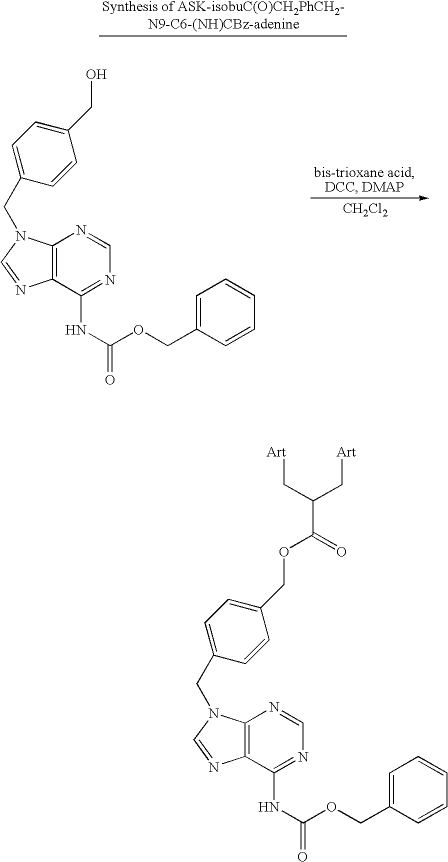

A 25 mL round bottom flask was charged with of bis-trioxane acid (48 mg, 0.08 mmol) in anhydrous dichloromethane (5 mL) and dimethylaminopyridine (DMAP, 9 mg, 0.08 mmol, 1.5 equiv) were added to the solution. To a dry pear shaped flask was added dicyclohexylcarbidimide (DCC, 20 mg, 0.08 mmol, 1.5 equiv) and anhydrous dichloromethane (3 mL). The DCC solution was cannulated into the bis-trioxane acid mixture at room temperature and is allowed to stir overnight. TLC analysis showed full consumption of starting material. Water (10 mL), saturated sodium bicarbonate solution (10 mL) and methylene chloride (10 mL) were added to the reaction and the organics were extracted with methylene chloride (3×20 mL), dried (MgSO₄) and concentrated in vacuo to give a sticky white solid. Flash column chromatography on silica eluting with (60% EtOAc in hexanes) yielded ASK-isobuC(O)CH₂PhCH₂—N-9-C6-(NH)CBz-adenine as a white solid (37 mg, 72%). [α]_{D}²³=+31 (CHCl₃, c=0.70), mp=178-181° C.; IR (film) 3330, 2929, 2865, 1752, 1728, 1613, 1584, 1463, 1376, 1320, 1208, 1155, 1092, 1051, 1010, 911, 878, 730 cm⁻¹; ¹H NMR (400 MHz, CDCl₃) δ 8.78 (s, 1H), 7.89 (s, 1H), 7.44-7.29 (m, 7H), 7.24 (s, 1H), 5.37 (s, 2H), 5.29 (s, 2H), 5.25 (s, 1H), 5.18 (s, 1H), 5.15 (s, 1H), 5.07-5.04 (m, 1H), 4.20-4.09 (m, 2H), 3.51-3.42 (m, 2H), 2.85-2.70 (m, 2H), 2.62-2.50, (m, 1H), 2.35-2.20 (m, 2H), 2.15-2.03 (m, 1H), 1.45-1.28 (m, 9H), 1.25 (s, 7H), 1.23-1.04 (m, 10H), 0.96-0.87 (m, 8H), 0.84 (d, J=8.0 Hz, 4H), 0.78 (d, J=8.0 Hz, 3H); ¹³C NMR (100 MHz, CDCl₃) δ176.6, 166.5, 155.3, 153.1, 152.6, 151.2, 137.2, 135.5, 135.3, 132.5, 131.6, 130.2, 130.0, 129.5, 129.2, 129.1, 128.7, 128.6, 128.5, 127.9, 103.4, 103.1, 88.9, 88.0, 81.1, 81.0, 80.7, 79.2, 78.4, 75.3, 74.6, 74.2, 73.8, 73.5, 73.3, 52.5, 52.2, 49.2, 44.7, 44.2, 37.4, 37.2, 36.5, 33.9, 30.3, 26.0, 25.8, 24.8, 24.7, 20.2, 20.1, 13.4, 12.7; HRMS (FAB) m/z calc'd for C₅₅H₇₀N₅O₁₂ (M+H⁺) 992.5021, found 992.5030.

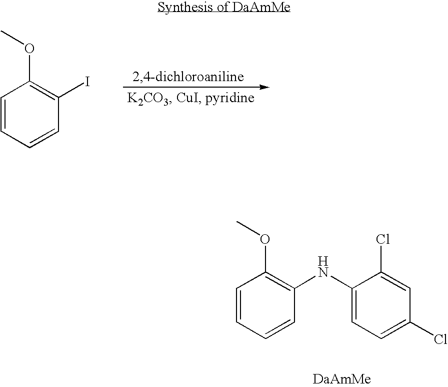

Into a 2-5 mL Biotage microwave vial was loaded a stir bar and iodoanisole (0.20 mL, 1.5 mmol), followed by the addition of pyridine (1 mL), 2,4-dichloroaniline (1.3 g, 7.7 mmol), potassium sulfate (1.5 g, 11.0 mmol) and copper iodide (0.22 g, 1.1 mmol). The vial was sealed and the mixture was then heated via microwave irradiation to 200° C. for 3 hours. After cooling, the mixture was directly applied to a silica gel column and purified by flash column chromatography (30:1=petroleum ether:ether) to give DaAmMe (0.376 g, 92%) as an oil: ¹HNMR (CDCl₃, 300 MHz) δ 7.41-7.39 (m, 1H), 7.31-7.28 (m, 2H), 7.16-7.11 (m, 1H), 7.00-6.92 (m, 3H), 6.42 (s, 1H), 3.92 (s, 3H); ¹³CNMR (CDCl₃, 75 MHz) δ 149.8, 138.8, 130.7, 129.4, 127.4, 124.4, 122.8, 122.3, 120.7, 117.5, 116.6, 111, 55.7 (A variation of the procedure found in Perozzo).

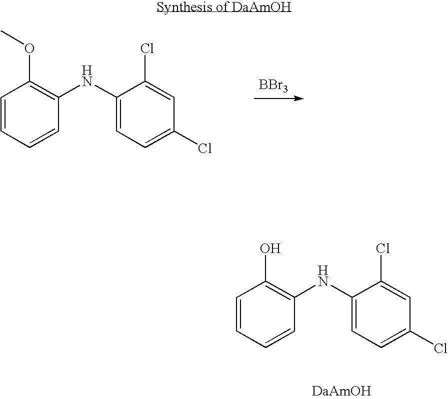

DaAmMe (100 mg, 0.40 mmol) and a stir bar were loaded together into a 100 mL round bottom flask. Under an argon balloon, dichloromethane (20 mL) was added and the system was stirred in an ice-water bath. After cooling, boron tribromide in CH₂Cl₂ (1 M, 0.45 mL, 0.45 mmol) was added over 24 minutes. During the addition, the color of the reaction became violet. TLC after stirring overnight showed no remaining staring material and the reaction was quenched with water (10 ml), causing the purple color to disappear and become colorless. It was then extracted with dichloromethane (3×50 mL), the organics were combined, washed with brine, dried with MgSO₄, filtered and evaporated. The residue was purified by flash silica column (10:1 pet ether:ether) to give DaAmOH (87 mg, 86%) as a white solid: ¹HNMR (CDCl₃, 300 MHz) δ 7.41-7.37 (m, 1H), 7.23-7.15 (m, 2H), 7.09-7.04 (m, 2H), 6.99-6.93 (m, 1H), 6.6-6.57 (d, J=8.7 Hz, 1H), 5.78 (s, 2H); ¹³CNMR (CDCl₃, 75 MHz) δ 151.8, 141, 129.1, 127.9, 127.7, 127.1, 126.2, 124.3, 121.3, 121.1, 115.8, 115.5 (¹ A variation of the procedure found in Perozzo, R.; Kuo, M.; Sidhu, A. B. S.; Valiyaveettil, J. T.; Bittnan, R.; Jacobs W. R. Jr.; Fidock, D. A.; Sacchettinim, J. C. Journal of Biological Chemistry 2002, 277, 13106-13114 was followed).

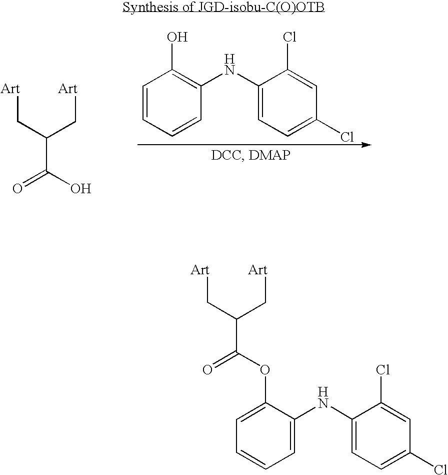

Bis-trioxane acid (35 mg, 0.06 mmol) was added to a 10 mL RBF with a stir bar. Then, methylene chloride was added, followed by DaAmOH (38 mg, 0.15 mmol), 4-dimethylamino pyridine (3 mg, 0.03 mmol) and dicyclohexylcarbodiimide (DCC, 28 mg, 0.13 mmol). The mixture was allowed to stir for 3 days at room temperature, then refluxed for a few hours. It was cooled and purified by gradient flash column:chromatography (3:1 then 2:1 petroleum ether:ether) to give JGD-isobu-C(O)OTB (26 mg, 54%) as an amorphous solid: ¹H NMR (CD₃OD, 300 MHz) δ 7.47-7.44 (m, 1H), 7.39-7.38 (d, 1H, J=3 Hz), 7.25-7.05 (m, 4H), 6.92-6.89 (d, 1H, J=9.0 Hz), 5.31 (s, 1H), 5.19 (s, 1H), 4.36-4.25 (m, 2H), 2.95 (m, 1H), 2.7-2.63 (q, 1H, J=6.0, 15.0 Hz), 2.57-2.5 (q, 1H, J=9.0, 15.0 Hz), 2.31-1.09 (m, 30H), 1.01-0.82 (m, 14H); ¹³C NMR (CD₃OD, 75 MHz) δ 174.8, 143.8, 140.3, 128.9, 127.3, 126.2, 124.4, 124.0, 123.5, 122.8, 117.8, 103.2, 102.9, 89.3, 88.7, 80.9, 74.8, 72.5, 52.4, 52.2, 44.4, 44.1, 42.6, 37.1, 36.2, 34.2, 31.6, 34.2, 31.6, 30.5, 30.2, 24.7, 24.5, 24.4, 19.2, 19.1, 12.1, 11.5; HRMS (FAB) m/z calc'd for C₄₆H₆₀Cl₂NO₁₀ (M+H⁺) 856.3594, found 856.3562.

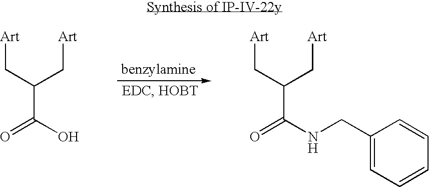

To a solution of bis-trioxane acid (15 mg, 0.024 mmol) in anhydrous dichloromethane (1.0 mL) was added 1-(3-(dimethylamino)propyl)-3-ethylcarbodiimide hydrochloride (EDC, 18 mg, 0.094 mmol, 4.0 equiv) and hydroxy benzotriazole (HOBt, 3.5 mg, 0.026 mmol, 1.1 equiv). A further 0.5 mL of anhydrous dichloromethane was added to wash down the flask walls, then the reaction mixture was treated with benzyl amine (0.010 mL, 0.094 mmol, 4.0 equiv) and triethylamine (0.013 mL, 0.094 mmol, 4.0 equiv). It was stirred at room temperature for 18 hours, at which time TLC analysis showed full consumption of starting material. 1% Hydrochloric acid (5 mL) and methylene chloride (10 mL) were added and organics were extracted with methylene chloride (3×20 mL), dried (MgSO₄) and concentrated in vacuo to give a sticky solid. Flash column chromatography on silica eluting with 30% ethyl acetate/hexanes isolated IP-IV-22y as a white solid (14 mg, 82%): [α]_{D}^{23.1} 110 (CHCl₃, c=0.43); mp=75-78° C.; IR (thin film) 2938, 2874, 1671, 1522, 1453, 1376, 1187, 1093, 1052, 1012, 941, 878, 826, 732, 700 cm⁻¹; ¹H NMR (CDCl₃, 400 MHz) δ 7.40-7.20 (m, 5H), 6.27 (t, br, J=5.6 Hz, 1H), 5.28 (s, 1H), 5.22 (s, 1H), 4.45 (d, br, J=5.2 Hz, 2H), 4.15-4.05 (m, 2H), 2.80-2.64 (m, 2H), 2.61-2.54 (m, 1H), 2.38-2.14 (m, 3H), 2.05-1.96 (m, 2H), 1.85-1.16 (m, 25H, including singlets at 1.38 and 1.27), 1.00-0.81 (m, 14H, including apparent triplet at 0.94 with J=5.6 Hz and two doublets at 0.86 with J=7.6 Hz and 0.83 with J=7.6 Hz); ¹³C NMR (CDCl₃, 100 MHz) δ 175.73, 138.40, 128.46, 128.12, 127.14, 103.43, 103.35, 88.58, 88.33, 81.19, 81.04, 76.41, 73.88, 52.57, 52.38, 44.73, 44.55, 44.39, 44.05, 37.42, 37.17, 36.53, 36.49, 34.50, 34.46, 33.13, 32.83, 30.17, 29.95, 26.20, 26.04, 24.85, 24.77, 24.64, 24.51, 20.20, 13.56, 13.06; HRMS (ESI) m/z for C₄₁H₅₉NO₉Na requires 732.4082, found 732.4080.

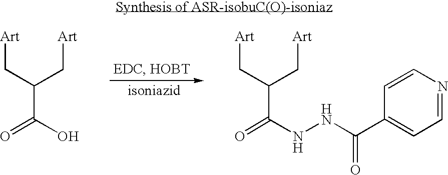

Bis-trioxane acid (100 mg, 0.16 mmol), N-(3-dimethylaminopropyl)-N′-ethylcarbodiimide hydrochloride (EDC, 37 mg, 0.19 mmol) and 1-hydroxybenzotriazole (HOBt, 26 mg, 0.19 mmol) were added to dichloromethane (7 mL) under argon. After stirring at room temperature for 3 h, isoniazid (44 mg, 0.32 mmol) and triethylamine (90 μL, 0.64 mmol) were added. The reaction was allowed to stir at room temperature overnight at which point the colorless solution turned pale yellow. The reaction was quenched with 1% HCl (5 mL). The organic layer was extracted with dichloromethane (3×20 mL). The organic layer was dried with MgSO₄, filtered, and concentrated in vacuo. The crude product was purified by flash silica gel column chromatography (100% EtOAc) to yield ASR-isobuC(O)-isoniaz as a white solid (70 mg, 0.094 mmol, 59%): [α]_{D}^{22.0}+680 (c=0.30, CHCl₃); mp=146-149° C.; IR (thin film) 3519, 3230, 2940, 2875, 1668, 1453, 1378, 1252, 1187, 1125, 1095, 1032, 1013, 940, 877, 826, 733 cm⁻¹; ¹HNMR (400 MHz, acetone-d₆) δ 8.77-8.75 (m, 2H), 7.80-7.78 (m, 2H), 5.38 (s, 1H), 5.33 (s, 1H), 4.23-4.20 (m, 1H), 4.15-4.10 (m, 1H), 2.68-2.55 (m, 2H), 2.29-2.09 (m, 3H), 1.93-1.76 (m, 7H), 1.71-1.60 (m, 4H), 1.59-1.35 (m, 10H), 1.32 (s, 3H), 1.28 (s, 3H), 1.23-1.14 (m, 4H), 0.97-0.93 (m, 9H), 0.89-0.85 (m, 8H); ¹³C NMR (100 MHz, CDCl₃) δ 174.5, 162.4, 150.0, 121.5, 103.7, 103.4, 88.9, 88.4, 81.1, 80.9, 76.5, 73.7, 52.4, 52.3, 44.7, 44.4, 43.1, 37.5, 37.2, 36.4, 34.4, 33.2, 32.6, 30.1, 30.0, 29.8, 26.0, 24.8, 24.6, 20.2, 13.5, 12.9; HRMS (FAB) m/z calc'd for C₄₀H₅₈N₃O₁₀ (M+H)⁺ 740.4122, found 740.4121; HPLC [Dynamax semi-preparative silica gel column (1×25 cm)], 5% MeOH in CH₂Cl₂, 2 mL/min, 270 nm, t_{R}=13.4 min.

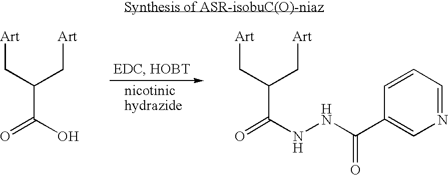

Bis-trioxane acid (100 mg, 0.16 mmol), N-(3-dimethylaminopropyl)-N′-ethylcarbodiimide hydrochloride (EDC, 37 mg, 0.19 mmol) and 1-hydroxybenzotriazole (HOBt, 26 mg, 0.19 mmol) were added to dichloromethane (7 mL) under argon. After stirring at 0° C. for 2 h, nicotinic hydrazide (33 mg, 0.32 mmol) and triethylamine (90 μL, 0.64 mmol) were added. The reaction was allowed to warm to room temperature and stirred overnight. The reaction was quenched with 1% HCl (5 mL). The combined organic layer was extracted with dichloromethane (3×20 mL). The organic layer was dried with MgSO₄, filtered, and concentrated in vacuo. The crude product was purified by flash silica gel column chromatography (5% MeOH in CH₂Cl₂) to yield ASR-isobuC(O)-niaz as a white solid (73 mg, 61%): [α]_{D}²²³+600 (c=0.10, CHCl₃); mp=140-142° C.; IR (thin film) 3263, 3053, 2939, 2875, 1668, 1592, 1454, 1378, 1265, 1188, 1125, 1053, 1013, 958, 878, 734, 704 cm⁻¹; ¹H NMR (400 MHz, (CD₃)₂CO) δ 9.09 (s, 1H), 8.74-8.73 (d, J=3.6 Hz, 1H), 8.25-8.23 (d, J=8.0 Hz, 1H), 7.52-7.49 (m, 1H), 5.37 (s, 1H), 5.33 (s, 1H), 4.23-4.19 (m, 1H), 4.14-4.10 (m, 1H), 2.66-2.57 (m, 2H), 2.27-2.11 (m, 4H), 1.89-1.77 (m, 7H), 1.70-1.62 (m, 4H), 1.55-1.33 (m, 10H), 1.31 (s, 3H), 1.28 (s, 3H), 1.23-1.17 (m, 3H), 1.16-0.82 (m, 16H); ¹³C NMR (100 MHz, (CD₃)₂CO) δ 175.7, 164.9, 153.2, 149.3, 135.8, 129.6, 124.2, 103.7, 103.5, 88.3, 88.2, 81.5, 81.4, 74.8, 74.3, 53.3, 53.3, 45.5, 45.4, 41.6, 37.8, 37.7, 37.3, 37.2, 35.2, 33.8, 31.7, 31.1, 31.0, 26.2, 26.2, 25.4, 25.2, 20.4, 20.4, 14.4, 13.4, 13.3; HRMS (FAB) m/z calc'd for C₄₀H₅₈N₃O₁₀ (M+H)⁺ 740.4122, found 740.4147; HPLC [Dynamax semi-preparative silica gel column (1×25 cm)], 5% MeOH in CH₂Cl₂, 2 mL/min, 270 nm, t_{R}=12.4 min.

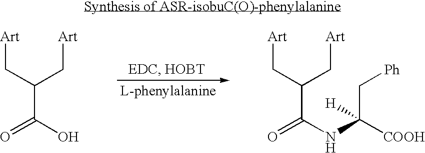

Bis-trioxane acid (100 mg, 0.16 mmol), N-(3-dimethylaminopropyl)-N′-ethylcarbodiimide hydrochloride (EDC, 37 mg, 0.19 mmol) and 1-hydroxybenzotriazole (HOBt, 26 mg, 0.19 mmol) were added to dichloromethane (7 mL) under argon. After stirring at room temperature for 3 h, L-phenylalanine (53 mg, 0.32 mmol) and triethylamine (90 μL, 0.64 mmol) were added. The reaction was allowed to stir at room temperature overnight. The reaction was quenched with 1% HCl (5 mL). The organic layer was extracted with dichloromethane (3×20 mL). The organic layer was dried with MgSO₄, filtered, and concentrated in vacuo. The crude product was purified by flash silica gel column chromatography (50% EtOAc in hexanes with 1% acetic acid) to yield ASR-isobuC(O)-phenylalanine as a white solid (47 mg, 50%): [α]_{D}²²⁰+86° (c=0.90, CHCl₃); mp=102-105° C.; IR (thin film) 3362, 2939, 2875, 1732, 1668, 1521, 1455, 1378, 1188, 1093, 1052, 1014, 912, 878, 733 cm⁻¹; ¹H NMR (400 MHz, CDCl₃) δ 7.35-7.28 (m, 4H), 7.23-7.20 (m, 1H), 6.56 (d, J=8.0 Hz, 1H), 5.29 (s, 1H), 5.24 (s, 1H), 4.88 (m, 1H), 4.01 (m, 2H), 3.31-3.20 (m, 2H), 2.77-2.67 (m, 2H), 2.50 (m, 1H), 2.34-2.27 (m, 2H), 2.13-1.96 (m, 4H), 1.88-1.19 (m, 29H), 0.95 (t, J=5.2 Hz, 6H), 0.87-0.85 (d, J=7.6 Hz, 3H), 0.82-0.80 (d, J=7.6 Hz, 3H); ¹³C NMR (100 MHz, CDCl₃) δ 175.8, 173.1, 136.8, 129.7, 128.4, 126.5, 104.0, 103.5, 88.4, 88.3, 81.1, 80.8, 76.1, 75.2, 53.3, 52.6, 52.5, 44.8, 44.6, 44.5, 37.4, 37.3, 37.1, 36.5, 36.4, 34.5, 34.4, 32.6, 32.4, 30.2, 29.8, 26.0, 25.6, 24.8, 24.7, 24.5, 20.2, 13.7, 13.2; HRMS (FAB) m/z calc'd for C₄₃H₆₂NO₁₁ (M+H)⁺ 768.4323, found 768.4313.

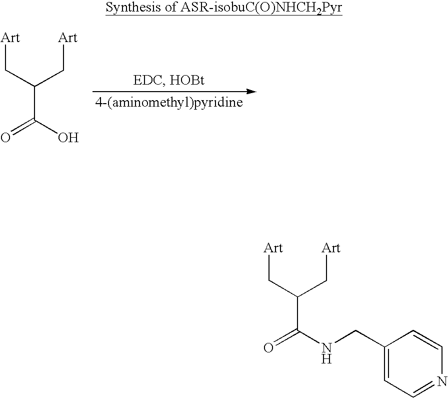

Bis-trioxane acid (50 mg, 0.08 mmol), N-(3-dimethylaminopropyl)-N′-ethylcarbodiimide hydrochloride (EDC, 19 mg, 0.10 μmol) and 1-hydroxybenzotriazole (HOBt, 13 mg, 0.10 mmol) were added to dichloromethane (4 mL) under argon. After stirring at 0° C. for 2 h, 4-(aminomethyl)pyridine (17 μL, 0.16 mmol) and triethylamine (45 μL, 0.32 mmol) were added. The reaction was allowed to stir at room temperature for 30 min. The reaction was quenched with 1% HCl (5 mL). The organic layer was extracted with dichloromethane (3×10 mL). The organic layer was dried with MgSO₄, filtered, and concentrated in vacuo. The crude product was purified by flash silica gel column chromatography (100% EtOAc) to yield ASR-isobuC(O)NHCH₂Pyr as a white solid (31 mg, 54%): [α]_{D}^{22.0} +1000 (c=0.05, CHCl₃); mp=100-110° C.; IR (thin film) 3311, 2938, 2875, 1669, 1603, 1530, 1453, 1417, 1377, 1253, 1187, 1093, 1052, 1012, 878, 734 cm⁻¹; ¹H NMR (400 MHz, CDCl₃) δ 8.63-8.62 (d, J=6.0 Hz, 2H), 8.00-7.99 (d, J=6.4 Hz, 2H), 6.74 (t, J=5.6 Hz, 1H), 5.32 (s, 1H), 5.20 (s, 1H), 4.92-4.87 (m, 1H), 4.51-4.45 (m, 1H), 4.27-4.23 (m, 1H), 4.11-4.05 (m, 1H), 2.72-2.61 (m, 3H), 2.38-2.30 (m, 3H), 2.40-2.17 (m, 1H), 2.04-1.23 (m, 26H), 0.97 (m, 8H), 0.90-0.82 (m, 6H); ¹³C NMR (100 MHz, CDCl₃) δ 176.3, 149.8, 147.9, 103.4, 88.7, 88.6, 81.2, 81.1, 73.5, 52.5, 52.4, 44.6, 44.5, 44.4, 42.9, 37.5, 37.3, 36.5, 34.5, 33.6, 33.0, 30.2, 29.9, 26.2, 26.2, 24.9, 24.8, 24.7, 24.6, 20.2, 13.5, 13.0; HRMS (FAB) m/z calc'd for C₄₀H₅₈N₂O₉ (M+H)⁺ 711.4221, found 711.4245; HPLC [Dynamax semi-preparative silica gel column (1×25 cm)], 5% MeOH in CH₂Cl₂, 2 mL/min, 270 nm, t_{R}=11.7 min).

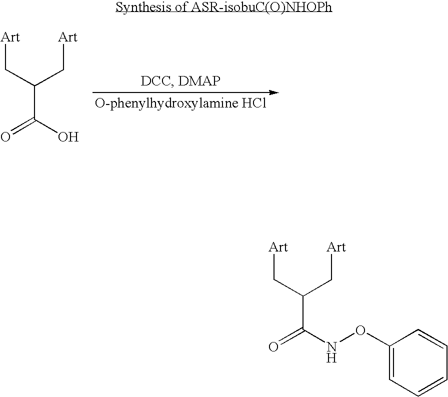

Bis-trioxane acid (77 mg, 0.12 mmol), N,N′-dicyclohexylcarbodiimde (DCC, 31 mg, 0.15 mmol), 4-(dimethylamino)pyridine (DMAP, 1 mg), and O-phenylhydroxylamine HCl (22 mg, 0.15 mmol) were added to dichloromethane (7 mL) under argon and stirred at room temperature for 18 h. The reaction was quenched with distilled water (10 mL). The organic layer was washed with brine. The aqueous layer was extracted with CH₂Cl₂ (3×20 mL). The organic layer was dried with MgSO₄, filtered, and concentrated in vacuo. The crude product was purified by flash silica gel column chromatography (30% EtOAc in hexanes) to yield ASR-isobuC(O)NHOPh as an amorphous solid (52 mg, 59%): [α]_{D}^{22.0} +130° (c=0.30, CHCl₃); IR (thin film) 3219, 2939, 2875, 1704, 1592, 1489, 1455, 1377, 1187, 1155, 1097, 1053, 1011, 941, 878, 753, 734, 691 cm⁻¹; ¹HNMR (400 MHz, CDCl₃) δ 9.26 (s, 1H), 7.29-7.25 (m, 2H), 7.20-7.15 (m, 2H), 7.00-6.97 (m, 1H), 5.36 (s, 1H), 5.33 (s, 1H), 4.45-4.35 (m, 1H), 4.30-4.25 (m, 1H), 2.70-2.62 (m, 3H), 2.36-2.26 (m, 2H), 2.21-2.13 (m, 1H), 2.04-1.85 (m, 6H), 1.82-1.71 (m, 3H), 1.69-1.51 (m, 6H), 1.48-1.20 (m, 16H including singlets at 1.41 and 1.36), 0.97-0.94 (m, 8H), 0.88-0.84 (t, J=7.6 Hz, 7H); ¹³C NMR (100 MHz, CDCl₃) δ 176.9, 159.8, 129.3, 122.3, 113.6, 103.3, 103.1, 89.7, 89.3, 81.2, 80.9, 74.5, 72.1, 52.2, 52.1, 44.4, 44.0, 40.6, 37.5, 37.3, 36.7, 36.6, 34.4, 33.8, 33.4, 30.5, 29.9, 26.1, 26.0, 24.8, 24.8, 24.8, 24.6, 20.1, 20.1, 12.8, 12.7; HRMS (FAB) m/z calc'd for C₄₀H₅₇NO₁₀ (M+H)⁺ 712.4061, found 712.4059; HPLC [Dynamax semi-preparative silica gel column (1×25 cm)], 30% EtOAc in hexanes, 2 mL/min, 264 nm, t_{R}=16.7 min.

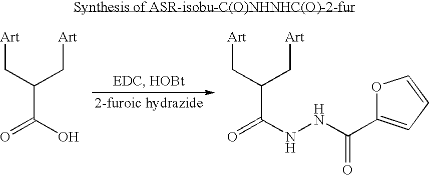

Bis-trioxane acid (100 mg, 0.16 mmol), N-(3-dimethylaminopropyl)-N′-ethylcarbodiimide hydrochloride (EDC, 37 mg, 0.19 mmol) and 1-hydroxybenzotriazole (HOBt, 26 mg, 0.19 mmol) were added to dichloromethane (7 mL) under argon. After stirring at 0° C. for 2 h, 2-fluoric hydrazide (33 mg, 0.32 mmol) and triethylamine (90 μL, 0.64 mmol) were added. The reaction was allowed to warn to room temperature and stirred overnight. The reaction was quenched with 1% HCl (5 mL). The organic layer was extracted with dichloromethane (3×20 mL). The combined organic layer was dried with MgSO₄, filtered, and concentrated in vacuo. The crude product was purified by flash silica gel column chromatography (50% EtOAc in hexanes) to yield ASR-isobu-C(O)NHNHC(O)-2-fur as a white solid (82 mg, 70%): [α]_{D}^{23.3}+87° (c=0.07, CHCl₃); MP=142-143° C.; IR (thin film) 3272, 2940, 1669, 1592, 1453, 1377, 1093, 1052, 1011, 878, 734 cm⁻¹; ¹H NMR (400 MHz, CDCl₃) δ 8.67 (s, 1H), 8.12 (s, 1H), 7.45 (s, 1H), 7.17-7.16 (d, J=3.2 Hz, 1H), 6.51 (m, 1H), 5.31 (s, 1H), 5.20 (s, 1H), 4.24-4.16 (m, 2H), 2.79-2.68 (m, 2H), 2.65-2.60 (m, 1H), 2.36-2.29 (m, 2H), 2.24-2.15 (m, 1H), 2.04-1.99 (m, 2H), 1.92-1.20 (m, 25H), 0.95 (t, 8H), 0.86 (t, 6H); ¹³C NMR (100 MHz, CDCl₃) δ 174.3, 155.5, 146.4, 144.2, 115.5, 112.1, 103.7, 103.4, 88.9, 88.3, 81.2, 80.9, 76.4, 73.8, 52.5, 52.3, 44.7, 44.4, 43.9, 43.3, 37.5, 37.2, 36.6, 36.5, 34.5, 33.0, 32.6, 30.1, 26.1, 25.9, 24.8, 24.7, 24.5, 20.2, 13.4, 12.9; HRMS (FAB) m/z calc'd for C₃₉H₅₆N₂O₁₁ (M+H)⁺ 729.3962, found 729.3954; HPLC [Dynamax semi-preparative silica gel column (1×25 cm)], 50% EtOAc in hexanes, 2 mL/min, 264 nm, t_{R}=41.4 min.

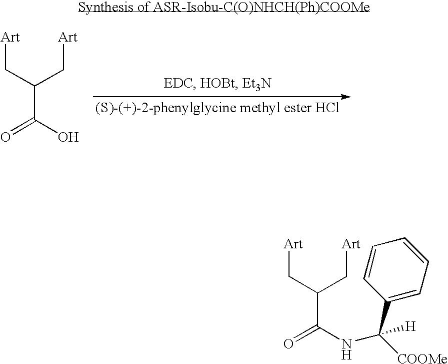

Bis-trioxane acid dimer (50 mg, 0.08 mmol), N-(3-dimethylaminopropyl)-N′-ethylcarbodiimide hydrochloride (EDC, 19 mg, 0.10 mmol) and 1-hydroxybenzotriazole (HOBt, 13 mg, 0.10 mmol) were added to dichloromethane (4 mL) under argon. After stirring at room temperature for 2 h, (S)-(+)-2-phenylglycine methyl ester hydrochloride (65 mg, 0.32 mmol) and triethylamine (45 μL, 0.32 mmol) were added. The reaction was allowed to stir at room temperature overnight. The reaction was quenched with 1% HCl (10 mL). The organic layer was extracted with dichloromethane (3×10 mL). The organic layer was dried with MgSO₄, filtered, and concentrated in vacuo. The crude product was purified by flash silica gel column chromatography (30% EtOAc in hexanes) to yield ASR-Isobu-C(O)NHCH(Ph)COOMe as a white solid (25 mg, 40%): [α]_{D}^{21.4}+150° (c 0.10, CHCl₃); mp=78-80° C.; IR (thin film) 3351, 2950, 1748, 1673, 1507, 1455, 1377, 1197, 1093, 1051, 1013, 879, 733 cm⁻¹; ¹H NMR (400 MHz, CDCl₃) δ 7.44-7.42 (m, 2H), 7.34-7.28 (m, 3H), 6.75-6.74 (d, J=5.6 Hz, 1H), 5.42-5.40 (d, J=5.6 Hz, 1H) 5.28 (s, 1H), 5.19 (s, 1H), 4.34-4.32 (m, 1H), 4.11-4.07 (m, 1H), 3.70 (s, 3H), 2.83-2.77 (m, 1H), 2.74-2.67 (m, 1H), 2.66-2.59 (m, 1H), 2.37-2.25 (m, 2H), 2.20-2.10 (m, 1H), 2.03-1.95 (m, 2H), 1.91-1.16 (m, 25H), 1.00-0.90 (m, including t at 0.94 with J=6.4 Hz, 8H), 0.84 (m, 6H); ¹³C NMR (100 MHz, CDCl₃) δ 175.6, 171.1, 136.1, 128.8, 128.5, 127.8, 103.5, 103.4, 88.4, 88.3, 81.1, 81.1, 76.1, 74.3, 57.7, 52.6, 52.4, 44.8, 43.5, 37.4, 37.2, 36.6, 36.5, 34.6, 32.7, 32.6, 30.0, 29.9, 26.2, 26.1, 24.9, 24.8, 24.7, 24.6, 20.3, 20.2, 13.5, 13.2; HRMS (FAB) m/z calc'd for C₄₃H₆₂NO₁₁ (M+H)⁺ 768.4323, found 768.4378; HPLC [Dynamax semi-preparative silica gel column (1×25 cm)], 30% EtOAc in hexanes, 2 mL/min, 264 nm, t_{R}=23.4 min.

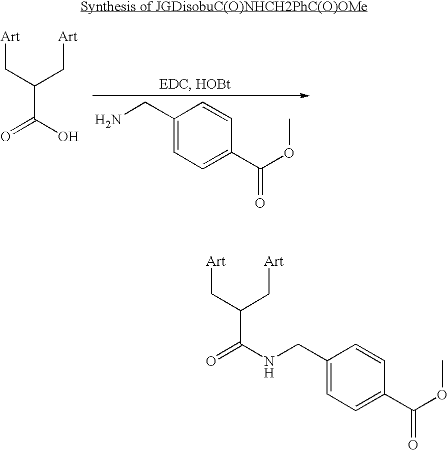

Bis-trioxane acid (50 mg, 0.08 mmol), were loaded together with a stir-bar in a 15 mL round bottom flask. The flask was charged with CH₂Cl₂ (5 mL), N-(3-dimethylaminopropyl)-N′ethylene carbodiimide hydrochloride (EDC, 61 mg, 0.32 mmol), and 1-hydroxybenzotriazole hydrate (HOBt, 12 mg, 0.09 mmol). After 90 minutes, methyl 4-(aminomethyl)benzoate hydrochloride (65 mg, 0.32 mmol) and Et₃N (44 μL, 0.39 mmol). The reaction was stirred overnight and then quenched by the addition of 1 N HCl (5 mL). The layers were separated and the aqueous layer was extracted with CH₂Cl₂ (3×50 mL). The combined organic layers were washed with brine, dried with magnesium sulfate, filtered and concentrated. The product was purified by flash gradient column chromatography (silica gel, 3:2, then 3:1 ether:petroleum ether) to give JGDisobuC(O)NHCH₂PhC(O)OMe as an amorphous solid (47 mg, 78%): IR (thin film) 2950, 2360, 1722, 1672, 1279, 1106, 1052, and 1012 cm⁻¹; ¹H NMR (CDCl₃): δ 7.95-7.93 (d, J=8.1 Hz, 2H), 7.41-7.39 (d, J=8.1 Hz, 2H), 6.41-6.39 (t, J=4.8 Hz, 1H), 5.26 (s, 1H), 5.15 (s, 1H), 4.49-4.47 (d, J=5.3 Hz, 2H), 4.15-4.06 (m, 3H), 3.87 (s, 3H), 2.74-2.57 (m, 3H), 2.31-2.114 (m, 5H), 2.01-1.16 (m, 24H), 0.93-0.82 (m, 14H); ¹³C NMR (CDCl₃): δ 176.1, 166.9, 144, 129.8, 129, 127.9, 103.4, 103.3, 88.7, 88.5, 81.2, 81.1, 76.3, 73.7, 52.5, 52.4, 52.0, 44.6, 44.5, 44.2, 43.7, 37.4, 37.2, 36.5, 34.5, 33.3, 32.98, 30.2, 29.96, 26.2, 26.1, 24.9, 24:8, 24.7, 24.5, 20.2, 13.5, 13.0; HRMS calculated for C₄₃H₆₂NO₁₁⁺768.4323, observed 768.4349.

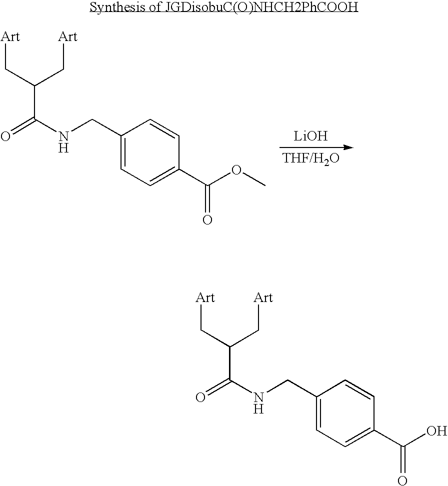

JGDisobuC(O)NHCH₂PhC(O)OMe (89 mg, 0.12 mmol) was placed in a 100 mL round bottom flask with water (7 mL) and THF (3 mL). To the stirred reaction mixture, LiOH.H₂O (500 mg, 12.00 mmol) was added. After 5 days more water (5 mL) was added. Two days later, the starting material finally disappeared and the reaction was acidified by the addition of 1 N HCl (20 mL). Dichloromethane (50 mL) and brine (20 mL) were then added and the layers were separated. The aqueous layer was extracted with CH₂Cl₂ (2×50 mL). The combined organic layers were washed with brine, dried with magnesium sulfate, filtered and concentrated. The residue was purified by flash column chromatography (4% MeOH in CH₂Cl₂) to give JGDisobuC(O)NHCH₂PhCOOH as a 110 white solid (64 mg, 70%): mp=118-124° C.; IR (thin film): 3348, 2940, 1712, 1654, 1613 cm⁻¹; ¹HNMR (CDCl₃) δ 7.66-7.65 (d, J=8.1 Hz, 2H), 7.31-7.29 (d, J=8.1 Hz, 2H), 6.89-6.87 (t, J=4.2 Hz, 1H), 5.39 (s, 1H), 5.35 (s, 1H), 4.45-4.44 (d, J=4.3 Hz, 2H), 4.30-4.18 (m, 2H), 2.76 (m, 3H), 2.38-2.32 (m, 3H), 2.04-1.20 (m, 27H), 1.01-0.81 (m, 14H); ¹³C NMR (CDCl₃) δ 176.3, 168.3, 142.9, 130.1, 128.7, 128.3, 103.9, 103.8, 88.6, 88.5, 81.3, 81.1, 75.0, 52.8, 52.5, 44.9, 44.6, 37.4, 37.0, 36.6, 36.5, 34.6, 34.5, 33.3, 32.7, 30.2, 30.1, 26.1, 26, 24.8, 24.4, 20.3, 20.2, 13.8, 13.2; HRMS (FAB) calculated for C₄₂H₆₀NO₁₁⁺754.4166, observed 754.4188.

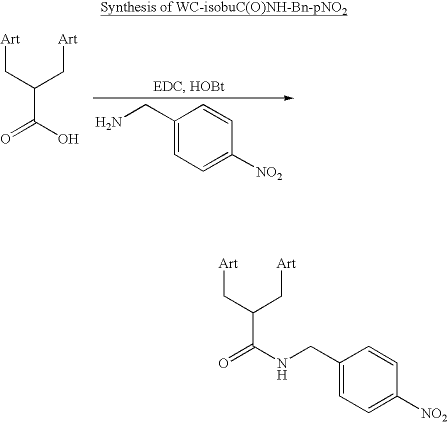

To a solution of bis-trioxane acid (71 mg, 0.12 mmol) in CH₂Cl₂ (1 mL) were added N-(3-dimethylamino-propyl)-N′-ethylcarbodiimide hydrochloride (EDC, 26 mg, 0.14 mmol) and 1-hydroxybenzotriazole (HOBt, 19 mg, 0.14 mmol), and it was stirred for 1 h at rt. To the reaction was added a solution of 4-nitrobenzylamine hydrochloride (43 mg, 0.23 mmol) and N,N-diisopropylethylamine (70 μL, 0.40 mmol) in CH₂Cl₂ (1 mL) dropwise and the solution was stirred for 16 h. It was quenched with water (2 mL). Layers were separated and aqueous layer was extracted with EtOAc (3×2 mL). The combined organic solution was dried (MgSO₄) and concentrated. The residue was purified by flash column chromatography (elution with EtOAc:hexanes=1:2) to give WC-isobuC(O)NH-Bn-pNO₂ (81 mg, 94%) as a white solid: [α]_{D}²⁴=+87 (c 0.93, CHCl₃); mp 97-99° C.; IR (thin film) 3312, 2939, 1670, 1520, 1345, 1052, 1012, 735 cm⁻¹; ¹H NMR (400 MHz, CDCl₃) δ 8.15 (m, 2H), 7.53 (m, 2H), 6.53 (bs, 1H), 5.29 (s, 1H), 5.18 (s, 1H), 4.60 (d, J=15.2 Hz, 1H), 4.49 (d, J=14.8 Hz, 1H), 4.15 (m, 1H), 4.09 (m, 1H), 2.73 (m, 1H), 2.63 (dm, J=7.8 Hz, 2H), 2.31 (t, J=13.2 Hz, 2H), 2.17 (m, 1H), 2.04-1.17 (m, 27H including at 1.38 and 1.29), 0.98-0.78 (m, 14H); ¹³C NMR (100 MHz, CDCl₃) δ 176.3, 147.2, 146.5, 128.5, 123.7, 103.4, 102.9, 100.8, 88.7, 88.5, 81.2, 81.1, 76.3, 73.5, 52.4, 52.3, 44.6, 44.4, 44.1, 43.3, 37.4, 37.3, 36.5, 36.5, 34.4, 33.5, 33.0, 30.2, 29.9, 26.2, 26.1, 24.9, 24.8, 24.6, 24.5, 20.2, 20.2, 13.4, 13.0; HRMS (FAB) calculated for C₄₁H₅₉N₂O₁₁ [(M+H)⁺] 755.4119, found 755.4156.

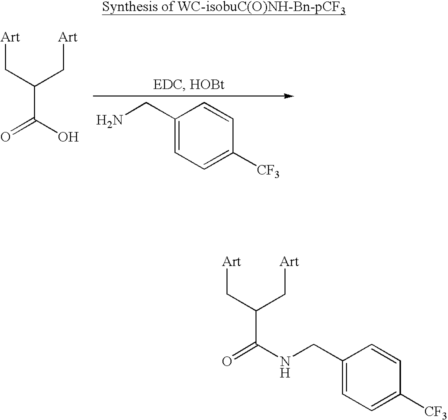

To a solution of bis-trioxane acid (64 mg, 0.10 mmol) in CH₂Cl₂ (1 mL) were added N-(3-dimethylamino-propyl)-NA-ethylcarbodiimide hydrochloride (EDC, 24 mg, 0.12 mmol) and 1-hydroxybenzotriazole (HOBt, 17 mg, 0.12 mmol) and it was stirred for 1 h at rt. To the reaction were added 4-(trifluoromethyl)benzylamine (30 μL, 0.21 mmol) and triethylamine (29 μL, 0.21 mmol) and the solution was stirred for 16 h. It was quenched with water (2 mL). Layers were separated and aqueous layer was extracted with EtOAc (3×2 mL). The combined organic solution was dried (MgSO₄) and concentrated. The residue was purified by flash column chromatography (elution with EtOAc:hexanes=1:5) to provide WC-isobuC(O)NH-Bn-pCF₃ (71 mg, 87%) as a white solid: [α]_{D}²⁴=+110 (c 0.99, CHCl₃); mp 105-108° C.; IR (thin film) 2924, 2875, 1654, 1378, 1326, 1065, 754 cm⁻¹; ¹H NMR (400 MHz, CDCl₃) δ 7.55 (d, J=8.0 Hz, 2H), 7.47 (d, J=8.4 Hz, 2H), 6.35 (t, J=6.0 Hz, 1H), 5.27 (s, 1H), 5.18 (s, 1H), 4.53 (dd, J=15.2, 5.6 Hz, 1H), 4.47 (dd, J=5.6, 14.8 Hz, 1H), 4.11 (m, 2H), 2.73 (dq, J=13.2, 6.0 Hz, 1H), 2.66 (dq, J=13.2, 6.4 Hz, 1H), 2.58 (octet, J=3.6 Hz, 1H), 2.31 (dt, J=4.0, 14.0 Hz, 2H), 2.18 (m, 1H), 2.00 (t, J=3.6 Hz, 1H), 1.96 (t, J=3.2 Hz, 1H), 1.92-1.17 (m, 25H including s at 1.36 and 1.25), 0.98-0.80 (m, 14H including d at 0.96 with J=7.2 Hz, 0.95 with J=6.4 Hz, 0.85 with J=7.6 Hz, and 0.82 with J=7.6 Hz); ¹³C NMR (100 MHz, CDCl₃) δ 176.1, 143.0, 142.8, 128.3, 125.4, 125.4, 125.3, 125.3, 103.3, 103.3, 100.8, 100.8, 88.7, 88.5, 81.1, 76.3, 73.6, 52.5, 52.4, 44.6, 44.5, 44.3, 43.5, 37.4, 37.2, 36.5, 36.5, 34.5, 33.4, 33.0, 30.2, 29.9, 26.2, 26.0, 24.9, 24.8, 24.7, 24.5, 20.2, 20.1, 13.4, 13.0; ¹⁹F NMR (282 MHz, CDCl₃) δ −63.1; HRMS (FAB) calculated for C₄₂H₅₉F₃NO₉ [(M+H)⁺] 778.4142, found 778.4095.

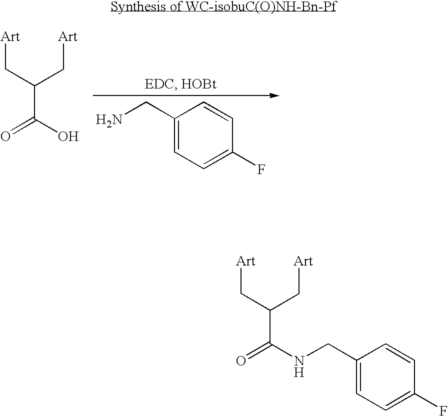

To a solution of bis-trioxane acid (100 mg, 0.16 mmol) in CH₂Cl₂ (1 mL) were added N-(3-dimethylamino-propyl)-N-ethylcarbodiimide hydrochloride (EDC, 37 mg, 0.19 mmol) and 1-hydroxybenzotriazole (HOBt, 26 mg, 0.19 mmol) and it was stirred for 1 h at rt. To the reaction were added 4-fluorobenzylamine (37 μL, 0.33 mmol) and triethylamine (45 μL, 0.32 mmol) and the solution was stirred for 16 h. It was quenched with water (2 mL). Layers were separated and aqueous layer was extracted with EtOAc (3×2 mL). The combined organic solution was dried (MgSO₄) and concentrated. The residue was purified by flash column chromatography (elution with EtOAc:hexanes=1:3) to provide WC-isobuC(O)NH-Bn-pF (99 mg, 84%) as a white solid: [α]_{D}²⁴=+82.1 (c 1.55, CHCl₃); mp 110-115° C.; IR (thin film) 3312, 2939, 1669, 1510, 1377, 1221, 1052, 1012, 735 cm⁻¹; ¹H NMR (400 MHz, CDCl₃) δ 7.32 (m, 2H), 6.97 (m, 2H), 6.23 (t, J=5.6 Hz, 1H), 5.27 (s, 1H), 5.20 (s, 1H), 4.41 (s, 1H), 4.39 (s, 1H), 4.09 (m, 2H), 2.76 (dq, J=13.2, 7.2 Hz, 1H), 2.66 (dq, J=13.6, 6.4 Hz, 1H), 2.54 (octet, J=4.0 Hz, 1H), 2.31 (m, 2H), 2.18 (m, 1H), 2.01-1.95 (m, 3H), 1.92-1.18 (m, 24H including s at 1.35 and 1.26), 0.98-0.79 (m, 14H including d at 0.95 with J=5.6 Hz, 0.93 with J=6.0 Hz, 0.85 with J=7.6 Hz, and 0.82 with J=7.2 Hz); ¹³C NMR (100 MHz, CDCl₃) δ 175.8, 160.8, 134.3, 129.8, 129.7, 115.3, 115.1, 103.4, 102.9, 100.8, 88.6, 88.4, 81.2, 81.1, 76.4, 73.7, 52.5, 52.4, 44.7, 44.5, 44.3, 43.3, 37.4, 37.2, 36.5, 34.5, 33.3, 32.9, 30.2, 29.9, 30.2, 29.9, 26.2, 26.0, 24.9, 24.8, 24.6, 24.5, 20.2, 13.5, 13.0; ¹⁹F NMR (282 MHz, CDCl₃) 6-115.7; HRMS (FAB) calculated for C₄₁H₅₉FNO₉ [(M+H)⁺] 728.4174, found 728.4177.

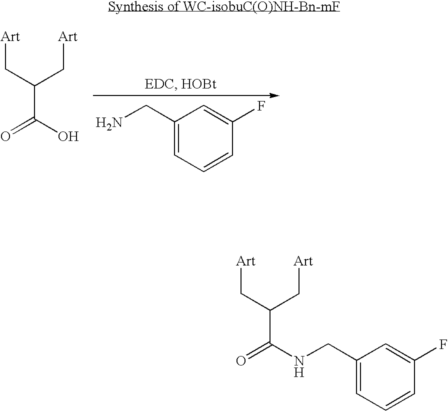

To a solution of bis-trioxane acid (106 mg, 0.17 mmol) in CH₂Cl₂ (1 mL) were added N-(3-dimethylamino-propyl)-N′-ethylcarbodiimide hydrochloride (EDC, 39 mg, 0.21 mmol) and 1-hydroxybenzotriazole (HOBt, 28 mg, 0.21 mmol) and it was stirred for 1 h at rt. To the reaction were added 3-fluorobenzylamine (39 μL 0.34 mmol) and triethylamine (48 μL, 0.34 mmol) and the solution was stirred for 16 h. It was quenched with water (2 mL). Layers were separated and aqueous layer was extracted with EtOAc (3×2 mL). The combined organic solution was dried (MgSO₄) and concentrated. The residue was purified by flash column chromatography (elution with EtOAc:hexanes=1:2) to provide WC-isobuC(O)NH-Bn-mF (93 mg, 75%) as a white solid: [α]_{D}²⁴=+90.2 (c 1.16, CHCl₃); mp 113-115° C.; IR (thin film) 3312, 2952, 1669, 1451, 1377, 1127, 1053, 1013, 735 cm⁻¹; ¹H NMR (400 MHz, CDCl₃) δ 7.24 (m, 1H), 7.12 (m, 1H), 7.06 (m, 1H), 6.91 (dt, J=2.0, 8.4 Hz, 1H), 6.32 (t, J=4.9 Hz, 1H), 5.27 (s, 1H), 5.21 (s, 1H), 4.44 (s, 1H), 4.43 (s, 1H), 4.12 (m, 2H), 2.73 (dq, J=13.6, 6.4 Hz, 1H), 2.67 (dq, J=13.6, 6.4 Hz, 1H), 2.57 (octet, J=3.6 Hz, 1H), 2.30 (dt, J=4.0, 14.0 Hz, 2H), 2.18 (m, 1H), 2.03-1.17 (m, 27H including s at 1.37 and 1.27), 0.97-0.80 (m, 14H including d at 0.95 with J=6.4 Hz, 0.93 with J=6.0 Hz, 0.85 with J=7.6 Hz, and 0.83 with J=7.6 Hz); ¹³C NMR (100 MHz, CDCl₃) δ 176.0, 164.1, 141.1, 129.9, 123.5, 114.9, 113.9, 103.4, 103.3, 88.6, 88.4, 81.1, 81.0, 76.2, 73.7, 52.5, 52.4, 44.6, 44.5, 44.2, 43.5, 37.4, 37.2, 36.5, 36.5, 34.4, 34.4, 33.2, 32.96, 30.2, 30.0, 26.1, 26.0, 24.9, 24.8, 24.6, 24.5, 20.2, 13.4, 13.0; ¹⁹F NMR (282 MHz, CDCl₃) δ−113.2; HRMS (FAB) calculated for C₄₁H₅₉FNO₉ [(M+H)⁺] 728.4174, found 728.4176.

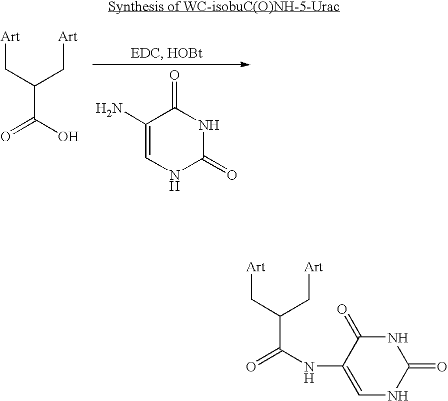

To a solution of bis-trioxane acid (102 mg, 0.16 mmol) in CH₂Cl₂ (1 mL) were added N-(3-dimethylamino-propyl)-N′-ethylcarbodiimide hydrochloride (EDC, 38 mg, 0.20 mmol) and 1-hydroxybenzotriazole (HOBt, 27 mg, 0.20 mmol). The solution was stirred for 2 h at rt. To the reaction was added a solution of 5-aminouracil (31 mg, 0.25 mmol) and N,N-diisopropylethylamine (57 μL, 0.33 mmol) in DMSO (1 mL) dropwise and the mixture was stirred for 12 h. It was diluted with ether (4 mL) and quenched with water (4 mL). Layers were separated and aqueous layer was extracted with ether (4×2 mL). The combined organic solution was dried (MgSO₄) and concentrated. The residue was purified by flash column chromatography (elution with EtOAc:hexanes=3:1) to give WC-isobuC(O)NH-5-Urac (98 mg, 81%) as a white solid: [α]_{D}²⁴=+52.7 (c 1.22, CHCl₃); mp 103-108° C.; IR (thin film) 2937, 1714, 1671, 1436, 1053 cm⁻¹; ¹H NMR (400 MHz, CDCl₃) δ 10.12 (bs, 1H), 9.60 (bs, 1H), 8.59 (bs, 1H), 8.55 (d, J=6.0 Hz, 1H), 5.34 (s, 1H), 5.25 (s, 1H), 4.21 (m, 2H), 2.96 (m, 1H), 2.71 (m, 2H), 2.34-1.16 (m, 29H including s at 1.39 and 1.34), 0.98-0.80 (m, 14H); ¹³C NMR (100 MHz, CDCl₃) δ 179.0, 174.9, 151.8, 150.3, 139.4, 103.5, 103.5, 103.2, 102.9, 88.6, 88.2, 81.2, 81.1, 52.5, 52.2, 44.7, 44.6, 44.3, 37.4, 37.3, 37.3, 37.2, 34.6, 34.5, 30.2, 30.0, 30.0, 26.1, 26.0, 25.8, 24.8, 24.7, 24.6, 20.2, 20.2, 20.1, 13.3, 13.1; HRMS (FAB) calculated for C₃₈H₅₅N₃O₁₁, [(M+H)⁺] 730.3915, found 730.3923.

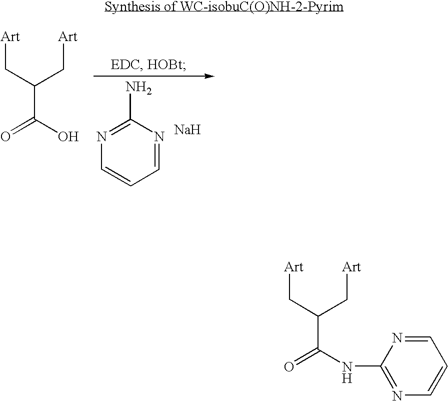

To a solution of bis-trioxane (95 mg, 0.15 mmol) in DMF (1 mL) were added N-(3-dimethylamino-propyl)-N′-ethylcarbodiimide hydrochloride (EDC, 35 mg, 0.18 mmol) and 1-hydroxybenzotriazole (HOBt, 25 mg, 0.18 mmol). The solution was stirred for 1 h at rt. To a solution of 2-aminopyrimidine (22 mg, 0.23 mmol) in DMF (1 mL) at −20° C. was added sodium hydride (60% dispersion in mineral oil, 31 mg, 0.77 mmol), and it was warmed to 0° C. and stirred for 20 min. The heterogeneous mixture was cooled down to −20° C. To the mixture was added the previously prepared benzotriazolyl ester solution dropwise. The reaction was warmed to 0° C. and stirred for 30 min. It was diluted with ether (5 mL) and quenched with water (5 mL). Layers were separated and aqueous layer was extracted with ether (3×3 mL). The combined organic solution was washed with 0.1 N aq. citric acid (2 mL) and brine (2 mL), dried (MgSO₄) and concentrated. The residue was purified by flash column chromatography (elution with EtOAc:hexanes=2:1) to afford WC-isobuC(O)NH-2-Pyrim (77 mg, 72%) as a white solid: [α]_{D}²⁴=+83.3 (c 2.24, CHCl₃); mp 108-110° C.; IR (thin film) 2939, 2875, 1716, 1677, 1626, 1579, 1435, 1096, 1054, 1011, 733 cm⁻¹; ¹H NMR (400 MHz, CDCl₃) δ 8.91 (bs, 1H), 8.60 (d, J=5.2 Hz, 2H), 6.94 (t, J=4.8 Hz, 1H), 5.30 (s, 1H), 5.18 (s, 1H), 4.22 (m, 2H), 2.97 (m, 1H), 2.67 (septet, J=7.2 Hz, 2H), 2.33-2.10 (m, 4H), 2.01-1.14 (m, 26H including s at 1.29 and 1.24), 0.97-0.80 (m, 14H including d at 0.92 with J=6.0 Hz, 0.90 with J=6.4 Hz, 0.84 with J=5.2 Hz, and 0.83 with J=4.8 Hz); ¹³C NMR (100 MHz, CDCl₃) δ 178.4, 174.6, 158.1, 157.4, 116.1, 103.1, 103.0, 88.6, 88.5, 81.1, 80.9, 74.8, 73.4, 52.3, 52.3, 44.5, 44.5, 44.4, 44.3, 37.3, 37.2, 36.5, 36.4, 34.4, 33.1, 32.2, 30.1, 30.0, 26.0, 26.0, 24.8, 24.7, 24.6, 24.5, 20.1, 20.9, 13.1, 12.9; HRMS (FAB) calculated for C₃H₅₆N₃O₉ [(M+H)⁺] 698.4017, found 698.4038.

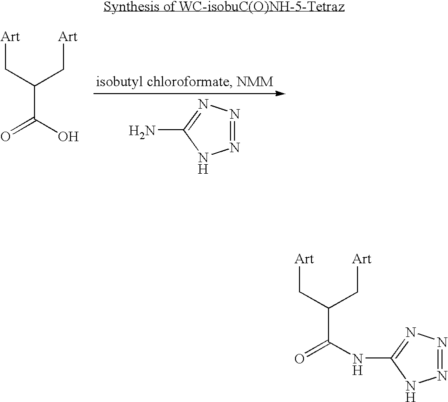

To a solution of bis-trioxane acid (144 mg, 0.23 mmol) in THF (2 mL) at −15° C. was added N-methylmorpholine (NMM, 51 μL, 0.46 mmol) and isobutyl chloroformate (36 μL, 0.28 mmol). After 30 min it was warmed to 0° C. and 5-aminotetrazole (39 mg, 0.46 mmol) in DMF (1 mL) was added to the solution. The reaction was warmed to rt and stirred for 6 h. It was diluted with ether (4 mL) and quenched with water (4 mL). Layers were separated and aqueous layer was extracted with ether (4×3 mL). The combined organic solution was washed with brine, dried (MgSO₄) and concentrated. The residue was purified by flash column chromatography (elution with EtOAc:hexanes=1:1) to give WC-isobuC(O)NH-5-Tetraz (127 mg, 80%) as a white solid: [α]_{D}²⁴=+49.2, (c 2.09, CHCl₃); mp 109-113° C.; IR (thin film) 2922, 1684, 1595, 1378, 1126, 1050, 1011, 754 cm⁻¹; ¹H NMR (400 MHz, CDCl₃) δ 13.51 (bs, 1H), 11.33 (s, 1H), 5.32 (s, 1H), 5.15 (s, 1H), 4.19 (m, 2H), 2.99 (m, 1H), 2.69 (dq, J=14.4, 7.2 Hz, 1H), 2.62 (dq, J=13.6, 6.8 Hz, 1H), 2.22 (m, 3H), 2.00-1.72 (m, 9H), 1.66-1.10 (m, 15H including s at 1.27), 1.08 (s, 3H), 0.98-0.78 (m, 14H including d at 0.92 with J=6.4 Hz, 0.91 with J=6.0 Hz, 0.84 with J=7.6 Hz, and 0.82 with J=8.0 Hz); ¹³C NMR (100 MHz, CDCl₃) δ 176.8, 150.2, 103.5, 103.2, 89.0, 88.0, 80.9, 80.8, 76.2, 72.3, 52.3, 52.1, 45.0, 44.5, 44.2, 37.4, 37.2, 36.5, 36.2, 34.3, 33.2, 32.8, 30.1, 30.0, 25.8, 25.5, 24.8, 24.8, 24.6, 24.5, 20.1, 20.1, 13.3, 12.8; HRMS (FAB) calculated for C₃₅H₅₄N₅O₉ [(M+H)⁺] 688.3922, found 688.3926.

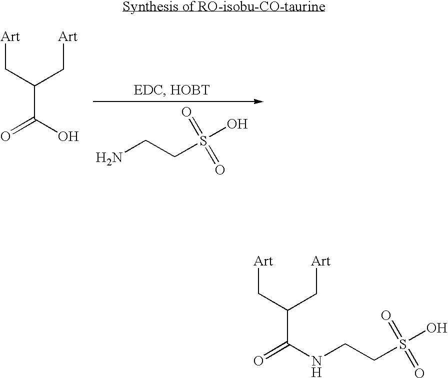

To a solution of bis-trioxane dimer (50 mg, 0.08 mmol) in dried DMF (3 mL) was added N-(3-dimethylamino-propyl)-N′-ethylcarbodiimide hydrochloride (EDC, 16 mg, 0.08 mmol) and 1-hydroxybenzotriazole (HOBt, 22 mg, 0.16 mmol) at 0° C., then stirred 30 min. The reaction mixture was further stirred 24 h at 25° C. Taurine (13 mg, 0.11 mmol) and Et₃N (161 μL, 1.16 mmol) were added and stirred for 24 h. The reaction mixture was diluted with ethyl acetate (30 mL), washed with 1N HCl (3×10 mL) and dried over MgSO₄. After filtration and concentration in vacuo, the residue was purified via flash column chromatography (CH₂Cl₂:MeOH=7:1 to 1:1) to give compound RO-isobu-CO-taurine (15 mg, 27%) as an amorphous solid: R_{f} 0.10 in CH₂Cl₂:MeOH=5:1; IR (thin film) 2988, 2875, 1730, 1410, 1375, 1166, 1086, 941 cm⁻¹; ¹H NMR (400 MHz, CDCl₃) δ 7.45 (bs, 1H), 5.30 (s, 2H), 4.45 (bs, 1H), 4.10 (m, 2H), 3.76 (m, 1H), 3.60 (m, 1H), 3.15 (m, 2H), 2.68 (m, 2H), 2.54 (m, 1H), 2.32 (m, 2H), 2.20 (m, 1H), 2.02 (m, 2H), 1.87-1.65 (m, 8H), 1.52-1.15 (m, 16H), 0.98-0.77 (m, 15H); ¹³C NMR (100 MHz, CDCl₃) δ 178.2, 102.5, 102.3, 89.5, 88.5, 80.5, 80.1, 74.4, 72.2, 56.2, 52.5, 52.0, 44.0, 43.8, 42.0, 37.9, 37.2, 36.6, 36.5, 34.5, 31.6, 31.4, 30.5, 30.3, 26.0, 25.8, 24.7, 24.5, 21.0, 20.4, 13.3, 12.7; HRMS (FAB) calc'd for C₃₆H₅₈NO₁₂S (M+H) 728.3680, found 728.3699.

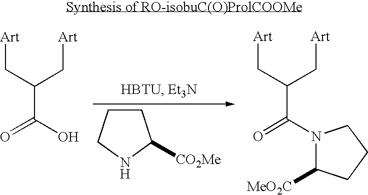

To a solution of bis-tiroxane acid (100 mg, 0.16 mmol) in dried dichloromethane (4 mL) was added O-(Benzotriazol-1-yl)-N,N,N′,N′-tetramethyluronium hexafluorophosphate (HBTU, 73 mg, 0.19 mmol) and triethylamine (57 μL, 0.20 mmol) at 0° C., then stirred 30 min. Proline methyl ester (32 mg, 0.20 mmol) was added and stirred for 12 h. The reaction mixture was diluted with ethyl acetate (30 mL), washed with 0.1 N HCl solution (3×10 mL) and dried over MgSO₄. After filtration and concentration in vacuo, the residue was purified by flash column chromatography (Hexanes:EtOAc=3:1) to give RO-isobuC(O)ProlCOOMe (64 mg, 55%) as a colorless oil: R_{f} 0.40 in Hexanes:EtOAc=2:1; IR (thin film) 2980, 2870, 1755, 1710, 1440, 1370, 1185, 1095, 940 cm⁻¹; ¹H NMR (400 MHz, CDCl₃) δ 5.33 (s, 1H), 5.14 (s, 1H), 4.60 (dd, J=8.0, 7.8 Hz, 1H), 4.07 (m, 2H), 3.82 (m, 2H), 3.67 (s, 3H), 2.95 (t, 1H, J=12.0 Hz), 2.76 (m, 2H), 2.42-2.31 (m, 3H), 2.15 (m, 1H), 2.04-1.71 (m, 8H), 1.68-1.52 (m, 4H), 1.50-1.10 (m, 16H), 0.98-0.77 (m, 16H); ¹³C NMR (100 MHz, CDCl₃) δ 180.5, 173.2, 103.0, 102.9, 88.6, 88.4, 80.4, 80.2, 74.4, 72.6, 59.8, 56.2, 52.1, 51.8, 50.8, 44.3, 43.8, 43.0, 42.1, 37.7, 37.1, 36.4, 35.2, 33.1, 31.4, 31.2, 30.5, 30.3, 26.0, 25.8, 24.6, 24.4, 23.8, 21.7, 21.5, 20.4, 13.0, 12.8; HRMS (FAB) calc'd for C₄₀H₆₂NO₁₁ (M+H) 732.4245, found 732.4240.

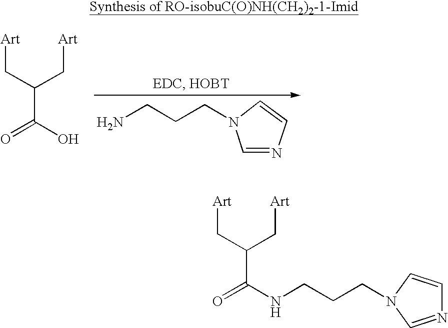

To a solution of bis-trioxane acid (50 mg, 0.08 mmol) in dried dichloromethane 3 mL was added N-(3-dimethylamino-propyl)-1′-ethylcarbodiimide hydrochloride (EDC, 19 mg, 0.10 mmol), triethylamine (57 μL, 0.20 mmol) and 1-hydroxybenzotriazole (HOBt, 13 mg, 0.20 mmol) at 0° C., then stirred 30 min. 3-Aminopropyl imidazole (20 μL, 0.17 mmol) was added to the reaction and stirred for 12 h. The reaction mixture was diluted with ethyl acetate (10 mL), washed with 0.1 N HCl solution (3×5 mL) and dried over MgSO₄. After filtration and concentration in vacuo, the residue was purified by flash column chromatography (CH₂Cl₂:MeOH=20:1) to give RO-isobuC(O)NH(CH₂)₂-1-Imid (35 mg, 60%) as an amorphous: R_{f} 0.30 in CH₂Cl₂:MeOH=10:1; IR (thin film) 2992, 2873, 1735, 1420, 1355, 1145, 1075, 935 cm⁻¹; ¹HNMR (400 MHz, CDCl₃) δ 8.20 (s, 1H), 7.18 (s, 1H), 7.15 (s, 1H), 6.22 (t, J=8.8 Hz, 1H), 5.29 (s, 1H), 5.24 (s, 1H), 4.15 (m, 4H), 3.26 (m, 2H), 2.72 (m, 1H), 2.62 (m, 1H), 2.49 (m, 1H), 2.32-2.11 (m, 3H), 2.04-1.71 (m, 8H), 1.68-1.42 (m, 4H), 1.40-1.15 (m, 16H), 0.98-0.77 (m, 15H); ¹³C NMR (100 MHz, CDCl₃) δ 179.5, 146.1, 130.2, 125.5, 103.1, 102.8, 89.6, 88.7, 80.5, 80.2, 73.4, 72.4, 56.6, 52.3, 51.9, 48.2, 44.3, 43.7, 42.2, 41.2, 37.8, 37.0, 36.5, 36.2, 34.1, 33.1, 31.3, 31.1, 30.7, 30.4, 26.1, 25.9, 24.7, 24.4, 21.1, 20.5, 13.1, 12.5; HRMS (FAB) calc'd for C₄₀H₆₃N₃O₉ (M+H) 727.4408, found 727.4402.

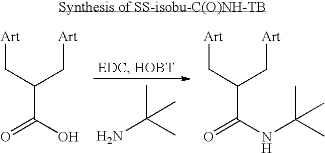

A flame-dried 20 mL recovery flask equipped with a magnetic stir bar, a septum along with an Ar balloon was charged with bis-trioxane acid (50 mg, 0.10 mmol) and dissolved in 2.0 mL freshly distilled CH₂Cl₂. The flask was cooled down to 0° C., and 1-(3-(dimethylamino)propyl)-3-ethylcarbodiimide hydrochloride (EDC, 23 mg, 0.12 mmol, 1.5 eq) and hydroxybenzotriazole (HOBT, 16 mg, 0.12 mmol, 1.5 eq) were added respectively. The mixture was allowed to stir for 2 hr. The tert-butylamine (0.020 mL, 0.19 mmol, 2.5 eq) and triethylamine (0.040 mL, 0.58 mmol) were added to the reaction at 0° C., and it was left stirring overnight warming up to room temperature. The reaction was quenched by addition of 10 mL distilled water and the mixture was placed into a separatory funnel with additional methylene chloride (S mL). The mixture was extracted with methylene chloride (3×30 mL). The combined extracts were washed with water (5 mL), and brine (5 mL), dried over Na₂SO₄ and filtered. The filtrate was concentrated in vacuo to give the crude product that was purified by flash column chromatography, which was eluted with 50% ethyl acetate in hexanes to afford SS-isobu-C(O)NH-TB (58 mg, 88%) as an amorphous solid: [α]²⁵_{D} +85.3 (c 1.00, CHCl₃); IR (thin film) 3404, 2954, 2875, 1668, 1512, 1453, 1377, 1225, 1126, 1094, 1051, 1011, 940.5, 878.2, 753.9 cm⁻¹; ¹H NMR (400 MHz, CDCl₃) δ 5.82 (s, 1H), 5.26 (s, 2H), 4.14-4.10 (m, 1H), 4.09-4.00 (m, 2H), 3.18-3.13 (m, 1H), 2.93-2.95 (m, 1H), 2.77-2.66 (m, 2H), 2.36-2.24 (m, 3H), 2.12-1.94 (m, 3H), 1.89-1.71 (m, 5H), 1.65-1.56 (m, 3H), 1.53-1.41 (m, 5H), 1.39-1.30 (m, 18H, including three singlets at 1.37, 1.35 and 1.32), 0.94-0.90 (m, 8H), 0.84-0.79 (m, 6H); ¹³C NMR (100 MHz, CDCl₃) δ 174.4, 103.4, 103.3, 88.23, 88.22, 81.13, 81.03, 76.55, 74.55, 60.27, 52.57, 52.49, 51.08, 45.23, 44.70, 44.68, 37.33, 37.14, 36.48, 36.39, 34.45, 33.22, 32.39, 30.21, 29.91, 28.64, 26.20, 26.10, 24.74, 24.65, 24.55, 24.50, 20.18, 20.16, 13.59, 13.15; HRMS (FAB) calc'd for C₃₈H₆₁NO₉H⁺ [M+H] 676.4425, found 676.4411.

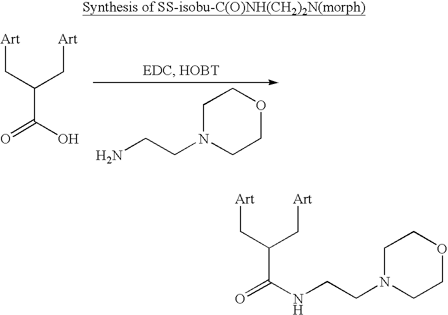

A flame-dried 20 mL recovery flask equipped with a magnetic stir bar, a septum along with an Ar balloon was charged with bis-trioxane acid (50 mg, 0.10 mmol) and it was dissolved in 2.0 mL freshly distilled CH₂Cl₂. Then the flask was cooled down to 0° C., and 1-(3-(dimethylamino)propyl)-3-ethylcarbodiimide hydrochloride (EDC, 23 mg, 0.12 mmol, 1.5 eq) and hydroxybenzotriazole (HOBT, 16 mg, 0.12 mmol, 1.5 eq) were added respectively. The mixture was allowed to stir for 2 hr. The 4-(2-Aminoethyl)morpholine (0.030 mL, 0.19 mmol, 2.0 eq) and Et₃N (0.040 mL, 0.58 mmol) were added to the reaction, at 0° C. and it was left stirring overnight as it warmed up to room temperature. The reaction was quenched by addition of 10 mL distilled water and the mixture was placed into a separatory funnel with additional methylene chloride (5 mL). The mixture was extracted with methylene chloride (3×30 mL). The combined extracts were washed with water (5 mL), and brine (5 mL), dried over Na₂SO₄ and filtered. The filtrate was concentrated in vacuo to give the crude product that was purified by flash column chromatography, which was eluted with 5% methanol in ethyl acetate to afford SS-isobu-C(O)NH(CH₂)₂N(morph) (58 mg, 82%) as an amorphous solid: [α]²⁵_{D} +130 (c 0.40, CHCl₃); IR (thin film) 3589, 3307, 2937, 2861, 1662, 1525, 1532, 1449, 1367, 1140, 1045, 1000, 914.2, 873.1, 726.2 cm⁻¹; ¹H NMR (400 MHz, CDCl₃) δ 6.23 (s, 1H), 5.28 (s, 1H), 5.20 (s, 1H), 4.07-4.03 (m, 2H), 3.69 (s, 4H), 3.39-3.27 (m, 4H), 2.77-2.64 (m, 2H), 2.48-2.45 (m, 3H), 2.35-2.22 (m, 2H), 2.02-1.94 (m, 1H), 1.91-1.73 (m, 6H), 1.68-1.59 (m, 3H), 1.45-1.15 (m, 17H, including two singlets at 1.38, and 1.35), 0.98-0.92 (m, 8H, including d at 0.94 ppm with J=6.4 Hz and d at 0.91 ppm with J=6.4 Hz), 0.84 (d, J=7.6 Hz, 3H), 0.81 (d, J=7.6 Hz, 3H); ¹³C NMR (100 MHz, CDCl₃) δ 175.4, 103.4, 103.3, 88.40, 88.20, 81.09, 81.02, 76.53, 74.45, 66.89, 60.29, 56.96, 52.59, 52.43, 44.76, 44.56, 37.35, 37.09, 36.50, 36.43, 35.92, 34.46, 34.41, 32.83, 32.63, 30.13, 29.94, 26.18, 24.75, 24.69, 24.56, 24.43, 20.97, 20.16, 20.15, 14.13, 13.62, 13.13; HRMS (FAB) calc'd for C₄₀H₆₄N₂O₁₀H⁺[M+H] 733.4639, found 733.4653.

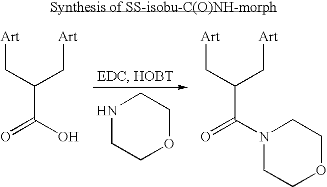

A flame-dried 20 mL recovery flask equipped with a magnetic stir bar, a septum along with an Ar balloon was charged with bis-trioxane acid (50 mg, 0.10 mmol) and it was dissolved in 2.0 mL freshly distilled CH₂Cl₂. Then the flask was cooled down to 0° C., and 1-(3-(dimethylamino)propyl)-3-ethylcarbodiimide hydrochloride (EDC, 23 mg, 0.12 mmol, 1.5 eq) and hydroxybenzotriazole (HOBT, 16 mg, 0.12 mmol, 1.5 eq) were added respectively. The mixture was allowed to stir for 2 hr. The morpholine (0.042 mL, 0.49 mmol, 5.0 eq) was then added to the reaction at 0° C., and it was left stirring overnight as it warmed up to room temperature. The reaction was quenched by addition of 10 mL distilled water and then rinsed into a reparatory funnel with methylene chloride (5 mL). The mixture was extracted with methylene chloride (3×30 mL). The combined extracts were washed with water (5 mL), and brine solution (5 mL), dried over Na₂SO₄ and filtered. The filtrate was concentrated in vacuo to give the crude product that was purified by flash column chromatography, which was eluted with 50% ethyl acetate in hexanes to afford SS-isobu-C(O)NH-morph (48 mg, 72%) as an amorphous solid: [α]²⁵_{D} +124 (c 1.00, CHCl₃); IR (thin film) 2952, 2874, 1630, 1447, 1378, 1240, 1119, 1094, 1050, 1013, 938, 878, 827, 752 cm⁻¹; ¹H NMR (400 MHz, CDCl₃) δ 5.24 (s, 1H), 5.13 (s, 1H), 4.02-3.97 (m, 1H), 3.89-3.72 (m, 4H), 3.69-3.53 (m, 5H), 3.69-3.53 (m, 1H), 3.04-2.99 (m, 1H), 2.74-2.65 (m, 2H), 2.34-2.12 (m, 3H), 2.05-1.93 (m, 3H), 1.87-1.13 (m, 23H, including two singlets at 1.36, and 1.35), 0.93-0.88 (m, 8H, including d at 0.92 with J=6.4 Hz and d at 0.89 with J=6.4 Hz), 0.82 (d, J=7.6 Hz, 3H), 0.78 (d, J=7.6 Hz, 3H); ¹³C NMR (100 MHz, CDCl₃) δ 174.6, 103.5, 103.4, 88.07, 87.84, 81.12, 80.88, 77.21, 74.51, 66.68, 66.45, 52.58, 52.52, 46.60, 44.81, 44.76, 42.35, 37.66, 37.36, 37.08, 36.40, 34.50, 34.46, 33.95, 33.76, 29.91, 29.64, 26.11, 26.07, 24.88, 24.65, 24.57, 24.44, 20.20, 20.13, 13.69, 13.33; HRMS (FAB) calc'd for C₃₈H₅₉NO₁₀H⁺[M+H] 690.4217, found 690.4231.

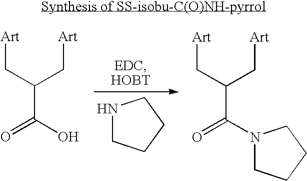

A flame-dried 20 mL recovery flask equipped with a magnetic stir bar, a septum along with an Ar balloon was charged with bis-trioxane acid (50 mg, 0.10 mmol) and it was dissolved in 2.0 mL freshly distilled CH₂Cl₂. Then the flask was cooled down to 0° C., where 1-(3-(dimethylamino)propyl)-3-ethylcarbodiimide hydrochloride (EDC, 23 mg, 0.12 mmol, 1.5 eq) and hydroxybenzotriazole (HOBT, 16 mg, 0.12 mmol, 1.5 eq) were added respectively. The mixture was allowed to stir for 2 hr. The pyrrolidine (0.041 mL, 0.49 mmol, 5.0 eq) was then added to the reaction at 0° C., and it was left stirring overnight as it warmed up to room temperature. The reaction was quenched by addition of 10 mL distilled water and then rinsed into a separatory funnel with methylene chloride (5 mL). The mixture was extracted with methylene chloride (3×30 mL). The combined extracts were washed with water (5 mL), and brine solution (5 mL), dried over Na₂SO₄ and filtered. The filtrate was concentrated in vacuo to give the crude product that was purified by flash column chromatography, which was eluted with 5% methanol in ethyl acetate to afford SS-isobu-C(O)NH-pyrrol (46 mg, 71%) as an amorphous solid: [0t]²⁵_{D} +110 (c 0.90, CHCl₃); IR (thin film) 2947, 2871, 1624, 1445, 1380, 1127, 1093, 1053, 1007 cm⁻¹; ¹H NMR (400 MHz, CDCl₃) δ 5.29 (s, 1H), 5.17 (s, 1H), 4.12-4.02 (m, 1H), 3.95-3.91 (m, 1H), 3.72-3.69 (m, 2H), 3.60-3.54 (m, 1H), 3.34-3.28 (m, 1F), 2.91-2.86 (m, 1H), 2.79-2.64 (m, 2H), 2.36-2.23 (m, 3H), 2.03-1.54 (m, 8H), 2.03-1.58 (m, 8H), 1.51-1.13 (m, 15H, including two singlets at 1.37, and 1.33), 0.94-0.79 (m, 14H); ¹³C NMR (100 MHz, CDCl₃) δ 174.1, 103.4, 103.2, 88.24, 87.71, 81.25, 81.02, 77.21, 74.83, 52.73, 52.52, 46.68, 45.81, 44.96, 44.78, 41.04, 37.41, 37.02, 36.45, 36.43, 34.58, 34.47, 33.72, 33.64, 30.08, 29.93, 26.16, 26.09, 25.85, 24.81, 24.77, 24.62, 24.42, 24.30, 20.22, 20.18, 13.77, 13.34; LRMS (FAB) calc'd for C₃₈H₅NO₁₀H⁺ [M+H] 674.5, found 674.5.

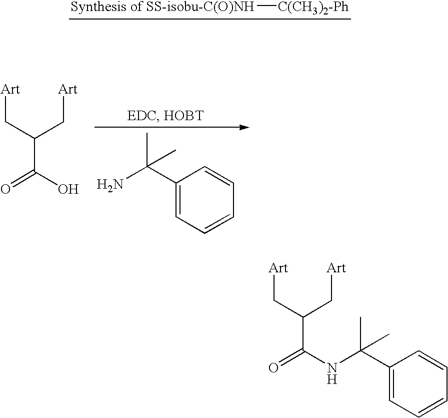

A flame-dried 20 mL recovery flask equipped with a magnetic stir bar, a septum along with an Ar balloon was charged with bis-trioxane acid (50 mg, 0.10 mmol) and it was dissolved in 2.0 mL freshly distilled CH₂Cl₂. Then the flask was cooled down to 0° C., and 1-(3-(dimethylamino)propyl)-3-ethylcarbodiimide hydrochloride (EDC, 23 mg, 0.12 mmol, 1.5 eq) and hydroxybenzotriazole (HOBT, 16 mg, 0.12 mmol, 1.5 eq) were added respectively. The mixture was allowed to stir for 2 hr. The cumylamine (0.071 mL, 0.49 mmol, 5.0 eq) was then added to the reaction at 0° C. and it was left stirring overnight as it warmed up to room temperature. The reaction was quenched by addition of 10 mL distilled water and then rinsed into a separatory funnel with methylene chloride (5 mL). The mixture was extracted with methylene chloride (3×30 mL). The combined extracts were washed with water (5 mL), and brine solution (5 mL), dried over Na₂SO₄ and filtered. The filtrate was concentrated in vacuo to give the crude product that was purified by flash column chromatography, which was eluted with 25% ethyl acetate in hexanes to afford SS-isobu-C(O)NH—C(CH₃)₂-Ph (57 mg, 80%) as an amorphous solid: [α]²⁵_{D} +93.3 (c 1.65, CHCl₃); IR (thin film) 3379, 2940, 2875, 1667; 1509, 1446, 1374, 1102, 1052, 1009, 751.4 cm⁻¹; ¹H NMR (400 MHz, CDCl₃) δ 7.56-7.49 (d, J=17.6 Hz, 2H), 7.31-7.27 (t, J=7.6 Hz, 2H), 7.21-7.16 (m, 1H), 6.36 (s, 1H), 5.30 (s, 1H), 5.25 (s, 1H), 4.14-4.10 (m, 1H), 4.05-4.01 (m, 1H), 2.79-2.63 (m, 2H), 2.51-2.44 (m, 1H), 2.37-2.27 (m, 2H), 2.13-1.83 (m, 10H), 1.80-1.81 (m, 24H, including four singlets at 1.79, 1.70, 1.41 and 1.35), 0.98-0.94 (m, 8H), 0.84 (d, 3H, J=7.2 Hz), 0.75 (d, 3H, J=7.6 Hz); ¹³C NMR (100 MHz, CDCl₃) δ 174.4, 147.4, 128.1, 126.4, 125.2, 103.4, 103.3, 88.43, 81.17, 81.07, 76.29, 74.03, 55.75, 52.57, 52.48, 44.69, 44.67, 44.64, 37.39, 37.25, 36.54, 36.47, 34.50, 33.33, 32.56, 30.23, 29.86, 29.69, 27.40, 26.18, 26.13, 24.82, 24.71, 24.65, 24.60, 20.21, 13.53, 13.03; LRMS (FAB) calc'd for C₄₃H₆₃NO₉H⁺ [M+H] 738.45, found 738.45.

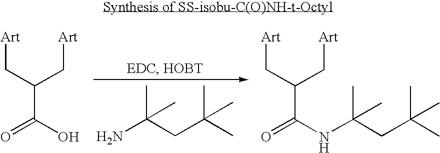

A flame-dried 20 mL recovery flask equipped with a magnetic stir bar, a septum along with an Ar balloon was charged with bis-trioxane acid (50 mg, 0.10 mmol) and it was dissolved in 2.0 mL freshly distilled CH₂Cl₂. Then the flask was cooled down to 0° C., and 1-(3-(dimethylamino)propyl)-3-ethylcarbodiimide hydrochloride (EDC, 23 mg, 0.12 mmol, 1.5 eq) and hydroxybenzotriazole (HOBT, 16 mg, 0.12 mmol, 1.5 eq) were added respectively. The mixture was allowed to stir for 2 hr. The tert-octylamine (0.059 mL, 0.49 mmol, 5.0 eq) was then added to the reaction at 0° C., and it was left stirring overnight as it warmed to room temperature. The reaction was quenched by addition of 10 mL distilled water and then rinsed into a separatory funnel with methylene chloride (5 mL). The mixture was extracted with methylene chloride (3×30 mL). The combined extracts were washed with water (5 mL), and brine (5 mL), dried over Na₂SO₄ and filtered. The filtrate was concentrated in vacuo to give the crude product that was purified by flash column chromatography, which was eluted with 5% methanol in ethyl acetate to afford SS-isobu-C(O)NH-t-Octyl (57 mg, 80%) as a white solid: [α]²⁵_{D} +89 (c 0.90, CHCl₃); IR (thin film) 3392, 2949, 2878, 1662, 1511, 1447, 1376, 1211, 1125, 1097, 1054, 1005, 918.1, 875.6, 725.2 cm⁻¹; ¹H NMR (400 MHz, CDCl₃) δ 5.87 (s, 1H), 5.26 (s, 1H), 4.08-4.01 (m, 2H), 2.79-2.68 (m, 2H), 2.40-2.26 (m, 3H), 2.16-2.26 (m, 1H), 2.03-1.94 (m, 2H), 1.90-1.63 (m, 10H), 1.54-1.39 (m, 24H, including three singlets at 1.45, 1.41 and 1.39), 1.01 (s, 9H), 0.98-0.82 (m, 14H); ¹³C NMR (100 MHz, CDCl₃) δ 174.2, 103.4, 103.3, 88.29, 88.25, 81.17, 81.08, 76.23, 74.70, 55.37, 53.00, 52.64, 52.61, 44.95, 44.80, 37.38, 37.30, 36.56, 36.48, 34.64, 34.55, 33.33, 32.56, 32.26, 31.64, 31.56, 30.18, 29.97, 28.53, 28.15, 26.25, 26.20, 25.25, 24.86, 24.74, 24.60, 20.25, 20.23, 13.62, 13.28; LRMS (FAB) calc'd for C₄₂H₆₉NO₉H⁺ [M+H] 732.49, found 738.49.

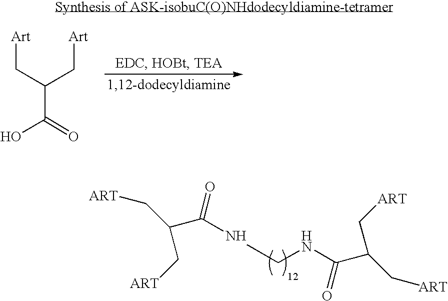

A 25 mL round bottom flask was charged with of bis-trioxane acid (81 mg, 0.13 mmol) in anhydrous dichloromethane (5 mL). 1-(3-(dimethylamino)propyl)-3-ethylcarbodiimide hydrochloride (EDC, 100 mg, 0.52 mmol, 4.0 equiv) and hydroxy benzotriazole (HOBt, 20 mg, 0.14 mmol, 1.1 equiv) were added to the solution. A further 2 mL of anhydrous dichloromethane was added to wash down the flask walls then the reaction mixture was treated with 1,12-dodecyldiamine (13 mg, 0.06 mmol, 0.5 equiv) and triethylamine (73 μL, 0.52 mmol, 4 equiv) and stirred at room temperature for 15 hours, at which time TLC analysis showed full consumption of starting material. Water (10 mL), saturated aqueous sodium bicarbonate solution (10 mL) and methylene chloride (10 mL) were added to the reaction. The organics were extracted with methylene chloride (3×20 mL), dried (MgSO₄) and concentrated in vacuo to give a crude solid. Flash column chromatography on silica eluting with (40% EtOAc in hexanes) yielded ASK-isobudiol-C(O)dodecyldiamine-tetramer as a white solid (74 mg, 83%): [□]_{D}²³=+32 (CHCl₃, C=0.60); mp=91-93° C.; IR (thin film) 3330, 2922, 2860, 1655, 1524, 1446, 1373, 1186, 1112, 1050, 1010, 915, 725 cm⁻¹; ¹H NMR (400 MHz, CDCl₃) δ 5.88 (t, J=4.0 Hz, 2H), 5.27 (s, 2H), 5.22 (s, 2H), 4.11-3.97 (m, 4H), 3.29-3.09 (m, 4H), 2.76-2.59 (m, 4H), 2.55-2.45 (m, 2H), 2.40-2.20 (m, 4H), 3.21 2.16-2.09 (m, 2H), 2.05-1.90 (m, 5H), 1.90-1.70 (m, 11H), 1.68-1.56 (m, 7H), 1.54-1.31 (m, 27), 1.29-1.10, (m, 25H), 0.96-0.80 (m, 27H); ¹³C NMR (100 MHz, CDCl₃) δ 175.5 103.3, 103.3, 88.5, 88.3, 81.2, 81.0, 74.0, 52.5, 52.4, 44.7, 44.5, 44.4, 39.8, 37.4, 37.2, 36.5, 34.5, 32.9, 32.7, 30.2, 30.2, 29.9, 29.6, 29.4, 29.3, 27.1, 26.1, 26.1, 24.8, 24.7, 24.6, 24.5, 20.2, 13.5, 13.0; HRMS (FAB) m/z calc'd for C₈₀H₁₂₉N₂O₁₈ (M+H⁺) 1405.9240, found 1405.9091.

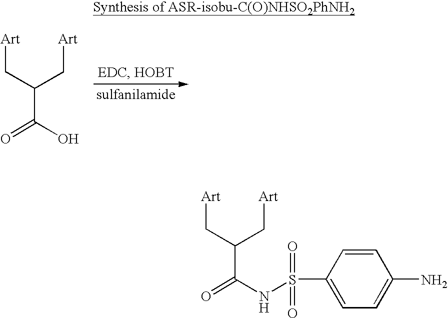

Bis-trioxane acid (50 mg, 0.08 mmol), N-(3-dimethylaminopropyl)-N′-ethylcarbodiimide hydrochloride (EDC, 19 mg, 0.10 mmol) and 1-hydroxybenzotriazole (HOBt, 13 mg, 0.10 mmol) were added to DMF (5 mL) under argon, and stirred at room temperature for 3 h. Sulfanilamide (56 mg, 0.32 mmol) was dissolved in DMF (5 mL) and NaH (8 mg, 0.30 mmol) was added. The amine solution was added via cannula to the reaction at room temperature. It was allowed to stir at room temperature overnight. The reaction was diluted with EtOAc (10 mL) and quenched at 0° C. with cold distilled water (10 mL). The organic layer was extracted with EtOAc (3×10 mL). The organic layer was dried with MgSO₄, filtered, and concentrated in vacuo. The crude product was purified by flash silica gel column chromatography (40% EtOAc in hexanes) to yield ASR-isobu-C(O)NHSO₂PhNH₂ as a yellow solid (26 mg, 41%); [α]_{D}^{23.3} +60 (c=0.08, CHCl₃); mp=125-133° C.; IR (thin film) 3476, 3378, 3246, 2953, 2876, 1714, 1632, 1596, 1504, 1453, 1378, 1343, 1190, 1166, 1089, 1051, 1010, 913, 878, 829, 733, 678 cm⁻¹; ¹H NMR (400 MHz, CDCl₃) δ 9.22 (s, 1H), 7.86-7.84 (d, J=8.4 Hz, 2H), 6.66-6.64 (d, J=8.4 Hz, 2H), 5.23 (s, 1H), 5.15 (s, 1H), 4.31-4.27 (m, 1H), 3.80-3.75 (m, 1H), 2.64-2.49 (m, 3H), 2.35-2.22 (m, 2H), 2.08-1.95 (m, 2H), 1.95-1.80 (m, 3H), 1.78-1.12 (m, 25H), 1.00-0.83 (m, 8H), 0.82-0.68 (m, 6H); ¹³C NMR (100 MHz, CDCl₃) δ 174.6, 151.2, 130.9, 127.1, 113.6, 103.4, 103.0, 100.8, 99.2, 89.6, 88.7, 81.1, 80.9, 73.4, 72.8, 52.2, 52.0, 44.4, 43.8, 43.6, 37.4, 37.3, 36.6, 34.5, 34.3, 32.7, 32.4, 30.3, 29.8, 26.1, 26.0, 24.8, 24.7, 20.1, 20.1, 12.7, 12.4; HRMS (FAB) m/z calc'd for C₄₀H₅₉N₂O₁₁S (M+H)⁺ 775.3840, found 775.3841.

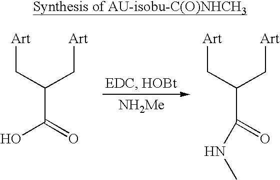

Bis-trioxane acid (39 mg, 0.06 mmol) was dissolved in CH₂Cl₂ (1.5 mL) in an oven-dried 10 ml round bottom flask charged with magnetic stir bar and argon balloon. 1-(3-(dimethylamino)propyl)-3-ethylcarbodiimide hydrochloride (EDC, 18 mg, 0.10 mmol) and 1-hydroxybenzotriazole (HOBt, 13 mg, 0.10 mmol) were added. After 1 hour, TLC showed complete conversion of the isobutyric acid to the HOBt ester. At this time, methyl amine in THF (2.0 M, 0.096 mL, 0.19 mmol) was added and the reaction stirred for 3 hours. The reaction was quenched with saturated aqueous ammonium chloride, and the organics were extracted with methylene chloride (1×10 mL) followed by ethyl acetate (2×10 mL). The organic layer was dried over magnesium sulfate, filtered, and concentrated. Purification of the crude product by column chromatography (50% EtOAc in Hex) gave AU-isobu-C(O)NHCH₃ (28 mg, 72%) as a white solid: [α]_{D}²¹=+96 (c=1.4, CHCl₃); IR (thin film) 3353(w), 2947(s), 2880(m), 1654(s), 1529(m), 1461(m), 1413(w), 1365(m), 1278(w), 1258(w), 1201(m), 1201(m), 1181(m), 1114(m), 1094(s), 1046(s), 988(s), 950(w), 940(m), 921(w), 882(m), 834(w), 815(w), 757(s); ¹H NMR (400 MHz, CDCl₃) δ 6.02 (m, 1H), 5.28 (s, 1H), 5.22 (s, 1H), 4.11-4.07 (m, 2H), 2.78 (d, 3H, J=4.8 Hz), 2.75-2.69 (m, 1H), 2.69-2.62 (m, 1H), 2.58-2.51 (m, 1H), 2.36-2.27 (m, 3H), 2.17-2.08 (m, 1H), 2.04-1.97 (m, 2H), 1.93-1.74 (m, 5H), 1.66-1.52 (m, 4H), 1.51-1.43 (m, 4H), 1.42-1.36 (m including singlets at 1.39 and 1.38, 6H), 1.35-1.18 (m, 5H), 1.01-0.89 (m, 8H), 0.87-0.79 (m, 6H); ¹³C NMR (100 MHz, CDCl₃) δ 176.4, 103.5, 103.3, 88.9, 88.3, 81.2, 81.1, 76.3, 73.2, 52.6, 52.3, 44.7, 44.4, 44.0, 37.5, 37.2, 36.6, 36.5, 34.5, 34.4, 33.0, 32.7, 30.2, 30.0, 26.5, 26.1, 26.0, 24.9, 24.7, 24.6, 24.5, 20.2, 20.1, 13.5, 13.0; HRMS (FAB) m/z calc'd for C₃₅H₅₆NO₉ (M+H)⁺ 634.3955, found 634.3921.

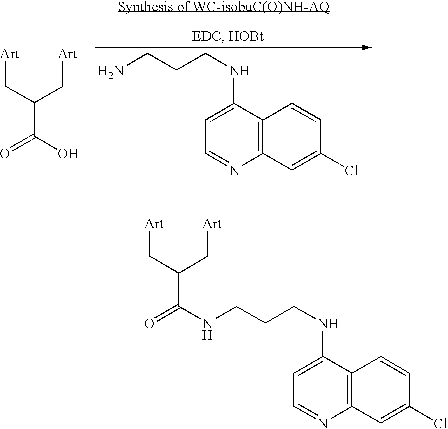

To a solution of bis-trioxane acid (66 mg, 0.11 mmol) in CH₂Cl₂ (2 mL) were added N-(3-dimethylamino-propyl)-N′-ethylcarbodiimide hydrochloride (EDC, 25 mg, 0.13 mmol) and 1-hydroxybenzotriazole (HOBt, 17 mg, 0.13 mmol) and it was stirred for 1 h at rt. To the reaction were added N-(7-Chloro-quinolin-4-yl)-propane-1,3-diamine¹ (38 mg, 0.16 mmol) and triethylamine (30 μL, 0.22 mmol) and the solution was stirred for 16 h. It was quenched with water (2 mL). Layers were separated and the aqueous layer was extracted with EtOAc (3×2 mL). The combined organic solution was dried (MgSO₄) and concentrated. The residue was purified by flash column chromatography (elution with EtOAc only) to provide WC-isobuC(O)NH-AQ (85 mg, 95%) as a white solid: [α]_{D}²⁴=+85 (c 0.81, CHCl₃); Mp 129-133° C.; IR (thin film) 2923, 1651, 1581, 1453, 1376, 1047, 754 cm⁻¹; ¹H NMR (400 MHz, CDCl₃) δ 8.47 (d, J=5.2 Hz, 1H), 8.11 (d, J=9.2 Hz, 1H), 7.92 (d, J=2.4 Hz, 1H), 7.34 (dd, J=8.8, 2.0 Hz, 11H), 6.67 (t, J=4.8 Hz, 1H), 6.35 (m, 2H), 5.29 (s, 1H), 5.28 (s, 1H), 4.13 (d, J=6.0 Hz, 1H), 4.10 (d, J=6.0 Hz, 1H), 3.58-3.41 (m, 2H), 3.39-3.23 (m, 2H), 2.78 (dq, J=12.8, 5.6 Hz, 1H), 2.65 (dq, J=13.6, 6.8 Hz, 1H), 2.49 (m, 2H), 2.36-2.18 (m, 3H), 2.02-1.16 (m, 28H including s at 1.36 and 1-24), 0.97-0.80 (m, 14H including d at 0.95 with J=6.0 Hz, 0.91 with J=6.0 Hz, 0.87 with J=7.6 Hz, and 0.82 with J=7.2 Hz); ¹³C NMR (100 MHz, CDCl₃) δ 176.7, 151.7, 150.4, 149.1, 134.8, 128.1; 125.0, 122.6, 117.7, 103.5, 103.4, 98.4, 88.9, 88.5, 81.2, 81.1, 73.4, 52.5, 52.3, 44.9, 44.7, 44.3, 40.3, 37.5, 37.3, 37.2, 36.5, 36.4, 34.5, 34.4, 34.0, 32.6, 30.6, 30.3, 30.0, 27.8, 26.2, 25.8, 24.9, 24.8, 24.6, 24.4, 20.2, 13.6, 13.0; HRMS (FAB) calculated for C₄₆H₆₅ClN₃O₉ [(M+H)⁺] 838.4409, found 838.4430 (Prepared by the reported procedure from commercially available 4,7-dichloroquinoline and 1,3-diaminopropane: Madrid, P. B.; Wilson, N. T.; DeRisi, J. L.; Guy, R. K. J. Comb. Chem. 2004, 6, 437).

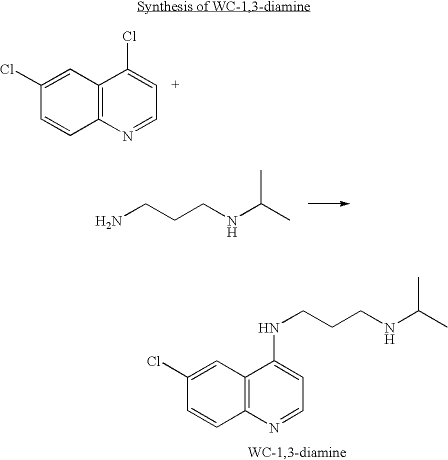

A mixture of 4,7-dichloroquinoline (500 mg, 2.52 mmol) and N-isoproyl-1,3-propanediamine (1.00 g, 8.61 mmol) was heated slowly from rt to 165° C. It was stirred for 6 h under reflux condition. Then the reaction was cooled down to rt and volatile component was removed under reduced pressure (ca. 0.1 mmHg) at 50° C. The residue was suspended in 10% aq. NaOH (40 mL) and it was extracted with CH₂Cl₂ (5×30 mL). The combined organic solution was dried (MgSO₄) and concentrated. The crude solid was dissolved in EtOAc and precipitated by addition of hexanes to give WC-1,3-diamine (475 mg, 68%) as a white solid: IR (thin film) 3227, 2964, 1583, 1366, 1138, 805 cm⁻¹; ¹H NMR (400 MHz, CDCl₃) δ 8.49 (d, J=5.6 Hz, 1H), 7.97 (bs, 1H), 7.92 (d, J=2.4 Hz, 1H), 7.77 (d, J=9.2 Hz, 1H), 7.31 (dd, J=7.2, 2.2 Hz, 1H), 6.29 (d, J=5.6 Hz, 1H), 3.37 (q, J=5.6 Hz, 2H), 2.91 (t, J=5.6 Hz, 2H), 2.84 (septet, J=6.0 Hz, 1H), 1.91 (quintet, J=5.6 Hz, 2H), 1.14 (d, J=6.0 Hz, 6H); ¹³C NMR (100 MHz, CDCl₃) δ 150.2, 150.6, 149.2, 134.6, 128.5, 124.6, 122.3, 117.6, 98.2, 49.2, 47.1, 44.3, 27.9, 23.1; HRMS (FAB) calculated for C₁₅H₂₁C1N₃ [(M+H)⁺] 278.1419, found 278.1421.

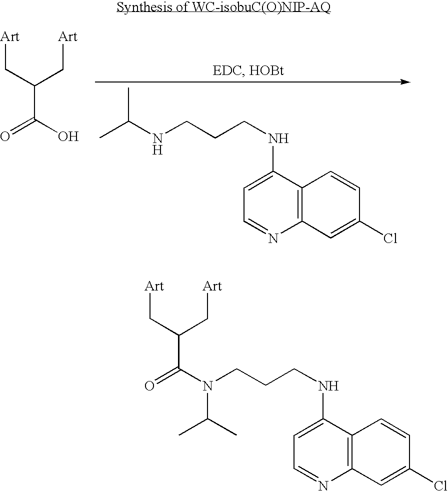

To a solution of bis-trioxane acid (79 mg, 0.13 mmol) in DMF (1 mL) at 0° C. were added N-(3-dimethylamino-propyl)-N′-ethylcarbodiimide hydrochloride (EDC, 29 mg, 0.15 mmol) and 1-hydroxybenzotriazole (HOBt, 21 mg, 0.15 mmol) and it was stirred for 30 min at rt. To the solution were added WC-1,3-diamine (53 mg, 0.19 mmol) and triethylamine (36 μL, 0.26 mmol). The reaction was stirred for 72 h at 50° C. It was diluted with ether (3 mL) and water (3 mL). Layers were separated and the aqueous layer was extracted with ether (3×2 mL). The combined organic solution was washed with water, dried (MgSO₄) and concentrated. The residue was purified by flash column chromatography (elution with EtOAc only) to provide WC-isobuC(O)NIP-AQ (46 mg, 41%) as a white solid: [α]_{D}²⁴=+72 (c 0-50, CHCl₃); mp 116° C.; IR (thin film) 3250, 2923, 1612, 1580, 1450, 1093, 1051, 878 cm⁻¹; ¹H NMR (400 MHz, CDCl₃) δ 8.48 (d, J=5.6 Hz, 1H), 8.06 (d, J=8.8 Hz, 1H), 7.94 (d, J=3.2 Hz, 1H), 7.35 (d, J=8.8 Hz, 1H), 6.59 (t, J=5.2 Hz, 1H), 6.33 (m, 1H), 5.30 (s, 1H), 5.29 (s, 1H), 4.14 (d, J=6.0 Hz, 1H), 4.11 (d, J=6.0 Hz, 1H), 3.94 (m, 1H), 3.59-3.40 (m, 2H), 3.38-3.21 (m, 2H), 2.78 (dq, J=12.4, 6.0 Hz, 1H), 2.63 (dq, J=13.2, 7.2 Hz, 1H), 2.49 (m, 2H), 2.36-2.18 (m, 3H), 2.02-1.16 (m, 34H including s at 1-35 and 1.24, and d at 1.28 with J=6.0 Hz), 0.97-0.80 (m, 14H including d at 0.96 with J=6.0 Hz, 0.90 with J=6.0 Hz, 0.87 with J=7.2 Hz, and 0.85 with J=7.6 Hz); ¹³CNMR (100 MHz, CDCl₃) δ 175.8, 151.8, 151.2, 151.0, 134.8, 128.3, 124.8, 123.2, 117.7, 103.6, 103.3, 97.9, 88.1, 87.7, 81.1, 81.0, 75.2, 72.3, 52.6, 52.5, 48.5, 44.9, 44.7, 41.6, 39.3, 38.3, 37.5, 37.4, 36.4, 36.3, 34.5, 33.8, 33.3, 31.4, 30.1, 29.9, 29.6, 26.1, 25.9, 25.0, 24.9; 24.7, 24.3, 21.9, 21.5, 20.3, 20.1, 13.9, 13.6; HRMS (FAB) calculated for C₄₉H₇₁ClN₃O₉ [(M+H)⁺] 880.4879, found 880.4901.

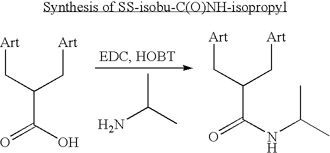

A flame-dried 20 mL recovery flask equipped with a magnetic stir bar, a septum along with an Ar balloon was charged with bis-trioxane acid (50 mg, 0.10 mmol) and it was dissolved in 2.0 mL freshly distilled CH₂Cl₂. Then the flask was cooled down to 0° C., and 1-(3-(dimethylamino)propyl)-3-ethylcarbodiimide hydrochloride (EDC, 23 mg, 0.12 mmol, 1.5 eq) and hydroxybenzotriazole (HOBT, 16 mg, 0.12 mmol, 1.5 eq) were added respectively. The mixture was allowed to stir for 2 hr. The isopropylamine (0.039 mL, 0.49 mmol, 5.0 eq) was then added to the reaction at 0° C., and it was left stirring overnight as it warmed up to room temperature. The reaction was quenched by addition of 10 mL distilled water and then rinsed into a separatory funnel with methylene chloride (5 mL). The mixture was extracted with methylene chloride (3×30 mL). The combined extracts were washed with water (5 mL), and brine solution (5 mL), dried over Na₂SO₄ and filtered. The filtrate was concentrated in vacuo to give the crude product that was purified by flash column chromatography, which was eluted with 5% methanol in ethyl acetate to afford SS-isobu-C(O)NH-isoprop (49 mg, 76%) as an amorphous solid: [α]²⁵_{D} +100 (c 0.45, CHCl₃); IR (thin film) 3313, 2943, 2875, 1647, 1524, 1449, 1373, 1209, 1119, 1092, 1044, 1003, 934.4, 872.7, 827.0 cm⁻¹; ¹H NMR (400 MHz, CDCl₃) δ 5.78 (d, J=7.2 Hz, 1H), 5.27 (s, 1H), 5.23 (s, 1H), 4.08-4.03 (m, 3H), 2.79-2.66 (m, 2H), 2.41-2.23 (m, 3H), 2.20-1.58 (m, 12H), 1.51-1.11 (m, 22H, including two singlets at 1.36, and 1.35), 0.94-0.90 (m, 8H), 0.83 (d, J=7.6 Hz, 3H), 0.81 (d, J=7.6 Hz, 3H); ¹³C NMR (100 MHz, CDCl₃) δ 174.6, 103.4, 88.42, 88.21, 81.19, 81.07, 77.21, 76.78, 74.41, 52.60, 52.44, 44.96, 44.78, 44.60, 41.53, 37.39, 37.15, 36.49, 36.44, 34.50, 34.45, 33.20, 32.54, 30.14, 29.97, 26.23, 26.13, 24.76, 24.70, 24.61, 24.48, 22.80, 22.23, 20.18, 13.63, 13.07; LRMS (FAB) calc'd for C₃₇H₅₉NO₉H⁺ [M+H] 662.42, found 662.42.

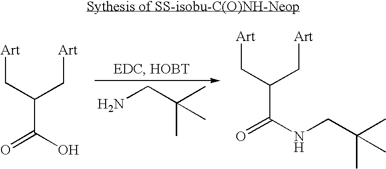

A flame-dried 20 mL recovery flask equipped with a magnetic stir bar, a septum along with an Ar balloon was charged with bis-trioxane acid (50 mg, 0.10 mmol) and it was dissolved in 2.0 mL freshly distilled CH₂Cl₂. Then the flask was cooled down to 0° C., and 1-(3-(dimethylamino)propyl)-3-ethylcarbodiimide hydrochloride (EDC, 23 mg, 0.12 mmol, 1.5 eq) and hydroxybenzotriazole (HOBt, 16 mg, 0.12 mmol, 1.5 eq) were added respectively. The mixture was allowed to stir for 2 hr. The neopentylamine (0.041 mL, 0.49 mmol, 5.0 eq) was then added to the reaction at 0° C., and it was left stirring overnight as it warmed to room temperature. The reaction was quenched by addition of 10 mL distilled water and then rinsed into a separatory funnel with methylene chloride (5 mL). The mixture was extracted with ethyl acetate (3×30 mL). The combined extracts were washed with water (5 mL), and brine (5 mL), dried over Na₂SO₄ and filtered. The filtrate was concentrated in vacuo to give the crude product that was purified by flash column chromatography, which was eluted with 50% ethyl acetate in hexanes to afford SS-isobu-C(O)NH-Neop (52 mg, 77%) as a white solid: [α]²⁵_{D} +110 (c 0.50, CHCl₃); IR (thin film) 3338, 2953, 2870, 1664, 1447, 1380, 1212, 1094, 1011, 935.8, 877.2, 751.6 cm⁻¹; ¹H NMR (400 MHz, CDCl₃) δ 6.11-6.08 (t, J=6.0 Hz, 1H), 5.27 (s, 1H), 5.25 (s, 1H), 4.14-4.10 (m, 1H), 4.07-4.04 (m, 1H), 3.18-3.13 (m, 1H), 2.93-2.95 (m, 1H), 2.78-2.70 (m, 2H), 2.59-2.52 (m, 1H), 2.36-2.26 (m, 2H), 2.18-2.07 (m, 1H), 2.03-1.97 (m, 2H), 1.90-1.71 (m, 6H), 1.68-1.61 (m, 2H), 1.55-1.18 (m, 17H, including two singlets at 1.39 and 1.36), 0.96-0.90 (m, 17H, including a singlet at 0.91), 0.85 (d, J=7.6 Hz, 6H), 0.82 (d, J=7.6 Hz, 6H); ¹³C NMR (100 MHz, CDCl₃) δ 175.7, 103.5, 103.4, 88.4, 88.2, 81.19, 81.02, 76.00, 74.53, 52.58, 52.53, 50.80, 44.83, 44.67, 44.19, 37.35, 37.26, 36.52, 36.49, 34.54, 34.49, 32.43, 31.51, 30.23, 29.77, 27.40, 26.16, 26.10, 24.89, 24.74, 24.60, 24.54, 20.23, 20.19, 13.46, 13.25; HRMS (FAB) calc'd for C₃₉H₆₃NO₉H⁺ [M+H] 690.4581, found 690.4595.

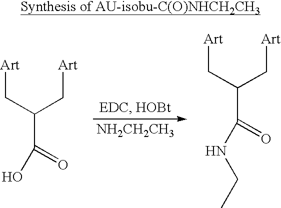

Bis-trioxane acid (35 mg, 0.06 mmol) was dissolved in CH₂Cl₂ (1.5 mL) in an oven-dried 10 ml round bottom flask charged with magnetic stir bar and argon balloon. 1-(3-(dimethylamino)propyl)-3-ethylcarbodiimide hydrochloride (EDC, 13 mg, 0.07 mmol) and 1-hydroxybenzotriazole (HOBt, 10 mg, 0.07 mmol) were added. After 1 hour, TLC showed complete conversion of the bis-trioxane acid to the HOBt ester. At this time, ethyl amine (2.0 M in THF, 87 mL, 0.17 mmol) was added and the reaction stirred for 3 hours at which time TLC showed complete consumption of the HOBt ester. The reaction was quenched with concentrated ammonium chloride, and the organics were extracted with methylene chloride (1×10 mL) followed by ethyl acetate (2×10 mL). The organic layer was dried over magnesium sulfate, filtered, and concentrated. Purification of the crude product by column chromatography (50% EtOAc in Hex) gave AU-isobu-C(O)NHCH₂CH₃ (31 mg, 85%) as an amorphous solid: [α]_{D}²²=+87 (c=0.68, CHCl₃); IR (thin film) 2936(s), 2880(m), 1645(s), 1558(m), 1538(s), 1521(m), 1509(m), 1442(s), 1365(s), 1278(w), 1249(w), 1210(m), 1181(m), 1133(m), 1094(m), 1046(s), 1008(s), 940(m), 872(m), 815(w), 737(s); ¹H NMR (400 MHz, CDCl₃) δ 5.88 (t, J=5.6 Hz, 1H), 5.27 (s, 1H), 5.22 (s, 1H), 4.09-4.05 (m, 2H), 3.36-3.28 (m, 1H), 3.24-3.15 (m, 1H), 2.76-2.70 (m, 1H), 2.70-2.63 (m, 1H), 2.48-2.42 (m, 1H), 2.35-2.26 (m, 2H), 2.17-2.09 (m, 1H), 2.03-1.95 (m, 2H), 1.90-1.83 (m, 1H), 1.83-1.73 (m, 5H), 1.65-1.59 (m, 3H), 1.56-1.40 (m, 5H), 1.38-1.37 (m, including singlets at 1.38 and 1.37, 6H), 1.35-1.18 (m, 6H), 1.13 (t, J=7.2 Hz, 3H), 0.97-0.90 (m, 7H), 0.86-0.80 (m, 6H); ¹³C NMR (100 MHz, CDCl₃) δ 175.5, 103.5, 103.4, 88.7, 88.3, 81.3, 81.1, 76.6, 73.8, 52.6, 52.4, 44.8, 44.5, 44.5, 37.5, 37.2, 36.5, 34.6, 34.5, 33.8, 33.2, 32.7, 30.2, 30.0, 28.9, 26.2, 25.3, 24.9, 24.8, 24.7, 24.6, 20.2, 20.2, 14.5, 13.6, 13.0; HRMS (FAB) calculated for C₃₆H₅₈NO₉ 648.4112, found 648.4123.

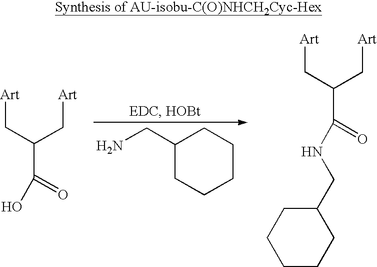

Bis-trioxane acid (35 mg, 0.06 mmol) was dissolved in CH₂Cl₂ (1.5 mL) in an oven dried 10 ml round bottom flask charged with magnetic stir bar and argon balloon. 1-(3-(dimethylamino)propyl)-3-ethylcarbodiimide hydrochloride (EDC, 13 mg, 0.07 mmol) and 1-hydroxybenzotriazole (HOBt, 10 mg, 0.07 mmol) were added. After 1 hour, TLC showed complete conversion of the bis-trioxane acid to the HOBt ester. At this time, cyclohexanemethyl amine (23 mg, 0.17 mmol) was added and the reaction stirred for 3 hours at which time TLC showed complete consumption of the HOBt ester. The reaction was quenched with concentrated ammonium chloride, and the organics were extracted with methylene chloride (1×10 mL) followed by ethyl acetate (2×10 mL). The organic layer was dried over magnesium sulfate, filtered, and concentrated. Purification of the crude product by column chromatography (30% EtOAc in Hex) gave AU-isobu-C(O)NHCH₂Cyc-Hex (39 mg, 93%) as an amorphous solid: [α]_{D}²²=102 (c=1.25, CHCl₃); IR (thin film) 2872(s), 2880(m), 2861(m), 1654(m), 1529(m), 1452(m), 1355(m), 1268(w), 1258(w), 1230(m), 1201(m), 1191(m), 1094(m), 1075(m), 1056(s), 1017(s), 959(w), 930(m), 863(m), 824(w), 815(m), 757(s); ¹H NMR (400 MHz, CDCl₃) δ 6.05 (t, J=5.6 Hz, 1H), 5.25 (s, 1H), 5.22 (s, 1H), 4.09-3.99 (m, 2H), 3.07 (t, J=6.0 Hz, 2H), 2.75-2.65 (m, 2H), 2.55-2.49 (m, 1H), 2.34-2.25 (m, 2H), 2.14-2.04 (m, 1H), 2.03-1.95 (m, 3H), 1.90-1.78 (m, 3H), 1.77-1.59 (m, 11H), 1.54-1.41 (m including singlets at 1.38 and 1.36, 6H), 1.31-1.14 (m, 8H), 0.96-0.90 (m, 9H), 0.85-0.79 (m, 6H); ¹³C NMR (100 MHz, CDCl₃) δ 175.7, 103.44, 88.5, 88.4, 81.2, 81.1, 76.1, 74.3, 52.6, 52.5, 46.0, 45.0, 44.7, 44.1, 37.7, 37.4, 37.3, 36.5, 34.7, 34.5, 32.8, 32.6, 31.6, 31.1, 31.0, 30.2, 29.9, 26.5, 26.3, 26.2, 25.9, 25.9, 25.3, 24.9, 24.8, 24.7, 24.6, 20.2, 14.3, 13.5, 13.2; HRMS (FAB) calculated for C₄₁H₆₅NO₉ 716.4638, found 716.4745.

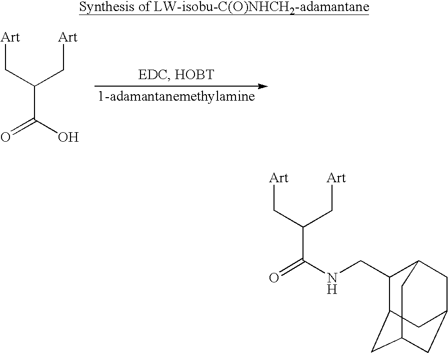

A 10 mL round bottom flask was charged with bis-trioxane acid (50 mg, 0.08 mmol), 1-(3-(dimethylamino)propyl)-3-ethylcarbodiimide hydrochloride (EDC, 62 mg, 0.32 mmol), and 1-hydroxybenzotriazole (HOBT, 12 mg, 0.09 mmol) in CH₂Cl₂ (4 mL). The mixture was then stirred at room temperature for one hour, at which time TLC confirmed the consumption of the dimer acid to form the HOBT ester. 1-Adamantane-methylamine (0.056 mL, 0.32 mmol) was then added to the reaction mixture via plastic syringe and the solution was stirred for an additional three hours. The solution was then washed with 0.1 N citric acid (10 mL), extracted with dichloromethane (3×10 mL), washed with brine, dried (MgSO₄), filtered and concentrated in vacuo. The crude product was purified by flash column chromatography (28% EtOAc in hexanes) to give LW-isobu-C(O)NHCH₂-adamantane as a white solid (0.043 g, 70%): mp=98-100° C.; IR (thin film) 3424, 2907, 2848, 2355, 2237, 2096, 1654, 1537, 1448, 1372 cm⁻¹; ¹H NMR (400 MHz, CDCl₃) δ 6.16 (t, 1H), 5.27 (s, 1H), 5.26 (s, 1H), 4.10 (m, 1H), 4.03 (m, 1H), 2.94 (d, J=6.4 Hz, 2H), 2.72 (m, 2H), 2.60 (m, 1H), 2.37-2.27 (m, 3H), 2.15-1.18 (m, 40H, including singlets at 1.40 and 1.36), 0.96-0.82 (m, 16H, including d at 0.94); ¹³C NMR (100 MHz, CDCl₃) δ 175.85, 103.43, 103.41, 88.36, 88.25, 81.14, 81.00, 75.79, 74.45, 52.50, 52.46, 51.05, 44.74, 44.61, 43.64, 40.20, 37.30, 37.28, 36.89, 36.45, 34.47, 34.44, 33.39, 32.75, 32.09, 30.13, 29.77, 28.21, 26.13, 26.03, 24.87, 24.75, 24.56, 24.52, 20.19, 20.13, 13.43, 13.20; HRMS (FAB) m/z calc'd for C₄₅H₇₀NO₉ (M+H)⁺ 768.5051, found 768.4960.

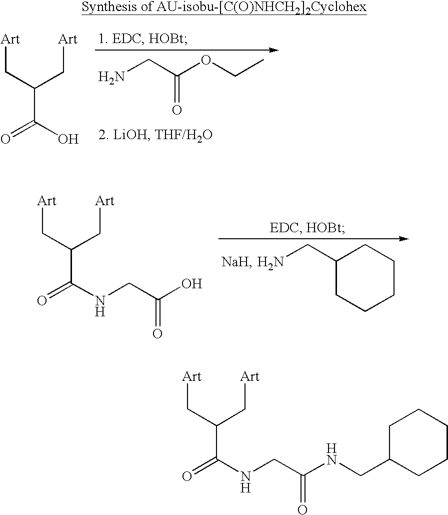

Bis-trioxane acid (40 mg, 0.07 mmol) was dissolved in CH₂Cl₂ (1.5 mL) in an oven-dried 10 ml round bottom flask charged with magnetic stir bar and argon balloon. 1-(3-(dimethylamino)propyl)-3-ethylcarbodiimide hydrochloride (EDC, 19 mg, 0.10 mmol) and 1-hydroxybenzotriazole (HOBt, 13 mg, 0.10 mmol) were added. After 1 hour, TLC showed complete conversion of the bis-trioxane acid to the HOBt ester. At this time, glycine ethyl ester hydrochloride (14 mg, 0.10 mmol) and triethylamine (20 mg, 0.02 mmol) were dissolved in CH₂Cl₂ (1 mL) in an oven-dried 10 mL pear-shaped flask. After stirring for 30 minutes, the solution was added to the reaction mixture via cannula. After 16 hours, the reaction was quenched with concentrated ammonium chloride, and the organics were extracted with methylene chloride (1×10 mL) followed by ethyl acetate (2×10 mL). The organic layer was dried over magnesium sulfate, filtered, and concentrated. Purification of the crude product by column chromatography (50% EtOAc in Hex) gave a crude amide ester.

The crude amide ester was dissolved in H₂O (4.3 mL) and THF (2 mL) in a 25 ml round bottom flask charged with a magnetic stir bar and argon balloon. Lithium hydroxide (LiOH.H₂O, 55 mg, 1.30 mmol) was added and the reaction stirred for 16 hours at which time no starting material remained. The reaction was quenched with concentrated ammonium chloride, and the organics were extracted with ethyl acetate (2×10 mL). The organic layer was dried over magnesium sulfate, filtered, and concentrated. Purification of the crude product by column chromatography (80% EtOAc in Hex) gave a crude amide acid (43 mg, 0.06 mmol) as a clear oil in 87% yield over 2 steps.

The acid (36 mg, 0.05 mmol) was dissolved in CH₂Cl₂ (1.5 mL) in an oven-dried 10 ml round bottom flask charged with magnetic stir bar and argon balloon. 1-(3-(dimethylamino)propyl)-3-ethylcarbodiimide hydrochloride (EDC, 12 mg, 0.06 mmol) and 1-hydroxybenzotriazole (HOBt, 9 mg, 0.06 mmol) were added. After 1 hour, TLC showed complete conversion of the acid to the HOBt ester. At this time, cyclohexanemethyl amine (18 mg, 0.16 mmol) was added and the reaction was stirred for 3 hours at which time TLC showed complete consumption of the HOBt ester. The reaction was quenched with concentrated ammonium chloride, and the organics were extracted with methylene chloride (1×10 mL) followed by ethyl acetate (2×10 mL). The organic layer was dried over magnesium sulfate, filtered, and concentrated. Purification of the crude product by column chromatography (70% EtOAc in Hex) gave AU-isobu-[C(O)NHCH₂]₂Cyclohex (22 mg, 0.030 mmol, 52%) as a white amorphous solid: [α]_{D}²¹=84.7 (c=1.25, CHCl₃); IR (thin film) 3517(w), 3401(m), 3305(m), 3102(w), 2919(s), 2880(m), 2851(m), 1645(s), 1529(m), 1452(m), 1384(m), 1345(w), 1268(w), 1278(w), 1239(m), 1210(m), 1191(m), 1123(m), 1094(m), 1046(s), 1008(s), 950(w), 930(w), 872(m), 843(w), 824(w), 737(s), 660(m); ¹H NMR (400 MHz, CDCl₃) δ 6.97-6.94 (m, 1H), 6.54-6.51 (m, 1H), 5.31 (s, 1H), 5.19 (s, 1H), 4.23-4.03 (m, 2H), 4.08-4.03 (m, 1H), 3.79-3.73 (m, 1H), 3.31-3.24 (m, 1H), 2.95-2.88 (m, 1H), 2.75-2.64 (m, 2H), 2.60-2.76 (m, 1H), 2.31-2.21 (m, 2H), 2.18-2.12 (m, 1H), 2.05-1.99 (m, 4H), 1.91-1.76 (m, 5H), 1.74-1.67 (m, 5H), 1.63-1.55 (m, 5H), 1.52-1.43 (m, 3H), 1.43-1.33 (m including singlets at 1.40 and 1.36, 8H), 1.31-1.12 (m, 7H), 0.99-0.95 (m, 8H), 0.89-0.84 (m, 7H); ¹³CNMR (100 MHz, CDCl₃) δ 176.6, 169.9, 103.5, 103.3, 88.8, 88.7, 81.2, 81.1, 76.0, 73.1, 52.4, 52.3, 45.6, 44.5, 44.4, 44.1, 44.0, 37.7, 37.5, 37.3, 36.5, 34.4, 33.5, 33.1, 31.6, 30.8, 30.7, 30.3, 29.8, 26.5, 26.1, 26.0, 25.9, 25.9, 25.0, 24.8, 24.7, 24.6, 22.7, 20.2, 20.1, 13.2, 12.9; HRMS (FAB) calculated for C₄₃H₆₉N₂O₁₀ 773.4952, found 773.4956.

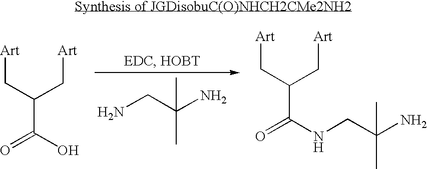

Bis-trioxane acid (100 mg, 0.16 mmol) was placed in a flame dried 25 mL round bottom flask with a stir bar, sealed with a septum and filled with argon. Dichloromethane (10 mL) was added to the flask followed by N-(3-dimethylaminopropyl)-N′ethylene carbodiimide hydrochloride (EDC, 128 mg, 0.64 mmol) and 1-hydroxybenzotriazole hydrate (HOBt, 29 mg, 0.18 mmol). The reaction mixture was stirred for 1.5 hr. Then, 1,2-diamino-2-methylpropane (0.70 mL, 0.64 mmol) was added and the reaction rapidly became cloudy. The reaction was allowed to stir overnight. The reaction was then quenched by the addition of 0.1 N citric acid (10 mL) and the aqueous layer was extracted with dichloromethane (3×50 mL). The combined organics were washed with brine, dried with MgSO₄, filtered and concentrated. The residue was purified by flash column chromatography (silica gel, 92:1.4:7.6=dichloromethane:NH₄OH:methanol) to give JGDisobuC(O)NHCH₂CMe₂NH₂ as an amorphous solid (100 mg, 90%): IR (thin film) 3583, 2955, 1673, 1656, 1055 cm⁻¹; ¹H NMR (DMSO, 400 MHz) δ: 7.75 (m, 1H), 5.36 (s, 1H), 5.28 (s, 1H), 4.05-4.01 (m, 1H), 3.83-3.79 (q, J=3.6 Hz, 1H), 2.98-2.96 (d, J=5.6 Hz, 2H), 2.56-2.45 (m, 3H), 2.19-1.49 (m, 15H), 1.46-1.02 (m, 18H), 0.97-0.74 (m, 20H); ¹³C NMR (DMSO, 75 MHz): 178.9, 105.0, 104.7, 89.9, 89.8, 77.1, 76.2, 54.1, 54.0, 52.4, 51.4, 46.2, 45.1, 38.5, 38.3, 37.5, 35.8, 33.7, 33.0, 31.6, 31.3, 30.7, 30.5, 30.2, 27.8, 27.5, 26.4, 26.3, 25.9, 25.8, 20.7, 13.8; HRMS calculated for C₃₈H₆₃N₂O₉+ 691.4534, found 691.4558.

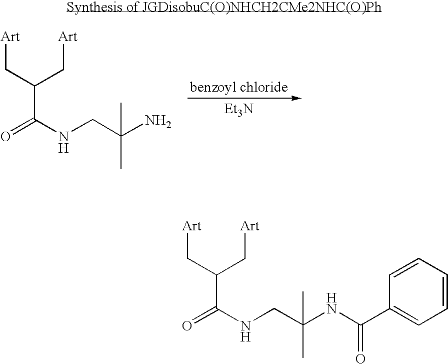

JGDisobuC(O)NHCH₂CMe₂NH₂ (21 mg, 0.03 mmol) was loaded into a 50 mL round bottom flask with a stir-bar. Dichloromethane (10 mL) was added to the flask, followed by triethylamine (30 μL, 0.20 mmol) and benzoyl chloride (15 μL, 0.10 mmol). After stirring for 20 hours, it was quenched by the addition of saturated sodium bicarbonate and extracted with dichloromethane (3×20 mL). The combined extracts were washed with brine, dried with magnesium sulfate, filtered and concentrated. The residue was purified by gradient flash column chromatography (silica gel, 1:1 to 5:1 ether:petroleum ether) to give the JGDisobuC(O)NHCH₂CMe₂NHC(O)Ph as an amorphous solid (22 mg, 100%): IR (thin film) 2923, 1666, 1644, 1380, 1094 cm⁻¹; ¹HNMR (CDCl₃, 300 MHz) δ 8.08 (s, 1H), 7.93-7.90 (m, 2H), 7.42-7.35 (m, 3H), 6.73-6.69 (t, J=6 Hz, 1H), 5.28 (s, 1H), 5.10 (s, 1H), 4.18-4.12 (m, 1H), 4.05-4.00 (m, 1H), 3.48-3.41 (m, 1H), 3.33-3.26 (m, 1H), 2.71-2.61 (m, 3H), 2.39-0.62 (m, 51H); ¹³C NMR (CDCl₃, 75 MHz) δ 178.6, 166.6, 135.2, 130.8, 128.3, 127.1, 103.5, 103.3, 88.6, 81.1, 81.0, 76.6, 75.9, 73.9, 55.9, 52.4, 52.3, 52.3, 44.5, 44.4, 37.4, 37.1, 36.5, 34.5, 33.3, 32.9, 30.3, 30.2, 29.8, 29.7, 26.2, 26.1, 24.6, 24.3, 24.0, 23.4, 20.2, 20.2, 13.3; HRMS (FAB) calculated for C₄₅H₆₇N₂O₁₀+795.4796, found 795.4814.

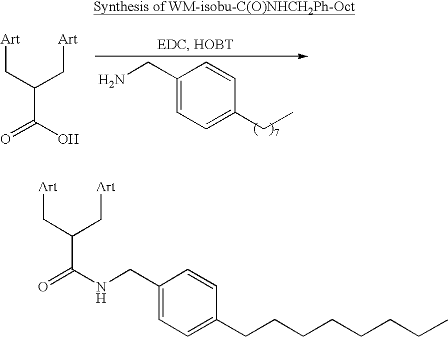

An oven dried 15 mL round bottom flask was charged with bis-trioxane acid (0.050 g, 0.08 mmol), 1-ethyl-3-(3-dimethylaminopropyl)-carbodiimide (EDC, 0.062 g, 0.32 mmol), N-hydroxybenzotriazole (HOBT, 0.012 g, 0.09 mmol) and dissolved in 6 mL of anhydrous CH₂Cl₂. This solution was allowed to stir for 2 hr before p-octylbenzyl amine (0.071 g, 0.32 mmol) in 1 mL of THF was added dropwise over the course of about 2 min. After stirring for 18 hr the reaction was quenched by the slow addition of H₂O (5 mL). The contents of the flask were extracted with ether (2×25 mL), washed with a saturated aqueous solution of NaHCO₃ and H₂O, dried over MgSO₄, and concentrated in vacuo. The crude product was purified by silica gel chromatography (20% ethyl acetate in hexanes) to give C(O)NHCH₂Ph-Oct as an amorphous solid (0.065 g, 98%): [□]_{D}²³=108 (c=1.65, CHCl₃); IR (thin film) 2925, 2854, 1655, 1514, 1452, 1376, 1052, 754 cm⁻¹; ¹H NMR (400 MHz, CDCl₃) δ 7.25-7.22 (m, 2H), 7.10-7.07 (m, 2H), 6.22 (t, J=14.0 Hz, 1H), 5.26 (s, 1H), 5.21 (s, 1H), 4.40 (s, 1H), 4.38 (s, 1H), 4.13-4.05 (m, 2H), 2.76-2.68 (m, 2H), 2.57-2.52 (m, 3H), 2.35-1.15 (m, 38H), 0.96-0.81 (m, 21H); ¹³C NMR (100 MHz, CDCl₃) δ 175.6, 141.8, 135.5, 128.4, 128.1, 103.4, 103.3, 88.5, 88.3, 81.1, 80.9, 76.3, 73.9, 52.5, 52.3, 44.7, 44.5, 44.3, 43.8, 37.4, 37.1, 36.5, 35.6, 34.6, 34.5, 34.4, 32.9, 32.8, 31.8, 31.5, 30.1, 29.9, 29.4, 29.23, 29.20, 26.2, 25.9, 25.2, 24.8, 24.7, 24.6, 24.5, 22.6, 20.6, 20.18, 20.16, 14.1; HRMS (FAB) calculated for C₄₉H₇₆NO₉+ 822.5520, found 822.5512.

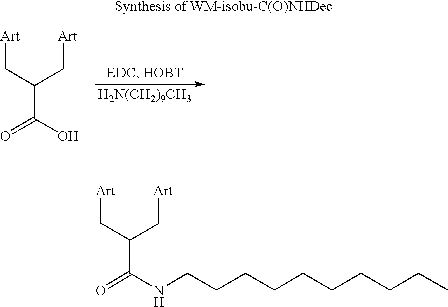

An oven dried 15 mL round bottom flask was charged with bis-trioxane acid (0.080 g, 0.13 mmol), 1-Ethyl-3-(3-dimethylaminopropyl)-carbodiimide (EDC, 0.099 g, 0.52 mmol), N-Hydroxybenzotriazole (HOBT, 0.019 g, 0.14 mmol) and dissolved in 6 mL of anhydrous CH₂Cl₂. This solution was allowed to stir for 2 hr before decyl amine (0.081 g, 0.52 mmol) was added dropwise over the course of about 2 min. After stirring for 18 hr the reaction was quenched by the slow addition of H₂O (5 mL). The contents of the flask were extracted with ether (2×25 mL), washed with a saturated aqueous solution of NaHCO₃ and H₂O, dried over MgSO₄, and concentrated in vacuo. The crude product was purified by silica gel chromatography (20% ethyl acetate in hexanes) to give WM-isobu-C(O)NHDec as an amorphous solid (0.096 g, 98%): [□]_{D}²³=88 (c=4.8, CHCl₃); IR (thin film) 2925, 2854, 1727, 1658, 1532, 1454, 1376 cm⁻¹; ¹H NMR (400 MHz, CDCl₃) δ 5.87 (s, 1H), 5.25 (s, 1H), 5.21 (s, 1H), 4.10-4.01 (m, 2H), 3.25-3.16 (m, 2H), 2.73-2.64 (m, 2H), 2.46-2.45 (m, 1H), 2.34-1.16 (m, 45H), 0.94-0.79 (m, 18H); ¹³C NMR (100 MHz, CDCl₃) δ 175.5, 103.3, 103.2, 88.5, 88.2, 81.1, 80.9, 76.3, 73.9, 65.7, 52.5, 52.3, 44.6, 44.5, 44.4, 39.7, 37.3, 37.1, 36.4, 34.4, 32.8, 32.6, 31.8, 30.1, 29.9, 29.5, 29.24, 29.20, 29.16, 27.0, 26.1, 24.8, 24.6, 24.6, 24.4, 22.6, 20.1, 15.2, 14.0, 13.5, 12.9; HRMS (FAB, M+1) calc. 760.53636 for C₄₄H₇₄NO₉, found 760.53845.

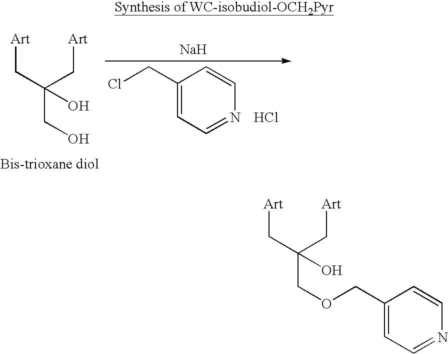

To a solution of 4-picolylchloride hydrochloride (85 mg, 0.52 mmol) in DMF (3 mL) at 0° C. was added sodium hydride (60% dispersion in mineral oil, 104 mg, 2.60 mmol) and the heterogeneous mixture was stirred at rt for 30 min. To the mixture was added a solution of bis-trioxane diol (162 mg, 0.26 mmol) in DMF (2 mL) dropwise. It was warmed to rt and stirred for 5 h. The reaction was cooled to 0° C. and quenched with water (0.5 mL) and saturated aq. NH₄Cl (2 mL). Ether (3 mL) was added and layers were separated. The aqueous layer was extracted with ether (5×2 mL). The combined organic solution was washed with saturated aq. CuSO₄ solution (1×1 mL), dried (MgSO₄), and concentrated. The crude oil was purified by flash column chromatography (elution with EtOAc:hexanes=2:1) on silica gel, that had been treated with Et₃N (1 mL per 100 mL gel) in hexanes before use. WC-isobudiol-OCH₂Pyr (135 mg, 73%) was afforded as a white solid: [α]_{D}²⁴=+37 (c 0.34, CHCl₃); mp 85-86° C.; IR (thin film) 3500, 2924, 1716, 1102, 1053, 1011; ¹H NMR (400 MHz, CDCl₃) δ 8.53 (d, J=5.2 Hz, 2H), 7.33 (d, J=6.0 Hz, 2H), 5.33 (s, 1H), 5.29 (s, 1H), 4.67 (d, J=13.6 Hz, 1H), 4.60 (m, 2H), 4.58 (d, J=13.6 Hz, 1H), 4.02 (bs, 1H), 3.80 (d, J=9.2 Hz, 1H), 3.66 (d, J=9.2 Hz, 1H), 2.66 (dq, J=13.6, 6.8 Hz, 1H), 2.58 (dq, J=13.6, 6.8 Hz, 1H), 2.29 (m, 2H), 2.03-1.73 (m, 9H), 1.68-1.53 (m, 5H), 1.40-1.18 (m, 14H including s at 1.38 and 1.32), 0.97-0.82 (m, 14H including d at 0.94 with J=5.2 Hz, and d at 0.87 with J=7.6 Hz and 0.85 with J=7.6 Hz); ¹³C NMR (100 MHz, CDCl₃) δ 149.1, 148.8, 122.2, 103.1, 102.9, 89.4, 88.7, 81.1, 74.3, 73.9, 71.4, 71.1, 70.9, 52.3, 52.0, 44.3, 43.8, 37.4, 37.4, 36.5, 36.5, 36.2, 35.1, 34.4, 34.3, 30.8, 30.7, 26.1, 26.0, 24.8, 24.7, 24.7, 24.7, 20.1, 20.0, 13.1, 12.7; HRMS (FAB) calculated for C₄₀H₆₀NO₁₀ [(M+H)⁺] 714.4217, found 714.4199; Anal. calculated for C₄₀H₅₉NO₁₀ C, 67.30; H, 8.33; N, 1.96; found C, 67.16; H, 8.42; N, 1.92.

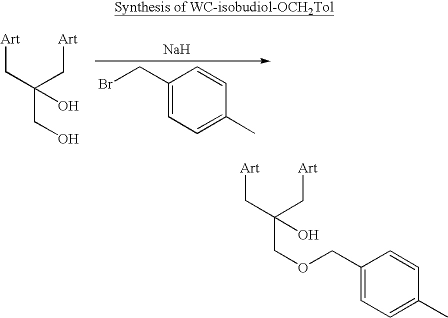

To a solution of bis-trioxane diol (98 mg, 0.16 mmol) in THF (1 mL) at 0° C. was added sodium hydride (60% dispersion in mineral oil, 16 mg, 0.40 mmol). After 30 min, to the heterogeneous mixture at 0° C. was added 4-methylbenzyl bromide (35 mg, 0.19 mmol) in THF (1 mL) dropwise. The reaction was warmed to rt and stirred for 16 h. It was quenched with water (1 mL) and layers were separated. The aqueous layer was extracted with EtOAc (3×2 mL). The combined organic solution was dried (MgSO₄), and concentrated. The purification of the crude product by column chromatography (elution with EtOAc:hexanes=1:5) afforded WC-isobudiol-OCH₂Tol (107 mg, 93%) as a colorless oil: [α]_{D}²⁴=+67 (c 0.34, CHCl₃), IR (neat) 3504, 2953, 2848, 1740, 1129 cm⁻¹; ¹H NMR (400 MHz, CDCl₃) δ 7.26 (d, J=8.0 Hz, 2H), 7.12 (d, J=8.0 Hz, 2H), 5.37 (s, 1H), 5.35 (s, 1H), 4.58 (d, J=7.2 Hz, 1H), 4.55 (m, 2H), 4.47 (d, J=7.2 Hz, 1H), 3.77 (d, J=9.2 Hz, 1H), 3.62 (d, J=9.2 Hz, 1H), 2.70 (dq, J=12.0, 6.0 Hz, 1H), 2.62 (dq, J=12.8, 6.4 Hz, 1H), 2.38-2.28 (m, 5H including s at 2.33), 2.04-1.84 (m, 6H), 1.81-1.73 (m, 3H), 1.65-1.52 (m, 5H), 1.48-1.17 (m, 15H including s at 1.41 and 1.37), 0.98-0.82 (m, 14H including dd at 0.93 with J=4.0, 6.0 Hz, and d at 0.86 with J=7.6 Hz); ¹³C NMR (100 MHz, CDCl₃) δ 136.9, 135.9, 128.8, 128.1, 103.2, 103.1, 99.1, 89.1, 88.6, 81.2, 81.1, 74.0, 73.9, 73.0, 71.7, 71.4, 52.4, 52.2, 44.6, 44.2, 37.3, 36.6, 35.9, 35.0, 34.5, 34.4, 30.7, 30.7, 26.2, 26.0, 24.8, 24.7, 21.2, 21.0, 20.2, 20.1, 14.2, 13.3, 13.0; HRMS (FAB) calculated for C₄₂H₆₃O₁₀ [(M+H)⁺] 727.4421, found 727.4412.

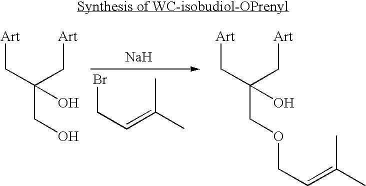

To a solution of bis-trioxane diol (92 mg, 0.15 mmol) in THF (1 mL) at −5° C. was added sodium hydride (60% dispersion in mineral oil, 30 mg, 0.74 mmol). After 30 min, to the heterogeneous mixture at 0° C. was added 3,3-dimethylallyl bromide (26 μL, 0.22 mmol) and DMSO (0.5 mL). The reaction was warmed to rt and stirred for 1 h. It was diluted with ether (3 mL), quenched with water (2 mL). Layers were separated and the aqueous layer was extracted with ether (3×2 mL). The combined organic solution was dried (MgSO₄) and concentrated. The purification of the crude product by column chromatography (elution with EtOAc:hexanes=1:5) provided WC-isobudiol-OPrenyl (98 mg, 96%) as a colorless oil: [α]_{D}²⁴=+64.9 (c 1.14, CHCl₃), IR (neat) 3505, 2922, 1452, 1377, 1091, 1052, 1010, 754 cm⁻¹; ¹H NMR (400 MHz, CDCl₃) δ 5.38 (s, 1H), 5.36 (t, J=1.2 Hz, 1H), 4.54 (dd, J=10.4, 6.4 Hz, 1H), 4.03 (m, 2H), 3.64 (d, J=9.2 Hz, 1H), 3.55 (d, J=9.2 Hz, 1H), 2.69 (dq, J=13.6, 6.0 Hz, 1H), 2.62 (dq, J=13.6, 6.8 Hz, 1H), 2.32 (m, 2H), 2.04-1.18 (m, 37H including s at 1.72, 1.66, 1.40 and 1.39), 0.98-0.84 (m, 14H including s at 0.95 and 0.94, and d at 0.86 with J=7.2 Hz, and at 0.84 with J=7.6 Hz); ¹³C NMR (100 MHz, CDCl₃) δ 135.9, 121.9, 103.1, 103.0, 98.2, 89.1, 88.6, 81.2, 81.1, 73.9, 71.6, 71.4, 67.6, 52.5, 52.2, 44.6, 44.2, 37.4, 37.4, 36.6, 35.8, 35.1, 34.5, 34.5, 30.8, 30.7, 26.1, 26.0, 25.8, 24.8, 24.7, 24.7, 24.7, 20.2, 20.2, 18.0, 13.2, 13.0; HRMS (FAB) calculated for C₃₉H₆₃O₁₀ [(M+H)⁺] 691.4421, found 691.4441.

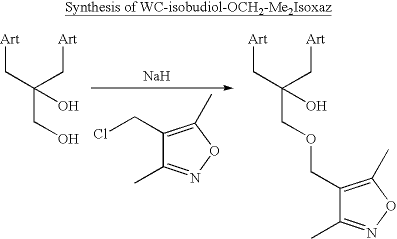

To a solution of bis-trioxane diol (88 mg, 0.14 mmol) in THF (1 mL) at 0° C. was added sodium hydride (60% dispersion in mineral oil, 28 mg, 0.70 mmol). After 30 min, to the heterogeneous mixture at 0° C. was added tetrabutylammonium iodide (5.2 mg, 0.014 mmol), 4-chloromethyl-3,5-dimethylisoxazole (21 μL, 0.17 mmol) and DMSO (0.5 mL). The reaction was warned to rt and stirred for 30 min. It was diluted with ether (3 mL), quenched with water (2 mL). Layers were separated and the aqueous layer was extracted with ether (3×2 mL). The combined organic solution was dried (MgSO₄) and concentrated. The purification of the crude product by column chromatography (elution with EtOAc:hexanes=1:3) gave WC-isobudiol-OCH₂-Me₂Isoxaz (101 mg, 98%) as a colorless oil: [α]_{D}²⁴=+46 (c 0.91, CHCl₃), IR (neat) 3504, 2923, 1454, 1377, 1094, 1010, 754 cm⁻¹; ¹H NMR (400 MHz, CDCl₃) δ 5.31 (s, 1H), 5.30 (s, 1H), 4.66 (m, 1H), 4.56 (dd, J=10.0, 6.0 Hz, 1H), 4.40 (d, J=12.0 Hz, 1H), 4.27 (d, J=11.6 Hz, 1H), 3.72 (d, J=8.8 Hz, 1H), 3.56 (d, J=8.8 Hz, 1H), 2.70 (dq, J=14.4, 7.2, Hz, 1H), 2.62 (dq, J=12.8, 6.4 Hz, 1H), 2.38 (s, 3H), 2.34 (m, 1H), 2.30 (m, 1H), 2.27 (s, 3H), 2.03-1.18 (m, 29H including s at 1.39 and 1.34), 0.98-0.82 (m, 14H including s at 0.86 and 0.84); ¹³C NMR (100 MHz, CDCl₃) δ 167.2, 160.2, 111.6, 103.2, 102.7, 100.8, 98.2, 89.7, 88.6, 81.1, 73.4, 71.1, 70.3, 61.5, 52.4, 51.9, 44.6, 43.6, 37.5, 37.4, 36.9, 36.6, 36.6, 35.2, 34.5, 34.3, 30.9, 30.6, 26.1, 25.9, 24.8, 24.7, 24.6, 20.2, 20.0, 13.3, 12.4, 10.9, 10.0; HRMS (FAB) calculated for C₄₀H₆₂NO₁₁ [(M+H)⁺] 732.4323, found 732.4332.

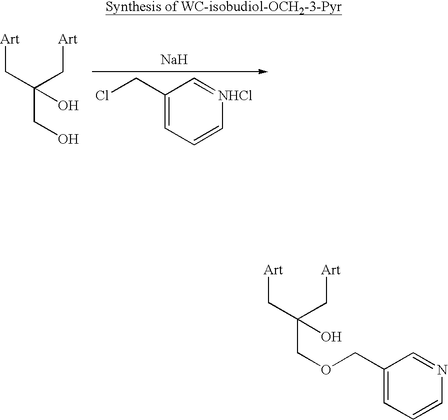

To a solution of 3-picolylchloride hydrochloride (55 mg, 0.33 mmol) in THF (2 mL) at 0° C. was added sodium hydride (60% dispersion in mineral oil, 67 mg, 1.7 mmol) and the heterogeneous mixture was stirred at rt for 10 min. To the mixture was added a solution of bis-trioxane diol (104 mg, 0.17 mmol) in THF (1 mL) dropwise. It was warmed to rt and stirred for 12 h. The reaction was cooled to 0° C., diluted with ether (3 mL), and quenched with water (0.5 mL) and saturated aq. NH₄Cl (3 mL). EtOAc (3 mL) was added and layers were separated. The aqueous layer was extracted with EtOAc (5×3 mL). The combined organic solution was washed with saturated aq. CuSO₄ solution (1×1 mL), dried (MgSO₄), and concentrated. The crude oil was purified by flash column chromatography (elution with EtOAc:hexanes=1:1) on silica gel, that had been treated with Et₃N (1 mL per 100 mL gel) in hexanes before use. WC-isobudiol-OCH₂-3-Pyr (107 mg, 90%) was afforded as a colorless oil: [α]_{D}²⁴=+41 (c 1.0, CHCl₃); IR (neat) 2922, 1377, 1093, 1010, 753; ¹H NMR (400 MHz, CDCl₃) δ 8.62 (s, 1H), 8.53 (d, J=3.6 Hz, 1H), 7.83 (dt, J=8.0, 2.0 Hz, 1H), 7.33 (dd, J=7.6, 4.8 Hz, 1H), 5.34 (s, 1H), 5.30 (s, 1H), 4.68 (d, J=11.6 Hz, 1H), 4.57 (m, 2H), 4.55 (d, J=12.0 Hz, 1H), 3.98 (s, 1H), 3.81 (d, J=8.0 Hz, 1H), 3.67 (d, J=8.8 Hz, 1H), 2.70 (dq, J=12.0, 6.0 Hz, 1H), 2.58 (dq, J=13.6, 6.8 Hz, 1H), 2.36-2.25 (m, 2H), 2.02 (m, 1H), 1.99 (m, 1H), 1.95-1.16 (m, 26H including s at 1.40 and 1.36), 0.98-0.83 (m, 14H including s at 0.94 and 0.92, and d at 0.87 with J=3.6 Hz and at 0.85 with J=4.0 Hz); ¹³C NMR (100 MHz, CDCl₃) δ 148.0, 147.3, 136.9, 135.2, 123.7, 103.2, 103.0, 101.8, 89.4, 88.7, 81.1, 74.1, 73.8, 71.2, 71.0, 70.3, 52.3, 52.1, 44.4, 43.9, 37.4, 37.4, 36.6, 36.6, 36.2, 35.1, 34.4, 34.3, 30.8, 30.7, 26.1, 26.0, 24.8, 24.7, 24.7, 20.2, 20.1, 13.1, 12.8; HRMS (FAB) calculated for C₄₀H₆₀NO₁₀ [(M+H)⁺] 714.4217, found 714.4231.

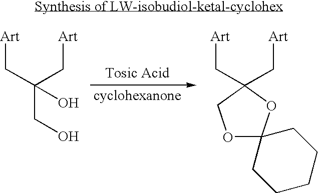

p-Toluenesulfonic acid monohydrate (3 mg, 0.02 mmol) was added to a solution of bis-trioxane diol (50 mg, 0.08 mmol) and cyclohexanone (0.020 mL, 0.16 mmol) in dichloromethane (2 mL). The reaction was stirred overnight at room temperature and progress was monitored by TLC. The solution was washed with saturated aqueous NaHCO₃ (5 mL), water (5 mL), and brine (5 mL), dried over MgSO₄, filtered and concentrated. The crude product was purified by flash chromatography (silica gel, 14% EtOAc in hexane) to afford LW-isobudiol-ketal-cyclohex as a white solid (0.039 g, 70%): mp=113-115° C.; IR (thin film) 2937, 2874, 1449, 1376, 1103, 1054 cm⁻¹; ¹H NMR (400 MHz, CDCl₃) δ 5.39 (s, 1H), 5.37 (s, 1H), 4.61 (m, 11H), 4.14 (m, 1H), 3.95 (d, J=8.8 Hz, 1H), 3.84 (d, J=8.4 Hz, 1H), 2.76 (m, 2H), 2.39-2.29 (m, 3H), 2.09-1.17 (m, 35H, including singlets at 1.38 and 1.35), 0.94-0.82 (m, 16H, including doublet at 0.92); ¹³C NMR (100 MHz, CDCl₃) δ 109.52, 103.44, 103.18, 88.05, 82.41, 81.14, 81.11, 77.23, 73.41, 72.94, 72.17, 52.61, 52.55, 44.96, 44.90, 37.55, 37.18, 37.07, 36.71, 36.47, 36.00, 35.04, 34.59, 34.50, 30.91, 30.65, 29.71, 26.21, 26.18, 25.21, 24.58, 24.51, 24.34, 24.00, 23.80, 20.29, 20.27, 13.91, 13.85; HRMS (FAB) m/z calc'd for C₄₀H₆₃O₁₀ (M+H)⁺ 703.4421, found 703.4415.

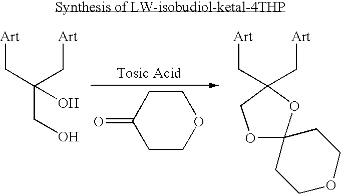

p-Toluenesulfonic acid monohydrate (3 mg, 0.2 mmol) was added to a solution of bis-trioxane diol (50 mg, 0.08 mmol) and tetrahydro-4H-pyran-4-one (0.015 mL, 0.16 mmol) in dichloromethane (2 mL). The reaction was stirred overnight at room temperature and progress was monitored by TLC. The solution was washed with saturated aqueous NaHCO₃ (5 mL), water (5 mL), and brine (5 mL), dried over MgSO₄, filtered and concentrated. The crude product was purified by flash chromatography (silica gel, 33% EtOAc in hexane) to afford LW-isobudiol-ketal-4THP as a white solid (0.030 g, 54%): mp=87-89° C.; IR (thin film) 2953, 2873, 1712, 1453, 1376 cm⁻¹; ¹H NMR (400 MHz, CDCl₃) δ 5.36 (s, 1H), 5.35 (s, 1H), 4.59 (m, 1H), 4.19 (m, 1H), 4.00 (d, J=8.8 Hz, 1H), 3.89 (d, J=8.8 Hz, 1H), 3.75 (m, 4H), 2.79-2.64 (m, 2H), 2.37-2.27 (m, 3H), 2.04-1.16 (m, 29H, including singlets at 1.38 and 1.36), 0.94-0.81 (m, 16H, including doublet at 0.93); ¹³C NMR (100 MHz, CDCl₃) δ 106.18, 103.14, 102.82, 88.21, 87.93, 82.72, 80.86, 80.84, 72.63, 72.53, 71.16, 65.76, 65.71, 52.25, 52.13, 44.58, 44.31, 37.93, 37.03, 36.94, 36.72, 36.68, 36.44, 36.40, 34.56, 34.28, 34.19, 30.65, 30.42, 25.90, 25.86, 24.35, 24.14, 20.01, 19.95, 13.49, 13.28; HRMS (FAB) m/z calc'd for C₃₉H₆₁O₁₁ (M+H)⁺ 705.4214, found 705.4214; HPLC [phenomenex semi-preparative silica gel column (1×25 cm), 30% EtOAc in hexanes, 2 mL/min, 270 nm, t_{R}=20.5 min].

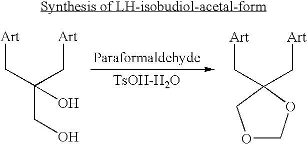

To a solution of bis-triozane diol (50 mg, 0.08 mmol) in CH₂Cl₂ (2 mL) was added paraformaldehyde (5 mg, 0.16 mmol) and p-toluenesulfonic acid monohydrate (TsOH—H₂O, 3 mg, 0.02 mmol). The reaction was stirred at room temperature for 12 h. The reaction was quenched with saturated aq NaHCO₃ (5 mL) and the layers were separated. The aqueous layer was extracted with EtOAc (3×10 mL). The combined organic solution was washed with brine, dried over MgSO₄ and concentrated in vacuo. The purification of the crude product by column chromatography (elution with 25% EtOAc in Hexanes) gave LH-isobudiol-acetal-form (38 mg, 72%) as an amorphous solid: ¹H NMR (400 MHz, CDCl₃) δ 5.37 (s, 1H), 5.34 (s, 1H) 4.99 (s, 1H), 4.96 (s, 1H), 4.47 (q, J=6.0, 8.0, 15.6 Hz, 1H), 4.29 (q, J=6.0, 8.0, 15.6 Hz, 1H), 3.86 (s, 1H), 2.73-2.64 (m, 1H), 2.35-2.20 (m, 3H), 2.02-1.96 (m, 3H), 1.90-1.71 (m, 7H), 1.65-1.17 (m, 21H including d at 1.35 with J=5.2 Hz and s at 1.23), 0.97-0.83 (m, 14H including dd at 0.91 with J=1.6, 6.0 Hz); ¹³C NMR (400 MHz, CDCl₃) δ 103.2, 103.1, 94.6, 88.5, 82.2, 81.09, 81.07, 73.4, 72.2, 71.3, 52.4, 52.3, 44.6, 44.5, 37.3, 37.2, 36.7, 36.6, 35.4, 34.5, 34.4, 33.8, 30.7, 30.6, 26.1, 26.09, 24.6, 24.56, 24.53, 24.4, 20.2, 20.1, 13.4, 13.2; HRMS (FAB) calculated for C₃₅H₅₄O₁₀ [(M+H)⁺] 634.3795, found 634.3765.

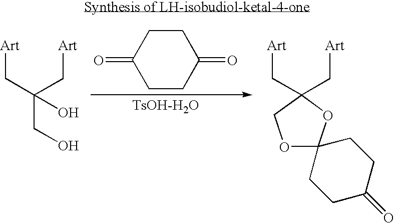

To a solution of bis-trioxane diol (50 mg, 0.08 mmol) in CH₂Cl₂ (3 mL) was added 1,4-cyclopentanedione (90 mg, 0.80 mmol) and p-toluenesulfonic acid monohydrate (TsOH—H₂O, 3 mg, 0.02 mmol). The reaction was stirred at room temperature for 12 h. The reaction was quenched with saturated aq NaHCO₃ (5 mL) and the layers were separated. The aqueous layer was extracted with EtOAc (3×10 mL). The combined organic solution was washed with brine, dried over MgSO₄ and concentrated in vacuo. The purification of the crude product by column chromatography (elution with 25% EtOAc in Hexanes) gave LH-isobudiol-ketal-4-one (45 mg, 78%) as a white solid: [α]_{D}²¹=+70 (c=0.75, CHCl₃); mp 104-106° C.; IR (thin film) 2938, 2880, 2359, 2320, 1712, 1635, 1587, 1558, 1442, 1374, 1316, 1249, 1220, 1181, 1114, 1046, 1008, 959, 921, 872, 834, 747; ¹H NMR (400 MHz, CDCl₃) δ 5.36 (s, 1H), 5.35 (s, 1H), 4.59-4.54 (m, 1H), 4.60 (q, J=6.0, 8.2, 16.4 Hz, 1H), 4.25 (t, J=6.4 Hz, 1H) 4.08 (d, J=8.8 Hz, 1H), 3.96 (d, J=8.8 Hz, 1H), 2.75 (sextet, J=7.2 Hz, 1H) 2.68-2.55 (m, 3H), 2.48-2.40 (m, 2H), 2.36-2.22 (m, 3H), 2.11-1.69 (m, 15H), 1.70-1.16 (m, 22H including s at 1.41 and d at 1.35 with J=10.0 Hz), 0.92-0.80 (m, 14H including d at 0.93 with J=6.0 Hz); ¹³C NMR (400 MHz, CDCl₃) δ 210.8, 107.2, 103.3, 102.8, 88.8, 88.21, 83.4, 81.1, 81.0, 73.3, 72.6, 70.8, 52.4, 52.2, 44.7, 44.2, 38.22, 38.2, 37.3, 37.2, 37.1, 36.59, 36.55, 35.9, 34.7, 34.5, 34.4, 34.3, 30.9, 30.7, 30.3, 26.1, 26.0, 24.6, 24.59, 24.4, 20.2, 13.4, 13.2, 11.1; HRMS (FAB) calculated for C₄₀H₆₁O₁₁ [(M+H)⁺] 717.4214, found 717.4181.

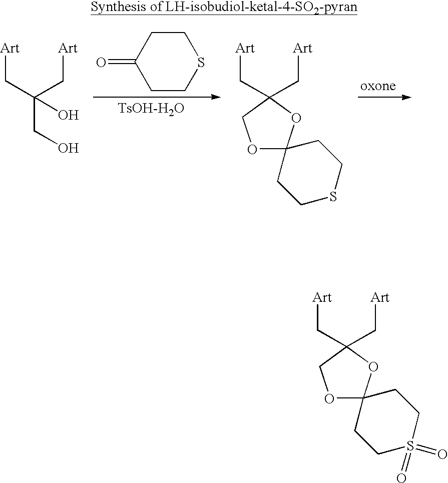

To a solution of bistrioxane diol (50 mg, 0.08 mmol) in CH₂Cl₂ (1 mL) was added tetrahydrothiopyran-4-one (18 mg, 0.16 mmol) and p-toluenesulfonic acid monohydrate (TsOH—H₂O, 3 mg, 0.02 mmol). The reaction was stirred at room temperature for 12 h. The reaction was quenched with saturated aq NaHCO₃ (3 mL) and the layers were separated. The aqueous layer was extracted with EtOAc (3×5 mL). The combined organic solution was washed with brine, dried over MgSO₄ and concentrated in vacuo. The purification of the crude product by column chromatography (elution with 25% EtOAc in Hexanes) gave the intermediate. To a mixture of oxone (410 mg, 0.69 mmol) in H₂O (2 mL) was cannulated the intermediate (49 mg, 0.07 mmol) in MeOH (4 mL). The reaction stirred at room temperature 1.5 h. The reaction was filtered and the aqueous solution was extracted with EtOAc (3×5 mL). The combined organic layer was washed with brine, dried over MgSO₄ and concentrated in vacuo. Purification by column chromatography (30% EtOAc in Hexanes) gave LH-isobudiol-ketal-4-SO₂-pyran (46 mg, 88%) as a white solid: [α]_{D}²⁴=+62 (c=0.34, CHCl₃); m.p. 138-140° C.; IR (thin film) 2938, 2880, 2851, 2465, 2224, 1712, 1587, 1558, 1452, 1374.1326, 1287, 1249, 1220, 1191, 1104, 1056, 1017, 940, 901, 882, 747, 660 cm⁻¹; ¹H NMR (400 MHz, CDCl₃) δ 5.29 (s, 1H), 5.27 (s, 1H), 4.59-4.54 (m, 1H), 4.28 (t, J=6.4, 13 Hz, 1H), 4.06 (d, J=9.0 Hz, 1H), 3.93 (d, J=9.0 Hz, 1H), 3.45-3.39 (m, 1H), 3.35-3.29 (m, 1H), 3.09-3.02 (m, 2H), 2.73 (sextet, J=7.2 Hz, 1H), 2.48 (sextet, J=7.2 Hz, 1H), 2.36-2.24 (m, 5H), 2.08-1.95 (m, 5H including s at 2.05), 1.91-1.69 (m, 7H), 1.65-1.09 (m, 18H including d at 1.32 with J=6.4 Hz), 0.92-0.80 (m, 14H including dd at 0.82 with J=7.6, 11.6 Hz); ¹³C NMR (400 MHz, CDCl₃) δ 104.8, 103.1, 1:02.3, 89.3, 88.0, 83.7, 80.8, 80.7, 73.3, 71.9, 68.9, 51.9, 51.6, 49.0, 48.7, 44.3, 43.3, 37.2, 37.14, 37.10, 36.2, 36.1, 34.2, 34.1, 33.9, 33.8, 33.6, 30.6, 30.4, 25.7, 25.6, 24.5, 24.4, 24.3, 24.1, 19.9, 19.7, 13.1, 12.2; HRMS (FAB) calculated for C₃₉H₆₁O₁₂S [(M+H)⁺] 753.3883, found 753.3875.

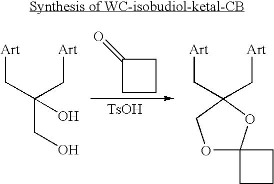

To a solution of bistrioxane diol (70 mg, 0.11 mmol) in CH₂Cl₂ (1 mL) was added cyclobutanone (100 μL, 1.30 mmol) and p-toluenesulfonic acid monohydrate (TsOH, 2 mg). The reaction was stirred at rt for 48 h. It was concentrated and purified by flash column chromatography (elution with EtOAc:hexanes=1:10) on silica gel to give WC-isobudiol-ketal-CB (73 mg, 96%) as an amorphous solid: ¹H NMR (400 MHz, CDCl₃) δ 5.52 (s, 1H), 5.40 (s, 1H), 4.86 (m, 1H), 4.53 (m, 1H), 4.24 (d, J=8.4 Hz, 1H), 3.98 (d, J=8.0 Hz, 1H), 2.90 (m, 1H), 2.78 (m, 1H), 2.63-2.27 (m, 6H), 2.13 (d, J=13.6 Hz, 1H), 1.85-0.55 (m, 42H including s at 1.40 and 0.75); ¹³C NMR (100 MHz, CDCl₃) δ 109.7, 103.2, 102.9, 101.1, 89.2, 89.2, 83.6, 81.0, 80.9, 73.0, 72.0, 71.3, 52.7, 52.6, 45.1, 44.8, 38.0, 37.9, 37.5, 37.4, 37.3, 37.1, 35.5, 34.8, 34.7, 31.2, 26.3, 25.2, 24.8, 24.8, 20.3, 20.2, 13.5, 13.2, 12.1; HRMS (FAB) calculated for C₃₈H₅₉O₁₀ [(M+H)⁺] 675.4108, found 675.4084.

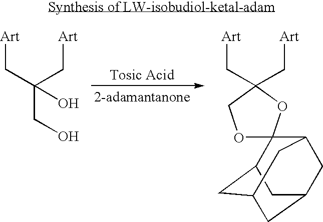

p-Toluenesulfonic acid monohydrate (1 mg) was added to a solution of bis-trioxane diol (20 mg, 0.03 mmol) and 2-adamantanone (20 mg, 0.13 mmol) in dichloromethane (1 mL). The reaction was stirred overnight at room temperature and progress was monitored by TLC. The solution was washed with saturated aqueous NaHCO₃ (5 mL), water (5 mL), and brine (5 mL), dried over MgSO₄, filtered and concentrated. The crude product was purified by flash chromatography (silica gel, 13% EtOAc in hexane) to afford LW-isobudiol-ketal-adam as a white solid (11 mg, 44%): mp 160-162° C.; IR (thin film) 2934, 2855, 1451, 1376, 1222, 1122, 1054, 1012 cm⁻¹; ¹HNMR (400 MHz, CDCl₃) δ 5.39 (s, 1H), 5.37 (s, 1H), 4.65 (m, 1H), 4.13 (m, 1H), 3.99 (d, J=8.8 Hz, 1H), 3.82 (d, J=8.8 Hz, 1H), 2.81-2.73 (m, 2H), 2.38-2.26 (m, 3H), 2.09-1.18 (m, 39H, including singlets at 1.39 and 1.37), 0.95-0.83 (m, 16H, including doublet at 0.94); ¹³C NMR (100 MHz, CDCl₃) δ 111.82, 103.32, 103.12, 88.02, 82.40, 81.07, 81.05, 73.31, 72.66, 72.31, 52.58, 52.52, 44.94, 44.91, 38.96, 37.19, 37.11, 36.74, 36.69, 34.93, 34.86, 34.60, 34.46, 34.28, 31.51, 30.95, 30.62, 27.03, 26.75, 26.14, 26.12, 25.21, 24.52, 24.46, 24.23, 22.57, 20.22, 20.16, 14.03, 13.81, 13.73; HRMS (FAB) m/z calc'd for C₄₄H₆₇O₁₀ (M+H)⁺ 755.4734, found 755.4718.

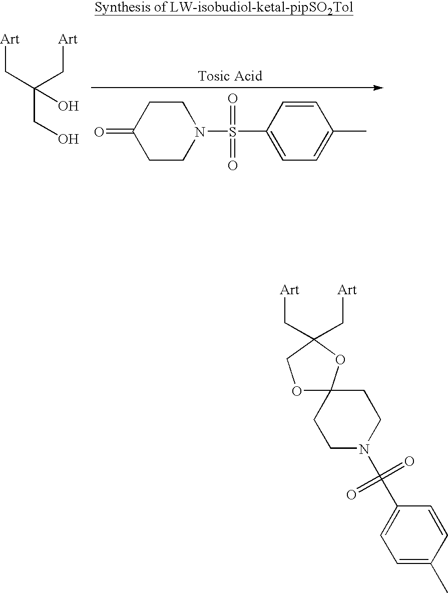

p-Toluenesulfonic acid monohydrate (2 mg, 0.01 mmol) was added to a solution of bis-trioxane diol (30 mg, 0.05 mmol), and 1-(toluene-4-sulfonyl)-piperidine-4-one (24 mg, 0.10 mmol) in dichloromethane (2 mL). The reaction was stirred overnight at room temperature and progress was monitored by TLC. The solution was washed with saturated aqueous NaHCO₃ (5 mL), water (5 mL), and brine (5 mL), dried over MgSO₄, filtered and concentrated. The crude product was purified by flash chromatography (silica gel, 33% EtOAc in hexane) to afford LW-isobudiol-ketal-pipSO₂Tol as a white solid (35 mg, 83%): mp=122-124° C.; ¹H NMR (400 MHz, CDCl₃) δ 7.62 (d, J=8.0 Hz, 2H), 7.31 (d, J=8.0 Hz, 2H), 5.30 (s, 2H), 4.47 (m, 1H), 4.11 (m, 1H), 3.92 (d, J−9.2 Hz, 1H), 3.82 (d, J=8.8 Hz, 1H), 3.26 (m, 2H), 2.98 (m, 2H), 2.71-2.61 (m, 2H), 2.45 (s, 3H), 2.34-2.18 (m, 3H), 1.97-1.07 (m, 29H, including singlets at 1.35 and 1.28), 0.93-0.78 (m, 16H, including doublet at 0.92); ¹³C NMR (100 MHz, CDCl₃) δ 143.34, 133.36, 129.68, 127.47, 106.14, 103.23, 102.89, 88.37, 88.08, 83.22, 80.98, 80.92, 72.80, 72.47, 71.09, 52.29, 52.20, 44.59, 44.33, 37.17, 37.08, 36.51, 36.39, 35.23, 34.58, 34.36, 34.27, 30.74, 30.54, 25.98, 25.88, 24.47, 24.28, 22.22, 21.45, 20.11, 20.05, 13.95, 13.47, 13.34; HRMS (FAB) m/z calc'd for C₄₆H₆₈NO₁₂S (M+H)⁺ 858.4462, found 858.4428.

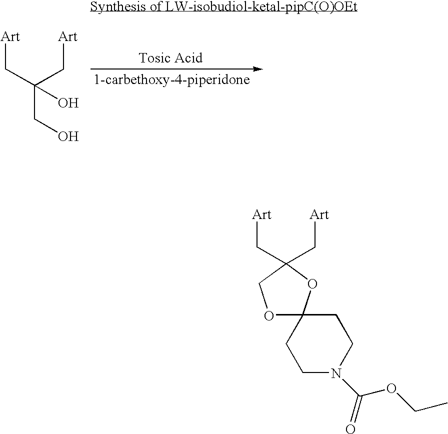

p-Toluenesulfonic acid monohydrate (1 mg) was added to a solution of bis-trioxane diol (20 mg, 0.03 mmol), and 1-carbethoxy-4-piperidone (10 μL, 0.06 mmol) in dichloromethane (1 mL). The reaction was stirred overnight at room temperature and progress was monitored by TLC. The solution was washed with saturated aqueous NaHCO₃ (5 mL), water (5 mL), and brine (5 mL), dried over MgSO₄, filtered and concentrated. The crude product was purified by flash chromatography (silica gel, 29% EtOAc in hexane) to afford LW-isobudiol-ketal-pipC(O)OEt as a white solid (17 mg, 67%): mp=83-85° C.; IR (thin film) 2927, 2875, 1697, 1435, 1378, 1350, 1279, 1240, 1112, 1055, 1011 cm⁻¹; ¹H NMR (400 MHz, CDCl₃) δ 5.36 (s, 1H), 5.35 (s, 1H), 4.58 (m, 1H), 4.20 (m, 1H), 4.01 (d, J=8.8 Hz, 1H), 3.89 (d, J=8.8 Hz, 1H), 3.61-3.47 (m, 4H), 2.77-2.65 (m, 2H), 2.37-2.23 (m, 3H), 2.04-1.14 (m, 34H, including singlets at 1.39 and 1.36), 0.95-0.84 (m, 16H, including doublet at 0.94); ¹³C NMR (100 MHz, CDCl₃) δ 155.40, 107.22, 103.36, 103.01, 88.52, 88.21, 83.09, 81.08, 81.05, 77.20, 72.75, 71.26, 61.25, 52.46, 52.31, 44.78, 44.47, 41.79, 37.28, 37.20, 36.97, 36.65, 36.62, 35.68, 34.50, 34.39, 30.89, 30.65, 29.67, 26.12, 26.08, 24.60, 24.38, 20.24, 20.16, 14.66, 14.17, 13.68, 13.44; HRMS (FAB) m/z calc'd for C₄₂H₆₆NO₁₂ (M+H)⁺ 776.4585, found 776.4597.

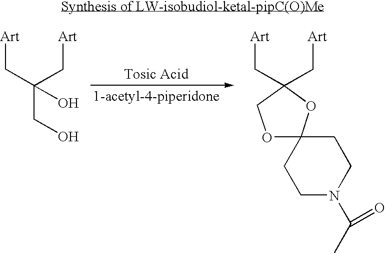

p-Toluenesulfonic acid monohydrate (1 mg) was added to a solution of bis-trioxane diol (20 mg, 0.03 mmol), and 1-acetyl-4-piperidone (8 μL, 0.06 mmol) in dichloromethane (1 mL). The reaction was stirred overnight at room temperature and progress was monitored by TLC. The solution was washed with saturated aqueous NaHCO₃ (5 mL), water (5 mL), and brine (5 mL), dried over MgSO₄, filtered and concentrated. The crude product was purified by flash chromatography (silica gel, 75% EtOAc in hexane) to afford LW-isobudiol-ketal-pipC(O)Me as a white solid (9 mg, 39%): mp=109-111° C.; IR (thin film) 2938, 2875, 1640, 1446, 1376, 1358, 1267, 1112, 1054, 1008 cm⁻¹; ¹H NMR (400 MHz, CDCl₃) δ 5.35 (s, 1H), 5.34 (s, 1H), 4.58 (m, 1H), 4.21 (m, 1H), 4.03 (d, J=8.8 Hz, 1H), 3.91 (d, J=8.8 Hz, 1H), 3.76 (m, 1H), 3.62-3.47 (m, 3H), 2.77-2.62 (m, 2H), 2.34-1.20 (m, 35H, including singlets at 1.39 and 1.35), 0.95-0.84 (m, 16H, including doublet at 0.94); ¹³C NMR (100 MHz, CDCl₃) δ 168.79, 106.97, 103.37, 102.96, 88.26, 81.09, 81.06, 77.21, 52.43, 52.26, 44.75, 44.34, 37.33, 37.25, 36.63, 34.49, 34.38, 30.91, 30.68, 26.12, 26.06, 24.63, 24.40, 21.38, 20.24, 20.15, 13.30; HRMS (FAB) m/z calc'd for C₄₁H₆₄NO₁₁ (M+H)⁺ 746.4479, found 746.4495.

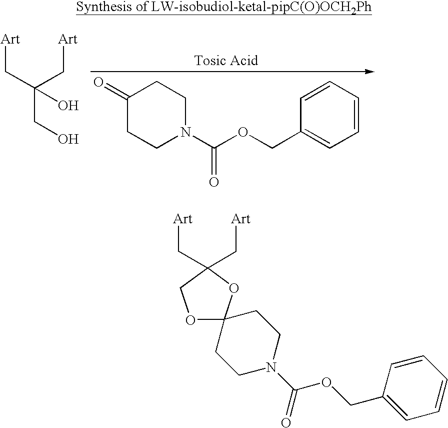

p-Toluenesulfonic acid monohydrate (2 mg, 0.01 mmol) was added to a solution of bis-trioxane vicinal diol (40 mg, 0.06 mmol), and benzyl 4-oxo-1-piperidine carboxylate (60 mg, 0.26 mmol) in dichloromethane (2.5 mL). The reaction was stirred overnight at room temperature and progress was monitored by TLC. The solution was washed with saturated aqueous NaHCO₃ (5 mL), water (5 mL), and brine (5 mL), dried over MgSO₄, filtered and concentrated. The crude product was purified by flash chromatography (silica gel, 29% EtOAc in hexane) to afford LW-isobudiol-ketal-pipC(O)OCH₂Ph as a white solid (44 mg, 0.052 mmol, 81%): mp=69-71° C.; IR (thin film) 2936, 2875, 1702, 1498, 1433, 1376, 1359, 1278, 1228, 1189, 1111, 1055, 1009 cm⁻¹; ¹H NMR (400 MHz, CDCl₃) δ 7.34 (m, 5H), 5.35 (s, 1H), 5.34 (s, 1H), 5.11 (s, 2H), 4.57 (m, 1H), 4.20 (m, 1H), 4.01 (d, J=9.2 Hz, 1H), 3.89 (d, J=8.8 Hz, 1H), 3.69-3.49 (m, 4H), 2.76-2.62 (m, 2H), 2.33-2.27 (m, 3H), 2.03-1.20 (m, 29H, including singlets at 1.38 and 1.35), 0.94-0.83 (m, 16H, including doublet at 0.93); ¹³C NMR (100 MHz, CDCl₃) δ 155.12, 136.86, 128.43, 127.88, 127.77, 107.11, 103.32, 102.96, 88.61, 88.28, 83.14, 81.06, 81.04, 71.13, 67.03, 52.46, 52.32, 44.77, 44.44, 41.99, 37.30, 37.22, 36.66, 34.52, 34.41, 30.89, 30.67, 26.09, 26.05, 24.62, 24.40, 20.20, 20.13, 13.60, 13.34; HRMS (FAB) m/z calc'd for C₄₇H₆₈NO₁₂ (M+H)⁺ 838.4742, found 838.4759.

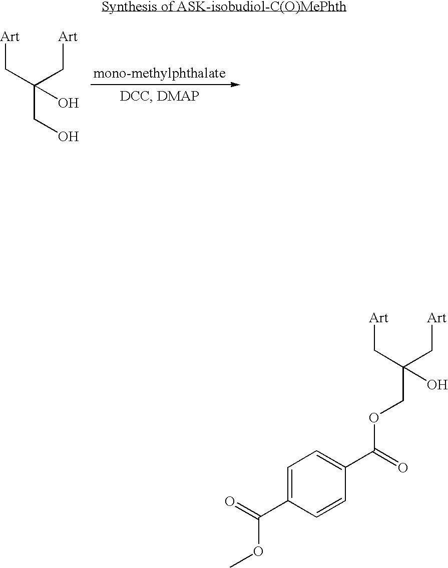

To a solution of bis-trioxane diol (86 mg, 0.14 mmol) in anhydrous dichloromethane (5 mL) was added N,N-dimethylaminopyridine (DMAP, 6 mg, 0.05 mmol, 0.35 equiv) and mono-methylphthalate (40 mg, 0.21 mmol, 1.5 equiv). The solution was allowed to stir for 5 mins at room temperature. To a dry pear shaped flask was added dicyclohexylcarbidimide (DCC, 45 mg, 0.21 mmol, 1.5 equiv) and anhydrous dichloromethane (3 mL). The DCC solution was cannulated into the bis-trioxane diol mixture at room temperature and is allowed to stir overnight. TLC analysis showed full consumption of starting material. The cloudy solution was concentrated under reduced pressure and purified by flash column chromatography on silica gel eluted with (40% EtOAc in hexanes) to give ASK-isobudiol-C(O)MePhth as a white solid (85 mg, 0.11 mmol, 78%): [□]_{D}^{22.6}=+82 (CHCl₃, C=1.7); mp=81-83° C.; IR (thin film) 2944, 2871, 1724, 1452, 1431, 1373, 1269, 1107, 1010, 730 cm⁻¹; ¹H NMR (400 MHz, CDCl₃) δ 8.13-8.06 (m, 4H), 5.33 (s, 1H), 5.31 (s, 1H) 4.74-4.49 (m, 4H), 4.28 (s, 1H), 3.94 (s, 3H), 2.63-2.54 (m, 211), 2.31-2.25 (m, 2H), 2.11-1.76 (m, 13H), 1.74-1.57 (m, 5H), 1.49-0.89 (m, 24H, including two singlets at 1.40 and 1.37); ¹³C NMR (100 MHz, CDCl₃) δ 166.3, 165.4, 134.6, 133.5, 129.3, 102.9, 89.5, 89.2, 88.4, 87.3, 86.6, 85.6, 85.5, 84.7, 81.0, 80.9, 73.8, 70.3, 70.2, 69.7, 52.3, 52.0, 51.9, 47.4, 43.8, 43.7, 37.5, 37.4, 36.5, 36.5, 35.1, 34.3, 30.8, 30.3, 25.9, 25.8, 24.8, 24.7, 20.0, 20.0, 12.6, 12.5; HRMS (FAB) m/z calc'd for C₄₃H₆₀NO₁₃Na (M+H⁺) 785.4112, found 785.4118; HPLC [Phenomenex semi-preparative silica column (1×25 cm)] 30% EtOAc in hexanes, 2 mL/min, 264 nm, t_{R}=28.3 min.

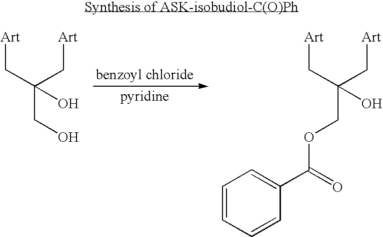

A 25 mL round bottom flask was charged with bis-trioxane diol (70 mg, 0.11 mmol, 1.0 eq.), CH₂Cl₂ (5 mL), anhydrous pyridine (22 μL, 0.56 mmol, 5.0 eq.) and benzoyl chloride (0.33 μL, 0.56 mmol, 5.0 eq). The reaction was stirred at room temperature for 2 hours. The pale yellow reaction mixture was quenched with ice cold water (5 mL) and stirred for 30 minutes. The mixture was poured into a separatory funnel containing Et₂O. The aqueous layer was extracted with Et₂O (3×30 mL) and neutralized with aqueous citric acid (5×50 mL). Combined organic layers were dried over MgSO₄, filtered, and concentrated under reduced. The crude product was purified by flash silica gel column chromatography (40% EtOAc in hexanes) to give 64 mg (80%) of ASK-isobudiol-C(O)Ph as a white solid; [α]_{D}^{22.6}=+70 (CHCl₃, c=1.0); mp=112-114° C.; IR (thin film) 3498, 2943, 2870, 1718, 1446, 1372, 1310, 1274, 1112, 907, 730, 710 cm⁻¹; ¹H NMR (400 MHz, CDCl₃) δ 8.05-8.02 (m, 2H), 7.55-7.51 (m, 1H), 7.43-7.34 (m, 2H), 5.34 (s, 1H), 5.31 (s, 1H), 4.71 (dd, J=10.0, 8.0 Hz, 1H), 4.61 (dd, J=10.0, 8.0 Hz, 1H), 4.55 (s, 2H), 4.24 (bs, 1H), 2.66-2.54 (m, 2H), 2.32-2.24 (m, 2H), 2.15-1.75 (m, 11H), 1.68-1.62 (m, 5H), 1.42-1.18 (m, 16H, including two singlets at 1.40 and 1.37), 0.96-0.86 (m, 7H); ¹³C NMR (100 MHz, CDCl₃) δ 166.1, 132.5, 130.7, 129.5, 128.1, 103.6, 102.9, 89.4, 89.1, 80.9, 80.9, 73.8, 70.5, 70.4, 52.1, 51.9, 43.9, 43.8, 37.4, 37.4, 36.5, 36.5, 36.3, 35.0, 34.3, 34.3, 30.8, 30.7, 30.2, 25.9, 25.8, 24.8, 24.8, 24.6, 24.6, 20.0, 20.0, 12.7, 12.6; HRMS (FAB) m/z calc'd for C₄₁H₅₉O₁₁ (M+H⁺) 727.4057, found 727.4031; HPLC Phenomenex semi-preparative silica column (1×25 cm), 30% EtOAc: 70% hexanes, 2 mL/min, 264 mm, t_{R}=15.9 min.

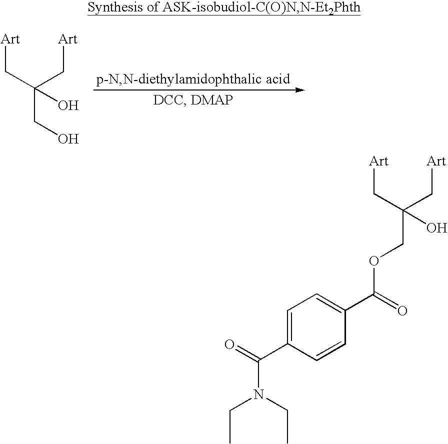

To a solution of bis-trioxane diol (117 mg, 0.19 mmol) in anhydrous dichloromethane (5.0 mL) was added N,N-dimethylaminopyridine (DMAP, 34 mg, 0.28 mmol, 1.5 equiv) and p-N,N-diethylamidophthalic acid (62 mg, 0.28 mmol, 1.5 equiv). The solution was allowed to stir for 5 mins at room temperature. To a dry pear shaped flask was added dicyclohexylcarbidimide (DCC, 58 mg, 0.28 mmol, 1.5 equiv) and additional anhydrous dichloromethane (4 mL). The DCC solution was cannulated into the bis-trioxane diol mixture at room temperature and is allowed to stir overnight. TLC analysis showed full consumption of starting material. The cloudy solution was concentrated under vacuum and purified by flash column chromatography on silica eluting with (60% EtOAc in hexanes) to give ASK-isobudiol-C(O)N,N-Et₂Phth as a white solid (115 mg, 74%); [□]_{D}²³=+66 (CHCl₃, c=1.0); mp=78-80° C.; IR (thin film) 3492, 3010, 2989, 2811, 1751, 1668, 1510, 1492, 1322, 1109, 988 cm⁻¹; ¹H NMR (400 MHz, CDCl₃) δ 8.09 (d, J=8.0 Hz, 2H), 7.41 (d, J=12.0 Hz, 2H), 5.35 (s, 1H), 5.33 (s, 1H),), 4.72 (dd, J=10.0, 8.0 Hz, 1H), 4.62 (dd, J=10.0, 8.0 Hz, 1H), 4.56 (s, 2H), 4.21 (bs, 1H), 3.55 (d, J=8.0 Hz, 2H), 3.21 (d, J=8.0 Hz, 2H), 2.65-2.50 (m, 2H), 2.37-2.25 (m, 2H), 2.10-1.87 (m, 7H), 1.82-1.75 (m, 3H), 1.70-1.62 (m, 5H), 1.42-0.75 (m, 33H, including two singlets at 1.41 and 1.37); ¹³C NMR (100 MHz, CDCl₃) δ 170.3, 141.2, 131.3, 129.8, 126.1, 102.9, 102.8, 89.9, 89.5, 89.2, 89.2, 86.4, 86.2, 85.5, 84.6, 81.0, 80.9, 73.8, 70.3, 69.4, 52.0, 51.9, 45.6, 45.4, 44.4, 44.3, 43.8, 43.7, 37.5, 37.4, 36.5, 36.5, 35.2, 34.3, 34.3, 30.8, 29.8, 25.9, 25.8, 24.8, 24.7, 20.1, 20.0, 12.6, 12.5; HRMS (FAB) m/z calc'd for C₄₆H₆₈NO₁₂ (M+H⁺) 826.4741, found 826.4768; HPLC [Phenomenex semi-preparative silica column (1×25 cm)] 50% EtOAc in hexanes, 2 mL/min, 264 nm, t_{R}=18.7 min.

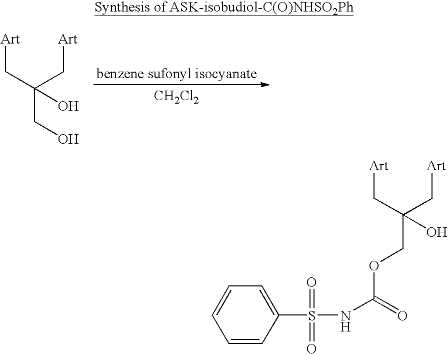

To a solution of benzene sulfonyl isocyanate (22 μL, 0.16 mmol, 1.1 equiv) in anhydrous dichloromethane (5 mL) at 0° C. was added slowly to a solution of bis-trioxane diol (92 mg, 0.15 mmol) in anhydrous dichloromethane (5 mL). After stirring for 30 min., the reaction was quenched with distilled water (1 mL) and the reaction mixture was poured into a mixture of dichloromethane (15 mL) and brine (15 mL). The organic layer was then separated, dried (MgSO₄) and concentrated in vacuo. Flash column chromatography on silica eluting with 30% ethyl acetate and 1% acetic acid in hexanes isolated ASK-isobudiol-C(O)NHSO₂Ph as a white solid (115 mg, 97%): mp=85-86° C.; IR (thin film) 3240, 2949, 2874, 1755, 1444, 1356, 1146, 1091, 989, 861 cm⁻¹; ¹HNMR (400 MHz, CDCl₃) δ 8.05-8.02 (m, 2H), 7.66-7.60 (m, 1H), 7.55-7.51 (m, 2H), 5.33 (s, 1H), 5.26 (s, 1H),), 4.85 (bs, 1H) 4.37-4.32 (m, 1H), 4.24 (dd, J=10.8, 4.8 Hz, 1H), 4.20-4.16 (m, 1H), 4.07 (dd, J=10.8, 7.2 Hz, 1H), 2.71-2.63 (m, 1H), 2.52-2.42 (m, 1H), 2.38-2.22 (m, 2H), 2.15-1.82 (m, 6H), 1.81-1.19 (m, 27H, including two singlets at 1.43 and 1.32), 0.96 (d, J=6.4 Hz, 3H), 0.95 (d, J=6.4 Hz, 3H), 0.84 (d, J=7.6 Hz 3H), 0.79 (d, J=7.6 Hz, 3H); ¹³C NMR (100 MHz, CDCl₃) δ 150.5, 138.8, 133.6, 128.9, 128.1, 103.6, 102.7, 89.6, 88.6, 81.2, 81.1, 74.6, 70.6, 70.5, 52.3, 51.9, 44.3, 43.9, 37.4, 37.3, 36.6, 36.5, 34.5, 34.3, 34.3, 31.3, 31.0, 30.5, 26.0, 25.8, 24.9, 24.8, 24.7, 24.7, 20.2, 20.0, 13.1, 12.4; HRMS (FAB) m/z calc'd for C₄₁H₅₉NO₁₃S (M+H)⁺ 805.3707, found 835.3710.

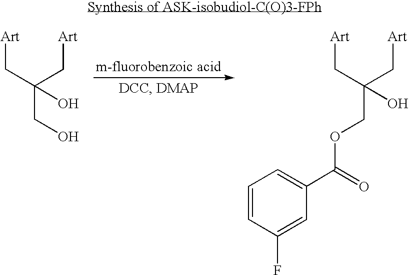

To a solution of bis-trioxane diol (56 mg, 0.09 mmol) in anhydrous dichloromethane (5.0 mL) was added N,N-dimethylaminopyridine (DMAP, 30 mg, 0.14 mmol, 1.5 equiv) and m-benzoic acid (20 mg, 0.14 mmol, 1.5 equiv). The solution was allowed to stir for 5 mins at room temperature. To a dry pear shaped flask was added dicyclohexylcarbidimide (DCC, 30 mg, 0.14 mmol, 1.5 equiv) and additional anhydrous dichloromethane (5 mL). The DCC solution was cannulated into the bis-trioxane diol mixture at room temperature and is allowed to stir overnight. TLC analysis showed fill consumption of starting material. The cloudy solution was concentrated under vacuum and purified by flash column chromatography on silica eluting with (30% EtOAc in hexanes) to give ASK-isobudiol-C(O)3-FPh as a white solid (57 mg, 85%); [□]_{D}²³=+74 (CHCl₃, c=1.0); mp=75-77° C.; IR (thin film) 3495, 2951, 2875, 1724, 1592, 1485, 1448, 1377, 1281, 1269, 1202, 1094, 1054, 1008, 910, 755, 732 cm⁻¹; ¹H NMR (400 MHz, CDCl₃) δ 7.85 (d, J=8.0 Hz, 1H), 7.74 (d, J=9.0 Hz, 1H), 7.42-7.35 (m, 1H) 7.25-7.20 (m, 1H) 5.33 (s, 1H), 5.32 (s, 1H),), 4.71 (dd, J=10.0, 8.0 Hz, 1H), 4.61-4.52 (m, 3H), 4.29 (s, 2H), 2.66-2.54 (m, 2H), 2.35-2.23 (m, 2H), 2.12-2.04 (m, 1H), 2.03-1.85 (m, 6H), 1.80-1.73 (m, 4H), 1.70-1.59 (m, 4H), 1.45-1.15 (m, 16H, including two singlets at 1.40 and 1.37), 1.00-0.80 (m, 16H); ¹³C NMR (100 MHz, CDCl₃) δ 165.1, 165.0, 163.7, 161.2, 133.0, 132.9, 129.7, 125.4, 125.3, 119.7, 119.5, 116.5, 116.3, 102.9, 89.5, 89.2, 81.0, 80.9, 77.2, 73.7, 70.5, 70.3, 69.6, 52.1, 51.9, 43.8, 43.7, 37.5, 37.4, 36.5, 36.3, 34.9, 34.3, 30.8, 29.8, 25.9, 24.7, 20.0, 12.5; ¹⁹F NMR (282 MHz, CDCl₃) 8-112.8; HRMS (FAB) m/z calc'd for C₄₁H₅₈FO₁₁ (M+H) 745.3963, found 745.4014.

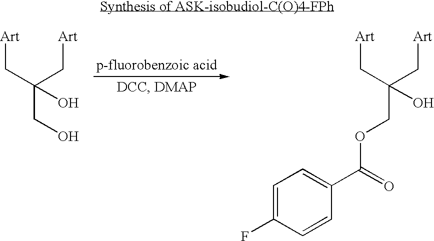

To a solution of bis-trioxane diol (62 mg, 0.10 mmol) in anhydrous dichloromethane (5.0 mL) was added N,N-dimethylaminopyridine (DMAP, 0.21 g, 1.71 mmol, 1.5 equiv) and p-benzoic acid (24 mg, 1.71 mmol, 1.5 equiv). The solution was allowed to stir for 5 mins at room temperature. To a dry pear shaped flask was added dicyclohexylcarbidimide (DCC, 40 mg, 1.71 mmol, 1.5 equiv) and additional anhydrous dichloromethane (5 mL). The DCC solution was cannulated into the bis-trioxane diol mixture at room temperature and is allowed to stir overnight. TLC analysis showed full consumption of starting material. The cloudy solution was concentrated under vacuum and purified by flash column chromatography on silica eluting with (80% EtOAc in hexanes) to give ASK-isobudiol-C(O)C(O)4-FPh as a white solid (50 mg, 70%); [□]_{D}^{22.6}=+75 (CHCl₃, c=1.0); mp=82-84° C.; IR (thin film) 3492, 2945, 2873, 1726, 1589, 1485, 1448, 1372, 1285, 1266, 1202, 1096, 1055, 1008, 910, 755, 732 cm⁻¹; ¹H NMR (400 MHz, CDCl₃) δ 8.12-8.03 (m, 2H), 7.11-7.04 (m, 2H), 5.32 (s, 1H), 5.31 (s, 1H), 4.70 (dd, J=10.0, 8.0 Hz, 1H), 4.59 (dd, J=10.0, 8.0 Hz, 1H) 4.52 (s, 1H), 4.27 (s, 1H), 2.66-2.54 (m, 2H), 2.35-2.23 (m, 2H), 2.09-1.80 (m, 7H), 1.80-1.75 (m, 2H), 1.70-1.60 (m, 2H), 1.45-1.15 (m, 16H, including two singlets at 1.40 and 1.37), 1.00-0.80 (m, 16H); ¹³C NMR (100 MHz, CDCl₃) δ 165.1, 165.0, 163.7, 161.2, 133.0, 132.9, 129.7, 125.4, 125.3, 119.7, 119.5, 116.5, 116.3, 102.9, 89.5, 89.2, 81.0, 80.9, 77.2, 73.7, 70.5, 70.3, 69.6, 52.1, 51.9, 43.8, 43.7, 37.5, 37.4, 36.5, 36.3, 34.9, 34.3, 30.8, 29.8, 25.9, 24.7, 20.0, 12.5; ¹⁹F NMR (282 MHz, CDCl₃) δ −106.4; HRMS (FAB) m/z calc'd for C₄₁H₅₈FO₁₁ (M+H) 745.3963, found 745.3983.

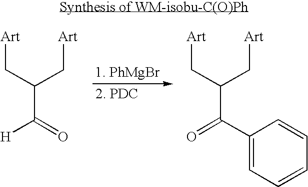

An oven dried 25 mL round bottom flask was charged with bis-trioxane aldehyde (0.91 g, 0.15 mmol) and dissolved with 2 mL of anhydrous ether. To this solution at 0° C. was added phenyl magnesium bromide (1.0 M in THF, 0.26 mL, 0.26 mmol). The reaction mixture was then allowed to ward to room temperature, and stir for 2 hr. The reaction was quenched by the slow addition of H₂O (5 mL). The contents of the flask were extracted with CH₂Cl₂ (2×25 mL), washed with a saturated aqueous solution of NaHCO₃ and H₂O, dried over MgSO₄, and concentrated in vacuo. The product was purified by silica gel chromatography (20% ethyl acetate, 80% hexanes) to give the crude product (0.069 g, 99%) which was taken on without further characterization.

The crude product was placed in a 25 mL round bottom flask, and dissolved in 6 mL of anhydrous CH₂Cl₂. To this solution was added pyridinium dichromate (PDC, 69 mg, 0.18 mmol) in one portion. After stirring overnight, the reaction mixture was filtered over celite and concentrated in vacuo. The crude product was purified by silica gel chromatography (20% ethyl acetate, 80% hexanes) to give the WM-isobu-C(O)Ph (39 mg, 56%) as an amorphous solid: [□]_{D}²³=55 (c=0.48, CHCl₃); IR (thin film) 2937, 1679, 1448, 1376, 1219, 1010 cm⁻¹; ¹H NMR (400 MHz, CDCl₃) δ 8.11-9.08 (m, 2H), 7.49-7.38 (m, 3H), 5.30 (s, 1H), 5.11 (s, 1H), 4.22-4.18 (m, 1H), 4.12-4.07 (m, 1H), 3.95-3.92 (m, 1H), 2.68-2.66 (m, 1H), 2.58-2.56 (m, 1H), 2.74-2.21 (m, 3H), 1.99-1.15 (m, 19H), 1.059 (s, 3H), 0.97-0.80 (m, 17H); ¹³C NMR (100 MHz, CDCl₃) δ 204.9, 138.0, 132.4, 129.0, 128.2, 103.2, 102.9, 89.0, 88.0, 81.2, 80.9, 75.1, 73.2, 52.4, 52.1, 44.5, 44.3, 42.9, 37.5, 37.3, 36.54, 36.46, 34.6, 34.5, 34.4, 33.6, 32.7, 30.3, 30.2, 25.8, 25.5, 24.78, 24.75, 24.72, 24.6, 22.6, 20.7, 20.2, 20.1, 14.1, 13.3, 12.8; HRMS (FAB) calc. 681.4003 for C₄₀H₅₇O₉ [(M+H)⁺], found 681.3955 (Prepared from bis-trioxane primary alcohol by the known procedure: Posner, G. H.; Shapiro, T. A.; Sur, S.; Labonte, T.; Borstnik, K.; Paik, I.-H.; McRiner, A. J. WO 2004028476).

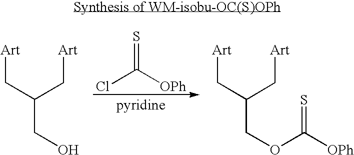

An oven dried 15 mL round bottom flask was charged with bis-trioxane alcohol (0.048 g, 0.08 mmol) and dissolved in 1 mL of anhydrous pyridine. To this solution at 0° C. was added phenylchloro thionoformate (53 mL, 0.40 mmol) dropwise over the course of 5 minutes. A white solid immediately precipitated from solution. The reaction mixture was allowed to warm to room temperature and stir for 16 hr before being quenched by the slow addition of 0.1N citric acid (5 mL). The contents of the flask were extracted with CH₂Cl₂ (2×25 mL), washed with an aqueous saturated solution of NaHCO₃ and H₂O, dried over MgSO₄, and concentrated in vacuo. The crude product was purified by silica gel chromatography (20% ethyl acetate in hexanes) to give WM-isobu-OC(S)OPh as an amorphous solid (25 mg, 42%): [□]_{D}²³=53.0, (c=1.02, CHCl₃); IR (thin film) 2939, 2875, 1592, 1490, 1454, 1377, 1280, 1201, 1105, 1009, 754 cm⁻¹; ¹H NMR (400 MHz, CDCl₃) δ 7.41-7.38 (m, 2H), 7.29-7.25 (m, 1H), 7.15-7.13 (m, 2H), 5.34 (s, 1H), 5.33 (s, 1H), 4.73-4.65 (m, 2H), 4.43-4.24 (m, 1H), 4.29-4.28 (m, 1H), 2.71-2.69 (m, 1H), 2.61-2.59 (m 1H), 2.37-2.28 (m, 3H), 2.04-2.00 (m, 2H), 1.91-1.25 (m, 24H), 0.98-0.94 (m, 8H), 0.88-0.83 (m, 8H); ¹³C NMR (100 MHz, CDCl₃) δ 194.8, 153.5, 129.3, 126.3, 122.1, 103.2, 102.9, 89.5, 88.8, 81.14, 81.11, 77.3, 76.4, 73.6, 70.9, 52.4, 52.1, 44.5, 44.1, 37.42, 37.40, 36.68, 36.62, 34.5, 34.4, 34.2, 30.5, 30.4, 29.9, 26.11, 26.10, 25.3, 24.82, 24.77, 24.7, 20.19, 20.09, 13.2, 12.7.

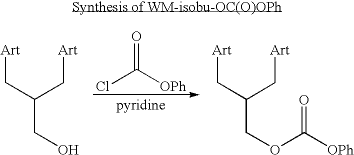

An oven dried 15 mL round bottom flask was charged with bis-trioxane alcohol (0.080 g, 0.13 mmol) and dissolved in 3 mL of anhydrous pyridine. To this solution at 0° C. was added phenylchloro formate (82 mL, 0.66 mmol) dropwise over the course of 5 minutes. A white solid immediately precipitated from solution. The reaction mixture was allowed to warm to room temperature and stir for 16 hr before being quenched by the slow addition of 0.1N citric acid (5 mL). The contents of the flask were extracted with CH₂Cl₂ (2×25 mL), washed with a saturated aqueous NaHCO₃ and H₂O, dried over MgSO₄, and concentrated in vacuo. The crude product was purified by silica gel chromatography (20% ethyl acetate in hexanes) to give WM-isobu-OC(O)OPh as a white solid (92 mg, 96%): [□]_{D}²³=64.0 (c=1.9, CHCl₃); mp=82-84° C.; IR (thin film) 2924, 1762, 1494, 1456, 1377, 1256, 1211, 1106, 1054, 1008 cm⁻¹; ¹H NMR (400 MHz, CDCl₃) δ 7.37-7.33 (m, 2H), 7.22-7.17 (m, 3H), 5.33 (s, 1H), 5.31 (s, 1H), 4.47-4.41 (m, 3H), 4.29-4.26 (m, 1H), 2.74-2.64 (m, 1H), 2.64-2.54 (m, 1H), 2.37-2.21 (m, 3H), 2.02-1.98 (m, 2H), 1.91-1.21 (m, 24H), 0.97-0.93 (m, 8H), 0.88-0.82 (m, 8H); ¹³C NMR (100 MHz, CDCl₃) δ153.5, 151.3, 129.2, 125.7, 121.1, 103.1, 102.8, 89.5, 88.8, 81.08, 81.07, 73.7, 70.8, 70.6, 52.3, 52.1, 44.4, 44.1, 37.38, 37.35, 36.63, 36.57, 34.6, 34.44, 34.37, 34.3, 31.5, 30.5, 30.3, 29.8, 26.0, 25.95, 25.2, 24.8, 24.73, 24.67, 22.58, 20.64, 20.2, 20.1, 14.1, 13.1, 12.6; HRMS (FAB) calc. 727.4057 for C₄₁H₅₉O₁₁ [(M+H)⁺], found 727.4058.

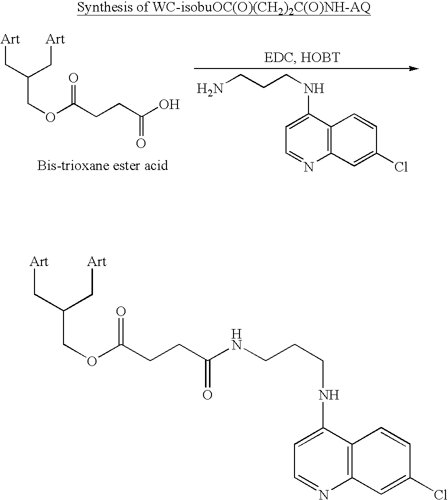

To a solution of bis-trioxane ester acid (54 mg, 0.08 mmol) in CH₂Cl₂ (1 mL) at 0° C. were added N-(3-dimethylamino-propyl)-N′-ethylcarbodiimide hydrochloride (EDC, 18 mg, 0.09 mmol) and 1-hydroxybenzotriazole (HOBt, 12 mg, 0.09 mmol) and it was stirred for 20 min at rt. To the reaction at 0° C. was added a solution of N-(7-Chloro-quinolin-4-yl)-propane-1,3-diamine (27 mg, 0.11 mmol) and triethylamine (21 μL, 0.15 mmol) in CH₂Cl₂ (1 mL) dropwise. The solution was warmed to rt and stirred for 30 min. It was diluted with ether (5 mL) and quenched with water (2 mL). Layers were separated and the aqueous layer was extracted with ether (4×3 mL). The combined organic solution was dried (MgSO₄) and concentrated. The residue was purified by flash column chromatography (elution with EtOAc only) to provide WC-isobuOC(O)(CH₂)₂C(O)NH-AQ (56 mg, 79%) as a white solid: [α]_{D}²⁴=+57 (c 0.79, CHCl₃); mp 120-125° C.; IR (thin film) 3277, 2923, 1733, 1654, 1581, 1375, 1010, 755 cm⁻¹; ¹H NMR (400 MHz, CDCl₃) δ 8.46 (d, J=5.2 Hz, 1H), 7.96 (d, J=8.8 Hz, 1H), 7.90 (d, J=2.4 Hz, 1H), 7.36 (dd, J=2.0, 8.8 Hz, 1H), 6.61-6.52 (m, 2H), 6.38 (d, J=5.2 Hz, 1H), 5.29 (s, 1H), 5.25 (s, 1H), 4.35 (m, 1H), 4.27-4.18 (m, 3H), 3.44-3.30 (m, 4H), 2.83-2.62 (m, 3H), 2.58-2.46 (m, 3H), 2.36-1.54 (m, 17H), 1.45-1.15 (m, 16H including s at 1.37 and 1.34), 0.95-0.78 (m, 14H including d at 0.85 with J=7.6 Hz and 0.80 with J=7.6 Hz); ¹³C NMR (100 MHz, CDCl₃) δ 173.2, 172.8, 151.8, 150.0, 149.3, 134.8, 128.4, 125.2, 122.1, 117.6, 103.3, 102.9, 98.4, 89.4, 89.0, 81.1, 81.1, 73.6, 71.0, 67.6, 52.3, 52.1, 44.3, 44.1, 39.0, 37.5, 37.4, 36.6, 36.5, 36.3, 34.4, 34.4, 33.8, 31.5, 31.2, 30.7, 30.5, 30.1, 28.2, 26.0, 25.9, 24.8, 24.7, 20.2, 20.1, 19.1, 14.1, 13.7, 13.1, 12.7; HRMS (FAB) calculated for C₅₀H₇₁ClN₃O₁₁ [(M+H)⁺] 924.4777, found 924.4813 (Prepared from bis-trioxane primary alcohol by the previously reported procedure: Posner, G. H.; Paik, I.-H.; Sur, S.; McRiner, A. J.; Borstnik, K.; xie, S.; Shapiro, T. A. J. Med. Chem. 2003, 46, 1060).

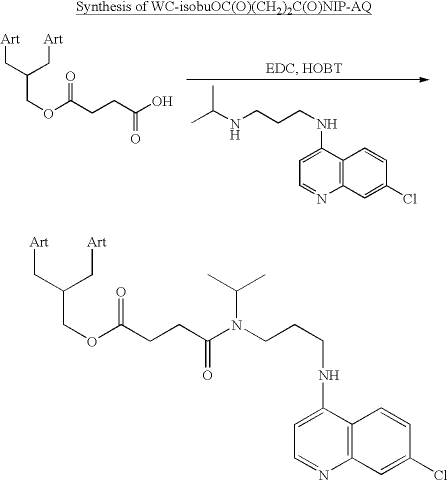

To a solution of bis-trioxane ester acid⁴ (72 mg, 0.10 mmol) in CH₂Cl₂ (1 mL) at 0° C. were added N-(3-dimethylamino-propyl)-N′-ethylcarbodiimide hydrochloride (EDC, 23 mg, 0.12 mmol) and 1-hydroxybenzotriazole (HOBt, 17 mg, 0.12 mmol) and it was stirred for 20 min at rt. To the reaction at 0° C. was added a solution of WC-1,3-diamine (57 mg, 0.20 mmol) and triethylamine (28 μL, 0.20 mmol) in CH₂Cl₂ (1 mL) dropwise. The solution was warmed to rt and stirred for 2 h. It was diluted with ether (5 mL) and quenched with water (2 mL). Layers were separated and the aqueous layer was extracted with ether (4×3 mL). The combined organic solution was dried (MgSO₄) and concentrated. The residue was purified by flash column chromatography (elution with EtOAc only) to provide WC-isobuOC(O)(CH₂)₂C(O)NIP-AQ (74 mg, 75%) as a white solid: [α]_{D}²⁴=+45 (c 0.60, CHCl₃); mp 89-91° C.; IR (thin film) 2923, 1733, 1581, 1451, 1374, 1009, 755 cm⁻¹; ¹H NMR (400 MHz, CDCl₃) δ 8.47 (d, J=5.2 Hz, 1H), 8.15 (d, J=9.2 Hz, 1H), 7.93 (d, J=2.0 Hz, 1H), 7.40 (dd, J=2.2, 9.2 Hz, 1H), 7.32 (m, 1H), 6.31 (d, J=5.6 Hz, 1H), 5.27 (s, 1H), 5.25 (s, 1H), 4.36 (m, 1H), 4.29-4.20 (m, 3H), 3.39 (t, J=6.4 Hz, 2H), 3.27 (q, J=6.0 Hz, 2H), 2.80-2.46 (m, 9H), 2.33-2.16 (m, 3H), 2.03-1.15 (m, 34H including s at 1.37 and 1.35), 0.97-0.78 (m, 14H); ¹³C NMR (100 MHz, CDCl₃) δ 173.0, 171.6, 151.8, 150.0, 149.3, 134.8, 128.4, 125.3, 122.7, 117.6, 103.0, 102.8, 98.0, 89.5, 89.0, 81.1, 81.1, 73.1, 71.2, 67.1, 52.3, 52.1, 48.3, 44.3, 44.1, 40.6, 37.7, 37.5, 37.4, 36.7, 36.6, 34.4, 34.1, 31.6, 31.0, 30.6, 30.1, 29.7, 29.6, 29.3, 28.4, 26.1, 26.0, 25.3, 24.8, 24.7, 21.4, 20.2, 20.1, 14.1, 13.0, 12.6; HRMS (FAB) calculated for C₅₃H₇₇C1N₃O₁1 [(M+H)⁺] 966.5247, found 966.5282.

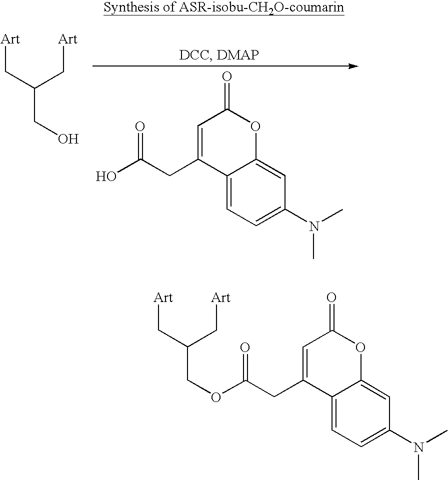

Bis-trioxane primary alcohol (98 mg, 0.16 mmol), 7-dimethylaminocoumarin-4-acetic acid (20 mg, 0.08 mmol) and DMAP (10 mg, 0.08 mmol) were added to dichloromethane (7 mL) under argon. N,N′-dicyclohexylcarbodiimde (DCC, 16.7 mg, 0.081 mmol) was added to the reaction and it was stirred at room temperature for 18 h. The orange solution was concentrated in vacuo. The crude product was purified by flash silica gel column chromatography (20 to 30% EtOAc in hexanes) to yield ASR-isobu-CH₂O-coumarin as a pale yellow amorphous solid (28 mg, 41%): [α]_{D}²²³+52° (c=0.18, CHCl₃); IR (thin film) 3057, 2951, 2876, 1719, 1619, 1532, 1453, 1403, 1375, 1268, 1145, 1105, 1053, 1008, 735, 702 cm⁻¹; ¹HNMR (400 MHz, CDCl₃) δ7.43-7.41 (d, J=8.8 Hz, 1H), 6.64-6.61 (m, 1H), 6.51-6.50 (d, J=2.0 Hz, 1H), 6.05 (s, 1H), 5.23 (s, 1H), 5.22 (s, 1H), 4.33-4.16 (m, 4H), 3.70 (s, 2H), 3.04 (s, 6H), 2.61-2.59 (m, 1H), 2.39-2.38 (m, 1H), 2.32-2.24 (m, 2H), 2.11-1.87 (m, 6H), 1.73-1.55 (m, 9H), 1.42-1.13 (m, 17H including singlets at 1.37 and 1.36), 0.95-0.85 (m, 9H), 0.78-0.76 (d, J=7.2 Hz, 3H), 0.71-0.69 (d, J=7.2 Hz, 3H); ¹³C NMR (100 MHz, CDCl₃) δ 168.9, 161.6, 155.9, 152.7, 148.9, 125.6, 110.8, 109.2, 108.9, 103.2, 102.8, 98.4, 89.6, 88.9, 81.2, 81.1, 73.6, 70.5, 67.1, 52.3, 52.1, 44.4, 44.1, 40.2, 38.6, 37.5, 36.7, 36.6, 34.5, 34.4, 34.0, 30.1, 30.6, 30.4, 30.2, 26.1, 26.0, 24.8, 20.3, 20.1, 13.0, 12.5; HRMS (FAB) m/z calc'd for C₄₇H₆₅NO₁₂ (M+H)⁺ 836.4585, found 836.4578; HPLC [semi-preparative silica gel column (1×25 cm)], 30% EtOAc in hexanes, 2 mL/min, 270 nm, t_{R}=43.6 min.

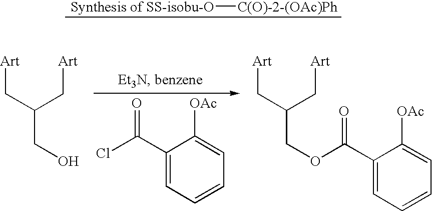

A flame-dried 20 mL round bottom flask equipped with a magnetic stir bar, a septum along with an Ar balloon was charged with bis-trioxane primary alcohol (25 mg, 0.04 mmol) and dissolved in 2 mL freshly distilled benzene. At room temperature, to the solution of primary alcohol, triethylamine (0.019 mL, 2.10 mmol, 5.0 eq) and acetylsalicyloyl chloride (15 mg, 0.03 mmol) were added respectively. The mixture was heated to 45° C. and was stirred for 24 h. The reaction was quenched by addition of 10 mL cold distilled water and then rinsed into a separatory funnel with ethyl ether (10 mL). The mixture was extracted with ethyl acetate (3×30 mL). The combined extracts were washed with water (5 mL), and 5% sodium carbonate in water (5 mL), dried over Na₂SO₄ and filtered. The filtrate was concentrated in vacuo to give the crude product that was purified by flash column chromatography, which was eluted with 20% ethyl acetate in hexanes to afford SS-isobu-O—C(±)-2-(OAc)Ph (27 mg, 87%) as an amorphous solid: [α]²⁵_{D} +69.4 (c 1.00, CHCl₃); IR (thin film) 2954, 2880, 1766, 1716, 1452, 1370, 1290, 1187, 1105, 1072, 1031, 1006, 924, 743 cm⁻¹; ¹H NMR (400 MHz, CDCl₃) δ 8.01-7.99 (dd, J=7.6, 1.6 Hz, 1H), 7.56-7.52 (m, 1H), 7.30-7.28 (m, 1H), 7.10-7.08 (dd, J=8.0, 0.80 Hz, 1H), 5.31 (d, J=2.8 Hz, 2H), 4.48-4.39 (m, 3H), 4.30-4.27 (m, 1H), 2.75-2.55 (m, 2H), 2.37-2.28 (m, 6H, including a singlet at 2.35), 2.04-1.97 (m, 2H), 1.89-1.51 (m, 12H), 1.46-1.20 (m, 13H, including two singlet at 1.40, and 1.39), 0.97-0.83 (m, 14H); ¹³C NMR (100 MHz, CDCl₃) δ 169.7, 164.24, 150.75, 133.47, 131.50, 125.8, 123.8, 103.2, 102.9, 89.34, 88.64, 81.15, 81.10, 77.20, 73.39, 71.28, 67.54, 52.41, 52.15, 44.49, 44.17, 37.37, 37.30, 36.65, 36.58, 34.45, 33.93, 30.56, 30.41, 30.30, 29.74, 26.11, 26.03, 24.91, 24.85, 24.71, 24.63, 21.04, 20.19, 20.11, 14.11, 13.21, 12.77.

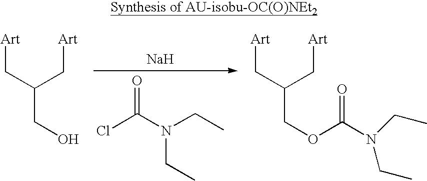

Bis-trioxane primary alcohol (40 mg, 0.07 mmol) was dissolved in CH₂Cl₂ (0.8 mL) in an oven-dried 10 ml round bottom flask charged with magnetic stir bar and argon balloon. Sodium hydride (60% in mineral oil, 4 mg, 0.10 mmol) was added which resulted in fizzing and a cloudy white solution. After 1 hour, diethylcarbamyl chloride (9 mg, 0.07 mmol) was added. The reaction stirred 16 hours at room temperature. Starting material was not consumed. Sodium hydride (60% in mineral oil, 4 mg, 0.10 mmol) was added and the reaction stirred for 16 hours more at which time TLC showed almost complete consumption starting material. The reaction was quenched with H₂O (10 mL), and the organics were extracted with methylene chloride (1×10 mL) followed by ethyl acetate (2×10 mL). The organic layer was dried over magnesium sulfate, filtered, and concentrated. Purification of the crude product by column chromatography (60% EtOAc in Hex) gave AU-isobu-OC(O)NEt₂ (32 mg, 70%) as an amorphous solid: [α]_{D}²¹=49.3 (c=2.43, CHCl₃); IR (thin film) 2928(s), 2870(m), 1693(s), 1480(m), 1423(m), 1374(m), 1268(m), 1230(w), 1191(m), 1114(m), 1056(m), 998(s), 958(w), 950(w), 872(w), 766(m); ¹H NMR (400 MHz, CDCl₃) δ 5.26 (d, J=1.6 Hz, 2H), 4.34-4.30 (m, 1H), 4.22-4.12 (m, 3H), 3.21 (s, br, 4H), 2.69-2.64 (m, 1H), 2.59-2.54 (m, 1H), 2.31-2.22 (m, 2H), 2.16 (m, 1H), 2.02-1.94 (m, 2H), 1.86-1.78 (m, 2H), 1.76-1.68 (m, 3H), 1.61-1.37 (m, 7H), 1.36-1.30 (m including singlets at 1.35 and 1.34, 7H), 1.31-1.11 (m, 6H), 1.06 (t, J=7.2 Hz, 6H), 0.92-0.89 (m, 8H), 0.83-0.79 (m, 7H); ¹³C NMR (100 MHz, CDCl₃) δ 156.0, 103.2, 103.0, 100.8, 88.2, 88.6, 81.2, 81.1, 73.6, 72.1, 67.4, 60.4, 52.5, 52.3, 44.6, 44.4, 37.4, 37.3, 36.7, 36.6, 34.5, 34.4, 34.3, 30.8, 30.5, 30.4, 29.8, 26.1, 26.0, 24.8, 24.7, 24.6, 20.3, 20.2, 14.2, 13.3, 12.9; HRMS (FAB) calculated for C₃₉H₆₄NO₁₀ [(M+H)⁺] 706.4530, found 706.4540.

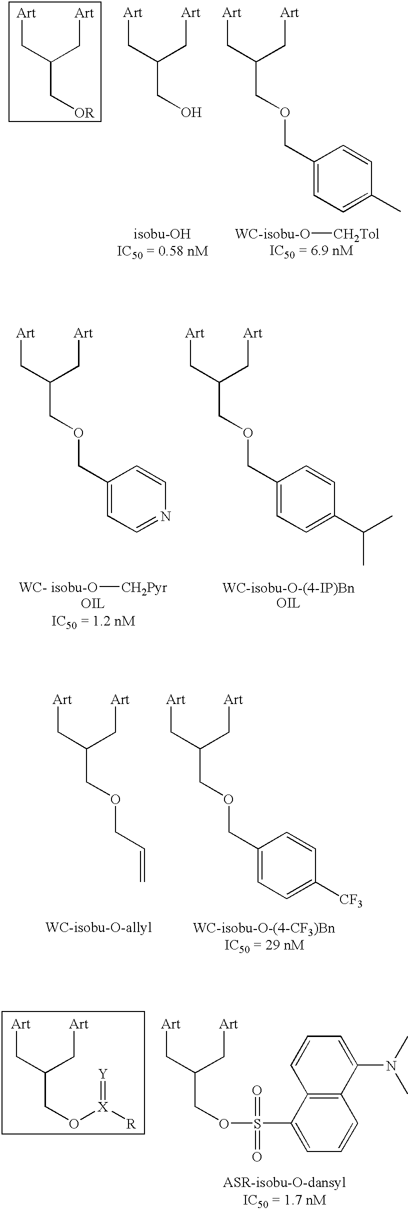

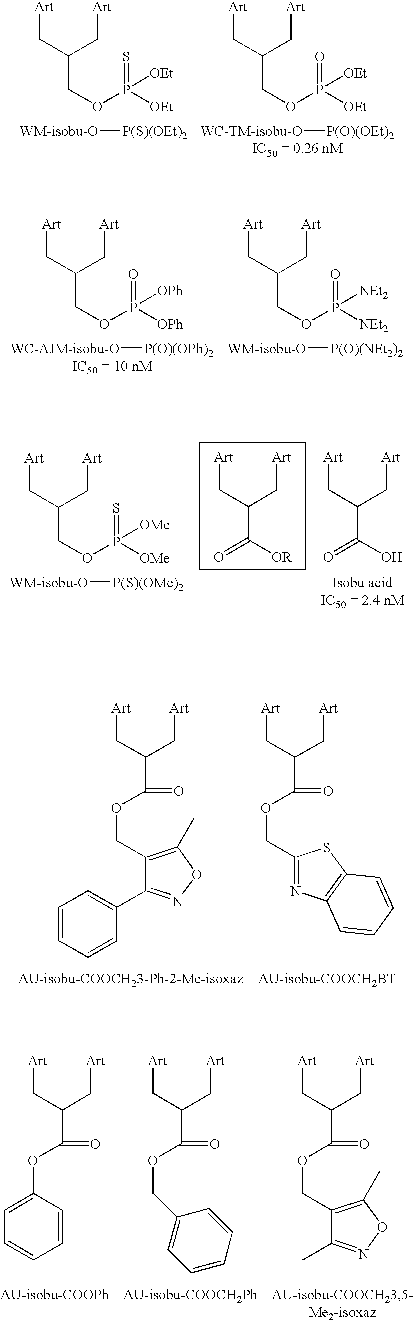

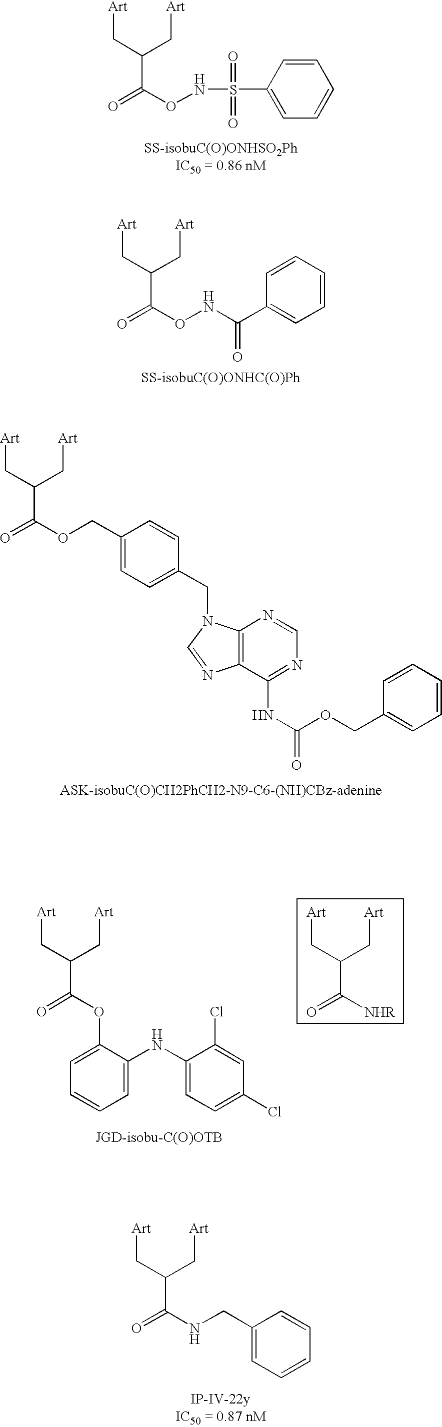

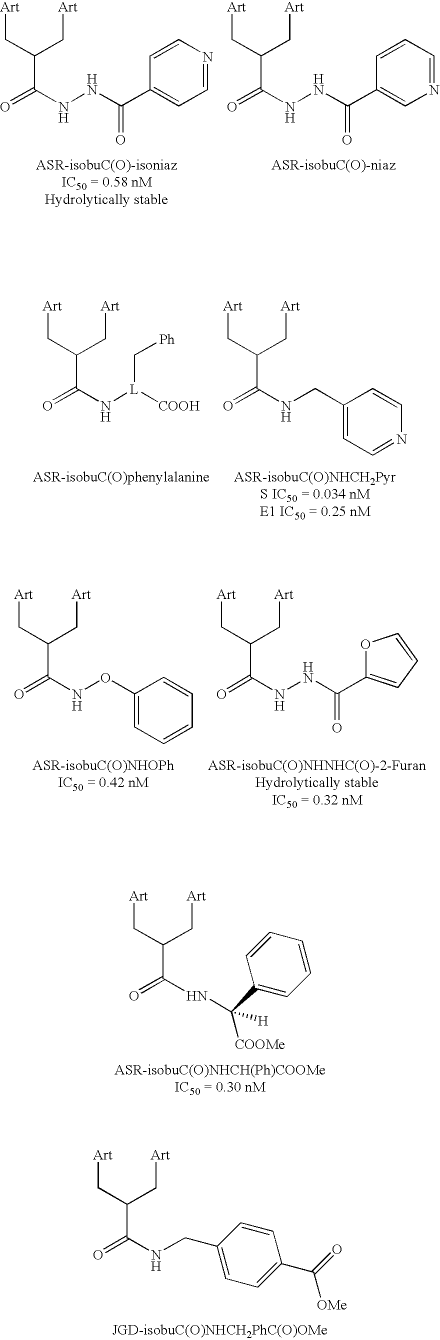

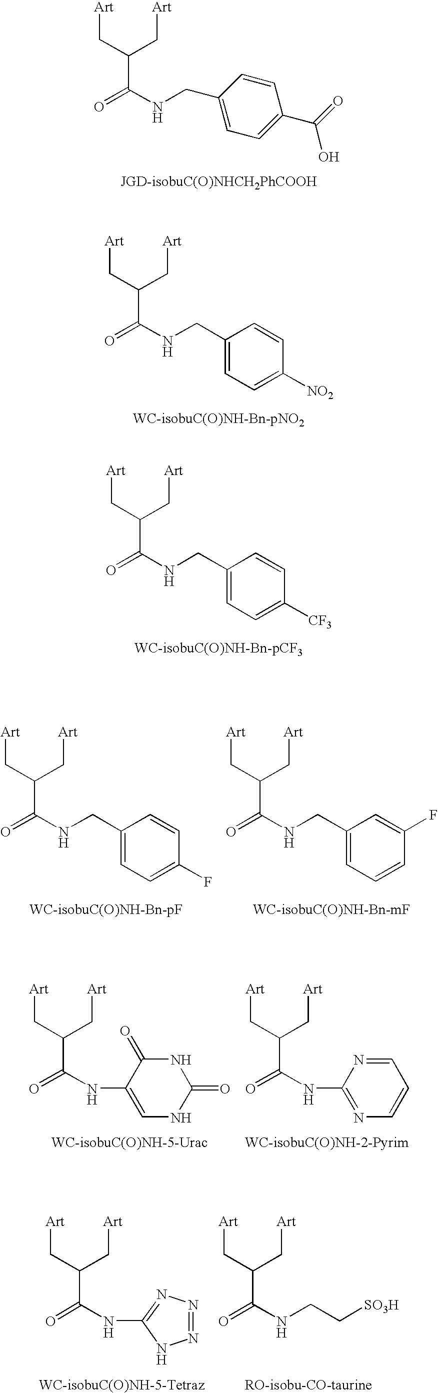

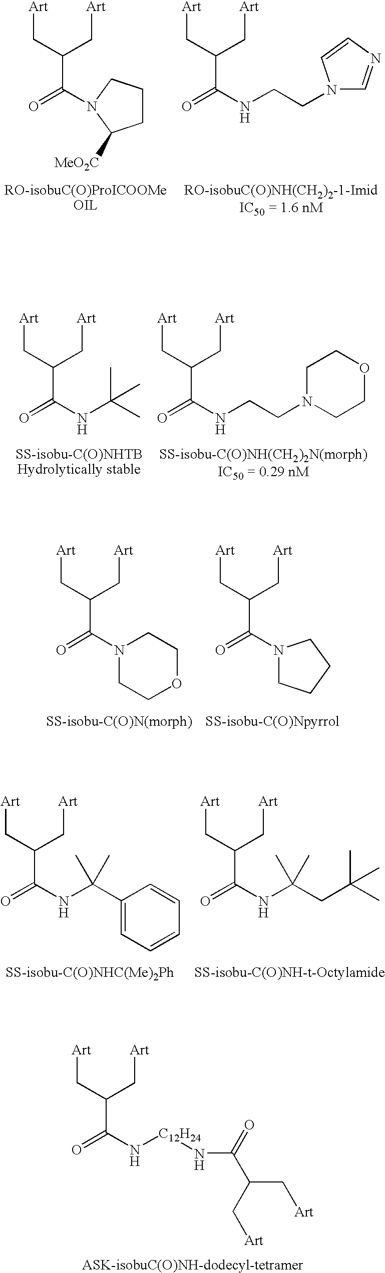

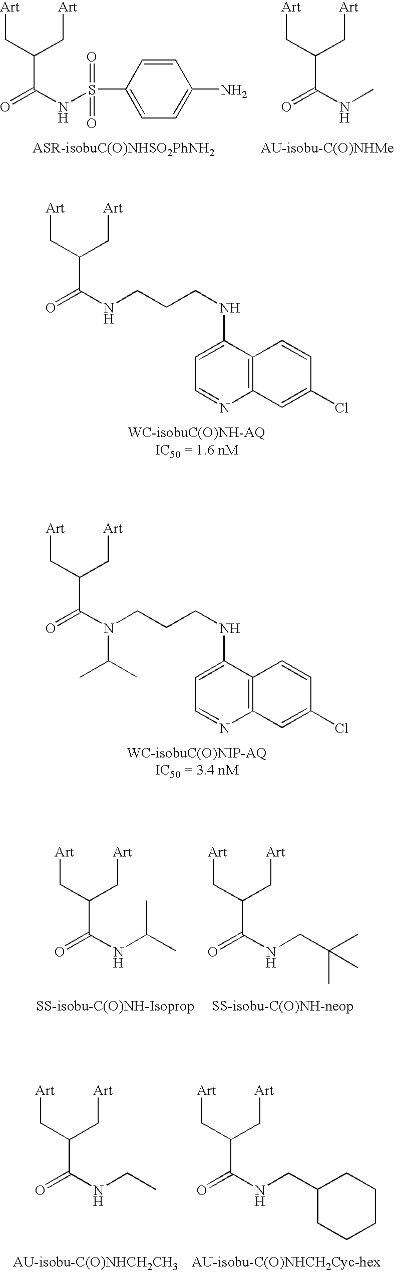

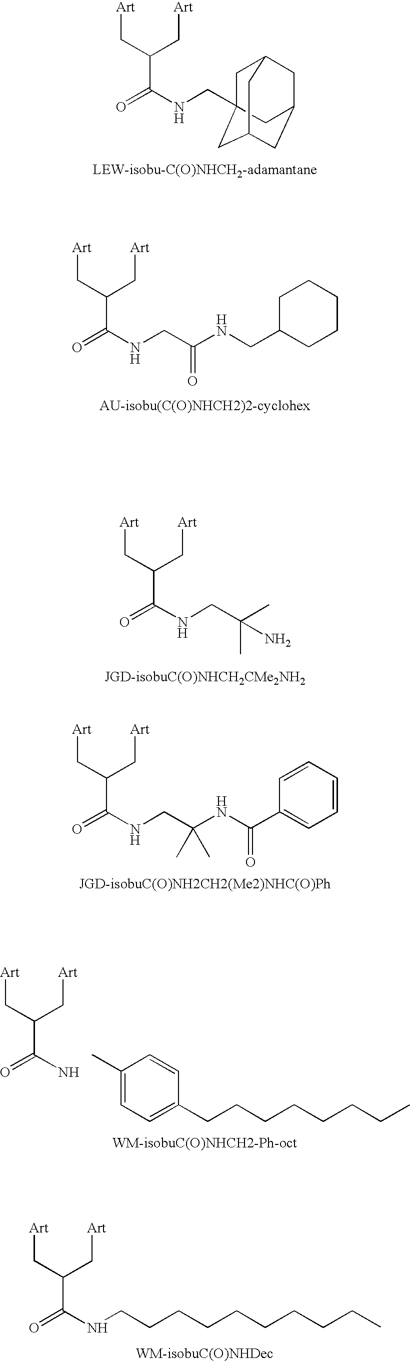

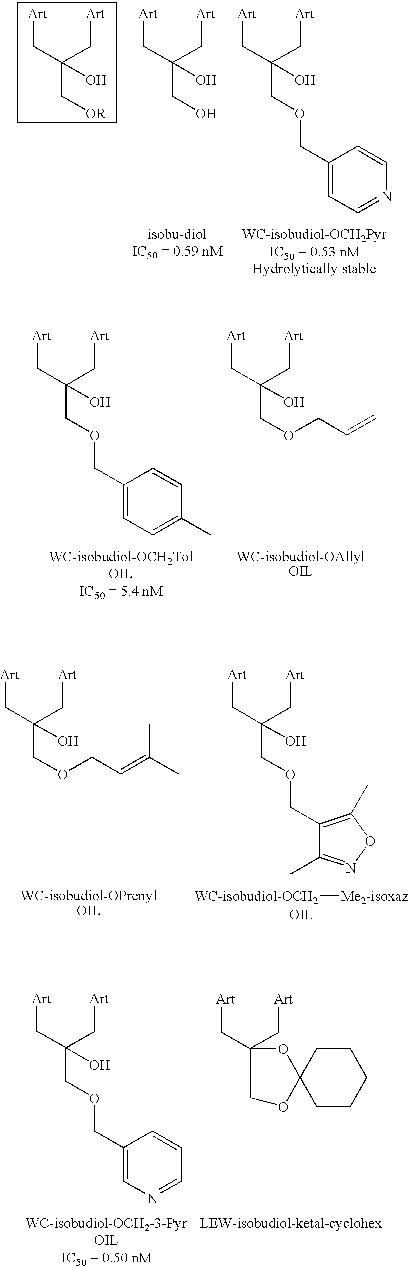

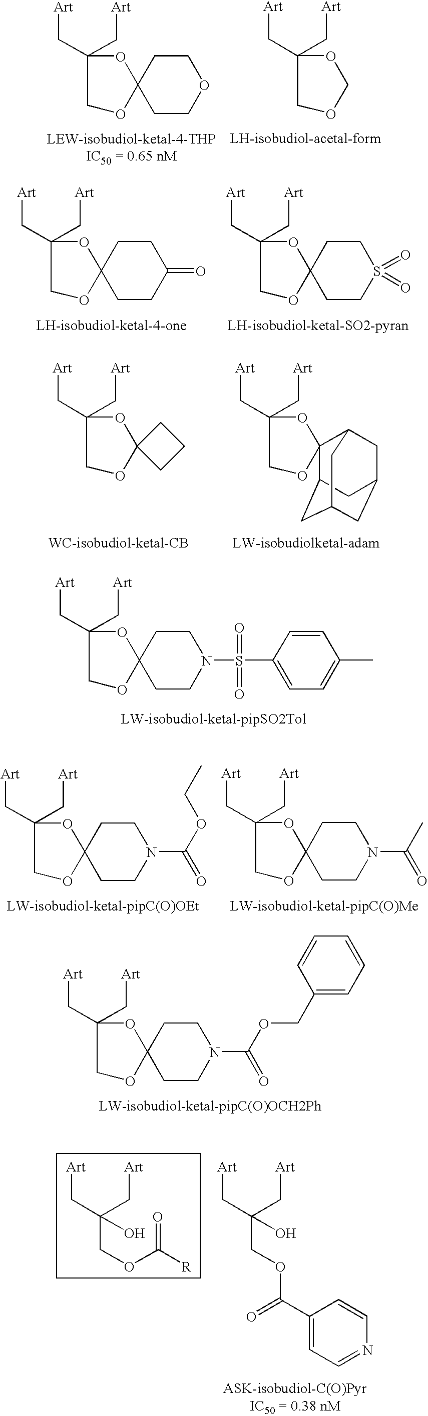

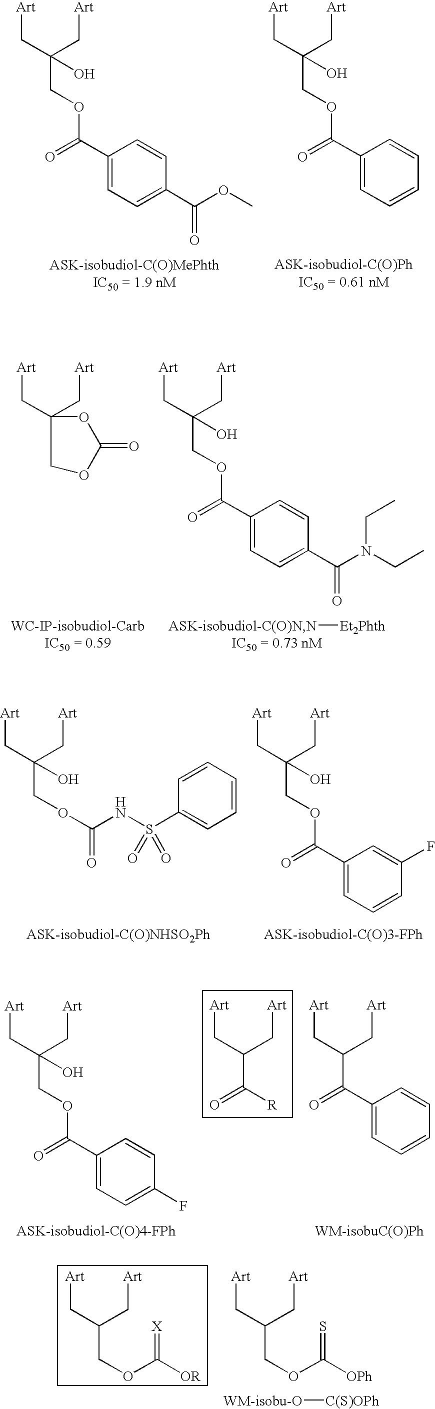

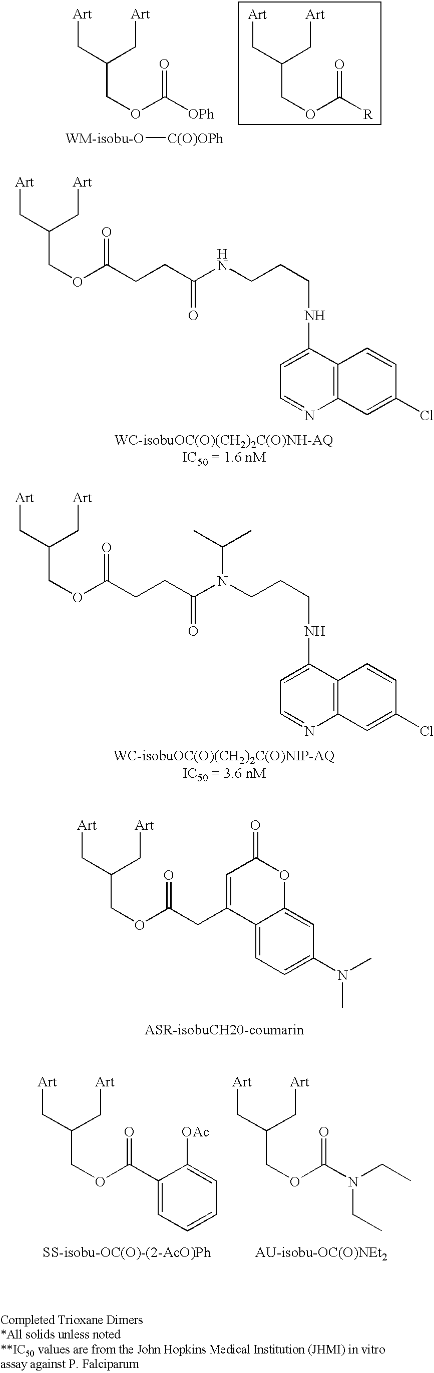

### Examples

### Trioxane Dimers

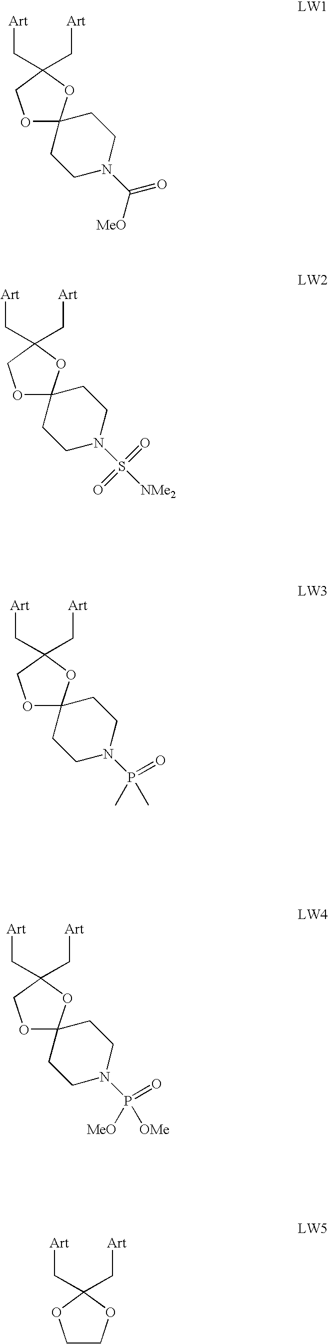

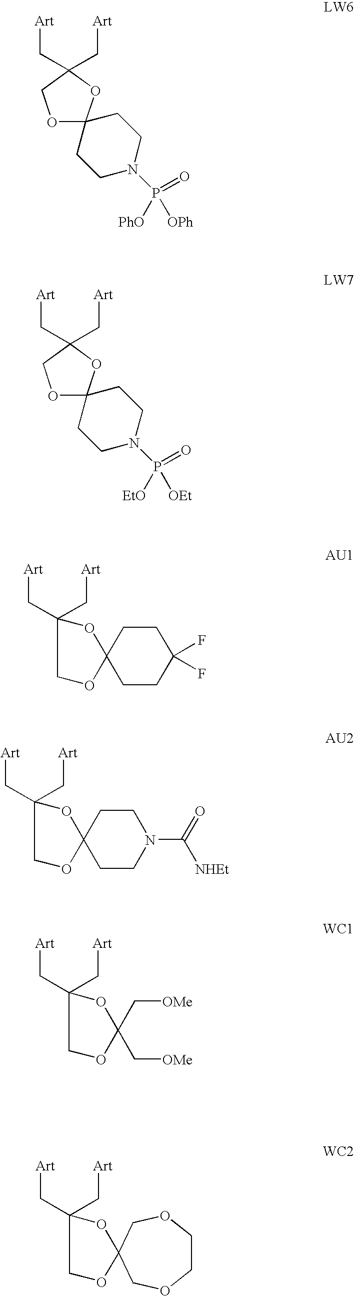

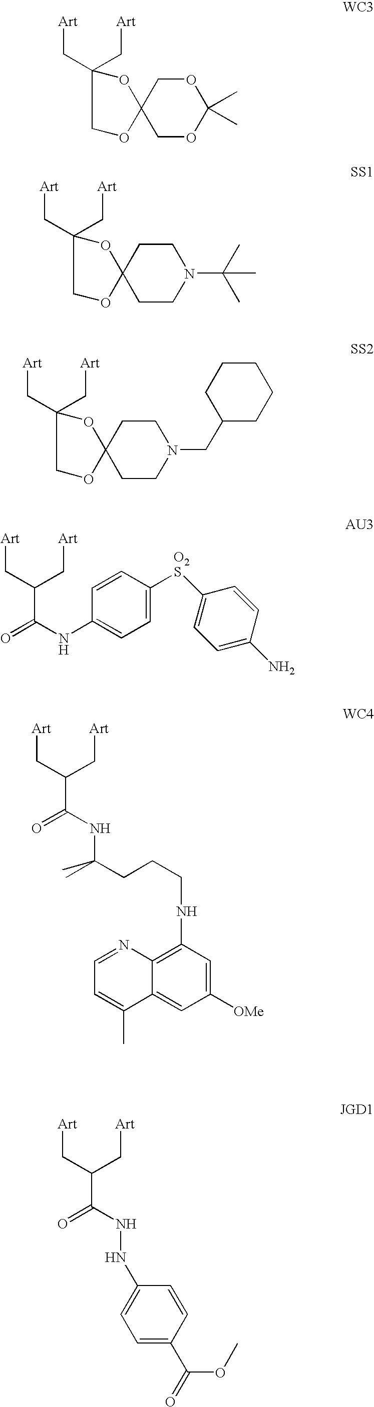

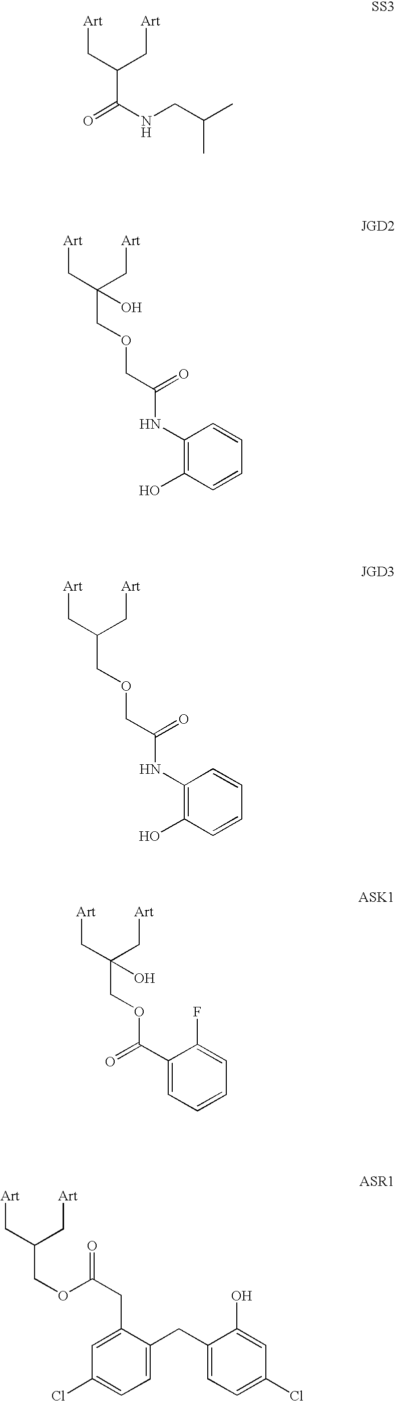

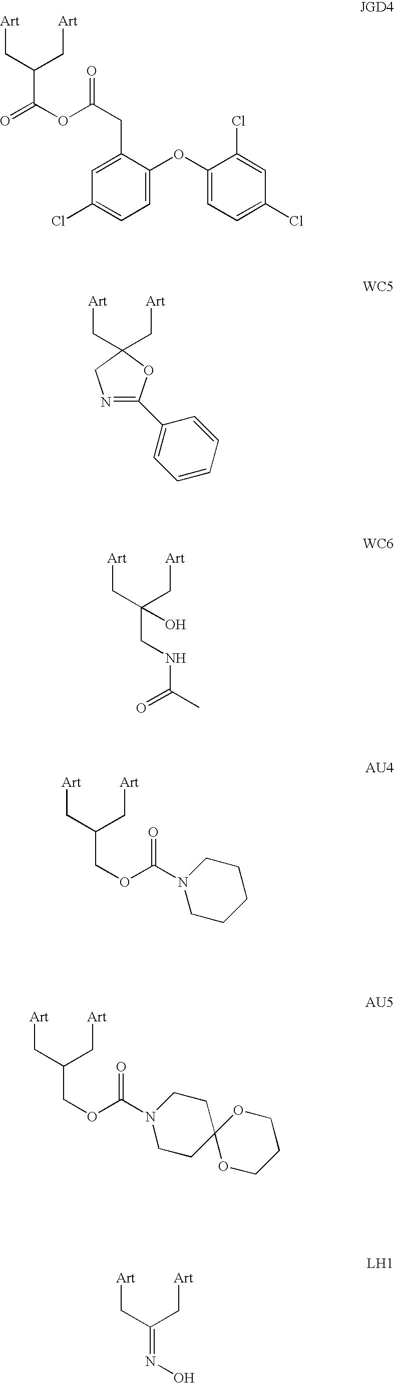

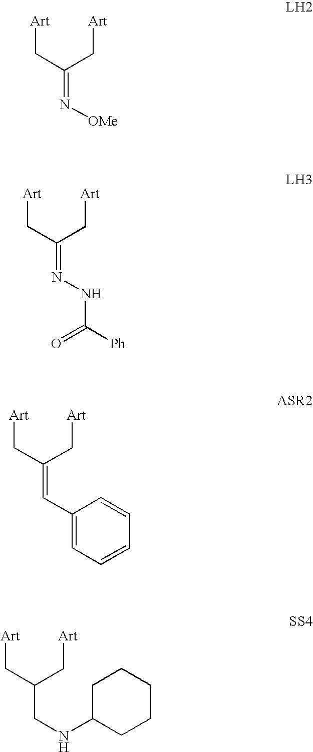

Bis-trioxanes with novel skeletons and functionalities were prepared (see New Trioxane Dimers) and many of them showed excellent antimalarial activity. For example, WM-isobu-O—P(S)(OMe)₂ has a new phosphorothioate moiety and it cured malaria-infected mice at 3×30 mg/kg oral dose.

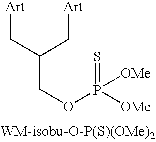

AU-isobu-C(O)NHCH₂Cyc-hex has an amide with a cyclohexyl chain and, unlike our previously prepared amides, it cured malaria-infected mice at 3×30 mg/kg oral dose.

LH-isobudiol-ketal-4-one has a new ketal moiety. At 3×30 mg/kg oral dose, it cured malaria-infected mice. At an even smaller oral dose (3×10 mg/kg), it prolonged the lives of malaria-infected mice up to 16.3 days.

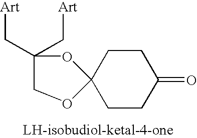

Newly prepared functionalities include carbonate, carbamate, ketone and phosphorodiamidate, as shown below.

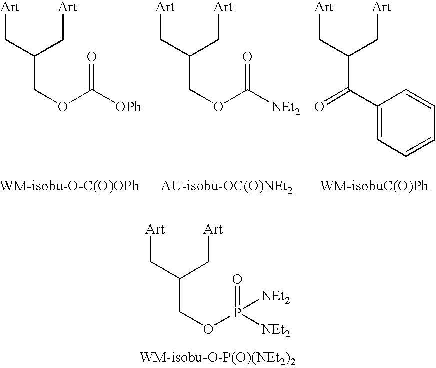

The trioxane dimers described herein demonstrate enhanced oral in vivo antimalarial activity. When it comes to the treatment of malaria, feasibility of oral administration is a decisive factor to determine usefulness of a therapeutic agent.

Compounds of the claimed invention gave unexpectedly high oral in vivo antimalarial activity in mouse model studies, higher than those of prior art: For example,

1) Cure (survival after 30 days of infection) of malaria-infected mice with Just three 3×30 mg/kg dose over three days was achieved with each of the following new dimers.

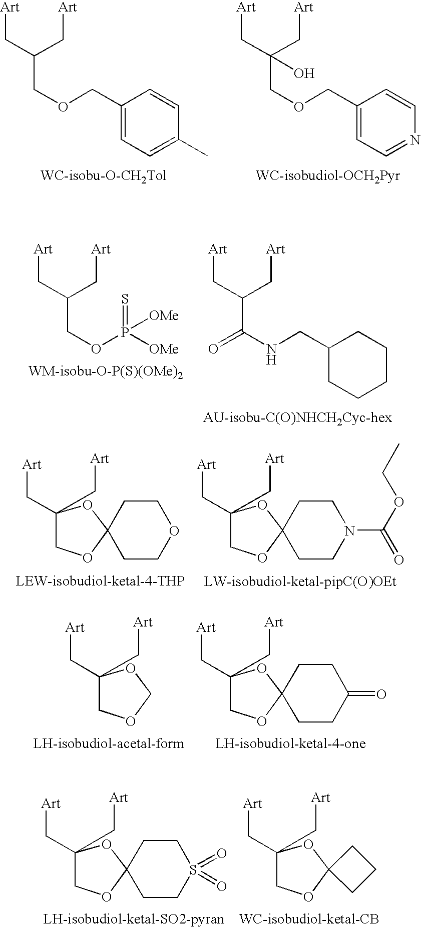

More than two week prolongation of lives of malaria-infected mice was achieved with even smaller oral doses (3×10 mg/kg):

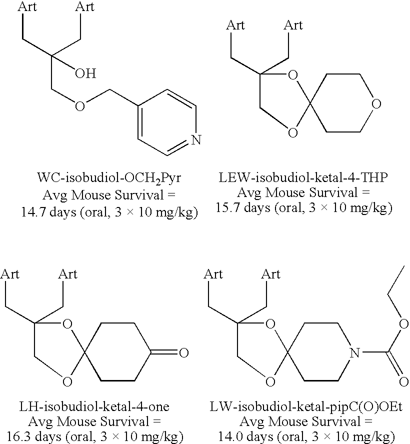

| | | Sodium | |
|---|---|---|---|
| | Control | Artesunate | OZ277 tosylate |
| Avg Mouse | 4.0 | 6.7 | 18.8 |
| Survival | | | |
| (oral, 3 × 10 mg/kg) | | | |

### Assays

Using our standard assay (Posner, G. H. et al., Tetrahedron 53:37-50 (1997)), we determined the antimalarial potencies of these dimers in vitro against chloroquine-sensitive Plasmodium falciparum (NF 54) parasites (Table 1). Except for water-soluble phthalic acid dimer 6, all of the other dimers in Table 1 are considerably more potent antimalarials than natural artemisinin (1, IC₅₀=6.6±0.76 nM). Bis-benzyl alcohol dimer 7 stands out as the most potent, being approximately 10-times more antimalarially active than artemisinin (1).

As measured in mice according to a published protocol involving single administration at dose of 3, 10, or 30 mg/kg, either subcutaneously (SC) or orally (PO) (Fidock, D. A. et al., Nat. Rev. Drug Discov. 3:509-520 (2004)), bis-ester dimer 5 has SC ED₅₀=0.71 mg/kg and diol dimer 7 has SC ED₅₀=0.06 mg/kg and PO ED₅₀=2.6 mg/kg. Under these test conditions, the clinically used monomeric trioxane sodium artesunate has SC ED₅₀=2.2 mg/kg and PO ED₅₀=4.0 mg/kg. Thus, these two dimers 5 and 7 are approximately 3-37 time, more efficacious than the antimalarial drug sodium artesunate administered SC, and diol dimer 7 is approximately 1.5 times more efficacious than sodium artesunate administered PO. Neither over toxicity nor behavioral modification was observed in the mice due to drug administration.

TABLE 1
Antimalarial Activities in vitro⁸

| trioxane dimer | IC₅₀ (nM) |
|---|---|
| 4 | 2.9 |
| 5 | 1.6 |
| 6 | 360 |
| 7 | 0.77 |
| 8 | 3.0 |
| 9 | 3.7 |
| Artemisinin | 6.6 |

⁸The standard deviation for each set of quadruplicates was an average of 7.8% (≦18%) of the mean. R² values for the fitted curves were ≧0.967.
Artemisinin activity is the mean ± standard deviation of the concurrent control (n = 6).

Preliminary growth inhibitory activities at nanomolar to micromolar concentrations; measured in vitro as described previously using a diverse panel of 60 human cancer cell lines in the National Cancer Institute's (NCI's) Development and Therapeutic Program (Boyd, M. R. et al., Drug Dev. Rev. 34:91-109 (1995)) showed phthalate dimer 5 to be extremely selective and highly potent at inhibiting the growth of only non-small cell lung carcinoma HOP-92 cells, melanoma SK-MEL-5 cells, and breast cancer BT-549 cells. Employing a tetrazolium salt (XTT) based calorimetric proliferation assay (Roche Diagnostics, Mannheim, Germany) and using a modified version of a recently reported protocol for in vitro evaluation of the growth inhibitory activity of DHA toward the human cervical cancer cell line HeLa (IC₅₀=5-10 micromolar) (Disbrow, G. L. et al., Cancer Res. 65:10854-10861 (2005)), we have found unexpectedly but importantly that trioxane phthalate dimer 5 (IC₅₀-500 nM) is approximately 10-20 times more potent than trioxane monomer DHA and that trioxane diol dimer 7 (IC₅₀=46.5 nM) is approximately 110-220 times more potent than DHA, without being toxic to primary normal cervical cells. Cell growth was inhibited in a dose-dependent manner.

Using a standard protocol in Plasmodium berghei infected mice, trioxane dimers IP-IV-22y and KB-06 were administered subcutaneously only once at a dose of 3, 10, or mg/kg body weight. Both dimers at the single dose of 30 mg/kg dose rapidly killed more than 98% of the malaria parasites. The currently used antimalarial drug sodium artesunate at 30 mg/kg was similarly efficacious. Sodium artesunate at 30 mg/kg prolonged the life of the mice from 7 days (no drug) to only 14 days. Unexpectedly but of great medical importance, both dimers at 30 mg/kg prolonged the life of the mice to at least 30 days at which time the mice were considered cured (i.e. no parasites detected in blood smears)! Neither overt toxicity nor behavioral modification was observed in the mice due to drug administration.

### Cells and Cell Culture

In vivo antimalarial testing was performed at the Swiss Tropical Institute. Compounds were formulated for subcutaneous and/or oral administration to NMRI mice that were infected on day 0 with GFP strain P. berghei. Animals were dosed at 24, and sometimes also 48 and 72 hours after infection. Parasitemia was measured on day 4 post infection and survival time was recorded for up to 30 days post infection. A compound was considered curative if the animal survived to day 30 post infection with no detectable parasitemia.

Primary human ectocervical keratinocytes were derived from fresh cervical tissue obtained from the Cooperative Human Tissue Network (CHTN) within 24 hours after removal from patients undergoing hysterectomies for benign non-cervical uterine diseases. Standard overnight dispase treatment and subsequent trypsinization procedures were used to isolate ectocervical epithelial cells, which were cultured in serum-free keratinocyte medium (KSFM) supplemented with bovine pituitary extract and epidermal growth factor according to the manufacturer's protocol (Invitrogen, Carlsbad, Calif.). The cervical cancer cell lines HeLa and C33A were obtained from the American Type Culture Collection (ATTC) and maintained in Dulbecco's Modified Eagle Medium (DMEM) (Invitrogen).

### Assessment of Cell Viability

2.5×10³ cells were plated in triplicates in 96 well tissue culture microplates in the appropriate culture medium and incubated for 24 hours in a humidified atmosphere at 37° C., 5% CO₂. The medium was subsequently replaced by 100_{—}1 medium containing either the solvent control ethanol or various concentrations of dimers dissolved in ethanol. After a 96 hour treatment period, 50_{—}1 of the XTT labeling mixture, prepared according to the manufacturer's protocol (Roche Diagnostics GmbH, Penzberg, Germany), was added to each well, followed by an additional 16 hour incubation period. Cell viability (absorbance) was measured using an ELISA reader at 450 nm with a reference wavelength at 650 nm. Results were calculated as the percentage of cultures exposed to solvent control only. The assay was repeated twice with similar results.

### Dimer Synthesis and Chemistry

Artemisinin derived trioxane dimers 1 and 2 were synthesized in good overall yield as described in Materials and Methods (Scheme 1). Both trioxane dimers 1 (a white solid) and 2 (a colorless oil) are stable at room temperature indefinitely and at 60° C. for at least 24 hours. Hydrolytically stable means stable in 4:1 DMSO-d₆/pH 7.4 D₂O at 60° C. for 12 h confirmed by ¹H NMR

### Effects of Artemisinin Derived Trioxane Dimers on Cell Viability

To evaluate the cytotoxic effects of our newly synthesized trioxane dimers, the cervical cancer cell lines HeLa and C33A were exposed to various concentrations of these compounds, and cell viability was quantified after a three day treatment period using a colorimetric XTT based assay as described in Materials and Methods. Dimer 1 and 2 were nearly equally potent, inducing rapid dose-dependent cell killing in both cervical cancer cell lines. At a drug concentration of 100 nM an approximate 90% loss of viability was determined after treatment with either dimer (FIG. 1). Based on the data in FIG. 1, IC₅₀ values for dimer 1 and 2 of approximately 7.5 nM and 8.6 nM for C3 3A cells and approximately 8.4 nM and 9 nM for HeLa cells were determined. In contrast, normal ectocervical cells HCX were, even at a dimer concentration of 100 nM, virtually unaffected. Cell death in treated cancer cells was also easily observed with a phase contrast microscope whereas normal cells showed no significant morphological changes (data not shown).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound of formula I: 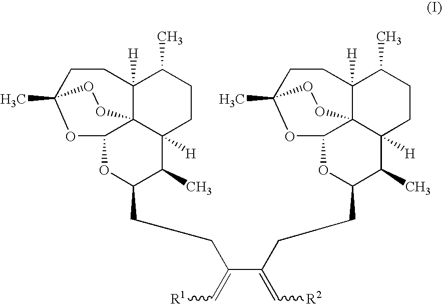
or a pharmaceutically acceptable, salt or solvate thereof, wherein:
R¹ and R² are each independently H, or substituted or unsubstituted alkyl, or R¹ and R² together form a substituted or unsubstituted aryl, or a substituted or unsubstituted cycloalkyl group.

2. The compound of claim 1, wherein R¹ and R² are hydrogen.

3. The compound of claim 1, wherein R¹ and R² form a substituted or unsubstituted phenyl group.

4. The compound of claim 3, wherein R¹ and R² form a substituted phenyl group, wherein the phenyl group is substituted with 1 or 2 R³ groups;
each R³ group is independently selected from —C(═O)OR⁴, —CH₂OR⁴, —C(═O)NR⁵R⁶, and —OP(═O)(OR⁴)₂, or
together each R³ group forms a cyclic ring with —OP(═O)O(R⁴)O—;
R⁴ hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted arylalkyl, or substituted or unsubstituted heteroaryl; and
R⁵ and R⁶ are each independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted arylalkyl, or substituted or unsubstituted heteroaryl.

5. The compound of claim 4, wherein the phenyl group is disubstituted with the same R³ group; and each R³ group is —C(═O)OH, —C(═O)OCH₃, —CH₂OH, —OP(═O)O(C₂H₅)₂, or together each R³ group forms a cyclic ring with —OP(═O)O(Ph)O—.

6. The compound of claim 1, having formula II:

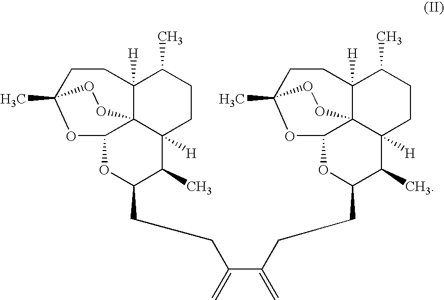

7. The compound of claim 1, having formula III: 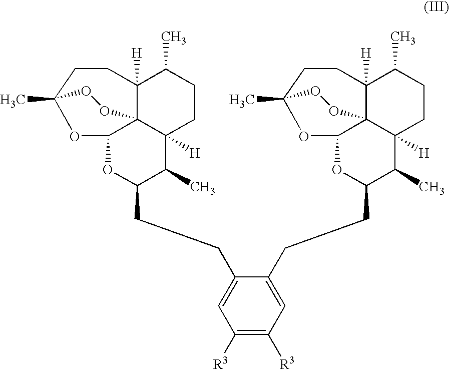
wherein each R³ group is —C(═O)OH, —C(═O)OCH₃, —CH₂OH, —OP(═O)O(C₂H₅)₂, or together each R³ group forms a cyclic ring with —OP(═O)O(Ph)O—.

8. The compound of claim 1, having formula:

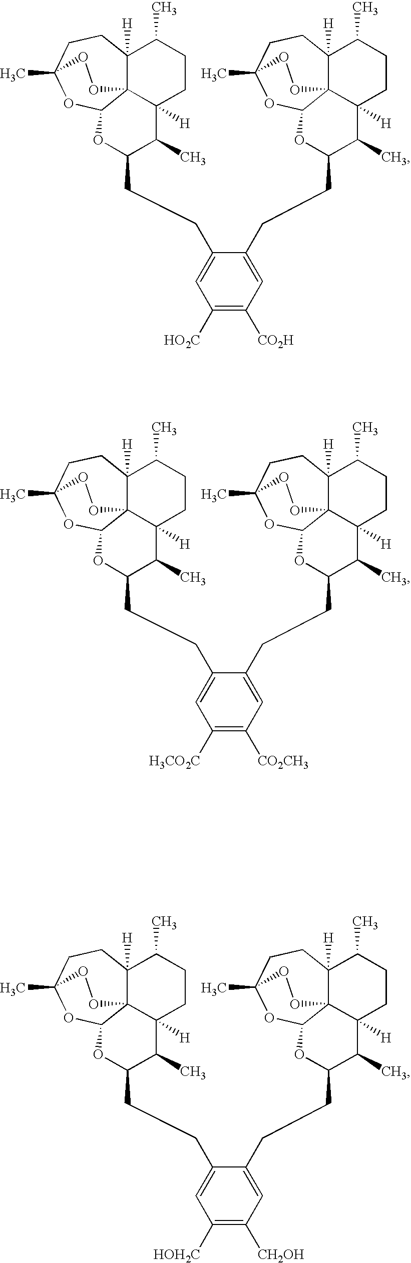

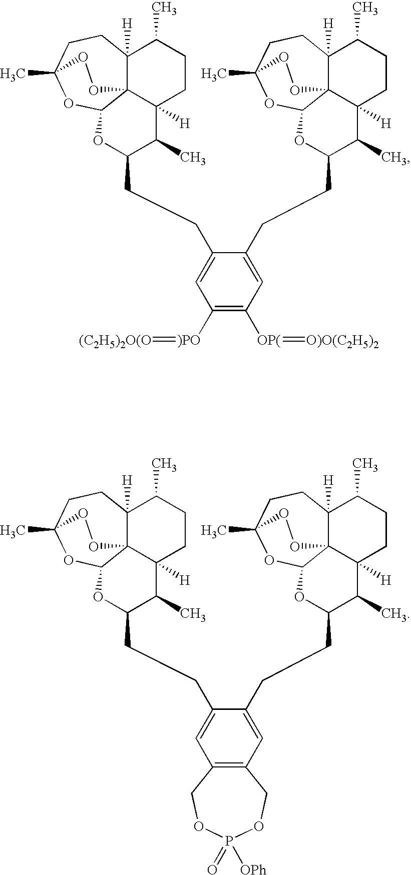

9. A compound of formula IV: 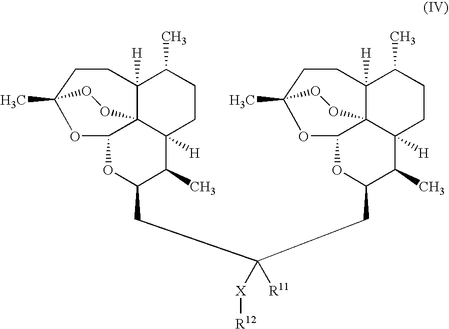
or a pharmaceutically acceptable, salt or solvate thereof, wherein:
X is (CH₂)ₘ—Y or is a direct bond;
Y is O, (CH₂)ₘO, C(═O), C(═O)(CH₂)ₘO, C(═O)O, OC(═O)O, OC(═O)NR¹³, NR¹³C(═O)NR¹³, C(═S), C(═O)S, C(═S)O, OC(═S)O, C(═O)(NR¹³), C(═O)O(NR¹³)ₙ, C(═O)O(NR¹³)ₙC(═O), C(═O)O(NR¹³)ₙC(═O), C(—O)(NR¹³)ₙC(═O), C(═O)(NR¹³)ₙ(CH₂)ₘC(═O), C(═O)(NR¹³)ₙ(CH₂)ₘC(═O)(NR¹³), (NR¹³)ₙ, (NR¹³)ₙO, C(═O)(NR¹³)ₙC(═O)(NR¹³)ₙS(O)ₚ, C(═O)O(NR¹³)ₙS(O)ₚ, OC(═O)(NR¹³)ₙS(O)ₚ; OP(═O)(OR¹³)₂, OP(═S)(OR¹³)₂, OP(═O)(NR¹³)₂, OP(═S)(NR¹³)₂, OS(O)ₚ, S(O)ₚNR¹³, (NR¹³)ₙCH₂C(═O)(NR¹³)ₙ, or Y is a direct bond;
m is an integer from 0, 1, 2 or 3;
n is an integer from 1 or 2;
p is an integer from 0, 1 or 2;
R¹¹ is H, OH, or R¹¹ together with R¹² forms a substituted or unsubstituted cyclic ring;
R¹² is optionally H, substituted or unsubstituted alkyl, substituted or unsubstituted heteroalkyl, substituted or unsubstituted aryl, substituted or unsubstituted heteroaryl, substituted or unsubstituted arylalkyl, substituted or unsubstituted heteroarylalkyl, or R¹² together with R¹¹ forms a substituted or unsubstituted cyclic ring; or
R¹¹ and R¹² form a substituted or unsubstituted double bond or a substituted or unsubstituted oxime group; and
R¹³ is H, substituted or unsubstituted alkyl, substituted or unsubstituted heteroalkyl, substituted or unsubstituted aryl, substituted or unsubstituted heteroaryl, substituted or unsubstituted arylalkyl, substituted or unsubstituted heteroarylalkyl, substituted or unsubstituted phosphonate, substituted or unsubstituted sulfonate.

10. The compound of claim 9, wherein X is CH₂—Y; and R¹¹ is H.

11. The compound of claim 10, wherein Y is O; and R¹² is H, CH₂CH═CH₂, CH₂(C₆H₄)CH₃, CH₂(C₆H₄N), CH₂(C₆H₄)CH(CH₃)₂, CH₂(C₆H₄)CF₃,

12. The compound of claim 10, wherein Y is O; and R¹² is P(═S)(OCH₂CH₃)₂, P(═O)(OC₆H₅)₂, P(═O)(NCH₂CH₃)₂ or P(═S)(OCH₃)₂.

13. The compound of claim 10, wherein Y is OC(═O)O or OC(═S)O; and R¹² is C₆H₅.

14. The compound of claim 10, wherein Y is O(C═O); and R¹² is C₆H₄C(═O)CH₃, N(CH₂CH₃)₂, N(C₅H₁₀)², N(C₅H₁₀),

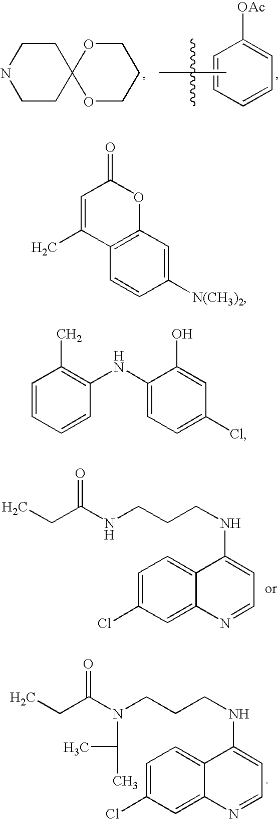

15. The compound of claim 10, wherein Y is NR¹³; and R¹³ is —C₅H₁₀—.

16. The compound of claim 10, wherein Y is OSO₂; and R¹² is

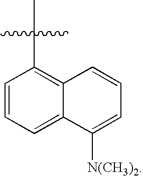

17. The compound of claim 9, wherein X is Y; and R¹¹ is H.

18. The compound of claim 17, wherein Y is C(═O) and R¹² is (C₆H₅).

19. The compound of claim 17, wherein Y is C(═O)O; and R¹² is H, (C₆H₅), CH₂(C₆H₅), NHSO₂(C₆H₅), NHC(═O)(C₆H₅),

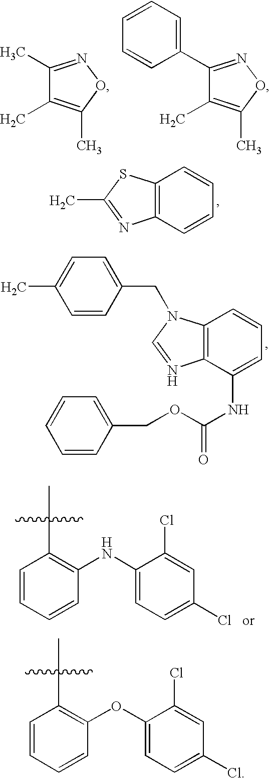

20. The compound of claim 17, wherein Y is C(═O)(NR¹³)ₙ; R¹³ is H or substituted or unsubstituted alkyl; and R¹² is (C₆H₅), CH₂(C₆H₅), CH(CO₂H)CH₂(C₆H₅), (C₆H₄N), CH₂(C₆H₄N), CH(CO₂CH₃)(C₆H₅), CH₂(C₆H₄)CO₂CH₃, CH₂(C₆H₄)C(═O)OH, CH₂(C₆H₄)NO₂, CH₂(C₆H₄)CF₃, CH₂(C₆H₄)F, (CH₂)₂SO₃H, C(CH₃)₃, C(CH₃)₂(C₆H₅), C(CH₃)₂CH₂C(CH₃)₃, CH₂C(CH₃)₂NHC(═O)(C₆H₅), CH₂CH₃, CH₂(C₆H₄)(CH₂)₇CH₃, CH₃, CH(CH₃)₂, CH₂C(CH₃)₂NH₂, (CH₂)₉CH₃, CH₂C(CH₃)₃, (CH₂)₃NHCH(CH₃)₂, CH₂C(═O)OH, C(CH₃)₂C(CH₃)₃, (C₆H₄)SO₂(C₆H₄)NH₂, CH₂CH(CH₃)₂, C(═O)(C₅H₄N), (C₆H₄)C(═O)CH₃,

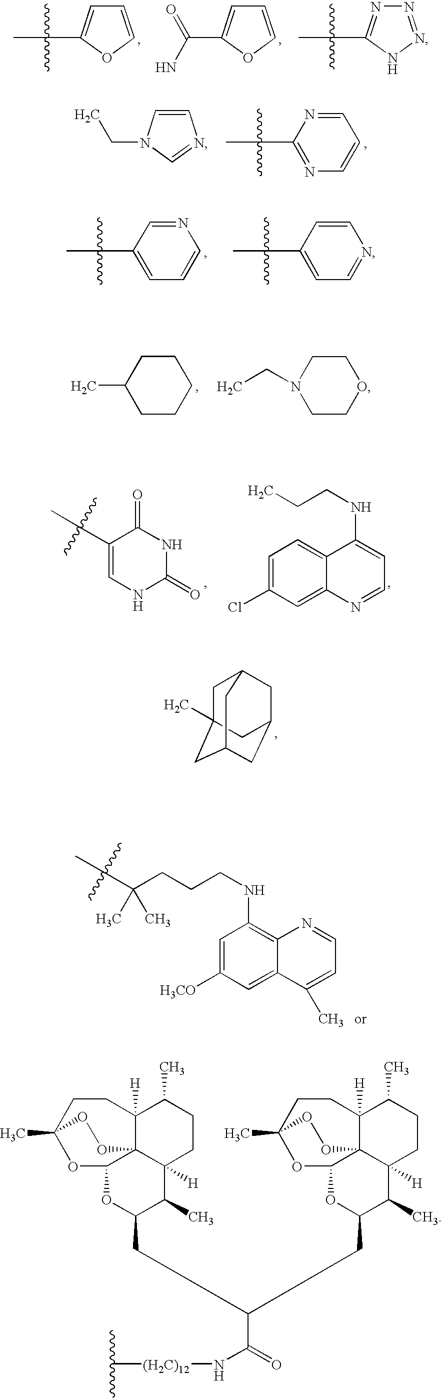

21. The compound of claim 17, wherein Y is C(═O—O)(NR¹³)ₙO; R¹³ is H or substituted or unsubstituted alkyl; and R¹² is (C₆H₅).

22. The compound of claim 17, wherein Y is C(═O)(NR¹³)ₙS(O)ₚ; R¹³ is H; and R¹² is (C₆H₅) or (C₆H₄)NH₂.

23. The compound of claim 17, wherein Y is C(═O)(NR¹³)ₙ; and R¹² and R¹³ together form a substituted or unsubstituted cyclic ring.

24. The compound of claim 22, the cyclic ring is

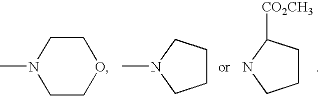

25. The compound of claim 17, wherein Y is (NR¹³)ₙC(═O)(NR¹³)ₙ or Y is (NR¹³)ₙCH₂C(═O)(NR¹³)ₙ; each R¹³ is H or substituted or unsubstituted alkyl; and R¹² is

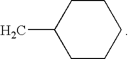

26. The compound of claim 9, wherein X is CH₂—Y; and R¹¹ is OH.

27. The compound of claim 26, wherein Y is O; and R¹² is H, (CH₂)(C₆H₄)CH₃, CH₂CH═CH₂, CH₂CH═C(CH₃)₂, CH₂(C₆H₄N), CH₂C(—O)NH(C₆H₄)OH

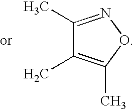

28. The compound of claim 26, wherein Y is C(═O); and R¹² is (C₆H₄)C(═O)OCH₃.

29. The compound of claim 26, wherein Y is C(═O)(NR¹³)ₙ; and R¹² is (CH₃).

30. The compound of claim 26, wherein Y is C(═O)O or Y is OC(═O); and R¹² is (C₆H₅), (C₆H₄)C(═O)N(CH₂CH₃)₂, (C₆H₄)F, (C₆H₄N), or (C₆H₄)OC(═O)CH₃.

31. The compound of claim 26, wherein Y is OC(═O)(NR¹³)ₙS(O)ₙ; R¹³ is H or substituted or unsubstituted alkyl; and R¹² is (C₆H₅).

32. The compound of claim 9, wherein X is a direct bond; and R¹¹ and R¹² together form a substituted or unsubstituted cyclic ring.

33. The compound of claim 32, having formula V: 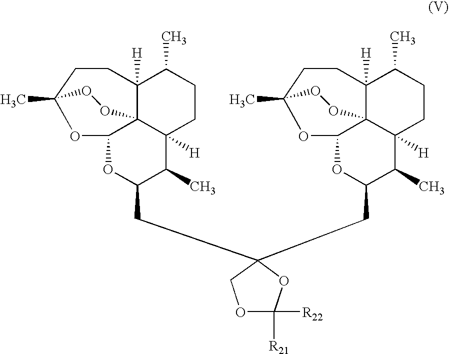
wherein:
R²¹ and R²² are each independently H, OH, OR¹³, substituted or unsubstituted alkyl, substituted or unsubstituted heteroalkyl, substituted or unsubstituted aryl, substituted or unsubstituted heteroaryl, substituted or unsubstituted arylalkyl, substituted or unsubstituted heteroarylalkyl, or R²¹ and R²² together form ═O, or R²¹ and R²² together form a substituted or unsubstituted cycloalkyl or substituted or unsubstituted heterocycloalkyl ring.

34. The compound of claim 33, wherein R²¹ and R²² together form a substituted or unsubstituted cyclobutyl ring, substituted or unsubstituted cyclohexyl ring, substituted or unsubstituted piperidinyl ring, substituted or unsubstituted tetrahydropyranyl ring; substituted or unsubstituted sulfonylcyclohexyl ring, substituted or unsubstituted 1,3-dioxanyl ring, or a substituted or unsubstituted 1,3-dioxepanyl ring.

35. The compound of claim 34, wherein R²¹ and R²² together form a substituted or unsubstituted cyclohexyl ring.

36. The compound of claim 35, wherein the cyclohexyl ring is substituted with 1 to 2 groups each independently selected from F, OH, ═O, C(═O)OCH₃, C(═O)OCH₂CH₃, C(═O)OCH₂(C₆H₅), C(═O)CH₃, C(═O)NHCH₂CH₃, C(CH₃)₃, CH₂(C₆H₅), SO₂N(CH₃)₂, SO₂(C₆H₄)CH₃, P(═O)(CH₃)₂, P(═O)(OCH₃)₂, P(═O)(OCH₂CH₃)₂, and P(═O)(OC₆H₅)₂.

37. The compound of claim 34, wherein R²¹ and R²² together form a cycloalkyl ring.

38. The compound of claim 37, wherein the compound is

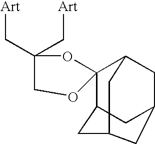

39. The compound of claim 34, wherein R²¹ and R²² together form a sulfonylcyclohexyl ring.

40. The compound of claim 39, wherein the compound is

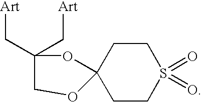

41. The compound of claim 34, wherein R²¹ and R²² form a substituted or unsubstituted piperidinyl ring.

42. The compound of claim 41, wherein the piperidinyl ring is substituted with 1 to 2 groups each independently selected from F, OH, ═O, C(═O)OCH₃, C(═O)OCH₂CH₃, C(═O)OCH₂(C₆H₅), C(═O)CH₃, C(═O)NHCH₂CH₃, C(CH₃)₃, CH₂(C₆H₅), SO₂CH₃, SO₂N(CH₃)₂, SO₂(C₆H₄)CH₃, P(═O)(CH₃)₂, P(═O)(OCH₃)₂, P(═O)(OCH₂CH₃)₂ and P(═O)(OC₆H₅)₂.

43. The compound of claim 41, having formulae:

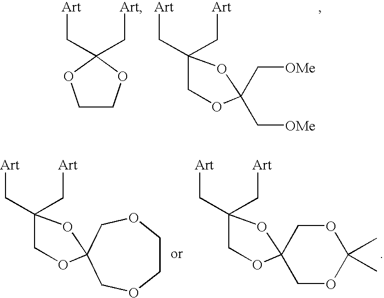

44. The compound of claim 9, having formulae:

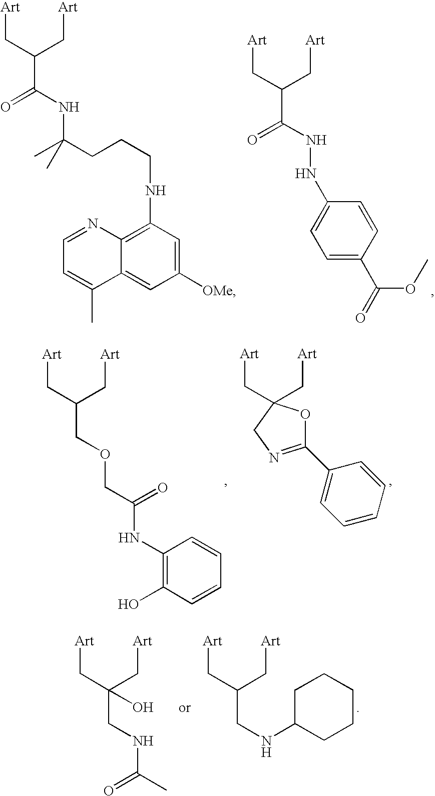

45. The compound of claim 9, wherein X is a direct bond; and R¹¹ and R¹² together form a substituted or unsubstituted double bond.

46. The compound of claim 45, wherein the double bond is substituted with a substituted or unsubstituted phenyl group.

47. The compound of claim 46, wherein the double bond is a substituted or unsubstituted oxime group.

48. The compound of claim 47, wherein the oxime group is substituted with CH₃ or NHC(═O)(C₆H₅).

49. A pharmaceutical composition comprising a pharmaceutically acceptable excipient and a compound of claim 1.

50. A pharmaceutical composition comprising a pharmaceutically acceptable excipient and a compound of claim 9.

51. A method of treating cancer in a subject in need of such treatment, said method comprising administering to the subject a therapeutically effective amount of a compound of claim 1.

52. The method of claim 51, wherein the cancer is cervical cancer.

53. A method of treating cancer in a subject in need of such treatment, said method comprising administering to the subject a therapeutically effective amount of a compound of claim 9.

54. The method of claim 53, wherein the cancer is cervical cancer.

55. A method of treating malaria in a subject in need of such treatment, said method comprising administering to the subject a therapeutically effective amount of a compound of claim 1.

56. A method of treating malaria in a subject in need of such treatment, said method comprising administering to the subject a therapeutically effective amount of a compound of claim 9.

57. The compound of claim 17, wherein Y is C(═O)(NR¹³)ₙC(═O) and
wherein n is 1, R¹³ is H, and R¹² is 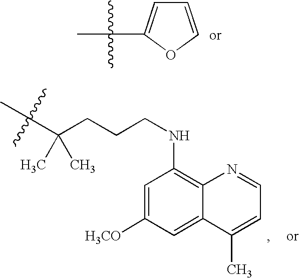
wherein n is 2, R¹³ is H, and R¹² is [chemical structure]
